(19) United States
(12) Patent Application Publication
(10) Pub. No.: US 2011/0118262 A1
(43) Pub. Date: May 19, 2011

(54) Pyrrolidinyl and Piperidinyl Compounds Useful as NHE-1 Inhibitiors

(75) Inventors: Joerg Martin Bentzien, White Plains, NY (US); Stephen James Boyer, Bethany, CT (US); Jennifer Burke, Cheshire, CT (US); Anne Bettina Eldrup, Newtown, CT (US); Xin Guo, Danbury, CT (US); John David Huber, New York, NY (US); Thomas Martin Kirrane, Middlebury, CT (US); Fariba Soleymanzadeh, Danbury, CT (US); Alan David Swinamer, Southbury, CT (US)

(73) Assignee: BOEHRINGER INGELHEIM INTERNATIONAL GMBH, Ingelheim am Rhein (DE)

(21) Appl. No.: 13/002,658

(22) PCT Filed: Jun. 24, 2009
(86) PCT No.: PCT/US09/48394

§ 371 Date: Jan. 18, 2011

Related U.S. Application Data
(60) Provisional application No. 61/078,867, filed on Jul. 8, 2008.

(51) Int. Cl. A61K 31/5377; C07D 401/14; A61K 31/4545; A61K 31/451; C07D 401/10; A61K 31/4525; C07D 401/06; A61K 31/506; A61K 31/4709; C07D 413/06; A61K 31/437; C07D 207/08; A61K 31/40; A61P 9/00

(52) U.S. Cl. 514/235.5; 546/194; 514/318; 546/226; 514/330; 546/214; 514/326; 546/196; 514/320; 544/335; 514/256; 544/332; 514/275; 546/168; 514/314; 544/130; 546/121; 514/300; 546/189; 514/316; 548/539; 514/423

(57) ABSTRACT

Disclosed are compounds of formula (I) and compositions of the present invention which are inhibitors of the sodium proton exchanger isoform-1 (NHE-I). Also disclosed are methods of using and making the same.

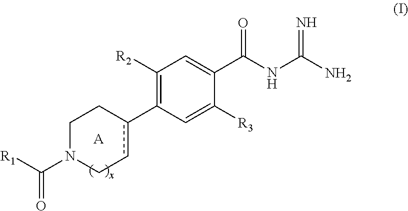

### APPLICATION DATA

This application claims benefit to U.S. provisional application Ser. No. 61/078,867 filed Jul. 8, 2008.

### BACKGROUND OF THE INVENTION

1. Technical Field

to This invention relates to NHE-1 inhibitors.

2. Background Information

The Na⁺/H⁺ exchanger (NHE) is a protein that is expressed in many mammalian cell types. NHE is an integral membrane glycoprotein expressed ubiquitously in mammalian cells, and it is responsible for regulating intracellular pH, the concentration of intracellular sodium (NaI) and cell volume regulation by extruding protons from and taking up sodium ions into cells. Nine isoforms of this exchanger have been described: NHE-1 to NHE-9. NHE-1, the first isoform to be cloned, is expressed ubiquitously in the plasma membrane and is considered to be the cardiac-specific isoform. The inward gradient of Na⁺, produced mainly by the Na⁺/K⁺ ATPase provides a constant driving force for H⁺ extrusion and Na⁺ influx through the NHE. NHE-1 is activated by growth factors and is expressed in several cell types, mainly in mammalian cardiomyocytes, platelets and on the basolateral membrane of renal tubules. Under normal physiological conditions, approximately 60% of the proton removal capabilities of the cardiac cell are accomplished via NHE-1, and NHE-1 carries about 50% of the Na⁺ entering the cell. Activation of the exchanger results in (1) decreased intracellular H⁺ (increase of pHi), (2) increased [Na⁺]ᵢ and (3) secondary to elevation of [Na⁺]i increased intracellular calcium ([Ca⁺⁺]i) levels mediated by the Na⁺/Ca⁺⁺-exchanger (NCX).

In cardiac ischemia, NHE-1 is activated by a decrease in intracellular pH. This results in an increase in intracellular Ca⁺⁺ levels as described above. Upon reperfusion, the intracellular/extracellular H⁺ gradient increases, again activating NHE-1 and increasing Ca⁺ levels. The increase of Ca⁺⁺ levels in ischemia/reperfusion results in cell injury, contributing to arrhythmia and cardiac tissue damage. Therefore, investigators were interested in discovering inhibitors of NHE-1. Several NHE-1 inhibitors have been reported in the literature and have demonstrated good activity in limiting ischemia/reperfusion injury in animal models (B. Masereel et al. An overview of inhibitors of Na⁺/H⁺ exchanger. Eur J Med Chem, 2003, 38: 547-554). The efficacy of NHE-1 inhibitors in experimental studies on ischemia/reperfusion led to clinical trials for the evaluation of some of these agents for example, in high risk patients with coronary artery disease (R. W. Erhardt, GUARD during ischemia against necrosis (GUARDIAN) trial in acute coronary syndromes. Am J Cardiol, 1999, 83: 23G-25G) and acute myocardial infarction (U. Zeymer et al., The Na⁺/H⁺ exchange inhibitor eniporide as an adjunct to early reperfusion therapy for acute myocardial infarction: results of the Evaluation of the Safety and Cardioprotective effects of eniporide in Acute Myocardial Infarction (ESCAMI) trial. J Am Coll Cardiol, 2001, 38: 1644-1650). Proof of concept was demonstrated for a cardiac protective effect of NHE-1 inhibitors in clinical studies such as these when acute treatment (up to 7 days) was begun early in an ischemic event in patients undergoing coronary artery bypass surgery. Despite the salutary effects, all NHE-1 inhibitors that have advanced to Phase II and III clinical trials in acute indications (e.g., CABG) have failed to demonstrate any improvement in overall mortality due either to inadequate efficacy or the occurrence of serious adverse events. As a result, all disclosed NHE-I clinical development programs for acute therapy appear to have been terminated.

More recent studies have suggested that inhibitors of NHE-1 may be beneficial as a chronic treatment to prevent structural and functional remodeling and increase survival in heart failure patients (M. Karmazyn, Role of sodium-hydrogen exchange in cardiac hypertrophy and heart failure: a novel and promising therapeutic target. Basic Res Cardio, 2001 96: 325-328).

Cardiac hypertrophy is a major risk factor for cardiac death and commonly precedes the development of heart failure. Hypertrophy is the cellular response to an increase in biomechanical stress. Cardiac hypertrophy eventually normalizes the increase in wall tension, there by abrogating the initial stimulus. Prolonged hypertrophy however is associated with an increased risk for the development of arrhythmias and heart failure.

Therefore, prevention of development of hypertrophy may be beneficial. Recent evidence suggests that NHE-1 is important in cardiac growth and that its activity is augmented by hypertrophic factors such as alpha-adrenergic and beta1-adrenergic activation, endothelin-1, and angiotensin II which has led to the hypothesis that NHE-1 may be a downstream mediator of these factors and that inhibition would prevent or reduce cellular hypertrophy and the HF process (L. Fliegel and M. Karmazyn, The cardiac Na—H exchanger: a key downstream mediator for the cellular hypertrophic effects of paracrine, autocrine and hormonal factors. Biochem Cell Biol, 2004, 82: 626-635). In addition, inhibition of NHE-1 also may prevent the increase in intracellular to Na⁺, Ca⁺⁺ and intracellular pH, all three parameters associated with cellular growth.

NHE-1 has been implicated in different models of hypertrophy, such as post-infarction myocardial hypertrophy, “hypertensive” myocardium, aortic constriction-induced hypertrophy, and pacing-induced hypertrophy. Different models of hypertrophy and heart failure have demonstrated the effect of chronic in vivo inhibition of NHE-1 (see for example, S. Aker et al, Inhibition of the Na⁺/H⁺-exchanger attenuates the deterioration of ventricular function during pacing-induced heart failure in rabbits. Cardiovasc Res, 2004, 63: 273-282; A. Baartscheer et al., Chronic inhibition of Na⁺/H⁺-exchanger attenuates cardiac hypertrophy and prevents cellular remodeling in heart failure. Cardiovasc Res, 2005, 65: 83-92; L. Chen, et al., Inhibition and reversal of myocardial infarction-induced hypertrophy and heart failure by NHE-1 inhibition. Am J Physiol Heart Circ Physiol, 2004, 286: H381-H387). The main focus of these studies was the development of hypertrophy and contractile function. It was found that chronic inhibition of NHE-1 attenuated development of cardiac hypertrophy and improved contractile performance, which was associated, in most of the studies, with less signs of heart failure. Also, it was found that the survival rate improved and that fibrotic structural changes and presence of apoptosis were decreased.

The above studies showed NHE-1 inhibition as a monotherapy provided extensive benefit in both structural and functional remodeling in various preclinical heart failure models, including many of the same models where ACE-I has also demonstrated a beneficial response. When treatments have been combined (NHE-1 inhibitor cariporide with ACE-I inhibitor ramapril), the benefit has been either additive or synergistic. For example, in post MI-rats, monotherapy with either cariporide or ramipril tended, but non-significantly, to decrease left ventricle dilation, whereas the combined treatment significantly reduced it. (H. Ruetten et al., Effects of combined inhibition of the Na⁺-H⁺ exchanger and angiotensin-converting enzyme in rats with congestive heart failure after myocardial infarction. British Journal of Pharmacology, 2005, 146: 723-731) Therefore, the beneficial effect of NHE-1 inhibition in addition to ACE-1 may have important clinical implications for patients with heart failure, particularly as the effect of NHE-1 inhibition may be independent of current treatment options. Studies such as those cited above support a role for NHE-1 in heart failure and provide a therapeutic rationale for the development of NHE-1 inhibitors that can be administered as a chronic to therapy.

### BRIEF SUMMARY OF THE INVENTION

It has been found that compounds of the present invention and certain derivatives thereof are inhibitors of the sodium proton exchanger isoform-1 (NHE-1).

It is therefore an object of the invention to provide compounds and compositions of the formula I as described herein below.

It is a further object of the invention to provide methods of using and making compounds of the formula I.

### DETAILED DESCRIPTION OF THE INVENTION

In the broadest generic embodiment, there is provided a compound of the formula (I):

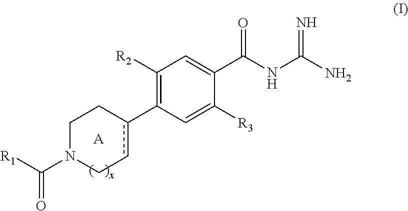

wherein:
X is 0 or 1 such that the A ring in the formula I is either a piperidinyl ring, a tetrahydropyridine ring or a pyrroldinyl ring;
R₁ is chosen from amino, C₁₋₅ alkyl, carbocycle-(CH₂)ₙ—, heterocyclyl-(CH₂)ₙ— and heteroaryl-(CH₂)ₙ— each R₁ is optionally substituted with up to three substituents independently chosen from halogen, oxo, hydroxyl, cyano, carboxy, carboxamido, C₁₋₄ alkyl, C₁₋₄ alkoxycarbonyl, C₁₋₄ alkylaminocarbonyl, C₁₋₄ dialkylaminocarbonyl, C₁₋₄ alkoxy-(CH₂)ₙ—, C₁₋₄ acyl, C₁₋₄acyloxy-(CH₂)ₙ—, C₁₋₄ alkyl-S(O)ₙ—, C₁₋₄ alkyl-S(O)ₘ—N(R₄)—, R₅—N(R₄)—S(O)ₘ—, C₃₋₇ cycloalkyl-(CH₂)ₙ—, heterocyclyl-(CH₂)ₙ—, aryl-(CH₂)ₙ-optionally substituted by C₁₋₄ alkyl, halogen, methoxy, trifluoromethoxy or cyano, heteroaryl-(CH₂)ₙ—, phenoxy optionally substituted by halogen, methoxy, C₁₋₄ alkyl-S(O)ₙ, C₁₋₄ alkyl-S(O)ₘ—N(R₄)—, cyano or trifluoromethoxy, —C(O)N(R₆)(R₇) and —(CH₂)ₙN(R₆)(R₇) each substituent on R₁ is optionally partially or fully halogenated where possible;
R₂ is chosen from halogen, hydrogen, C₁₋₅ alkyl, C₁₋₄ alkyl S(O)ₘ—N(R₄)—, C₁₋₄ alkyl-N(R₄)—S(O)ₘ— and C₁₋₄ alkyl-S(O)ₙ— each R₂ is optionally partially or fully halogenated where possible;
R₃ is chosen from hydrogen, C₁₋₅ alkyl, C₁₋₅ alkoxy, C₁₋₅ thioalkyl, C₁₋₅ acyl, C₁₋₅ alkoxycarbonyl, halogen, hydroxyl and amino optionally mono- or di-substituted by C₁₋₅ alkyl, C₁₋₅ acyl or C₃₋₇ cycloalkyl-(CH₂)ₙ;
each R₄ and R₅ are independently chosen from hydrogen, C₁₋₅ alkyl, C₁₋₅ acyl, C₃₋₇ cycloalkyl-(CH₂)ₙ—, phenyl and benzyl, or
R₄ and R₅ taken together with the nitrogen to which they are attached form a heterocyclyl ring;
each R₆ and R₇ are independently chosen from hydrogen, hydroxyl, C₁₋₅ alkyl, C₁₋₅ acyl, C₃₋₇ cycloalkyl-(CH₂)ₙ—, phenyl and benzyl, or
R₆ and R₇ taken together with the nitrogen to which they are attached form a heterocyclyl ring;
m is 1 or 2;
n is 0-2;
or a pharmaceutically acceptable salt thereof.

In another embodiment of the invention there is provided a compound of the formula (I) as provided immediately above, and wherein

R₂ is chosen from hydrogen, C₁₋₅ alkyl and C₁₋₄ alkyl-S(O)ₙ— each R₂ is optionally partially or fully halogenated where possible;
R₃ is chosen from hydrogen, C₁₋₅ alkyl, C₁₋₅ alkoxy, halogen and hydroxyl;
each R₄ and R₅ are independently chosen from hydrogen, C₁₋₅ alkyl, C₁₋₅ acyl, C₃₋₇ cycloalkyl-(CH₂)ₙ—, phenyl and benzyl;
each R₆ and R₇ are independently chosen from hydrogen, hydroxyl, C₁₋₅ alkyl, C₁₋₅ acyl, C₃₋₇ cycloalkyl-(CH₂)ₙ—, phenyl and benzyl.

In another embodiment of the invention there is provided a compound of the formula (I) as provided immediately above, and wherein

R₁ is chosen from amino, C₁₋₅ alkyl, C₃₋₇ cycloalkyl-(CH₂)ₙ—, phenyl-(CH₂)ₙ—, indanyl-(CH₂)ₙ, naphthyl-(CH₂)ₙ, -heterocyclyl-(CH₂)ₙ— wherein the heterocyclyl is azetidinyl, tetrahydrofuranyl, pyrrolidinyl, pyrrolidinonyl, tetrahydropyranyl, piperidinyl, piperazinyl, thiomorpholinyl, 1,1-dioxo-1λ⁶-thiomorpholinyl, tetrahydrothiopyran 1,1-dioxide or morpholinyl and heteroaryl-(CH₂)ₙ— wherein the heteroaryl is pyridyl, pyrimidinyl, pyridazinyl, pyrazinyl, triazinyl, tetrazinyl, pyrrolyl, pyridinonyl, imidazolyl, oxazolyl, isoxazolyl, thiazolyl, thienyl, thiadiazolyl, pyrazolyl, furanyl, pyranyl, indolyl, indolizinyl, purinyl, quinolinyl, dihydro-2H-quinolinyl, 2-oxo-1,2,3,4-tetrahydroquinolinyl, tetrahydroquinolinyl, isoquinolinyl, quinazolinyl, indazolyl, isoindolyl, benzoxazolyl, benzothiazolyl, benzimidazolyl, benzofuranyl, benzopyranyl, 2,3-dihydro-1,4-benzodioxinyl, benzodioxolyl, 1,8-napthyridyl, 1,5-napthyridyl, 2,3-dihydrobenzofuryl, imidazo[1,2-a]pyridyl or 4-methyl-3,4-dihydro-2H-benzo[1,4]oxazinyl each R₁ is optionally substituted with up to three substituents independently chosen from halogen, hydroxyl, cyano, carboxy, carboxamido, acetoxy-(CH₂)ₙ—, C₁₋₄ alkyl, C₁₋₄ alkoxycarbonyl, C₁₋₄ alkoxy-(CH₂)ₙ—, C₁₋₄ acyl, C₁₋₄ alkyl-S(O)—, C₁₋₄ alkyl S(O)ₘ—N(R₄)—, R₅—N(R₄)—S(O)ₘ—, —C(O)N(R₆)(R₇), —(CH₂)ₙN(R₆)(R₇), pyridyl, pyrimidinyl, imidazolyl, pyrazolyl, pyrrolidinyl, oxazolyl, furyl, phenyl optionally substituted by halogen, methoxy, trifluoromethoxy or cyano and phenoxy optionally substituted by C₁₋₄ alkyl-S(O)ₙ—, C₁₋₄ alkyl S(O)ₘ—N(R₄)—, cyano or trifluoromethoxy, each substituent on R₁ is optionally partially or fully halogenated where possible;
each R₄ and R₅ are independently chosen from hydrogen, C₁₋₅ alkyl and C₃₋₇ cycloalkyl,
each R₆ and R₇ are independently chosen from hydrogen, C₁₋₅ alkyl and C₃₋₇ cycloalkyl.

In another embodiment of the invention there is provided a compound of the formula (I) as provided immediately above, and wherein

R₁ is chosen from amino, C₁₋₅ alkyl, C₃₋₇ cycloalkyl-(CH₂)ₙ—, phenyl-(CH₂)ₙ—, indanyl-(CH₂)ₙ, naphthyl-(CH₂)ₙ, -heterocyclyl-(CH₂)ₙ— wherein the heterocyclyl is tetrahydrofuranyl, pyrrolidinyl, pyrrolidinonyl, tetrahydropyranyl, piperidinyl, tetrahydrothiopyran 1,1-dioxide or morpholinyl and heteroaryl-(CH₂)ₙ— wherein the heteroaryl is pyridyl, pyrimidinyl, pyrrolyl, pyridinonyl, imidazolyl, oxazolyl, thiazolyl, thienyl, pyrazolyl, furanyl, indolyl, quinolinyl, 2-oxo-1,2,3,4-tetrahydroquinolinyl, benzothiazolyl, 2,3-dihydro-1,4-benzodioxinyl, benzodioxolyl, 1,8-napthyridyl, 1,5-napthyridyl, 2,3-dihydrobenzofuryl, imidazo[1,2-a]pyridyl or 4-methyl-3,4-dihydro-2H-benzo[1,4]oxazinyl each R₁ is optionally substituted with up to three substituents independently chosen from halogen, hydroxyl, cyano, carboxy, carboxamido, acetoxy-(CH₂)ₙ—, C₁₋₄ alkyl, C₁₋₄ alkoxycarbonyl, C₁₋₄ alkoxy-(CH₂)ₙ—, C₁₋₄ alkyl-S(O)ₙ—, R₃—N(R₄)—S(O)ₘ—, —C(O)N(R₆)(R₇), —(CH₂)ₙN(R₆)(R₇), pyridyl, pyrimidinyl, imidazolyl, pyrazolyl, pyrrolidinyl, oxazolyl, furyl, phenyl optionally substituted by halogen, methoxy, trifluoromethoxy or cyano and phenoxy optionally substituted by C₁₋₄ alkyl-S(O)ₘ—, C₁₋₄ alkyl S(O)ₘ—N(R₄)—, cyano or trifluoromethoxy; each substituent on R₁ is optionally partially or fully halogenated where possible;
R₂ is chosen from halogenated C₁₋₃ alkyl and C₁₋₄ alkyl-S(O)ₘ—;
R₃ is chosen from hydrogen, C₁₋₅ alkyl and C₁₋₅ alkoxy;
each R₄, R₃, R₆, and R₇ are independently chosen from hydrogen and C₁₋₅ alkyl.

In another embodiment of the invention there is provided a compound of the formula (I) as provided immediately above, and wherein

X is 0 or 1 such that the A ring in the formula I is either a piperidinyl ring or a pyrrolidinyl ring;
R₁ is chosen from amino, C₁₋₅ alkyl, C₃₋₇ cycloalkyl-(CH₂)ₙ—, phenyl-(CH₂)ₙ—, indanyl-(CH₂)ₙ, naphthyl-(CH₂)ₙ, -heterocyclyl-(CH₂)ₙ— wherein the heterocyclyl is pyrrolidinyl, pyrrolidinonyl, tetrahydropyranyl, piperidinyl, tetrahydrothiopyran 1,1-dioxide or morpholinyl and heteroaryl-(CH₂)ₙ— wherein the heteroaryl is pyridyl, pyrimidinyl, pyrrolyl, pyridinonyl, imidazolyl, oxazolyl, thiazolyl, thienyl, pyrazolyl, furanyl, indolyl, quinolinyl, 2-oxo-1,2,3,4-tetrahydroquinolinyl, benzothiazolyl, 2,3-dihydro-1,4-benzodioxinyl, 1,8-napthyridyl, 2,3-dihydrobenzofuryl, imidazo[1,2-a]pyridyl or 4-methyl-3,4-dihydro-2H-benzo[1,4]oxazinyl each R₁ is optionally substituted with up to three substituents independently chosen from halogen, hydroxyl, cyano, carboxy, carboxamido, acetoxy, C₁₋₄ alkyl, C₁₋₄ alkoxycarbonyl, C₁₋₄ alkoxy-(CH₂)ₙ—, C₁₋₄ alkyl-S(O)ₙ—, R₅—N(R₄)—S(O)ₘ—, —C(O)N(R₆)(R₇), —N(R₆)(R₇), pyridyl, pyrimidinyl, imidazolyl, pyrazolyl, pyrrolidinyl, oxazolyl, phenyl optionally substituted by halogen or methoxy and phenoxy optionally substituted by C₁₋₄ alkyl-S(O)ₙ— or C₁₋₄ alkyl-S(O)ₙ—N(R₄)—; each substituent on R₁ is optionally partially or fully halogenated where possible;
R₂ is trifluoromethyl or methylsulfonyl;
R₃ is chosen from hydrogen, methyl and methoxy;
each R₄, R₅, R₆, and R₇ are independently chosen from hydrogen and methyl.

In another embodiment of the invention there is provided a compound of the formula (I) as provided immediately above, and wherein

R₁ is chosen from

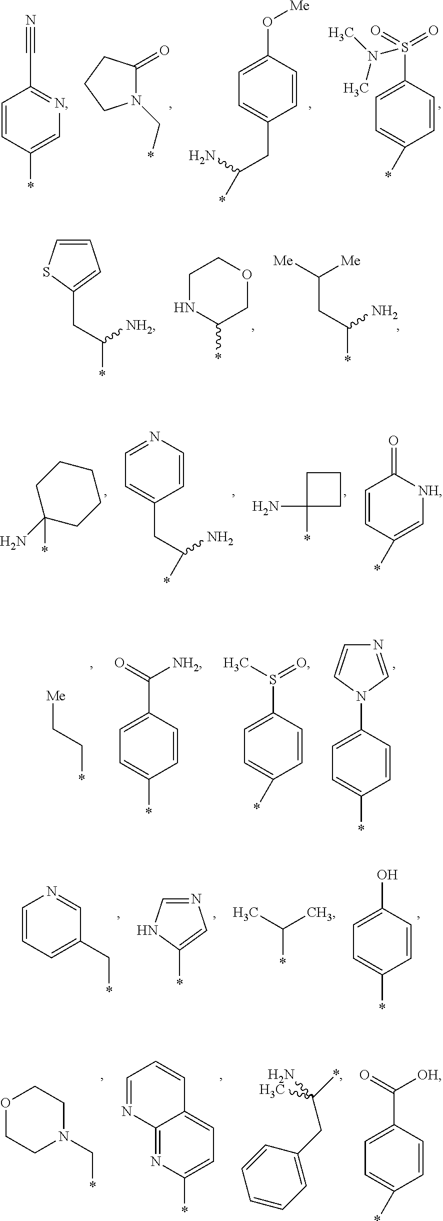

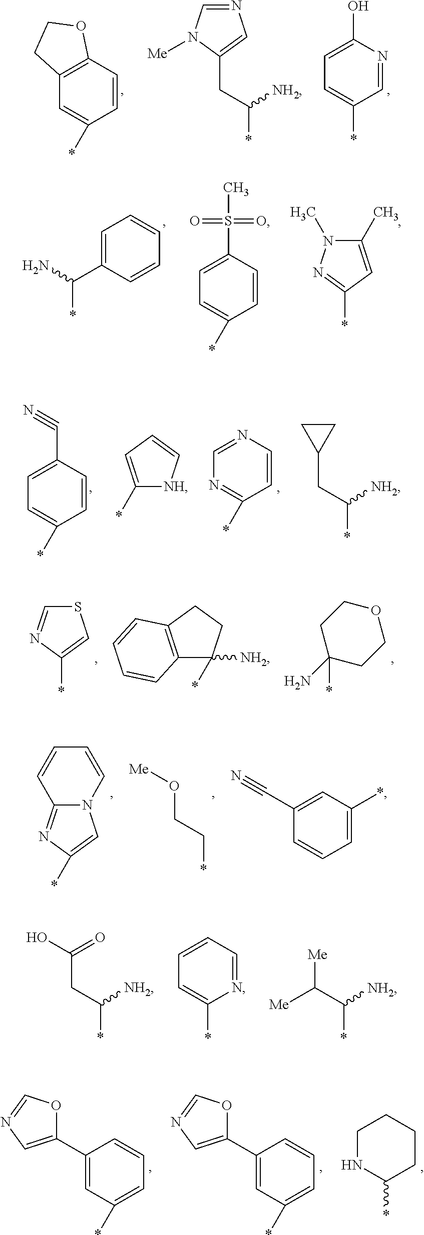

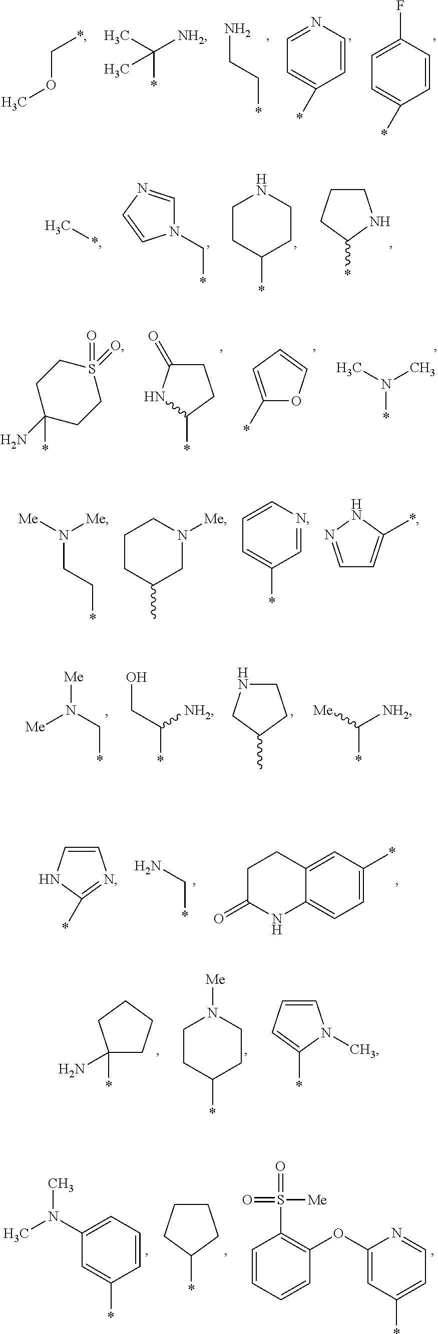

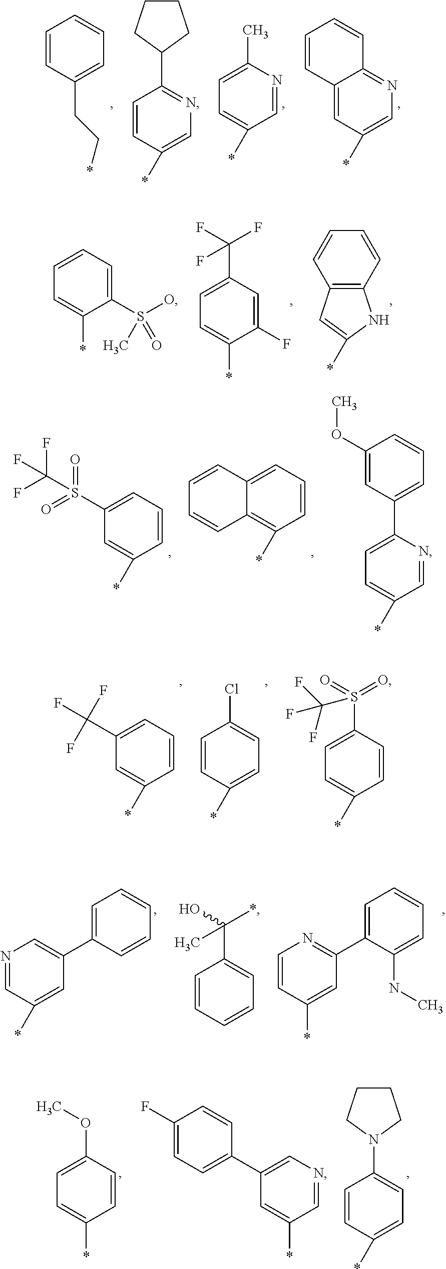

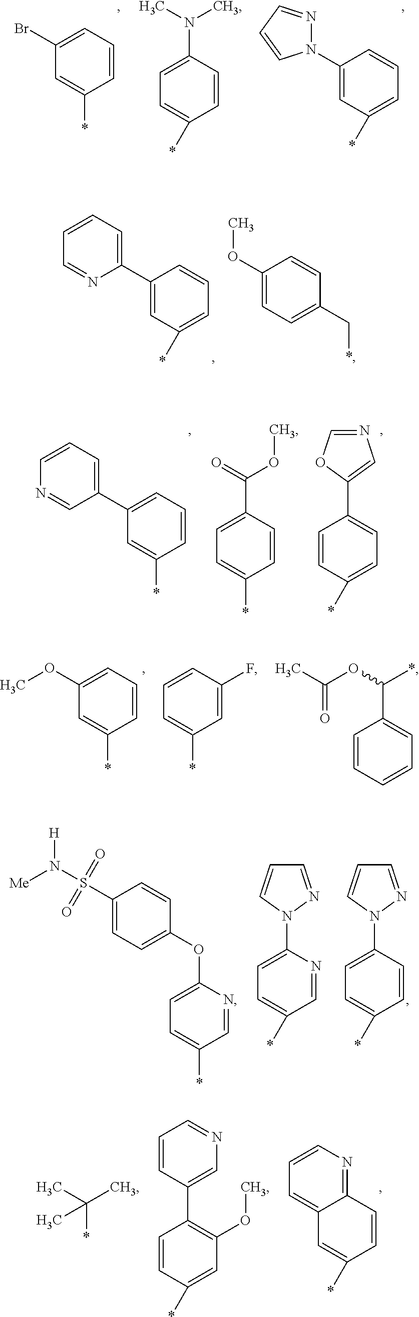

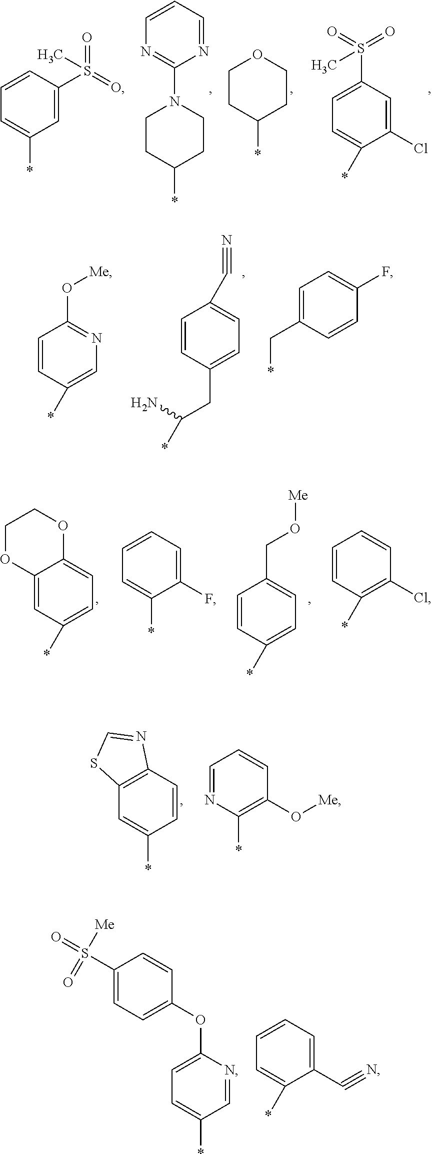

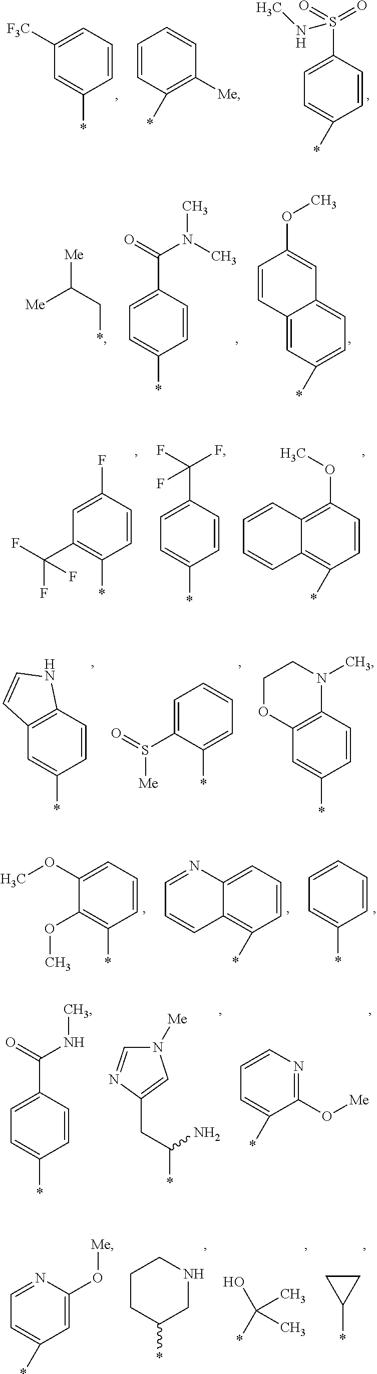

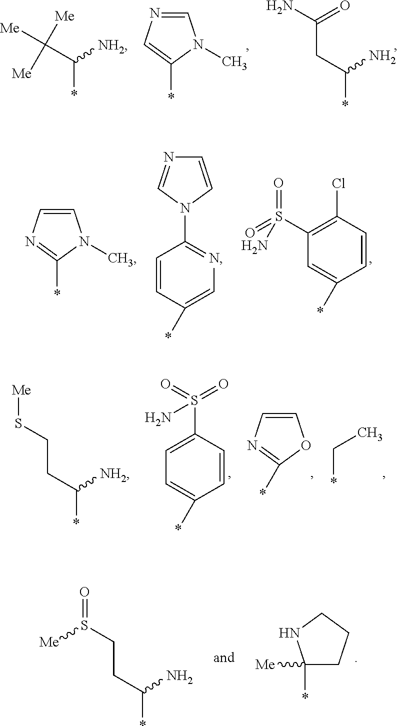

In another embodiment of the invention there is provided a compound of the formula (I) as provided in any embodiment above, and wherein

X is 0 such that the A ring in the formula I is a pyrroldinyl ring.

In another embodiment of the invention there is provided a compound of the formula (I) as provided in any embodiment above, and wherein

X is 1 such that the A ring in the formula I is a piperidinyl ring.

In another embodiment, the invention provides compounds in Table I which can be made in view of the general schemes, examples and methods known in the art.

TABLE I

| Structure | Name |
|---|---|
| 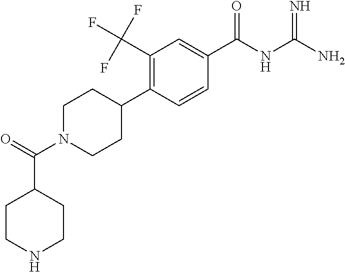 | N-{4-[1-(2-Amino-3-phenyl- propionyl)-piperidin-4-yl]-3- trifluoromethyl-benzoyl}-guanidine |
| 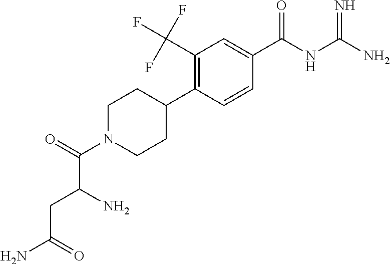 | 3-Amino-4-[4-(4-guanidinocarbonyl- 2-trifluoromethyl-phenyl)-piperidin-1- yl]-4-oxo-butyramide |
| 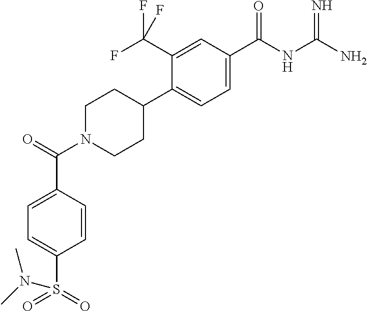 | 4-[4-(4-Guanidinocarbonyl-2- trifluoromethyl-phenyl)-piperidine-1- carbonyl]-N,N-dimethyl- benzenesulfonamide |
| 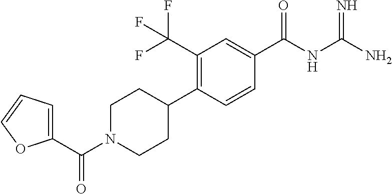 | N-{4-[1-(Furan-2-carbonyl)-piperidin- 4-yl]-3-trifluoromethyl-benzoyl}- guanidine |
| 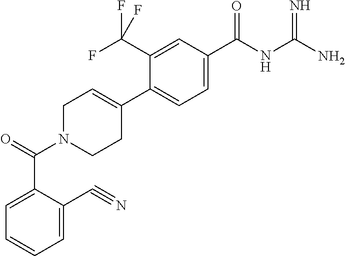 | N-{4-[1-(2-Cyano-benzoyl)-1,2,3,6- tetrahydro-pyridin-4-yl]-3- trifluoromethyl-benzoyl}-guanidine |
| 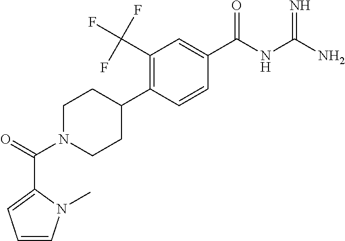 | N-{4-[1-(1-Methyl-1H-pyrrole-2- carbonyl)-piperidin-4-yl]-3- trifluoromethyl-benzoyl}-guanidine |
| 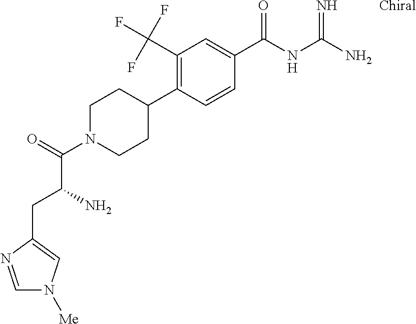 | (R)-N-(4-{1-[-2--Amino-3-(1-methyl- 1H-imidazol-4-yl)-propionyl]- piperidin-4-yl}-3-trifluoromethyl- benzoyl)-guanidine |
| 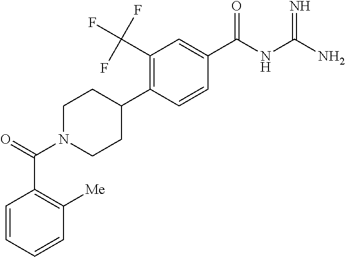 | N-{4-[1-(2-Methyl-benzoyl)- piperidin-4-yl]-3-trifluoromethyl- benzoyl}-guanidine |
| 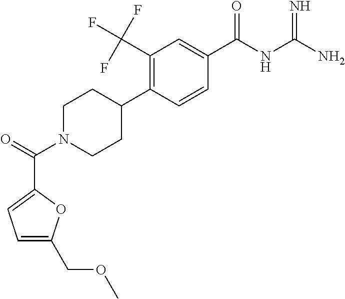 | N-{4-[1-(5-Methoxymethyl-furan-2- carbonyl)-piperidin-4-yl]-3- trifluoromethyl-benzoyl}-guanidine |
| 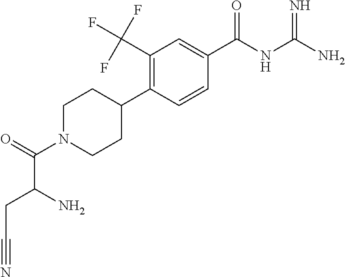 | N-{4-[1-(2-Amino-3-cyano- propionyl)-piperidin-4-yl]-3- trifluoromethyl-benzoyl}-guanidine |
| 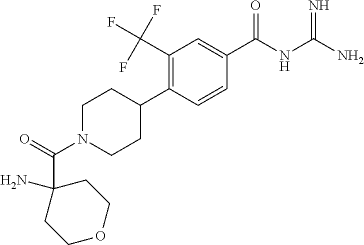 | N-{4-[1-(4-Amino-tetrahydro-pyran- 4-carbonyl)-piperidin-4-yl]-3- trifluoromethyl-benzoyl}-guanidine |
| 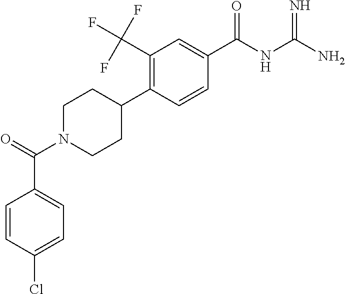 | N-{4-[1-(4-Chloro-benzoyl)- piperidin-4-yl]-3-trifluoromethyl- benzoyl}-guanidine |
| 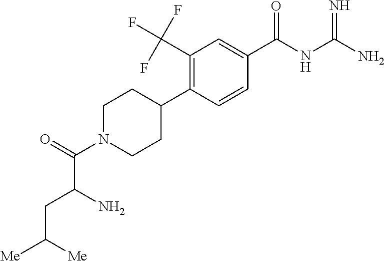 | N-{4-[1-(2-Amino-4-methyl- pentanoyl)-piperidin-4-yl]-3- trifluoromethyl-benzoyl}-guanidine |
| 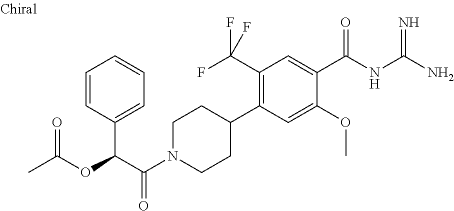 | (S)-Acetic acid 2--[4-(4- guanidinocarbonyl-5-methoxy-2- trifluoromethyl-phenyl)-piperidin-1- yl]-2-oxo-1-phenyl-ethyl ester |
| 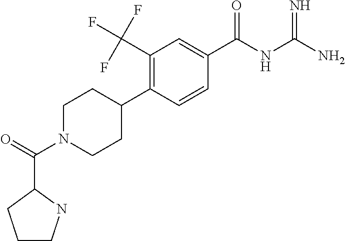 | N-{4-[1-(Pyrrolidine-2-carbonyl)- piperidin-4-yl]-3-trifluoromethyl- benzoyl}-guanidine |
| 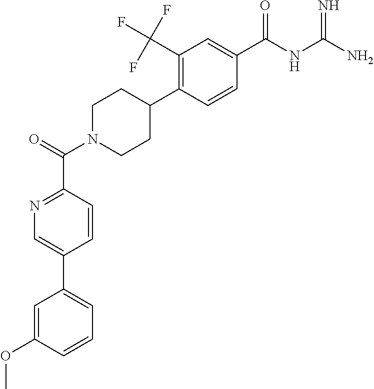 | N-(4-{1-[5-(3-Methoxy-phenyl)- pyridine-2-carbonyl]-piperidin-4-yl}- 3-trifluoromethyl-benzoyl)-guanidine |
| 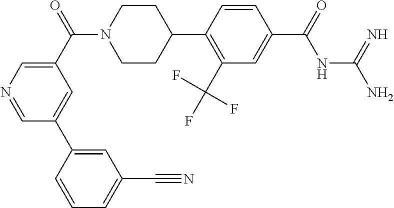 | N-(4-{1-[5-(3-Cyano-phenyl)- pyridine-3-carbonyl]-piperidin-4-yl}- 3-trifluoromethyl-benzoyl)-guanidine |
| 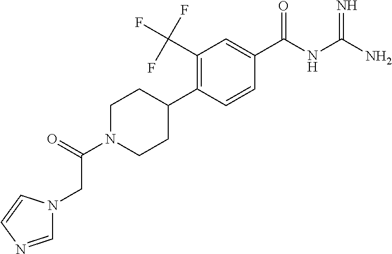 | N-{4-[1-(2-Imidazol-1-yl-acetyl)- piperidin-4-yl]-3-trifluoromethyl- benzoyl}-guanidine |
| 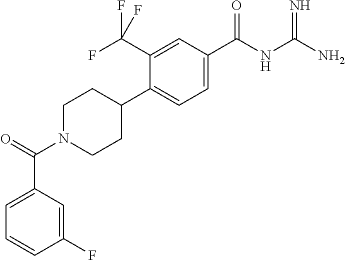 | N-{4-[1-(3-Fluoro-benzoyl)-piperidin- 4-yl]-3-trifluoromethyl-benzoyl}- guanidine |
| 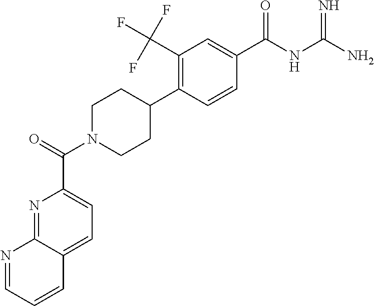 | N-{4-[1-([1,8]Naphthyridine-2- carbonyl)-piperidin-4-yl]-3- trifluoromethyl-benzoyl}-guanidine |
| 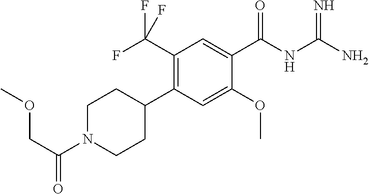 | N-{2-Methoxy-4-[1-(2-methoxy- acetyl)-piperidin-4-yl]-5- trifluoromethyl-benzoyl}-guanidine |
| 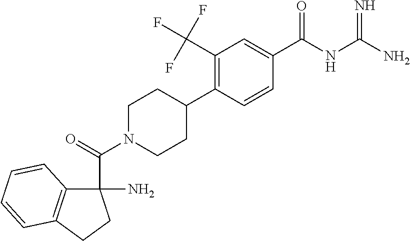 | N-{4-[1-(1-Amino-indane-1- carbonyl)-piperidin-4-yl]-3- trifluoromethyl-benzoyl}-guanidine |
| 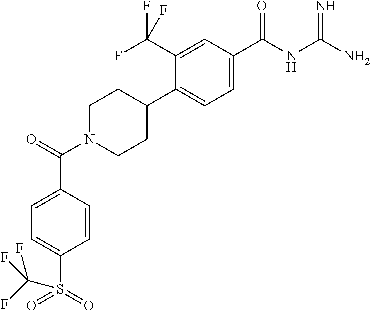 | N-{4-[1-(4-Trifluoromethanesulfonyl- benzoyl)-piperidin-4-yl]-3- trifluoromethyl-benzoyl}-guanidine |
| 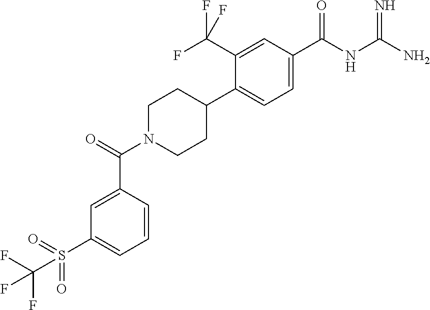 | N-{4-[1-(3-Trifluoromethanesulfonyl- benzoyl)-piperidin-4-yl]-3- trifluoromethyl-benzoyl}-guanidine |
| 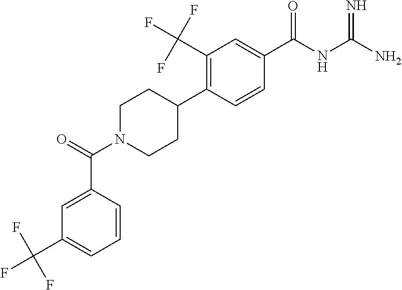 | N-{3-Trifluoromethyl-4-[1-(3- trifluoromethyl-benzoyl)-piperidin-4- yl]-benzoyl}-guanidine |
| 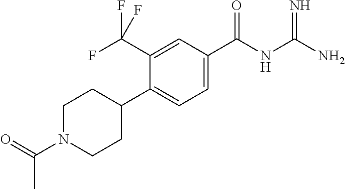 | N-[4-(1-Acetyl-piperidin-4-yl)-3- trifluoromethyl-benzoyl]-guanidine |
| 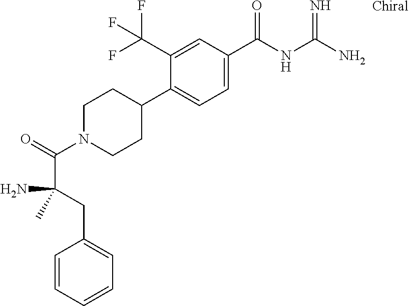 | (R)-N-{4-[1-(-2--Amino-2-methyl-3- phenyl-propionyl)-piperidin-4-yl]-3- trifluoromethyl-benzoyl}-guanidine |
| 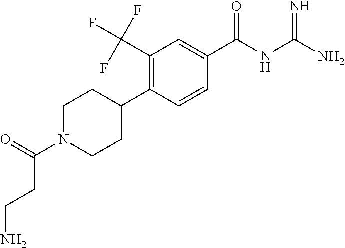 | N-{4-[1-(3-Amino-propionyl)- piperidin-4-yl]-3-trifluoromethyl- benzoyl}-guanidine |
| 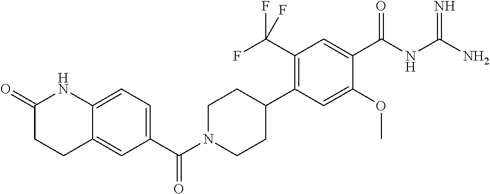 | N-{2-Methoxy-4-[1-(2-oxo-1,2,3,4- tetrahydro-quinoline-6-carbonyl)- piperidin-4-yl]-5-trifluoromethyl- benzoyl}-guanidine |
| 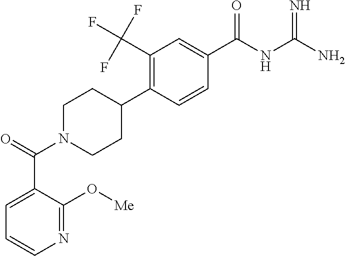 | N-{4-[1-(2-Methoxy-pyridine-3- carbonyl)-piperidin-4-yl]-3- trifluoromethyl-benzoyl}-guanidine |
| 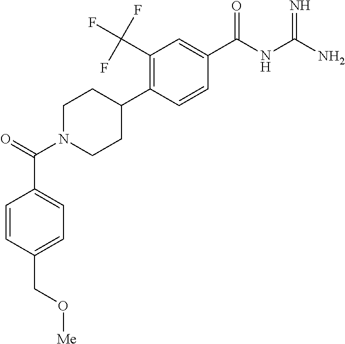 | N-{4-[1-(4-Methoxymethyl-benzoyl)- piperidin-4-yl]-3-trifluoromethyl- benzoyl}-guanidine |
| 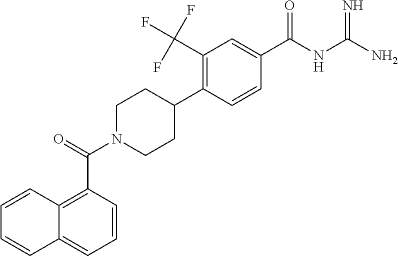 | N-{4-[1-(Naphthalene-1-carbonyl)- piperidin-4-yl]-3-trifluoromethyl- benzoyl}-guanidine |
| 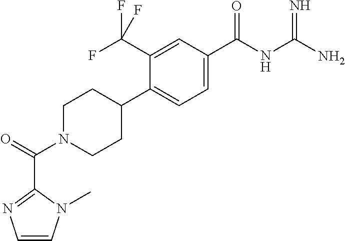 | N-{4-[1-(1-Methyl-1H-imidazole-2- carbonyl)-piperidin-4-yl]-3- trifluoromethyl-benzoyl}-guanidine |
| 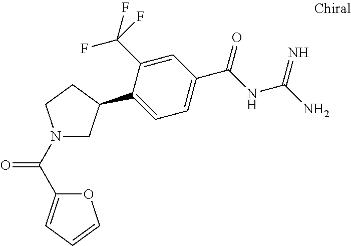 | (S)-N-{4-[-1-(Furan-2-carbonyl)- pyrrolidin-3--yl]-3-trifluoromethyl- benzoyl}-guanidine |
| 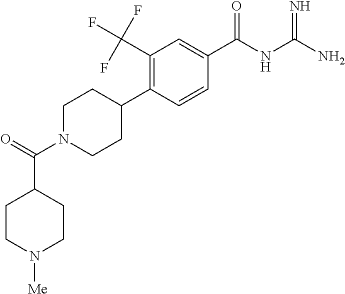 | N-{4-[1-(1-Methyl-piperidine-4- carbonyl)-piperidin-4-yl]-3- trifluoromethyl-benzoyl}-guanidine |
| 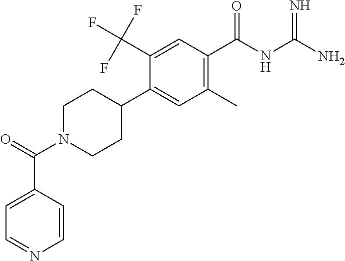 | N-{2-Methyl-4-[1-(pyridine-4- carbonyl)-piperidin-4-yl]-5- trifluoromethyl-benzoyl}-guanidine |
| 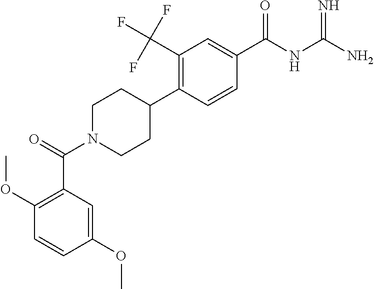 | N-{4-[1-(2,5-Dimethoxy-benzoyl)- piperidin-4-yl]-3-trifluoromethyl- benzoyl}-guanidine |
| 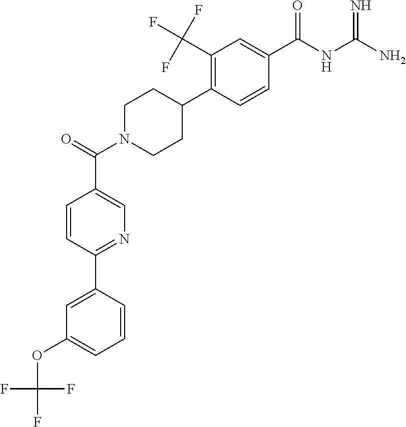 | N-(4-{1-[6-(3-Trifluoromethoxy- phenyl)-pyridine-3-carbonyl]- piperidin-4-yl}-3-trifluoromethyl- benzoyl)-guanidine |
| 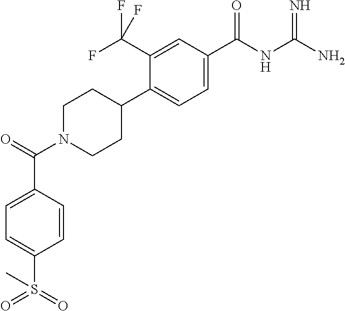 | N-{4-[1-(4-Methanesulfonyl- benzoyl)-piperidin-4-yl]-3- trifluoromethyl-benzoyl}-guanidine |
| 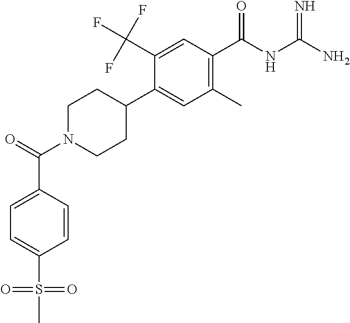 | N-{4-[1-(4-Methanesulfonyl- benzoyl)-piperidin-4-yl]-2-methyl-5- trifluoromethyl-benzoyl}-guanidine |
| 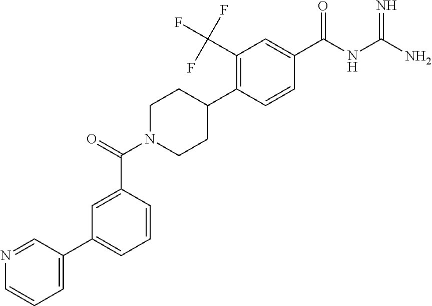 | N-{4-[1-(3-Pyridin-3-yl-benzoyl)- piperidin-4-yl]-3-trifluoromethyl- benzoyl}-guanidine |
| 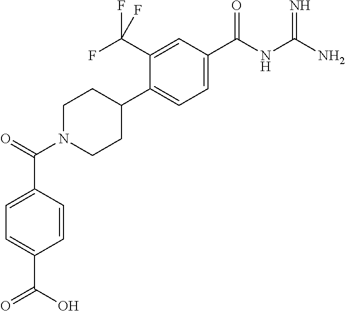 | 4-[4-(4-Guanidinocarbonyl-2- trifluoromethyl-phenyl)-piperidine-1- carbonyl]-benzoic acid |
| 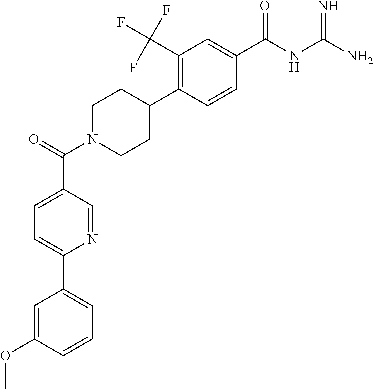 | N-(4-{1-[6-(3-Methoxy-phenyl)- pyridine-3-carbonyl]-piperidin-4-yl}- 3-trifluoromethyl-benzoyl)-guanidine |
| 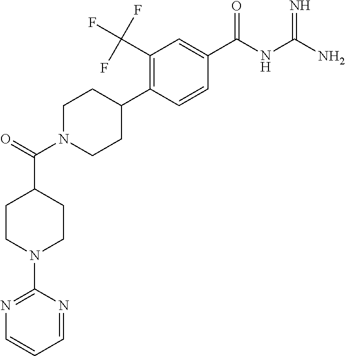 | N-{4-[1-(1-Pyrimidin-2-yl-piperidine- 4-carbonyl)-piperidin-4-yl]-3- trifluoromethyl-benzoyl}-guanidine |
| 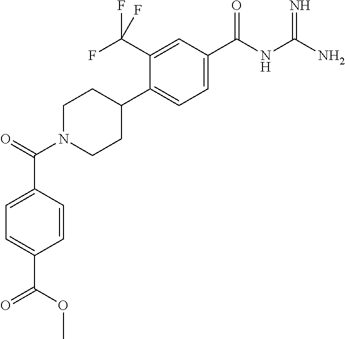 | 4-[4-(4-Guanidinocarbonyl-2- trifluoromethyl-phenyl)-piperidine-1- carbonyl]-benzoic acid methyl ester |
| 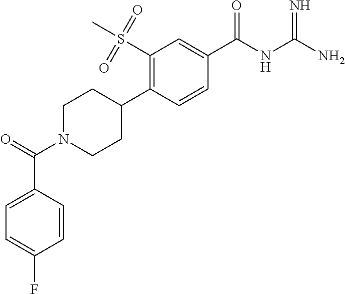 | N-{4-[1-(4-Fluoro-benzoyl)-piperidin- 4-yl]-3-methanesulfonyl-benzoyl}- guanidine |
| 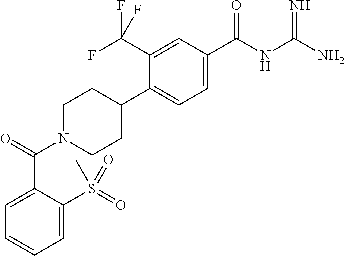 | N-{4-[1-(2-Methanesulfonyl- benzoyl)-piperidin-4-yl]-3- trifluoromethyl-benzoyl}-guanidine |
| 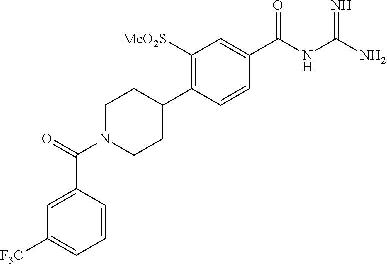 | N-{3-methanesulfonyl-4-[1-(3- trifluoromethyl-benzoyl)-piperidin-4- yl]-benzoyl}-guanidine |
| 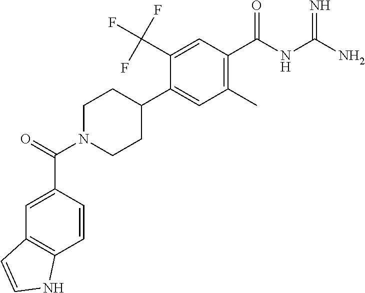 | N-{4-[1-(1H-Indole-5-carbonyl)- piperidin-4-yl]-2-methyl-5- trifluoromethyl-benzoyl}-guanidine |
| 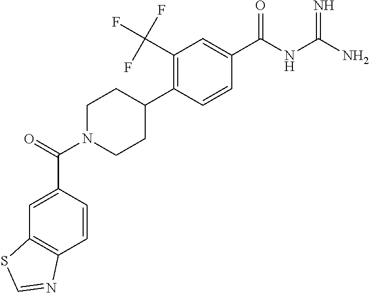 | N-{4-[1-(Benzothiazole-6-carbonyl)- piperidin-4-yl]-3-trifluoromethyl- benzoyl}-guanidine |
| 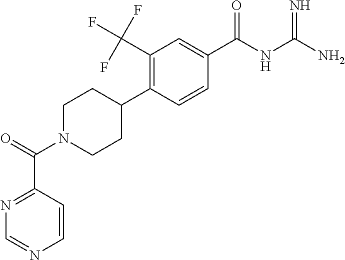 | N-{4-[1-(Pyrimidine-4-carbonyl)- piperidin-4-yl]-3-trifluoromethyl- benzoyl}-guanidine |
| 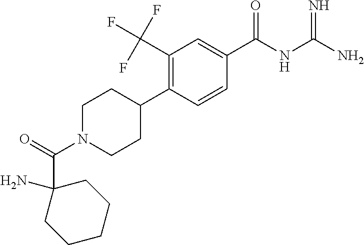 | N-{4-[1-(1-Amino- cyclohexanecarbonyl)-piperidin-4-yl]- 3-trifluoromethyl-benzoyl}-guanidine |
| 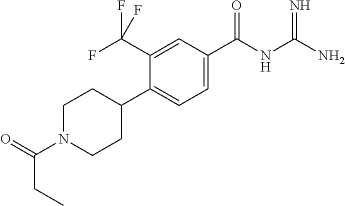 | N-[4-(1-Propionyl-piperidin-4-yl)-3- trifluoromethyl-benzoyl]-guanidine |
| 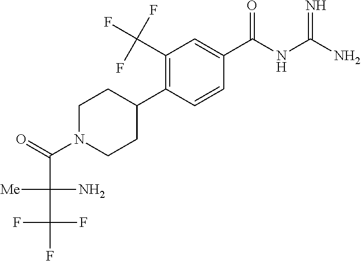 | N-{4-[1-(2-Amino-3,3,3-trifluoro-2- methyl-propionyl)-piperidin-4-yl]-3- trifluoromethyl-benzoyl}-guanidine |
| 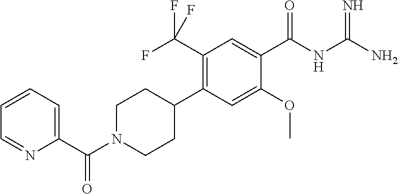 | N-{2-Methoxy-4-[1-(pyridine-2- carbonyl)-piperidin-4-yl]-5- trifluoromethyl-benzoyl}-guanidine |
| 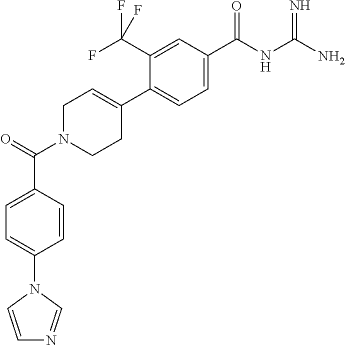 | N-{4-[1-(4-Imidazol-1-yl-benzoyl)- 1,2,3,6-tetrahydro-pyridin-4-yl]-3- trifluoromethyl-benzoyl}-guanidine |
| 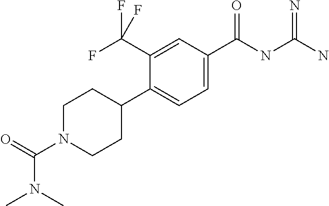 | 4-(4-Guanidinocarbonyl-2- trifluoromethyl-phenyl)-piperidine-1- carboxylic acid dimethylamide |
| 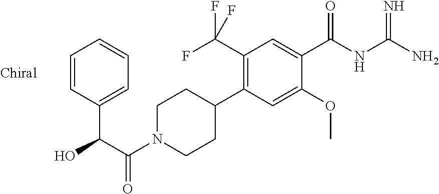 | (S)-N-{4-[1-(-2-Hydroxy-2-phenyl- acetyl)-piperidin-4-yl]-2-methoxy-5- trifluoromethyl-benzoyl}-guanidine |
| 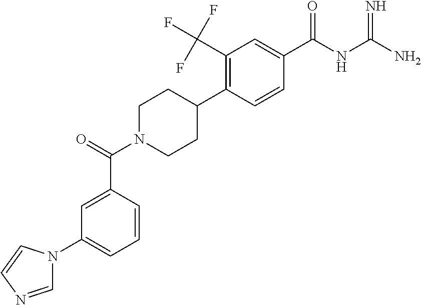 | N-{4-[1-(3-Imidazol-1-yl-benzoyl)- piperidin-4-yl]-3-trifluoromethyl- benzoyl}-guanidine |
| 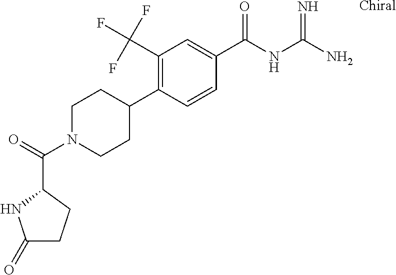 | (S)-N-{4-[1-(-5-Oxo-pyrrolidine-2- carbonyl)-piperidin-4-yl]-3- trifluoromethyl-benzoyl}-guanidine |
| 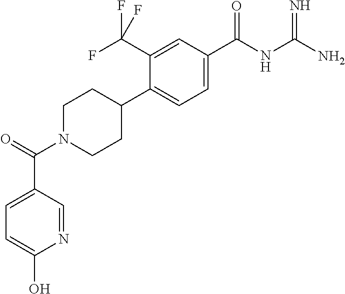 | N-{4-[1-(6-Hydroxy-pyridine-3- carbonyl)-piperidin-4-yl]-3- trifluoromethyl-benzoyl}-guanidine |
| 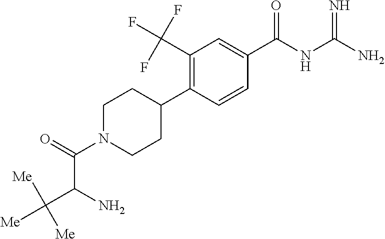 | N-{4-[1-(2-Amino-3,3-dimethyl- butyryl)-piperidin-4-yl]-3- trifluoromethyl-benzoyl}-guanidine |
| 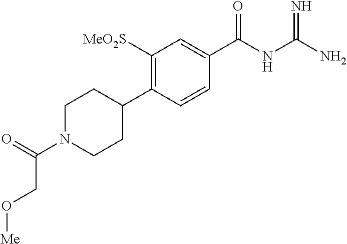 | N-{3-methanesulfonyl-4-[1-(2- methoxy-acetyl)-piperidin-4-yl]- benzoyl}-guanidine |
| 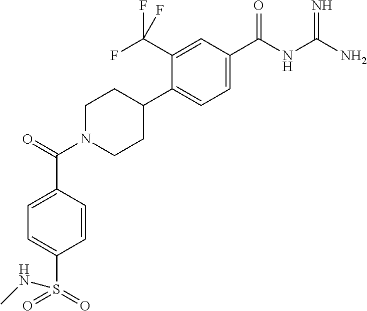 | 4-[4-(4-Guanidinocarbonyl-2- trifluoromethyl-phenyl)-piperidine-1- carbonyl]-N-methyl- benzenesulfonamide |
| 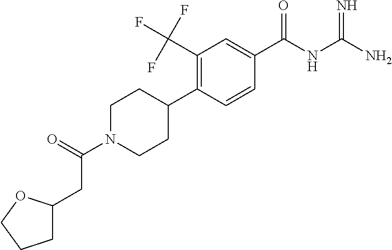 | N-{4-[1-(2-Tetrahydro-furan-2-yl- acetyl)-piperidin-4-yl]-3- trifluoromethyl-benzoyl}-guanidine |
| 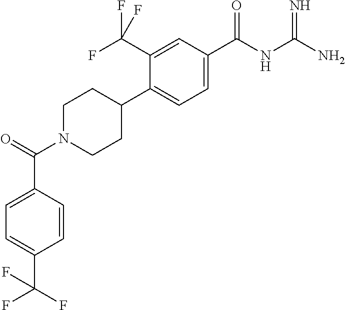 | N-{3-Triuoromethyl-4-[1-(4- trifluoromethyl-benzoyl)-piperidin-4- yl]-benzoyl}-guanidine |
| 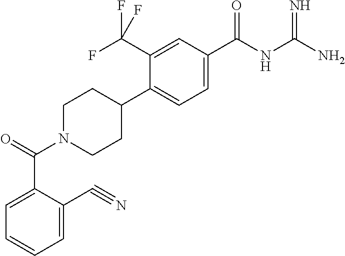 | N-{4-[1-(2-Cyano-benzoyl)-piperidin- 4-yl]-3-trifluoromethyl-benzoyl}- guanidine |
| 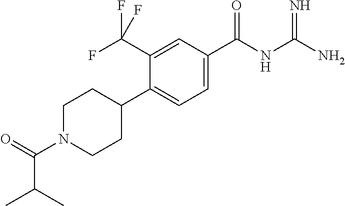 | N-[4-(1-Isobutyryl-piperidin-4-yl)-3- trifluoromethyl-benzoyl]-guanidine |
| 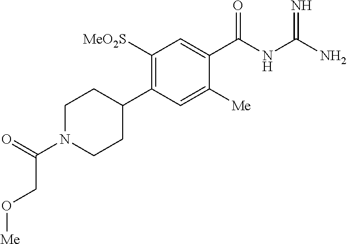 | N-{5-methanesulfonyl-4-[1-(2- methoxy-acetyl)-piperidin-4-yl]-2- methyl-benzoyl}-guanidine |
| 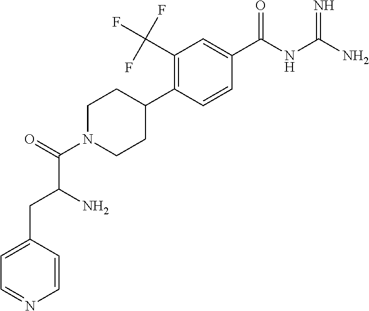 | N-{4-[1-(2-Amino-3-pyridin-4-yl- propionyl)-piperidin-4-yl]-3- trifluoromethyl-benzoyl}-guanidine |
| 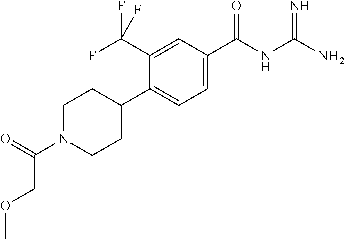 | N-{4-[1-(2-Methoxy-acetyl)- piperidin-4-yl]-3-trifluoromethyl- benzoyl}-guanidine |
| 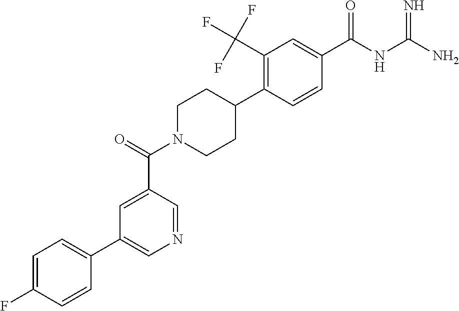 | N-(4-{1-[5-(4-Fluoro-phenyl)- pyridine-3-carbonyl]-piperidin-4-yl}- 3-trifluoromethyl-benzoyl)-guanidine |
| 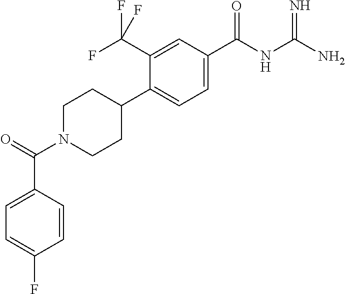 | N-{4-[1-(4-Fluoro-benzoyl)-piperidin- 4-yl]-3-trifluoromethyl-benzoyl}- guanidine |
| 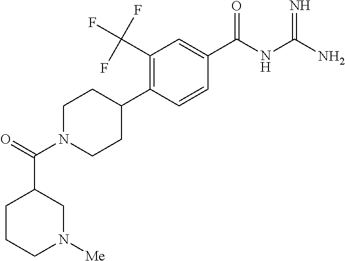 | N-{4-[1-(1-Methyl-piperidine-3- carbonyl)-piperidin-4-yl]-3- trifluoromethyl-benzoyl}-guanidine |
| 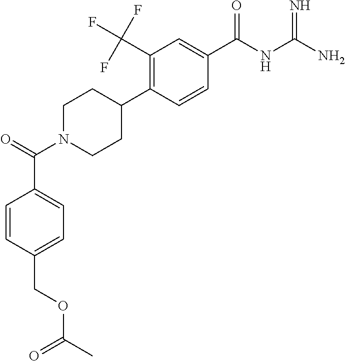 | Acetic acid 4-[4-(4- guanidinocarbonyl-2-trifluoromethyl- phenyl)-piperidine-1-carbonyl]-benzyl ester |
| 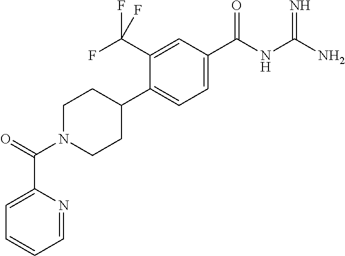 | N-{4-[1-(Pyridine-2-carbonyl)- piperidin-4-yl]-3-trifluoromethyl- benzoyl}-guanidine |
| 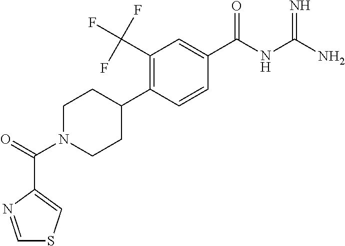 | N-{4-[1-(Thiazole-4-carbonyl)- piperidin-4-yl]-3-trifluoromethyl- benzoyl}-guanidine |
| 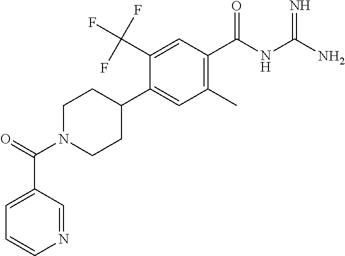 | N-{2-Methyl-4-[1-(pyridine-3- carbonyl)-piperidin-4-yl]-5- trifluoromethyl-benzoyl}-guanidine |
| 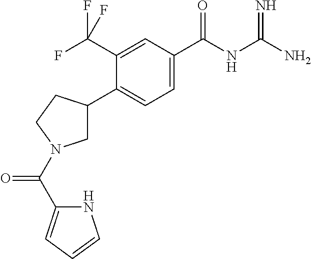 | N-{4-[1-(1H-Pyrrole-2-carbonyl)- pyrrolidin-3-yl]-3-trifluoromethyl- benzoyl}-guanidine |
| 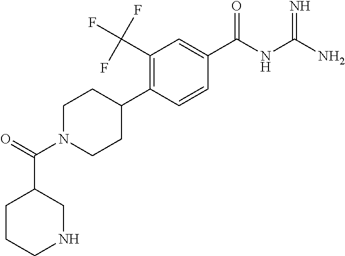 | N-{4-[1-(Piperidine-3-carbonyl)- piperidin-4-yl]-3-trifluoromethyl- benzoyl}-guanidine |
| 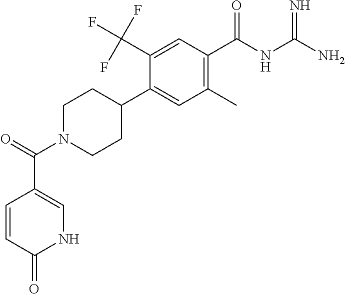 | N-{2-Methyl-4-[1-(6-oxo-1,6- dihydro-pyridine-3-carbonyl)- piperidin-4-yl]-5-trifluoromethyl- benzoyl}-guanidine |
| 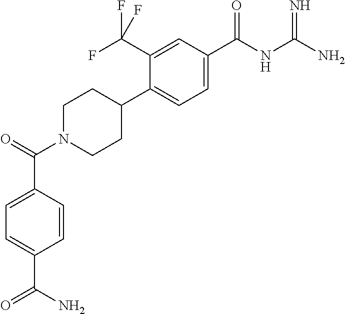 | 4-[4-(4-Guanidinocarbonyl-2- trifluoromethyl-phenyl)-piperidine-1- carbonyl]-benzamide |
| 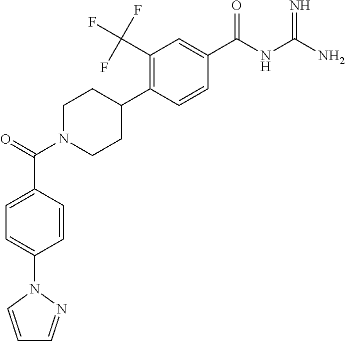 | N-{4-[1-(4-Pyrazol-1-yl-benzoyl)- piperidin-4-yl]-3-trifluoromethyl- benzoyl}-guanidine |
| 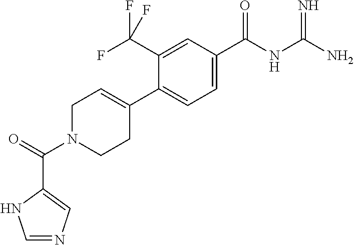 | N-{4-[1-(3H-Imidazole-4-carbonyl)- 1,2,3,6-tetrahydro-pyridin-4-yl]-3- trifluoromethyl-benzoyl}-guanidine |
| 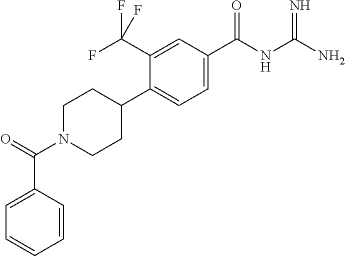 | N-[4-(1-Benzoyl-piperidin-4-yl)-3- trifluoromethyl-benzoyl]-guanidine |
| 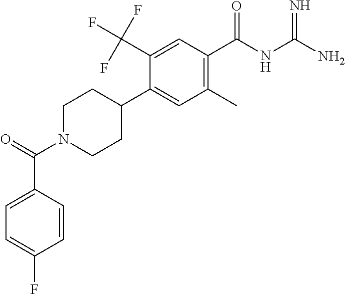 | N-{4-[1-(4-Fluoro-benzoyl)-piperidin- 4-yl]-2-methyl-5-trifluoromethyl- benzoyl}-guanidine |
| 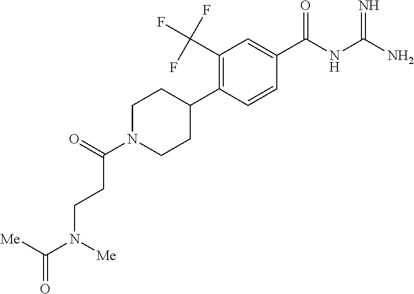 | N-{3-[4-(4-Guanidinocarbonyl-2- trifluoromethyl-phenyl)-piperidin-1- yl]-3-oxo-propyl}-N-methyl- acetamide |
| 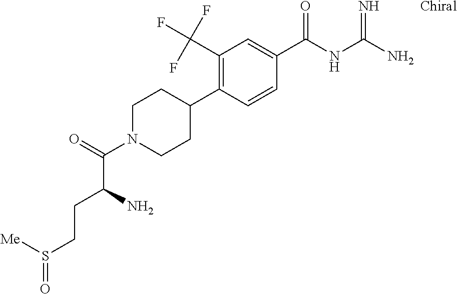 | (S)-N-{4-[1-(-2-Amino-4- methanesulfinyl-butyryl)-piperidin-4- yl]-3-trifluoromethyl-benzoyl}- guanidine |
| 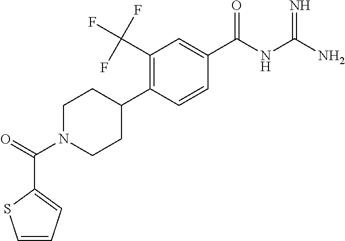 | N-{4-[1-(Thiophene-2-carbonyl)- piperidin-4-yl]-3-trifluoromethyl- benzoyl}-guanidine |
| 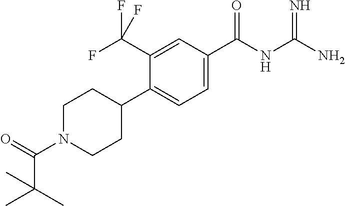 | N-{4-[1-(2,2-Dimethyl-propionyl)- piperidin-4-yl]-3-trifluoromethyl- benzoyl}-guanidine |
| 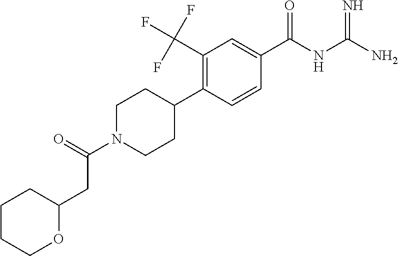 | N-{4-[1-(2-Tetrahydro-pyran-2-yl- acetyl)-piperidin-4-yl]-3- trifluoromethyl-benzoyl}-guanidine |
| 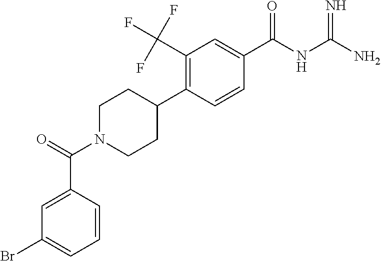 | N-{4-[1-(3-Bromo-benzoyl)- piperidin-4-yl]-3-trifluoromethyl- benzoyl}-guanidine |
| 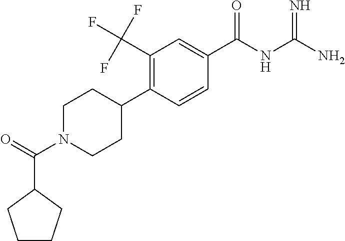 | N-[4-(1-Cyclopentanecarbonyl- piperidin-4-yl)-3-trifluoromethyl- benzoyl]-guanidine |
| 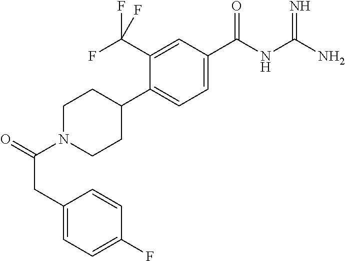 | N-(4-{1-[2-(4-Fluoro-phenyl)-acetyl]- piperidin-4-yl}-3-trifluoromethyl- benzoyl)-guanidine |
| 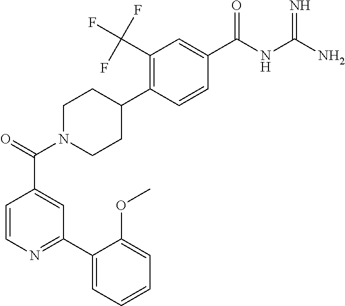 | N-(4-{1-[2-(2-Methoxy-phenyl)- pyridine-4-carbonyl]-piperidin-4-yl}- 3-trifluoromethyl-benzoyl)-guanidine |
| 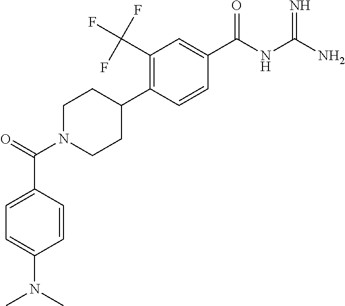 | N-{4-[1-(4-Dimethylamino-benzoyl)- piperidin-4-yl]-3-trifluoromethyl- benzoyl}-guanidine |
| 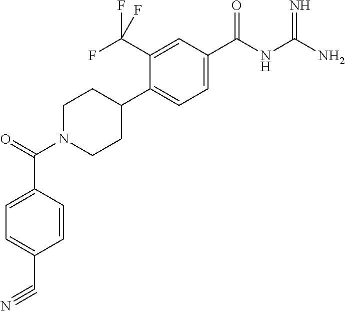 | N-{4-[1-(4-Cyano-benzoyl)-piperidin- 4-yl]-3-trifluoromethyl-benzoyl}- guanidine |
| 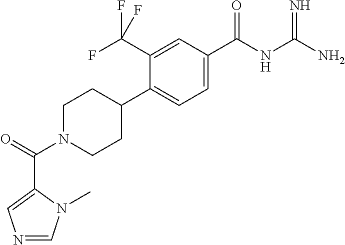 | N-{4-[1-(3-Methyl-3H-imidazole-4- carbonyl)-piperidin-4-yl]-3- trifluoromethyl-benzoyl}-guanidine |
| 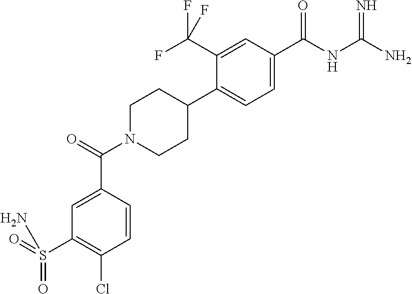 | 2-Chloro-5-[4-(4-guanidinocarbonyl- 2-trifluoromethyl-phenyl)-piperidine- 1-carbonyl]-benzenesulfonamide |
| 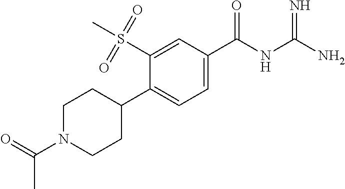 | N-[4-(1-Acetyl-piperidin-4-yl)-3- methanesulfonyl-benzoyl]-guanidine |
| 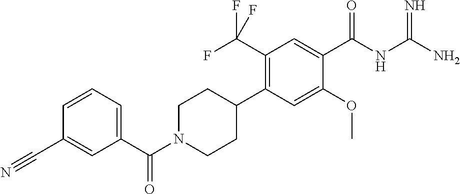 | N-{4-[1-(3-Cyano-benzoyl)-piperidin- 4-yl]-2-methoxy-5-trifluoromethyl- benzoyl}-guanidine |
| 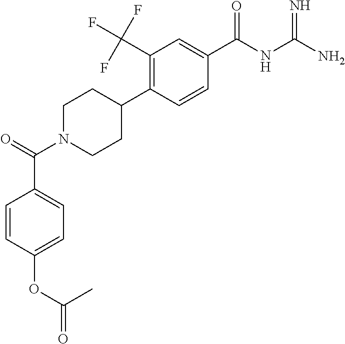 | Acetic acid 4-[4-(4- guanidinocarbonyl-2-trifluoromethyl- phenyl)-piperidine-1-carbonyl]- phenyl ester |
| 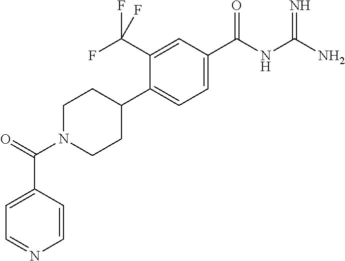 | N-{4-[1-(Pyridine-4-carbonyl)- piperidin-4-yl]-3-trifluoromethyl- benzoyl}-guanidine |
| 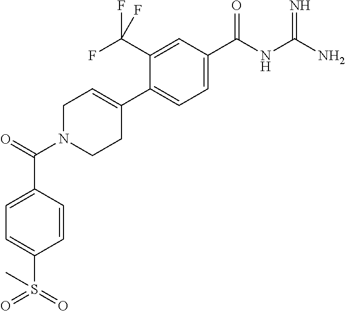 | N-{4-[1-(4-Methanesulfonyl- benzoyl)-1,2,3,6-tetrahydro-pyridin-4- yl]-3-trifluoromethyl-benzoyl}- guanidine |
| 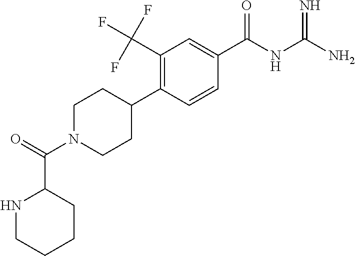 | N-{4-[1-(Piperidine-2-carbonyl)- piperidin-4-yl]-3-trifluoromethyl- benzoyl}-guanidine |
| 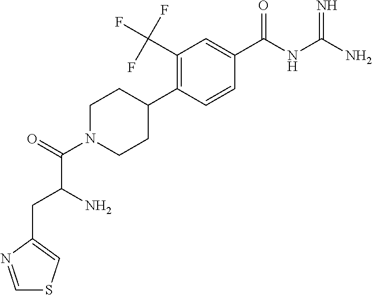 | N-{4-[1-(2-Amino-3-thiazol-4-yl- propionyl)-piperidin-4-yl]-3- trifluoromethyl-benzoyl}-guanidine |
| 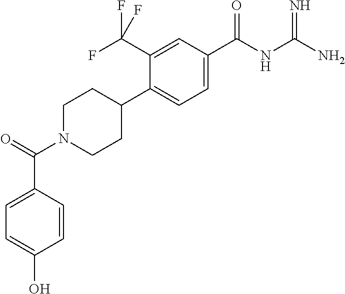 | N-{4-[1-(4-Hydroxy-benzoyl)- piperidin-4-yl]-3-trifluoromethyl- benzoyl}-guanidine |
| 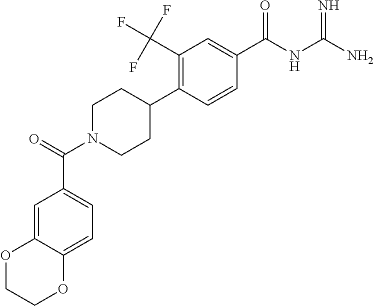 | N-{4-[1-(2,3-Dihydro- benzo[1,4]dioxine-6-carbonyl)- piperidin-4-yl]-3-trifluoromethyl- benzoyl}-guanidine |
| 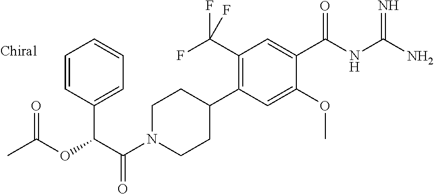 | (R)-Acetic acid 2-[4-(4- guanidinocarbonyl-5-methoxy-2- trifluoromethyl-phenyl)-piperidin-1- yl]-2-oxo-1-phenyl-ethyl ester |
| 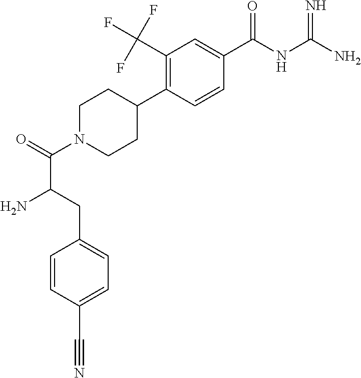 | N-(4-{1-[2-Amino-3-(4-cyano- phenyl)-propionyl]-piperidin-4-yl}-3- trifluoromethyl-benzoyl)-guanidine |
| 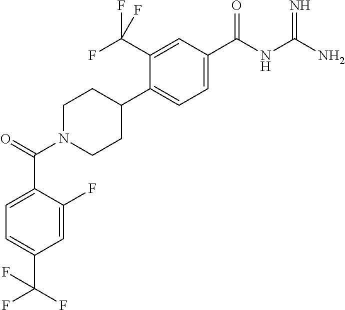 | N-{4-[1-(2-Fluoro-4-trifluoromethyl- benzoyl)-piperidin-4-yl]-3- trifluoromethyl-benzoyl}-guanidine |
| 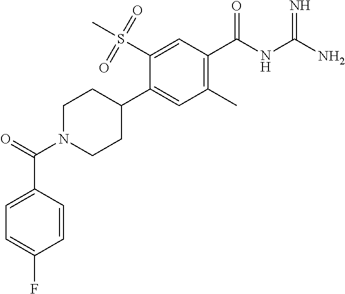 | N-{4-[1-(4-Fluoro-benzoyl)-piperidin- 4-yl]-5-methanesulfonyl-2-methyl- benzoyl}-guanidine |
| 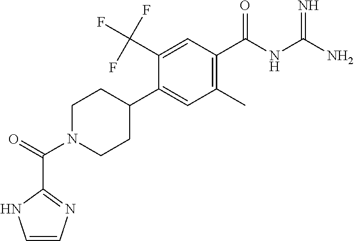 | N-{4-[1-(1H-Imidazole-2-carbonyl)- piperidin-4-yl]-2-methyl-5- trifluoromethyl-benzoyl}-guanidine |
| 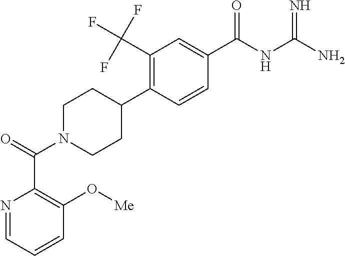 | N-{4-[1-(3-Methoxy-pyridine-2- carbonyl)-piperidin-4-yl]-3- trifluoromethyl-benzoyl}-guanidine |
| 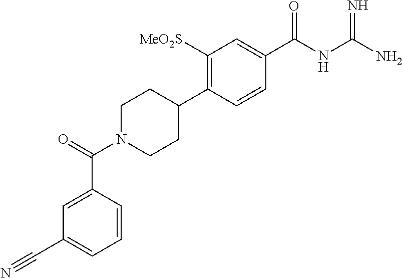 | N-{4-[1-(3-cyano-benzoyl)-piperidin- 4-yl]-3-methanesulfonyl-benzoyl}- guanidine |
| 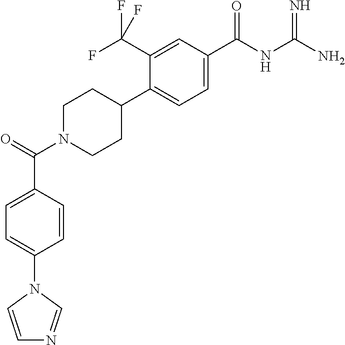 | N-{4-[1-(4-Imidazol-1-yl-benzoyl)- piperidin-4-yl]-3-trifluoromethyl- benzoyl}-guanidine |
| 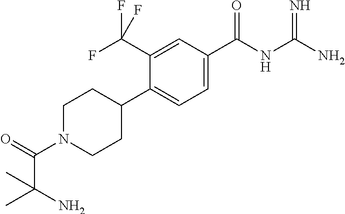 | N-{4-[1-(2-Amino-2-methyl- propionyl)-piperidin-4-yl]-3- trifluoromethyl-benzoyl}-guanidine |
| 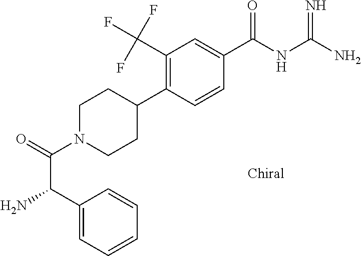 | (S)-N-{4-[1-(-2-Amino-2-phenyl- acetyl)-piperidin-4-yl]-3- trifluoromethyl-benzoyl}-guanidine |
| 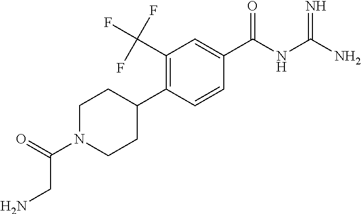 | N-{4-[1-(2-Amino-acetyl)-piperidin- 4-yl]-3-trifluoromethyl-benzoyl}- guanidine |
| 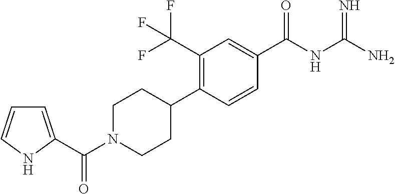 | N-{4-[1-(1H-Pyrrole-2-carbonyl)- piperidin-4-yl]-3-trifluoromethyl- benzoyl}-guanidine |
| 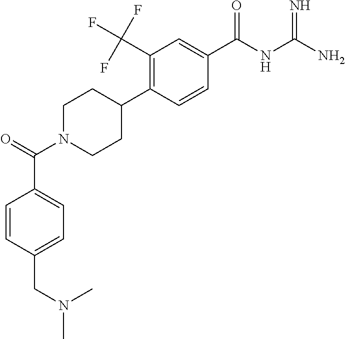 | N-{4-[1-(4-Dimethylaminomethyl- benzoyl)-piperidin-4-yl]-3- trifluoromethyl-benzoyl}-guanidine |
| 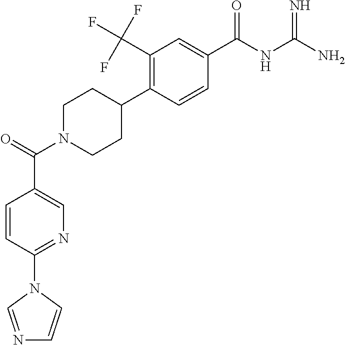 | N-{4-[1-(6-Imidazol-1-yl-pyridine-3- carbonyl)-piperidin-4-yl]-3- trifluoromethyl-benzoyl}-guanidine |
| 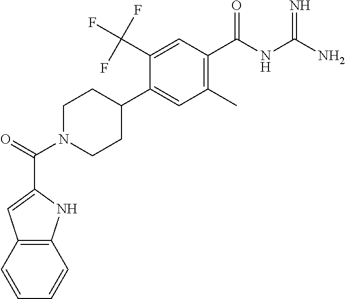 | N-{4-[1-(1H-Indole-2-carbonyl)- piperidin-4-yl]-2-methyl-5- trifluoromethyl-benzoyl}-guanidine |
| 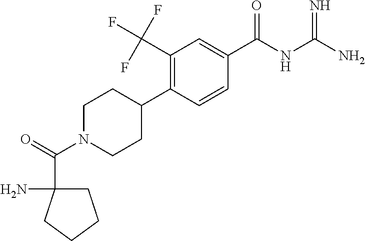 | N-{4-[1-(1-Amino- cyclopentanecarbonyl)-piperidin-4- yl]-3-trifluoromethyl-benzoyl}- guanidine |
| 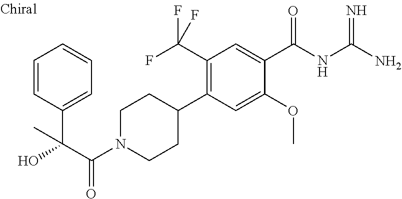 | (R)-N-{4-[1-(2-Hydroxy-2-phenyl- propionyl)-piperidin-4-yl]-2-methoxy- 5-trifluoromethyl-benzoyl}-guanidine |
| 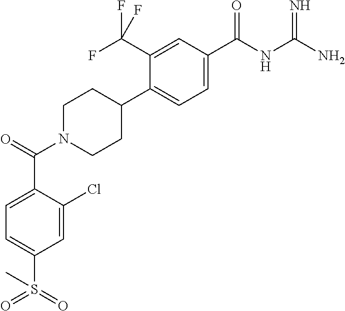 | N-{4-[1-(2-Chloro-4- methanesulfonyl-benzoyl)-piperidin- 4-yl]-3-trifluoromethyl-benzoyl}- guanidine |
| 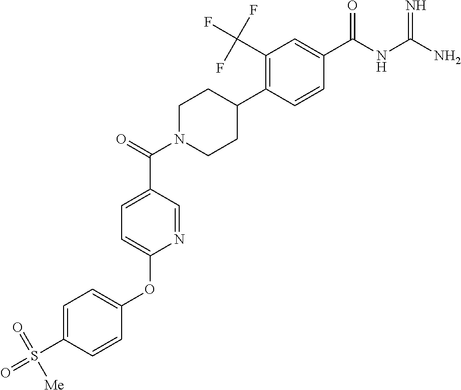 | N-(4-{1-[6-(4-Methanesulfonyl- phenoxy)-pyridine-3-carbonyl]- piperidin-4-yl}-3-trifluoromethyl- benzoyl)-guanidine |
| 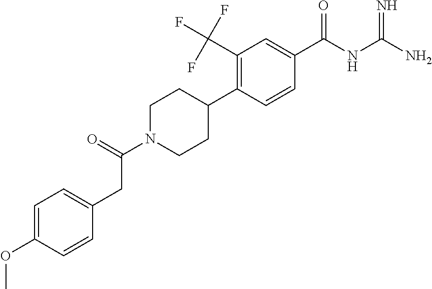 | N-(4-{1-[2-(4-Methoxy-phenyl)- acetyl]-piperidin-4-yl}-3- trifluoromethyl-benzoyl)-guanidine |
| 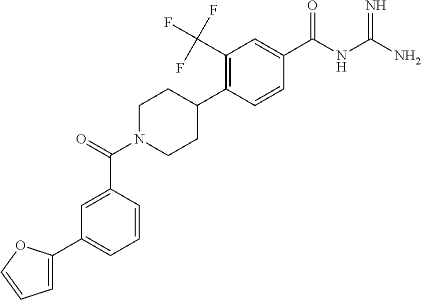 | N-{4-[1-(3-Furan-2-yl-benzoyl)- piperidin-4-yl]-3-trifluoromethyl- benzoyl}-guanidine |
| 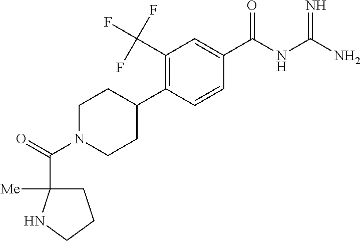 | N-{4-[1-(2-Methyl-pyrrolidine-2- carbonyl)-piperidin-4-yl]-3- trifluoromethyl-benzoyl}-guanidine |
| 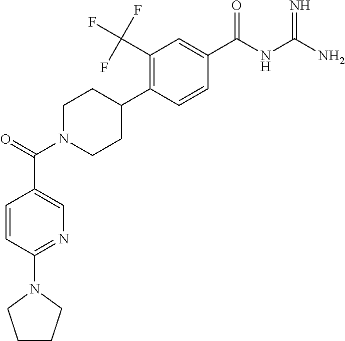 | N-{4-[1-(6-Pyrrolidin-1-yl-pyridine- 3-carbonyl)-piperidin-4-yl]-3- trifluoromethyl-benzoyl}-guanidine |
| 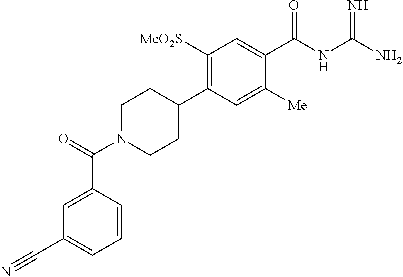 | N-{4-[1-(3-cyano-benzoyl)-piperidin- 4-yl]-5-methanesulfonyl-2-methyl- benzoyl}-guanidine |
| 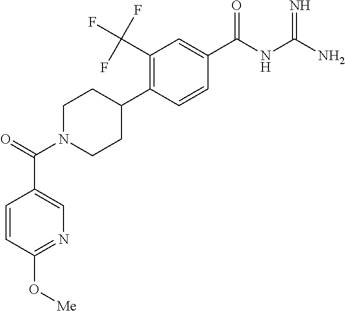 | N-{4-[1-(6-Methoxy-pyridine-3- carbonyl)-piperidin-4-yl]-3- trifluoromethyl-benzoyl}-guanidine |
| 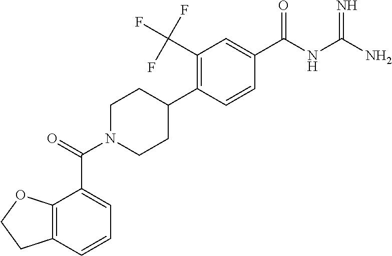 | N-{4-[1-(2,3-Dihydro-benzofuran-7- carbonyl)-piperidin-4-yl]-3- trifluoromethyl-benzoyl}-guanidine |
| 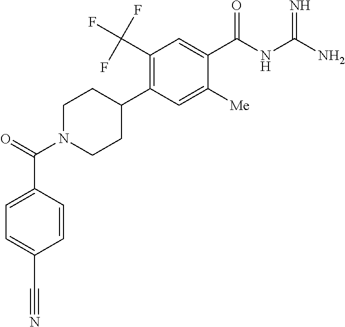 | N-{4-[1-(4-cyano-benzoyl)-piperidin- 4-yl]-2-methyl-5-trifluoromethyl- benzoyl}-guanidine |
| 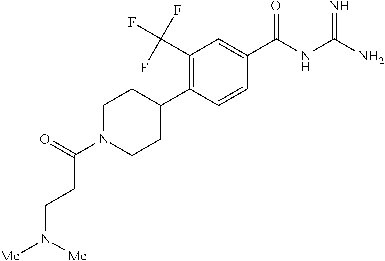 | N-{4-[1-(3-Dimethylamino- propionyl)-piperidin-4-yl]-3- trifluoromethyl-benzoyl}-guanidine |
| 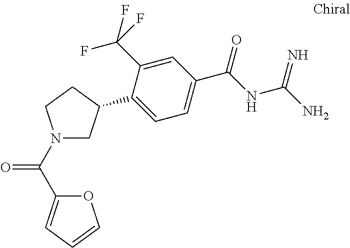 | (R)-N-{4-[1-(Furan-2-carbonyl)- pyrrolidin-3-yl]-3-trifluoromethyl- benzoyl}-guanidine |
| 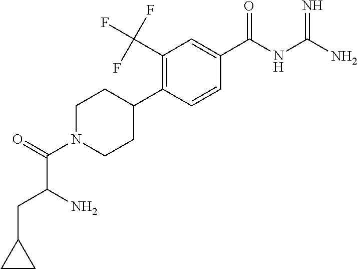 | N-{4-[1-(2-Amino-3-cyclopropyl- propionyl)-piperidin-4-yl]-3- trifluoromethyl-benzoyl}-guanidine |
| 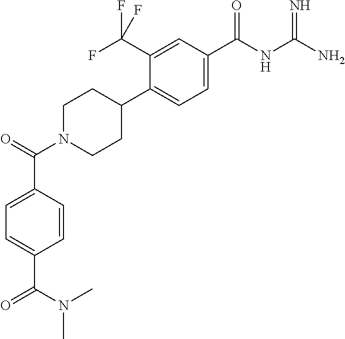 | 4-[4-(4-Guanidinocarbonyl-2- trifluoromethyl-phenyl)-piperidine-1- carbonyl]-N,N-dimethyl-benzamide |
| 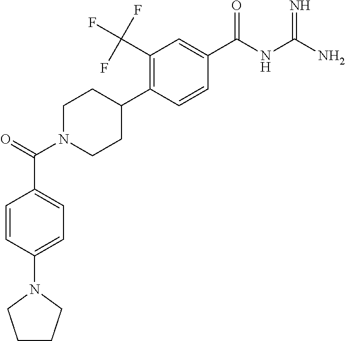 | N-{4-[1-(4-Pyrrolidin-1-yl-benzoyl)- piperidin-4-yl]-3-trifluoromethyl- benzoyl}-guanidine |
| 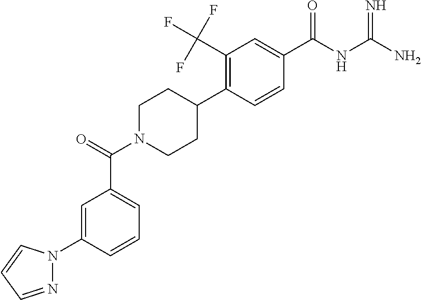 | N-{4-[1-(3-Pyrazol-1-yl-benzoyl)- piperidin-4-yl]-3-trifluoromethyl- benzoyl}-guanidine |
| 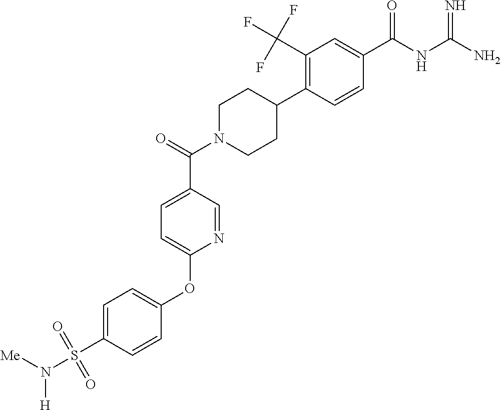 | 4-{5-[4-(4-Guanidinocarbonyl-2- trifluoromethyl-phenyl)-piperidine-1- carbonyl]-pyridin-2-yloxy}-N-methyl- benzenesulfonamide |
| 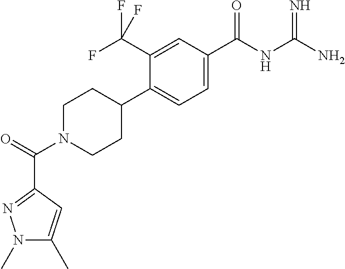 | N-{4-[1-(1,5-Dimethyl-1H-pyrazole- 3-carbonyl)-piperidin-4-yl]-3- trifluoromethyl-benzoyl}-guanidine |
| 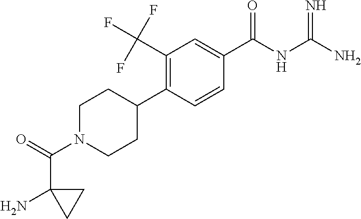 | N-{4-[1-(1-Amino- cyclopropanecarbonyl)-piperidin-4- yl]-3-trifluoromethyl-benzoyl}- guanidine |
| 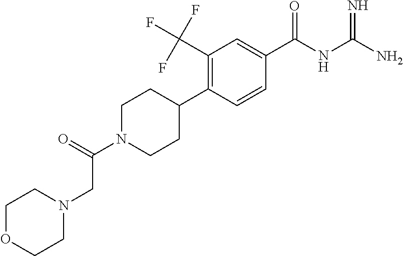 | N-{4-[1-(2-Morpholin-4-yl-acetyl)- piperidin-4-yl]-3-trifluoromethyl- benzoyl}-guanidine |
| 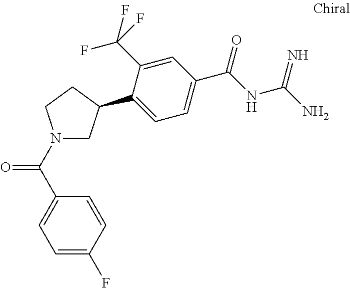 | (S)-N-{4-[-1-(4-fluoro-benzoyl)- pyrrolidin-3-yl]-3-trifluoromethyl- benzoyl}-guanidine |
| 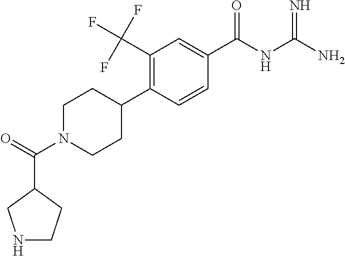 | N-{4-[1-(Pyrrolidine-3-carbonyl)- piperidin-4-yl]-3-trifluoromethyl- benzoyl}-guanidine |
| 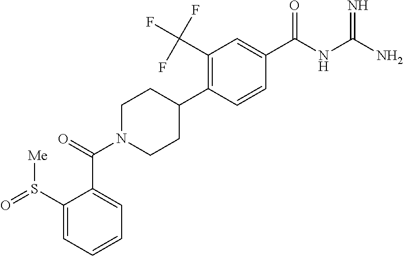 | N-{4-[1-(2-Methanesulfinyl-benzoyl)- piperidin-4-yl]-3-trifluoromethyl- benzoyl}-guanidine |
| 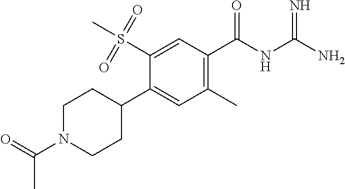 | N-[4-(1-Acetyl-piperidin-4-yl)-5- methanesulfonyl-2-methyl-benzoyl]- guanidine |
| 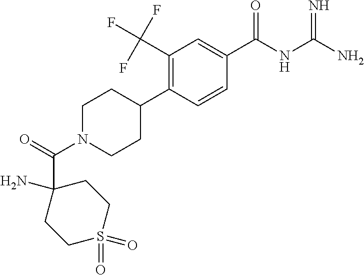 | N-{4-[1-(4-Amino-1,1-dioxo- hexahydro-g-thiopyran-4-carbonyl)- piperidin-4-yl]-3-trifluoromethyl- benzoyl}-guanidine |
| 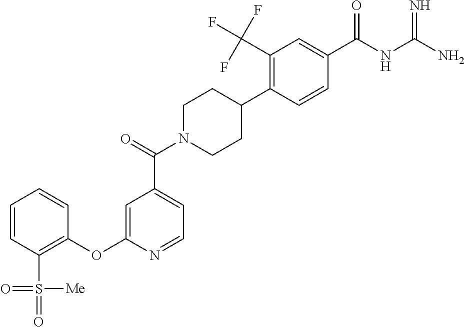 | N-(4-{1-[2-(2-Methanesulfonyl- phenoxy)-pyridine-4-carbonyl]- piperidin-4-yl}-3-trifluoromethyl- benzoyl)-guanidine |
| 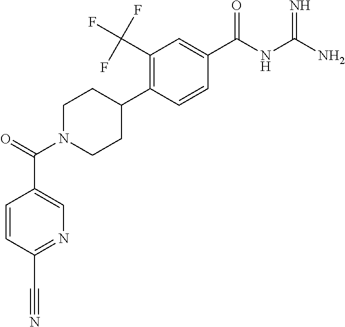 | N-{4-[1-(6-Cyano-pyridine-3- carbonyl)-piperidin-4-yl]-3- trifluoromethyl-benzoyl}-guanidine |
| 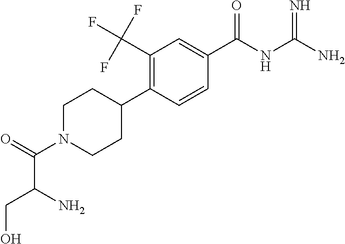 | N-{4-[1-(2-Amino-3-hydroxy- propionyl)-piperidin-4-yl]-3- trifluoromethyl-benzoyl}-guanidine |
| 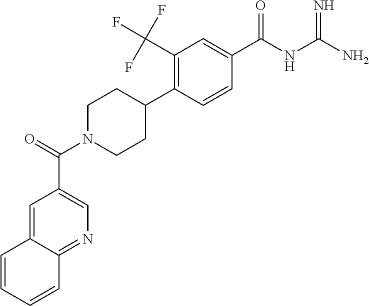 | N-{4-[1-(Quinoline-3-carbonyl)- piperidin-4-yl]-3-trifluoromethyl- benzoyl}-guanidine |
| 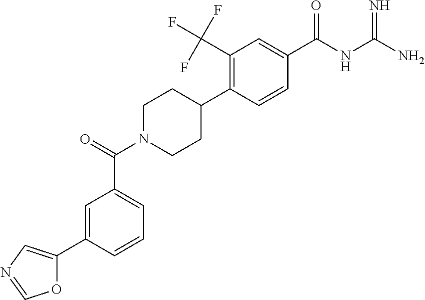 | N-{4-[1-(3-Oxazol-5-yl-benzoyl)- piperidin-4-yl]-3-trifluoromethyl- benzoyl}-guanidine |
| 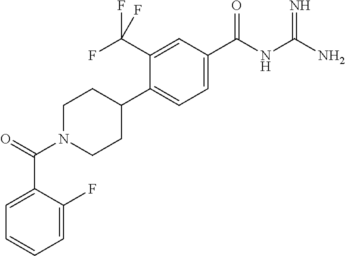 | N-{4-[1-(2-Fluoro-benzoyl)-piperidin- 4-yl]-3-trifluoromethyl-benzoyl}- guanidine |
| 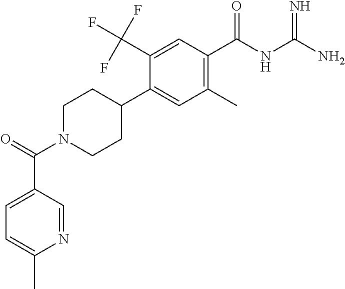 | N-{2-Methyl-4-[1-(6-methyl- pyridine-3-carbonyl)-piperidin-4-yl]- trifluoromethyl-benzoyl}-guanidine |
| 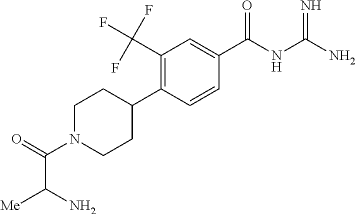 | N-{4-[1-(2-Amino-propionyl)- piperidin-4-yl]-3-trifluoromethyl- benzoyl}-guanidine |
| 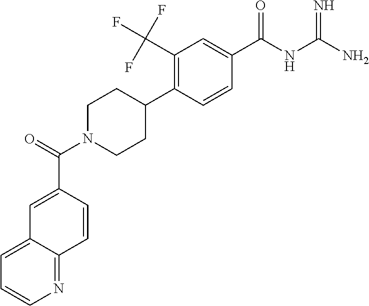 | N-{4-[1-(Quinoline-6-carbonyl)- piperidin-4-yl]-3-trifluoromethyl- benzoyl}-guanidine |
| 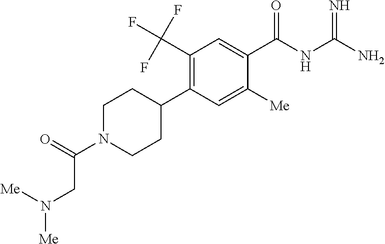 | N-{4-[1-(2-dimethylamino-acetyl)- piperidin-4-yl]-2-methyl-5- trifluoromethyl-benzoyl}-guanidine |
| 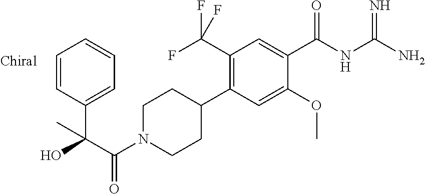 | (S)-N-{4-[1-(2-Hydroxy-2-phenyl- propionyl)-piperidin-4-yl]-2-methoxy- 5-trifluoromethyl-benzoyl}-guanidine |
| 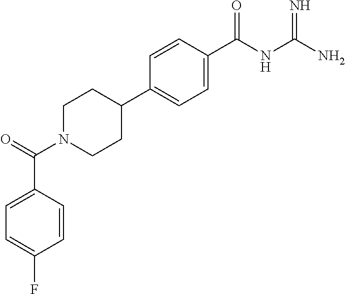 | N-{4-[1-(4-Fluoro-benzoyl)-piperidin- 4-yl]-benzoyl}-guanidine |
| 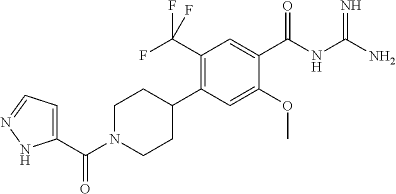 | N-{2-Methoxy-4-[1-(2H-pyrazole-3- carbonyl)-piperidin-4-yl]-5- trifluoromethyl-benzoyl}-guanidine |
| 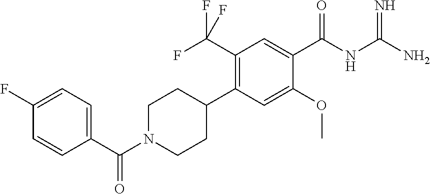 | N-{4-[1-(4-Fluoro-benzoyl)-piperidin- 4-yl]-2-methoxy-5-trifluoromethyl- benzoyl}-guanidine |
| 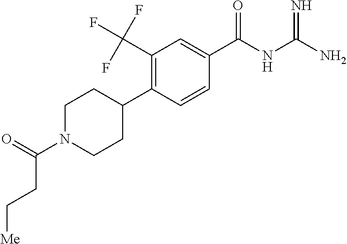 | N-[4-(1-Butyryl-piperidin-4-yl)-3- trifluoromethyl-benzoyl]-guanidine |
| 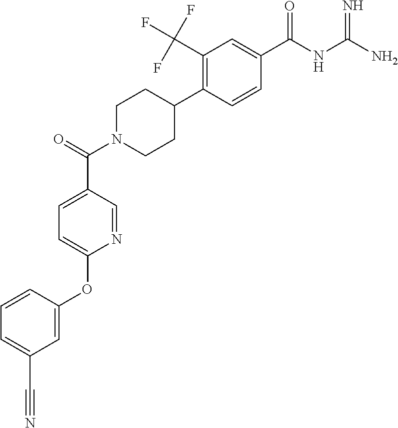 | N-(4-{1-[6-(3-Cyano-phenoxy)- pyridine-3-carbonyl]-piperidin-4-yl}- 3-trifluoromethyl-benzoyl)-guanidine |
| 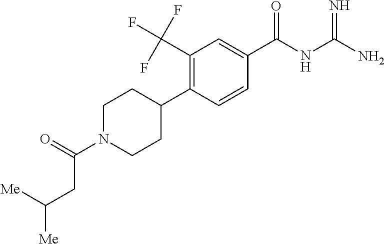 | N-{4-[1-(3-Methyl-butyryl)-piperidin- 4-yl]-3-trifluoromethyl-benzoyl}- guanidine |
| 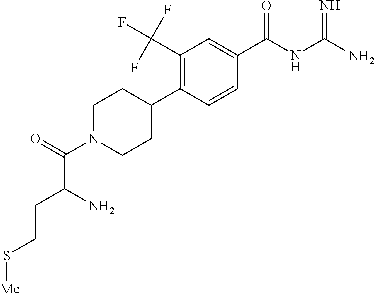 | N-{4-[1-(2-Amino-4-methylsulfanyl- butyryl)-piperidin-4-yl]-3- trifluoromethyl-benzoyl}-guanidine |
| 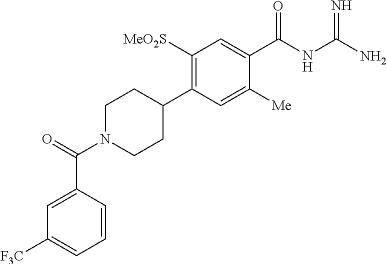 | N-{5-methanesulfonyl-2-methyl-4-[1- (3-trifluoromethyl-benzoyl)-piperidin- 4-yl]-benzoyl}-guanidine |
| 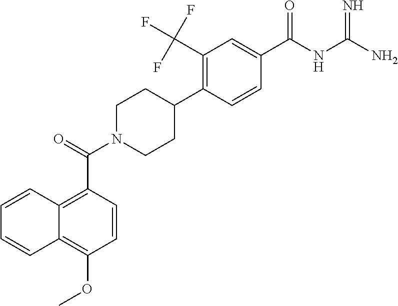 | N-{4-[1-(4-Methoxy-naphthalene-1- carbonyl)-piperidin-4-yl]-3- trifluoromethyl-benzoyl}-guanidine |
| 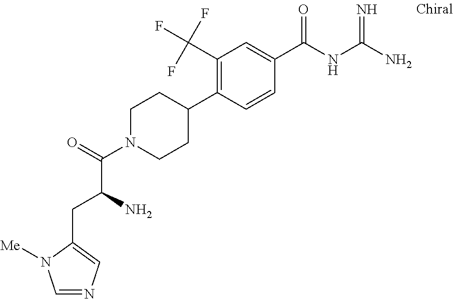 | (S)-N-(4-{1-[2-Amino-3-(3-methyl- 3H-imidazol-4-yl)-propionyl]- piperidin-4-yl}-3-trifluoromethyl- benzoyl)-guanidine |
| 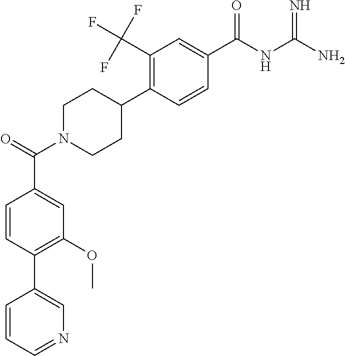 | N-{4-[1-(3-Methoxy-4-pyridin-3-yl- benzoyl)-piperidin-4-yl]-3- trifluoromethyl-benzoyl}-guanidine |
| 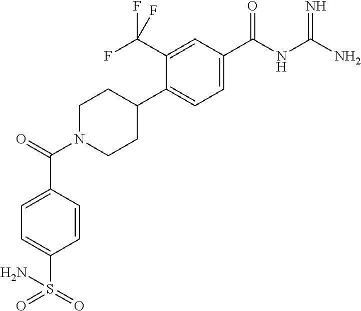 | 4-[4-(4-Guanidinocarbonyl-2- trifluoromethyl-phenyl)-piperidine-1- carbonyl]-benzenesulfonamdide |
| 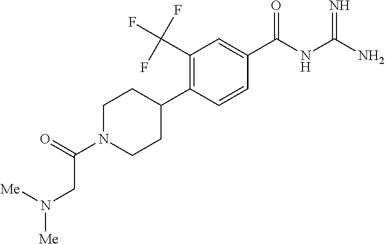 | N-{4-[1-(2-Dimethylamino-acetyl)- piperidin-4-yl]-3-trifluoromethyl- benzoyl}-guanidine |
| 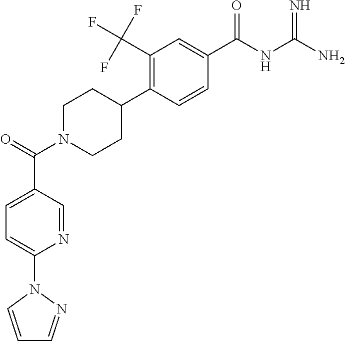 | N-{4-[1-(6-Pyrazol-1-yl-pyridine-3- carbonyl)-piperidin-4-yl]-3- trifluoromethyl-benzoyl}-guanidine |
| 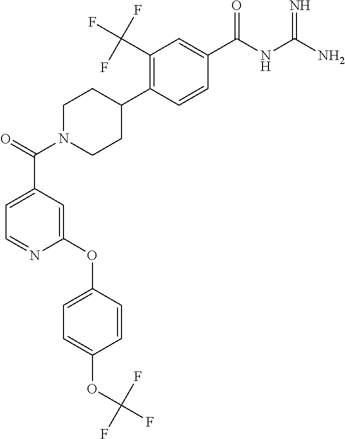 | N-(4-{1-[2-(4-Trifluoromethoxy- phenoxy)-pyridine-4-carbonyl]- piperidin-4-yl}-3-trifluoromethyl- benzoyl)-guanidine |
| 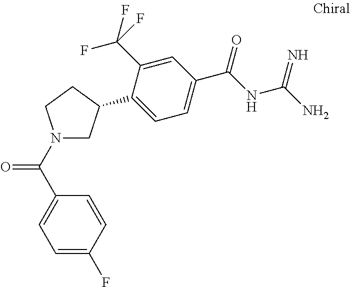 | (R)-N-{4-[1-(4-fluoro-benzoyl)- pyrrolidin-3-yl]-3-trifluoromethyl- benzoyl}-guanidine |
| 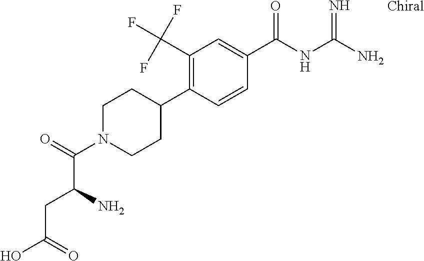 | (S)-3-Amino-4-[4-(4- guanidinocarbonyl-2-trifluoromethyl- phenyl)-piperidin-1-yl]-4-oxo-butyric acid |
| 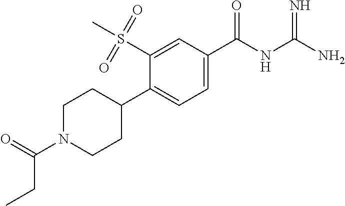 | N-{3-methanesulfonyl-4-[1- propionyl-piperidin-4-yl]-benzoyl}- guanidine |
| 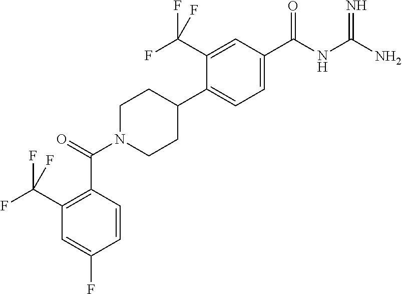 | N-{4-[1-(4-Fluoro-2-trifluoromethyl- benzoyl)-piperidin-4-yl]-3- trifluoromethyl-benzoyl}-guanidine |
| 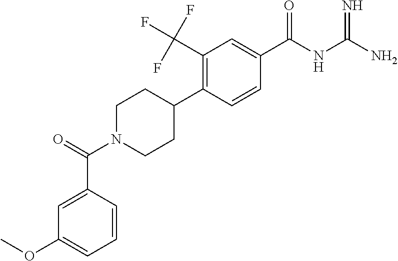 | N-{4-[1-(3-Methoxy-benzoyl)- piperidin-4-yl]-3-trifluoromethyl- benzoyl}-guanidine |
| 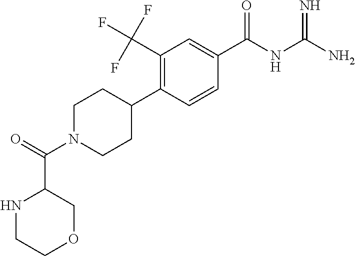 | N-{4-[1-(Morpholine-3-carbonyl)- piperidin-4-yl]-3-trifluoromethyl- benzoyl}-guanidine |
| 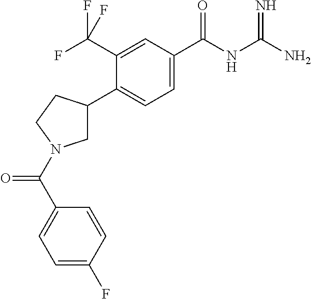 | N-{4-[1-(4-Fluoro-benzoyl)- pyrrolidin-3-yl]-3-trifluoromethyl- benzoyl}-guanidine |
| 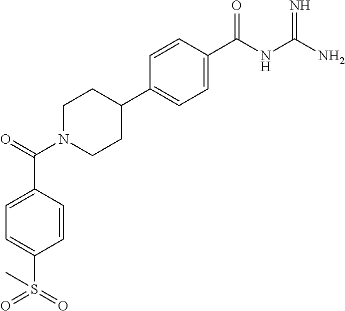 | N-{4-[1-(4-Methanesulfonyl- benzoyl)-piperidin-4-yl]-benzoyl}- guanidine |
| 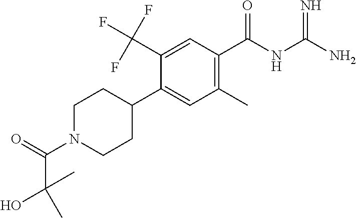 | N-{4-[1-(2-Hydroxy-2-methyl- propionyl)-piperidin-4-yl]-2-methyl- 5-trifluoromethyl-benzoyl}-guanidine |
| 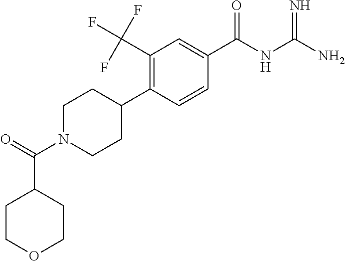 | N-{4-[1-(Tetrahydro-pyran-4- carbonyl)-piperidin-4-yl]-3- trifluoromethyl-benzoyl}-guanidine |
| 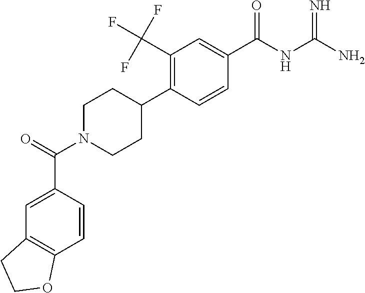 | N-{4-[1-(2,3-Dihydro-benzofuran-5- carbonyl)-piperidin-4-yl]-3- trifluoromethyl-benzoyl}-guanidine |
| 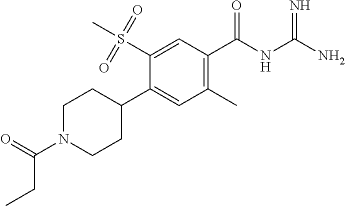 | N-{5-methanesulfonyl-4-[1- propionyl-piperidin-4-yl]-2-methyl- benzoyl}-guanidine |
| 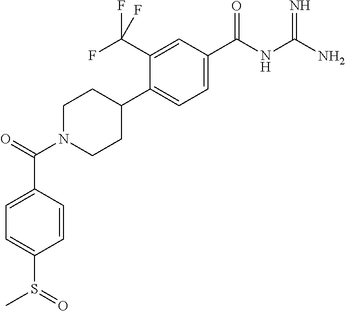 | N-{4-[1-(4-Methanesulfinyl-benzoyl)- piperidin-4-yl]-3-trifluoromethyl- benzoyl}-guanidine |
| 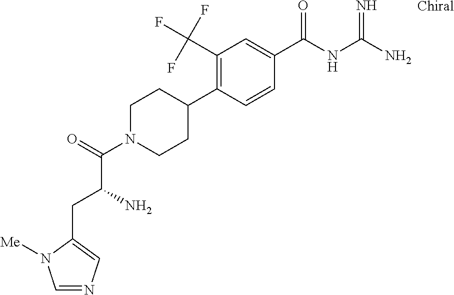 | (R)-N-(4-{1-[2-Amino-3-(3-methyl- 3H-imidazol-4-yl)-3-propionyl]- piperidin-4-yl}-3-trifluoromethyl- benzoyl)-guanidine |
| 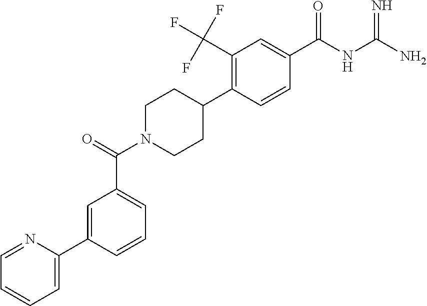 | N-{4-[1-(3-Pyridin-2-yl-benzoyl)- piperidin-4-yl]-3-trifluoromethyl- benzoyl}-guanidine |
| 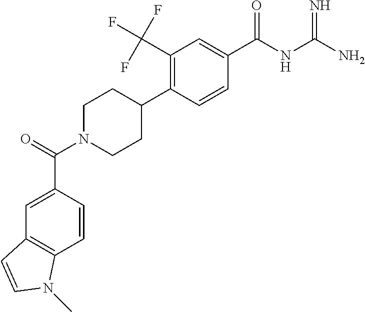 | N-{4-[1-(1-Methyl-1H-indole-5- carbonyl)-piperidin-4-yl]-3- trifluoromethyl-benzoyl}-guanidine |
| 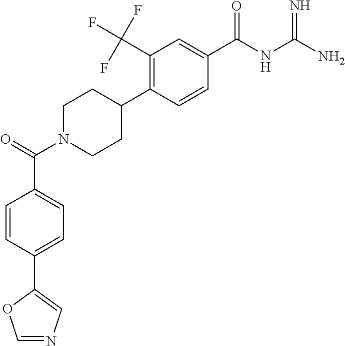 | N-{4-[1-(4-Oxazol-5-yl-benzoyl)- piperidin-4-yl]-3-trifluoromethyl- benzoyl}-guanidine |
| 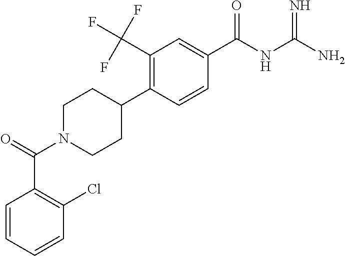 | N-{4-[1-(2-Chloro-benzoyl)- piperidin-4-yl]-3-trifluoromethyl- benzoyl}-guanidine |
| 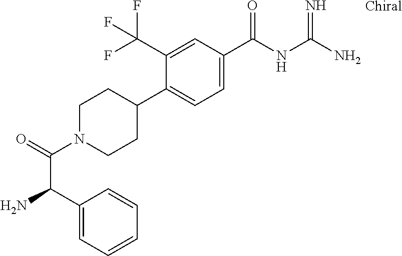 | (R)-N-{4-[1-(2-Amino-2-phenyl- acetyl)-piperidin-4-yl]-3- trifluoromethyl-benzoyl}-guanidine |
| 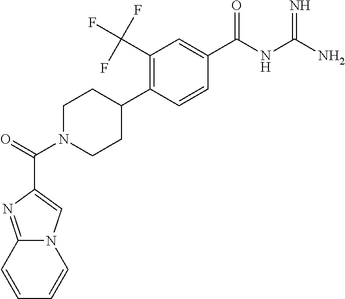 | N-{4-[1-(Imidazo[1,2-a]pyridine-2- carbonyl)-piperidin-4-yl]-3- trifluoromethyl-benzoyl}-guanidine |
| 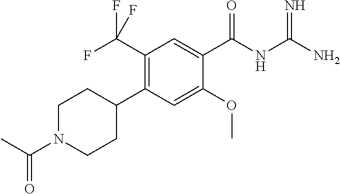 | N-[4-(1-Acetyl-piperidin-4-yl)-2- methoxy-5-trifluoromethyl-benzoyl]- guanidine |
| 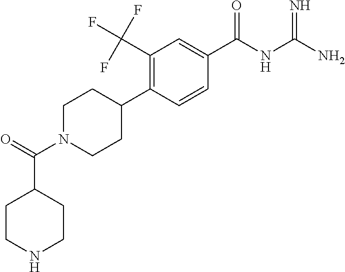 | N-{4-[1-(Piperidine-4-carbonyl)- piperidin-4-yl]-3-trifluoromethyl- benzoyl}-guanidine |
| 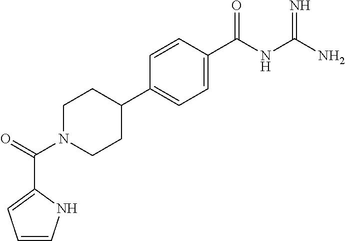 | N-{4-[1-(1H-Pyrrole-2-carbonyl)- piperidin-4-yl]-benzoyl}-guanidine |
| 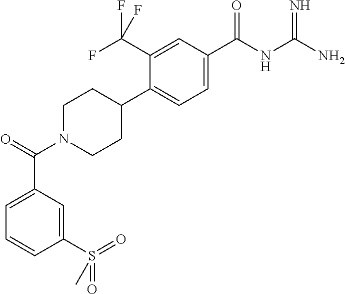 | N-{4-[1-(3-Methanesulfonyl- benzoyl)-piperidin-4-yl]-3- trifluoromethyl-benzoyl}-guanidine |
| 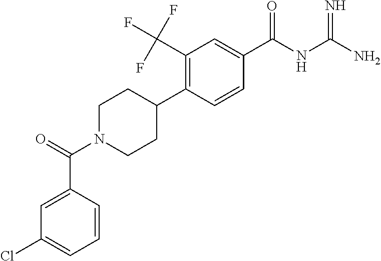 | N-{4-[1-(3-Chloro-benzoyl)- piperidin-4-yl]-3-trifluoromethyl- benzoyl}-guanidine |
| 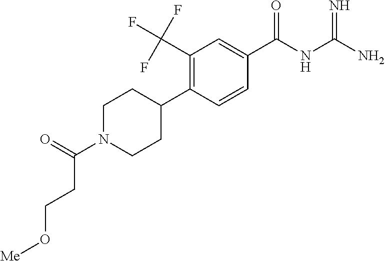 | N-{4-[1-(3-Methoxy-propionyl)- piperidin-4-yl]-3-trifluoromethyl- benzoyl}-guanidine |
| 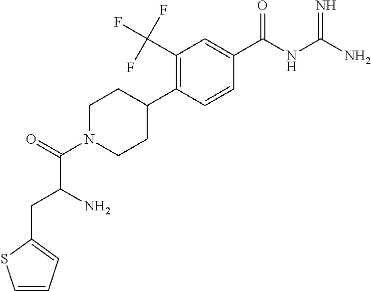 | N-{4-[1-(2-Amino-3-thiophen-2-yl- propionyl)-piperidin-4-yl]-3- trifluoromethyl-benzoyl}-guanidine |
| 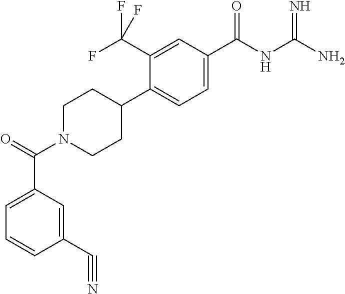 | N-{4-[1-(3-Cyano-benzoyl)-piperidin- 4-yl]-3-trifluoromethyl-benzoyl}- guanidine |
| 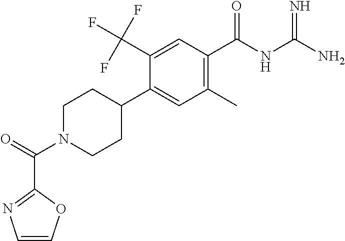 | N-{2-Methyl-4-[1-(oxazole-2- carbonyl)-piperidin-4-yl]-5- trifluoromethyl-benzoyl}-guanidine |
| 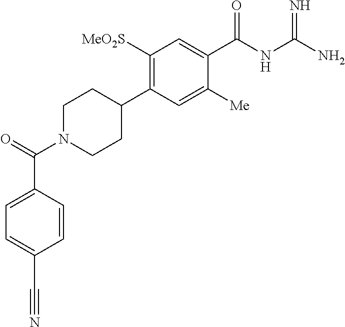 | N-{4-[1-(4-cyano-benzoyl)-piperidin- 4-yl]-5-methanesulfonyl-2-methyl- benzoyl}-guanidine |
| 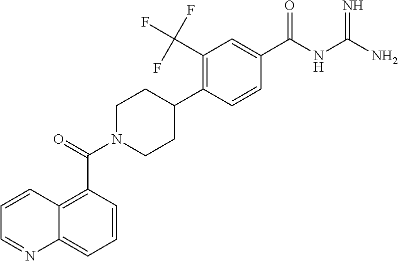 | N-{4-[1-(Quinoline-5-carbonyl)- piperidin-4-yl]-3-trifluoromethyl- benzoyl}-guanidine |
| 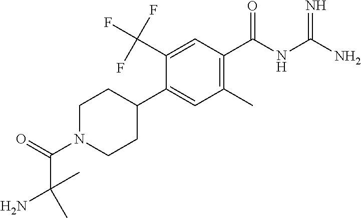 | N-{4-[1-(2-Amino-2-methyl- propionyl)-piperidin-4-yl]-2-methyl- 5-trifluoromethyl-benzoyl}-guanidine |
| 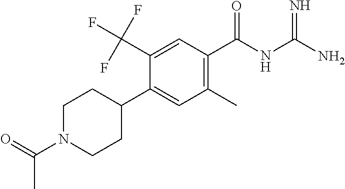 | N-[4-(1-Acetyl-piperidin-4-yl)-2- methyl-5-trifluoromethyl-benzoyl]- guanidine |
| 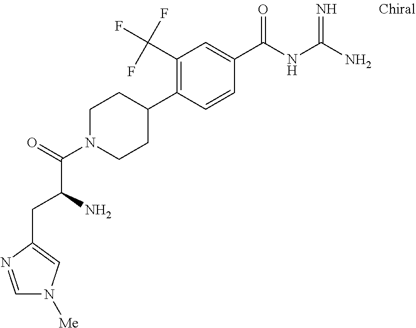 | ( S)-N-(4-{1-[2-Amino-3-(1-methyl- 1H-imidazol-4-yl)-propionyl]- piperidin-4-yl}-3-trifluoromethyl- benzoyl)-guanidine |
| 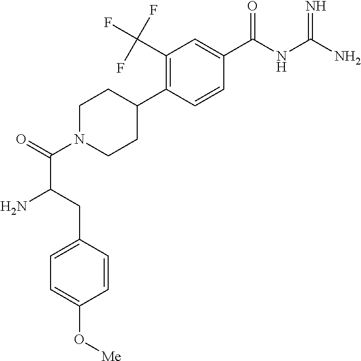 | N-(4-{1-[2-Amino-3-(4-methoxy- phenyl)-propionyl]-piperidin-4-yl}-3- fluoromethyl-benzoyl)-guanidine |
| 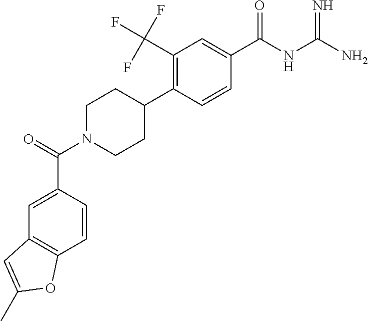 | N-{4-[1-(2-Methyl-benzofuran-5- carbonyl)-piperidin-4-yl]-3- trifluoromethyl-benzoyl}-guanidine |
| 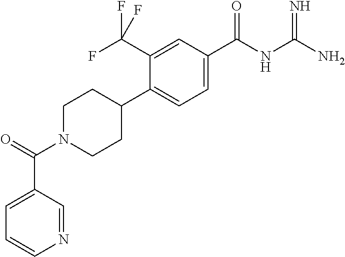 | N-{4-[1-(Pyridine-3-carbonyl)- piperidin-4-yl]-3-trifluoromethyl- benzoyl}-guanidine |
| 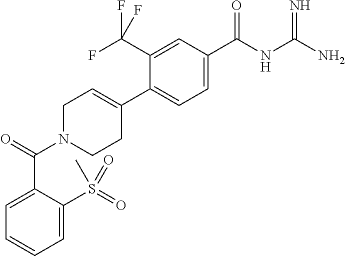 | N-{4-[1-(2-Methanesulfonyl- benzoyl)-1,2,3,6-tetrahydro-pyridin-4- yl]-3-trifluoromethyl-benzoyl}- guanidine |
| 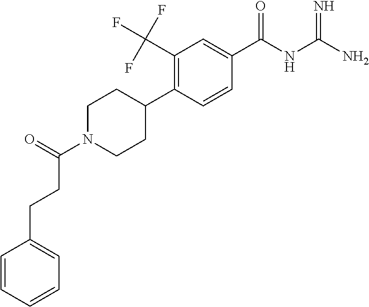 | N-{4-[1-(3-Phenyl-propionyl)- piperidin-4-yl]-3-trifluoromethyl- benzoyl}-guanidine |
| 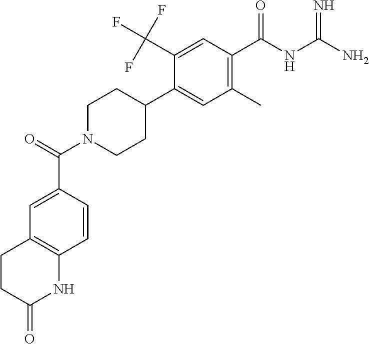 | N-{2-Methyl-4-[1-(2-oxo-1,2,3,4- tetrahydro-quinoline-6-carbonyl)- piperidin-4-yl]-5-trifluoromethyl- benzoyl}-guanidine |
| 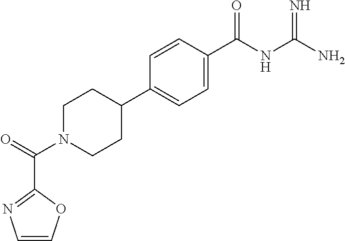 | N-{4-[1-(Oxazole-2-carbonyl)- piperidin-4-yl]-benzoyl}-guanidine |
| 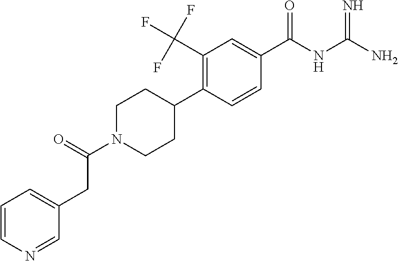 | N-{4-[1-(2-Pyridin-3-yl-acetyl)- piperidin-4-yl]-3-trifluoromethyl- benzoyl}-guanidine |
| 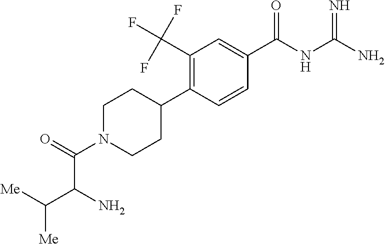 | N-{4-[1-(2-Amino-3-methyl-butyryl)- piperidin-4-yl]-3-trifluoromethyl- benzoyl}-guanidine |
| 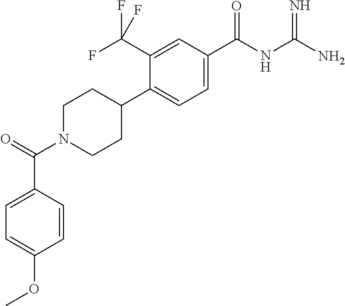 | N-{4-[1-(4-Methoxy-benzoyl)- piperidin-4-yl]-3-trifluoromethyl- benzoyl}-guanidine |
| 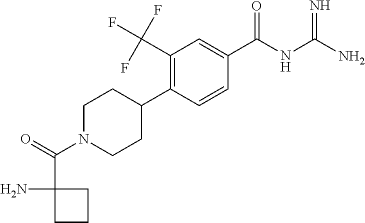 | N-{4-[1-(1-Amino- cyclobutanecarbonyl)-piperidin-4-yl]- 3-trifluoromethyl-benzoyl}-guanidine |
| 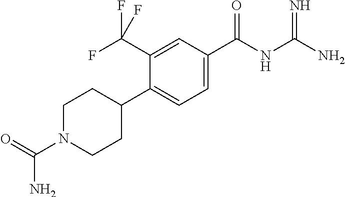 | 4-(4-Guanidinocarbonyl-2- trifluoromethyl-phenyl)-piperidine-1- carboxylic acid amide |
| 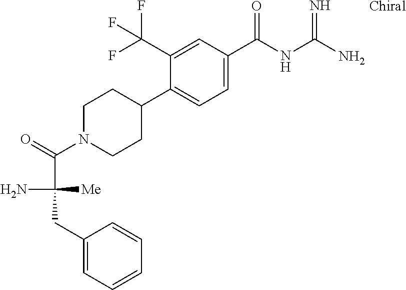 | (S)-N-{4-[1-(2-Amino-2-methyl-3- phenyl-propionyl)-piperidin-4-yl]-3- trifluoromethyl-benzoyl}-guanidine |
| 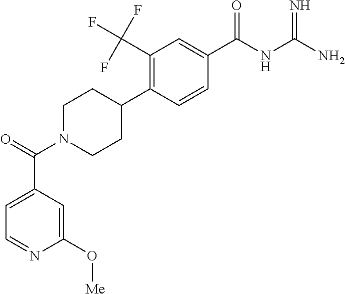 | N-{4-[1-(2-Methoxy-pyridine-4- carbonyl)-piperidin-4-yl]-3- trifluoromethyl-benzoyl}-guanidine |
| 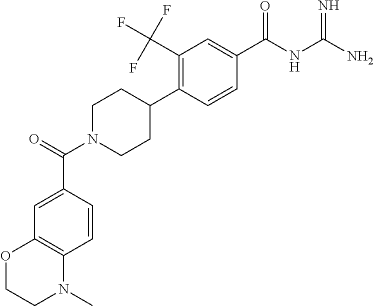 | N-{4-[1-(4-Methyl-3,4-dihydro-2H- benzo[1,4]oxazine-7-carbonyl)- piperidin-4-yl]-3-trifluoromethyl- benzoyl}-guanidine |
| 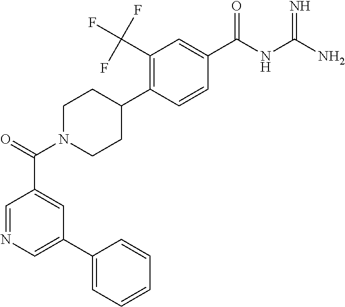 | N-{4-[1-(5-Phenyl-pyridine-3- carbonyl)-piperidin-4-yl]-3- trifluoromethyl-benzoyl}-guanidine |
| 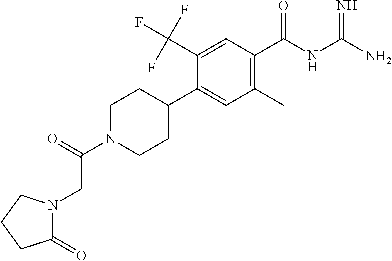 | N-(2-Methyl-4-{1-[2-(2-oxo- pyrrolidin-1-yl)-acetyl]-piperidin-4- yl}-5-trifluoromethyl-benzoyl)- guanidine |
| 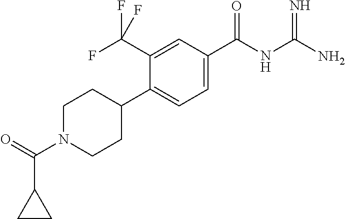 | N-[4-(1-Cyclopropanecarbonyl- piperidin-4-yl)-3-trifluoromethyl- benzoyl]-guanidine |
| 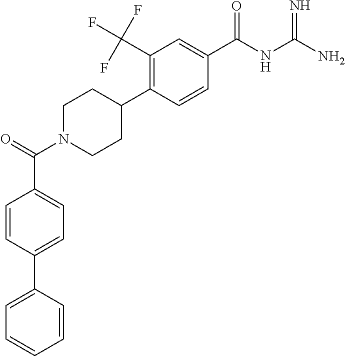 | N-{4-[1-(Biphenyl-4-carbonyl)- piperidin-4-yl]-3-trifluoromethyl- benzoyl}-guanidine |
| 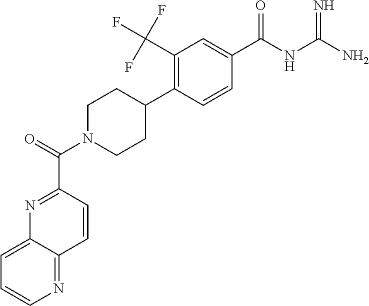 | N-{4-[1-([1,5]Naphthyridine-2- carbonyl)-piperidin-4-yl]-3- trifluoromethyl-benzoyl}-guanidine |
| 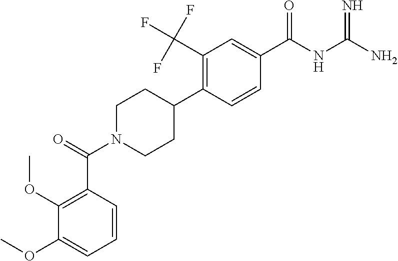 | N-{4-[1-(2,3-Dimethoxy-benzoyl)- piperidin-4-yl]-3-trifluoromethyl- benzoyl}-guanidine |
| 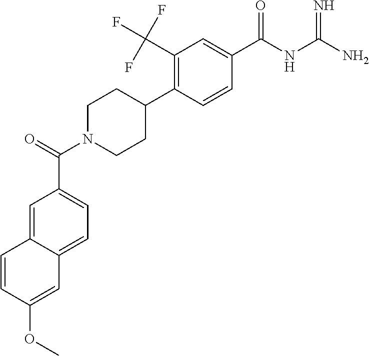 | N-{4-[1-(6-Methoxy-naphthalene-2- carbonyl)-piperidin-4-yl]-3- trifluoromethyl-benzoyl}-guanidine |
| 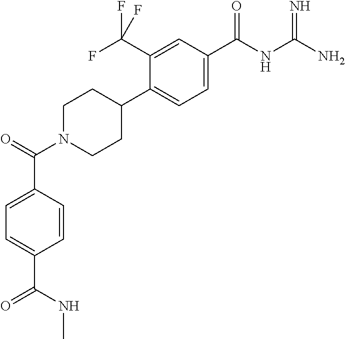 | 4-[4-(4-Guanidinocarbonyl-2- trifluoromethyl-phenyl)-piperidine-1- carbonyl]-N-methyl-benzamide |
| 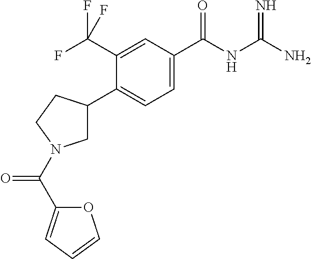 | N-{4-[1-(Furan-2-carbonyl)- pyrrolidin-3-yl]-3-trifluoromethyl- benzoyl}-guanidine |
| 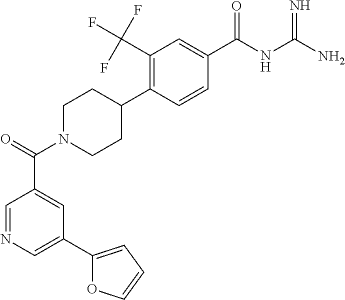 | N-{4-[1-(5-Furan-2-yl-pyridine-3- carbonyl)-piperidin-4-yl]-3- trifluoromethyl-benzoyl}-guanidine |
| 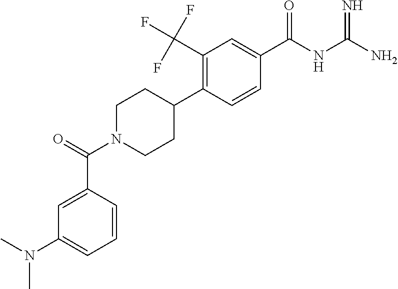 | N-{4-[1-(3-Dimethylamino-benzoyl)- piperidin-4-yl]-3-trifluoromethyl- benzoyl}-guanidine |

or a pharmaceutically acceptable salt thereof.

The following are preferred NHE-1 inhibitors:

TABLE II

| | NHE1 HT29 |
|---|---|
| Name | pHi IC50 (nM) |
| N-{2-Methyl-4-[1-(pyridine-4-carbonyl)-piperidin-4-yl]-5- | 7 |
| trifluoromethyl-benzoyl}-guanidine | |
| N-{4-[1-(2-Amino-4-methyl-pentanoyl)-piperidin-4-yl]-3- | 40 |
| trifluoromethyl-benzoyl}-guanidine | |
| N-{4-[1-(2-Cyano-benzoyl)-1,2,3,6-tetrahydro-pyridin-4-yl]-3- | 93 |
| trifluoromethyl-benzoyl}-guanidine | |
| N-{4-[1-(2-Fluoro-benzoyl)-piperidin-4-yl]-3-trifluoromethyl- | 125 |
| benzoyl}-guanidine | |
| (S)—N-{4-[1-(-5-Oxo-pyrrolidine-2-carbonyl)-piperidin-4-yl]-3- | 11 |
| trifluoromethyl-benzoyl}-guanidine | |
| N-{4-[1-(4-Fluoro-benzoyl)-piperidin-4-yl]-3-methanesulfonyl- | 18 |
| benzoyl}-guanidine\ | |
| N-[4-(1-Butyryl-piperidin-4-yl)-3-trifluoromethyl-benzoyl]- | 30 |
| guanidine | |
| N-{4-[1-(3-cyano-benzoyl)-piperidin-4-yl]-3-methanesulfonyl- | 34 |
| benzoyl}-guanidine | |
| N-{4-[1-(3-Methyl-3H-imidazole-4-carbonyl)-piperidin-4-yl]-3- | 15 |
| trifluoromethyl-benzoyl}-guanidine | |
| 4-{5-[4-(4-Guanidinocarbonyl-2-trifluoromethyl-phenyl)-piperidine- | 92 |
| 1-carbonyl]-pyridin-2-yloxy}-N-methyl-benzenesulfonamide | |
| N-{4-[1-(2-Methyl-benzoyl)-piperidin-4-yl]-3-trifluoromethyl- | 51 |
| benzoyl}-guanidine | |
| N-{4-[1-(Benzothiazole-6-carbonyl)-piperidin-4-yl]-3- | 114 |
| trifluoromethyl-benzoyl}-guanidine | |
| N-[4-(1-Acetyl-piperidin-4-yl)-2-methoxy-5-trifluoromethyl- | 14 |
| benzoyl]-guanidine | |
| N-(4-{1-[2-(4-Methoxy-phenyl)-acetyl]-piperidin-4-yl}-3- | 140 |
| trifluoromethyl-benzoyl)-guanidine | |
| N-(4-{1-[2-Amino-3-(4-cyano-phenyl)-propionyl]-piperidin-4-yl}-3- | 58 |
| trifluoromethyl-benzoyl)-guanidine | |
| N-{4-[1-(3-Pyridin-2-yl-benzoyl)-piperidin-4-yl]-3-trifluoromethyl- | 147 |
| benzoyl}-guanidine | |
| N-{4-[1-(4-Cyano-benzoyl)-piperidin-4-yl]-3-trifluoromethyl- | 24 |
| benzoyl}-guanidine | |
| N-[4-(1-Acetyl-piperidin-4-yl)-3-trifluoromethyl-benzoyl]-guanidine | 9 |
| N-{4-[1-(2-Methanesulfonyl-benzoyl)-piperidin-4-yl]-3- | 69 |
| trifluoromethyl-benzoyl}-guanidine | |
| N-{4-[1-(Pyridine-3-carbonyl)-piperidin-4-yl]-3-trifluoromethyl- | 10 |
| benzoyl}-guanidine | |
| N-{4-[1-(2-Pyridin-3-yl-acetyl)-piperidin-4-yl]-3-trifluoromethyl- | 27 |
| benzoyl}-guanidine | |
| N-{4-[1-(3-Methoxy-benzoyl)-piperidin-4-yl]-3-trifluoromethyl- | 106 |
| benzoyl}-guanidine | |
| (S)—N-{4-[1-(-2-Amino-4-methanesulfinyl-butyryl)-piperidin-4-yl]-3- | 12 |
| trifluoromethyl-benzoyl}-guanidine | |
| N-{4-[1-(1H-Indole-2-carbonyl)-piperidin-4-yl]-2-methyl-5- | 290 |
| trifluoromethyl-benzoyl}-guanidine | |
| N-{2-Methoxy-4-[1-(2H-pyrazole-3-carbonyl)-piperidin-4-yl]-5- | 6 |
| trifluoromethyl-benzoyl}-guanidine | |
| N-{4-[1-(4-Methanesulfonyl-benzoyl)-piperidin-4-yl]-2-methyl-5- | 20 |
| trifluoromethyl-benzoyl}-guanidine | |
| 4-[4-(4-Guanidinocarbonyl-2-trifluoromethyl-phenyl)-piperidine-1- | 13 |
| carbonyl]-benzenesulfonamide | |
| N-{2-Methyl-4-[1-(6-oxo-1,6-dihydro-pyridine-3-carbonyl)- | 30 |
| piperidin-4-yl]-5-trifluoromethyl-benzoyl}-guanidine | |
| N-{4-[1-(4-Methoxy-naphthalene-1-carbonyl)-piperidin-4-yl]-3- | 320 |
| trifluoromethyl-benzoyl}-guanidine | |
| N-{4-[1-(1H-Indole-5-carbonyl)-piperidin-4-yl]-2-methyl-5- | 47 |
| trifluoromethyl-benzoyl}-guanidine | |
| N-{4-[1-(2-Chloro-benzoyl)-piperidin-4-yl]-3-trifluoromethyl- | 119 |
| benzoyl}-guanidine | |
| N-{4-[1-(2,3-Dihydro-benzofuran-5-carbonyl)-piperidin-4-yl]-3- | 23 |
| trifluoromethyl-benzoyl}-guanidine | |
| N-{4-[1-(Pyrimidine-4-carbonyl)-piperidin-4-yl]-3-trifluoromethyl- | 19 |
| benzoyl}-guanidine | |
| N-(2-Methyl-4-{1-[2-(2-oxo-pyrrolidin-1-yl)-acetyl]-piperidin-4-yl}- | 37 |
| 5-trifluoromethyl-benzoyl)-guanidine | |
| N-[4-(1-Acetyl-piperidin-4-yl)-2-methyl-5-trifluoromethyl-benzoyl]- | 9 |
| guanidine | |
| N-{4-[1-(Furan-2-carbonyl)-pyrrolidin-3-yl]-3-trifluoromethyl- | 11 |
| benzoyl}-guanidine | |
| (R)—N-{4-[-1-(Furan-2-carbonyl)-pyrrolidin-3-yl]-3-trifluoromethyl- | 28 |
| benzoyl}-guanidine | |
| N-{4-[1-(Imidazo[1,2-a]pyridine-2-carbonyl)-piperidin-4-yl]-3- | 17 |
| trifluoromethyl-benzoyl}-guanidine | |
| N-{4-[1-(2-Hydroxy-2-methyl-propionyl)-piperidin-4-yl]-2-methyl- | 16 |
| 5-trifluoromethyl-benzoyl}-guanidine | |
| N-{4-[1-(4-Methoxy-benzoyl)-piperidin-4-yl]-3-trifluoromethyl- | 195 |
| benzoyl}-guanidine | |
| Acetic acid (S)-2-[4-(4-guanidinocarbonyl-5-methoxy-2- | 92 |
| trifluoromethyl-phenyl)-piperidin-1-yl]-2-oxo-1-phenyl-ethyl ester | |
| N-{4-[1-(Morpholine-3-carbonyl)-piperidin-4-yl]-3-trifluoromethyl- | 35 |
| benzoyl}-guanidine | |
| (S)—N-{4-[1-(2-Hydroxy-2-phenyl-propionyl)-piperidin-4-yl]-2- | 200 |
| methoxy-5-trifluoromethyl-benzoyl}-guanidine | |
| N-{4-[1-(3-cyano-benzoyl)-piperidin-4-yl]-5-methanesulfonyl-2- | 23 |
| methyl-benzoyl}-guanidine | |
| (S)—N-{4-[-1-(Furan-2-carbonyl)-pyrrolidin-3-yl]-3-trifluoromethyl- | 17 |
| benzoyl}-guanidine | |
| 4-[4-(4-Guanidinocarbonyl-2-trifluoromethyl-phenyl)-piperidine-1- | 183 |
| carbonyl]-benzoic acid methyl ester | |
| N-{4-[1-(6-Pyrrolidin-1-yl-pyridine-3-carbonyl)-piperidin-4-yl]-3- | 75 |
| trifluoromethyl-benzoyl}-guanidine | |
| N-{4-[1-(3-Phenyl-propionyl)-piperidin-4-yl]-3-trifluoromethyl- | 75 |
| benzoyl}-guanidine | |
| N-{4-[1-(4-Methanesulfonyl-benzoyl)-piperidin-4-yl]-3- | 21 |
| trifluoromethyl-benzoyl}-guanidine | |
| N-{4-[1-(Furan-2-carbonyl)-piperidin-4-yl]-3-trifluoromethyl- | 55 |
| benzoyl}-guanidine | |
| N-{4-[1-(1-Amino-cyclohexanecarbonyl)-piperidin-4-yl]-3- | 33 |
| trifluoromethyl-benzoyl}-guanidine | |
| N-{4-[1-(3-Methoxy-pyridine-2-carbonyl)-piperidin-4-yl]-3- | 55 |
| trifluoromethyl-benzoyl}-guanidine | |
| N-{4-[1-(6-Methoxy-naphthalene-2-carbonyl)-piperidin-4-yl]-3- | 390 |
| trifluoromethyl-benzoyl}-guanidine | |
| N-{4-[1-(6-Methoxy-pyridine-3-carbonyl)-piperidin-4-yl]-3- | 58 |
| trifluoromethyl-benzoyl}-guanidine | |
| N-{4-[1-(4-Fluoro-benzoyl)-piperidin-4-yl]-2-methyl-5- | 7 |
| trifluoromethyl-benzoyl}-guanidine | |
| N-{4-[1-(3-Cyano-benzoyl)-piperidin-4-yl]-2-methoxy-5- | 19 |
| trifluoromethyl-benzoyl}-guanidine | |
| N-{4-[1-(4-cyano-benzoyl)-piperidin-4-yl]-2-methyl-5- | 36 |
| trifluoromethyl-benzoyl}-guanidine | |
| N-{4-[1-(1-Pyrimidin-2-yl-piperidine-4-carbonyl)-piperidin-4-yl]-3- | 70 |
| trifluoromethyl-benzoyl}-guanidine | |
| (S)—N-(4-{1-[2-Amino-3-(3-methyl-3H-imidazol-4-yl)-propionyl]- | 22 |
| piperidin-4-yl}-3-trifluoromethyl-benzoyl)-guanidine | |
| N-{4-[1-(1-Methyl-1H-imidazole-2-carbonyl)-piperidin-4-yl]-3- | 15 |
| trifluoromethyl-benzoyl}-guanidine | |
| N-{4-[1-(3-Bromo-benzoyl)-piperidin-4-yl]-3-trifluoromethyl- | 180 |
| benzoyl}-guanidine | |
| (R)—N-{4-[1-(4-fluoro-benzoyl)-pyrrolidin-3-yl]-3-trifluoromethyl- | 24 |
| benzoyl}-guanidine | |
| (R)—N-(4-{1-[2-Amino-3-(1-methyl-1H-imidazol-4-yl)-propionyl]- | 43 |
| piperidin-4-yl}-3-trifluoromethyl-benzoyl)-guanidine | |
| N-{4-[1-(Tetrahydro-pyran-4-carbonyl)-piperidin-4-yl]-3- | 61 |
| trifluoromethyl-benzoyl}-guanidine | |
| N-{4-[1-(3-Dimethylamino-propionyl)-piperidin-4-yl]-3- | 10 |
| trifluoromethyl-benzoyl}-guanidine | |
| N-{4-[1-(3-Methoxy-propionyl)-piperidin-4-yl]-3-trifluoromethyl- | 20 |
| benzoyl}-guanidine | |
| N-{5-methanesulfonyl-2-methyl-4-[1-(3-trifluoromethyl-benzoyl)- | 51 |
| piperidin-4-yl]-benzoyl}-guanidine | |
| N-(4-{1-[2-(4-Fluoro-phenyl)-acetyl]-piperidin-4-yl}-3- | 135 |
| trifluoromethyl-benzoyl)-guanidine | |
| N-{4-[1-(3-Dimethylamino-benzoyl)-piperidin-4-yl]-3- | 77 |
| trifluoromethyl-benzoyl}-guanidine | |
| N-{4-[1-(4-cyano-benzoyl)-piperidin-4-yl]-5-methanesulfonyl-2- | 27 |
| methyl-benzoyl}-guanidine | |
| N-(4-{1-[6-(3-Methoxy-phenyl)-pyridine-3-carbonyl]-piperidin-4- | 250 |
| yl}-3-trifluoromethyl-benzoyl)-guanidine | |
| N-{4-[1-(Naphthalene-1-carbonyl)-piperidin-4-yl]-3-trifluoromethyl- | 260 |
| benzoyl}-guanidine | |
| N-{4-[1-(4-Fluoro-2-trifluoromethyl-benzoyl)-piperidin-4-yl]-3- | 350 |
| trifluoromethyl-benzoyl}-guanidine | |
| 4-[4-(4-Guanidinocarbonyl-2-trifluoromethyl-phenyl)-piperidine-1- | 36 |
| carbonyl]-N,N-dimethyl-benzenesulfonamide | |
| N-{4-[1-(2-Dimethylamino-acetyl)-piperidin-4-yl]-3-trifluoromethyl- | 5 |
| benzoyl}-guanidine | |
| N-{4-[1-(6-Imidazol-1-yl-pyridine-3-carbonyl)-piperidin-4-yl]-3- | 14 |
| trifluoromethyl-benzoyl}-guanidine | |
| N-{4-[1-(3-Cyano-benzoyl)-piperidin-4-yl]-3-trifluoromethyl- | 65 |
| benzoyl}-guanidine | |
| N-{4-[1-(2-Amino-3-pyridin-4-yl-propionyl)-piperidin-4-yl]-3- | 32 |
| trifluoromethyl-benzoyl}-guanidine | |
| N-[4-(1-Benzoyl-piperidin-4-yl)-3-trifluoromethyl-benzoyl]- | 45 |
| guanidine | |
| N-{3-methanesulfonyl-4-[1-(3-trifluoromethyl-benzoyl)-piperidin-4- | 54 |
| yl]-benzoyl}-guanidine | |
| N-{4-[1-(1-Methyl-piperidine-4-carbonyl)-piperidin-4-yl]-3- | 0.3 |
| trifluoromethyl-benzoyl}-guanidine | |
| N-{4-[1-(2-Fluoro-4-trifluoromethyl-benzoyl)-piperidin-4-yl]-3- | 305 |
| trifluoromethyl-benzoyl}-guanidine | |
| N-{2-Methyl-4-[1-(pyridine-3-carbonyl)-piperidin-4-yl]-5- | 30 |
| trifluoromethyl-benzoyl}-guanidine | |
| N-{4-[1-(2-Methanesulfinyl-benzoyl)-piperidin-4-yl]-3- | 54 |
| trifluoromethyl-benzoyl}-guanidine | |
| N-{4-[1-(2-Methoxy-pyridine-4-carbonyl)-piperidin-4-yl]-3- | 40 |
| trifluoromethyl-benzoyl}-guanidine | |
| N-{4-[1-(4-Oxazol-5-yl-benzoyl)-piperidin-4-yl]-3-trifluoromethyl- | 109 |
| benzoyl}-guanidine | |
| N-[4-(1-Acetyl-piperidin-4-yl)-5-methanesulfonyl-2-methyl- | 12 |
| benzoyl]-guanidine | |
| (S)—N-(4-{1-[2-Amino-3-(1-methyl-1H-imidazol-4-yl)-propionyl]- | 49 |
| piperidin-4-yl}-3-trifluoromethyl-benzoyl)-guanidine | |
| N-{4-[1-(4-Imidazol-1-yl-benzoyl)-piperidin-4-yl]-3-trifluoromethyl- | 71 |
| benzoyl}-guanidine | |
| N-{3-Trifluoromethyl-4-[1-(3-trifluoromethyl-benzoyl)-piperidin-4- | 250 |
| yl]-benzoyl}-guanidine | |
| N-{4-[1-(5-Phenyl-pyridine-3-carbonyl)-piperidin-4-yl]-3- | 204 |
| trifluoromethyl-benzoyl}-guanidine | |
| N-{4-[1-(2-Methanesulfonyl-benzoyl)-1,2,3,6-tetrahydro-pyridin-4- | 73 |
| yl]-3-trifluoromethyl-benzoyl}-guanidine | |
| N-{4-[1-(2-Amino-2-methyl-propionyl)-piperidin-4-yl]-2-methyl-5- | 10 |
| trifluoromethyl-benzoyl}-guanidine | |
| N-{4-[1-(Piperidine-2-carbonyl)-piperidin-4-yl]-3-trifluoromethyl- | 9 |
| benzoyl}-guanidine | |
| N-{4-[1-(1-Amino-cyclopentanecarbonyl)-piperidin-4-yl]-3- | 2 |
| trifluoromethyl-benzoyl}-guanidine | |
| (S)—N-{4-[1-(-2-Amino-2-phenyl-acetyl)-piperidin-4-yl]-3- | 21 |
| trifluoromethyl-benzoyl}-guanidine | |
| 4-[4-(4-Guanidinocarbonyl-2-trifluoromethyl-phenyl)-piperidine-1- | 43 |
| carbonyl]-N-methyl-benzamide | |
| N-{4-[1-(Pyridine-2-carbonyl)-piperidin-4-yl]-3-trifluoromethyl- | 10 |
| benzoyl}-guanidine | |
| N-{4-[1-(2-Morpholin-4-yl-acetyl)-piperidin-4-yl]-3-trifluoromethyl- | 25 |
| benzoyl}-guanidine | |
| N-[4-(1-Isobutyryl-piperidin-4-yl)-3-trifluoromethyl-benzoyl]- | 25 |
| guanidine | |
| N-{4-[1-(2-Amino-3-phenyl-propionyl)-piperidin-4-yl]-3- | 19 |
| trifluoromethyl-benzoyl}-guanidine | |
| N-{5-methanesulfonyl-4-[1-propionyl-piperidin-4-yl]-2-methyl- | 16 |
| benzoyl}-guanidine | |
| N-{4-[1-([1,8]Naphthyridine-2-carbonyl)-piperidin-4-yl]-3- | 25 |
| trifluoromethyl-benzoyl}-guanidine | |
| N-{3-Trifluoromethyl-4-[1-(4-trifluoromethyl-benzoyl)-piperidin-4- | 337 |
| yl]-benzoyl}-guanidine | |
| N-{4-[1-(3-Pyrazol-1-yl-benzoyl)-piperidin-4-yl]-3-trifluoromethyl- | 150 |
| benzoyl}-guanidine | |
| N-{4-[1-(4-Fluoro-benzoyl)-piperidin-4-yl]-2-methoxy-5- | 16 |
| trifluoromethyl-benzoyl}-guanidine | |
| N-{2-Methoxy-4-[1-(2-methoxy-acetyl)-piperidin-4-yl]-5- | 9 |
| trifluoromethyl-benzoyl}-guanidine | |
| N-{4-[1-(2-Chloro-4-methanesulfonyl-benzoyl)-piperidin-4-yl]-3- | 60 |
| trifluoromethyl-benzoyl}-guanidine | |
| N-{4-[1-(2,2-Dimethyl-propionyl)-piperidin-4-yl]-3-trifluoromethyl- | 68 |
| benzoyl}-guanidine | |
| N-{4-[1-(4-Imidazol-1-yl-benzoyl)-1,2,3,6-tetrahydro-pyridin-4-yl]- | 33 |
| 3-trifluoromethyl-benzoyl}-guanidine | |
| N-{4-[1-(1-Methyl-1H-pyrrole-2-carbonyl)-piperidin-4-yl]-3- | 79 |
| trifluoromethyl-benzoyl}-guanidine | |
| N-{4-[1-(3-Methoxy-4-pyridin-3-yl-benzoyl)-piperidin-4-yl]-3- | 66 |
| trifluoromethyl-benzoyl}-guanidine | |
| 3-Amino-4-[4-(4-guanidinocarbonyl-2-trifluoromethyl-phenyl)- | 15 |
| piperidin-1-yl]-4-oxo-butyramide | |
| N-{4-[1-(3H-Imidazole-4-carbonyl)-1,2,3,6-tetrahydro-pyridin-4-yl]- | 26 |
| 3-trifluoromethyl-benzoyl}-guanidine | |
| N-{4-[1-(6-Cyano-pyridine-3-carbonyl)-piperidin-4-yl]-3- | 39 |
| trifluoromethyl-benzoyl}-guanidine | |
| (R)—N-{4-[1-(2-Amino-2-phenyl-acetyl)-piperidin-4-yl]-3- | 24 |
| trifluoromethyl-benzoyl}-guanidine | |
| N-{2-Methyl-4-[1-(2-oxo-1,2,3,4-tetrahydro-quinoline-6-carbonyl)- | 63 |
| piperidin-4-yl]-5-trifluoromethyl-benzoyl}-guanidine | |
| N-{4-[1-(4-Chloro-benzoyl)-piperidin-4-yl]-3-trifluoromethyl- | 320 |
| benzoyl}-guanidine | |
| 4-[4-(4-Guanidinocarbonyl-2-trifluoromethyl-phenyl)-piperidine-1- | 27 |
| carbonyl]-benzoic acid | |
| 4-[4-(4-Guanidinocarbonyl-2-trifluoromethyl-phenyl)-piperidine-1- | 48 |
| carbonyl]-N,N-dimethyl-benzamide | |
| N-{4-[1-(2,3-Dimethoxy-benzoyl)-piperidin-4-yl]-3-trifluoromethyl- | 46 |
| benzoyl}-guanidine | |
| N-(4-{1-[2-(2-Methoxy-phenyl)-pyridine-4-carbonyl]-piperidin-4- | 200 |
| yl}-3-trifluoromethyl-benzoyl)-guanidine | |
| N-[4-(1-Acetyl-piperidin-4-yl)-3-methanesulfonyl-benzoyl]- | 18 |
| guanidine | |
| N-{4-[1-(1-Amino-indane-1-carbonyl)-piperidin-4-yl]-3- | 18 |
| trifluoromethyl-benzoyl}-guanidine | |
| N-{4-[1-(1,5-Dimethyl-1H-pyrazole-3-carbonyl)-piperidin-4-yl]-3- | 20 |
| trifluoromethyl-benzoyl}-guanidine | |
| N-{4-[1-(3-Methanesulfonyl-benzoyl)-piperidin-4-yl]-3- | 63 |
| trifluoromethyl-benzoyl}-guanidine | |
| N-(4-{1-[5-(4-Fluoro-phenyl)-pyridine-3-carbonyl]-piperidin-4-yl}- | 185 |
| 3-trifluoromethyl-benzoyl)-guanidine | |
| N-{4-[1-(4-Hydroxy-benzoyl)-piperidin-4-yl]-3-trifluoromethyl- | 25 |
| benzoyl}-guanidine | |
| N-{4-[1-(2-Methoxy-acetyl)-piperidin-4-yl]-3-trifluoromethyl- | 22 |
| benzoyl}-guanidine | |
| N-{4-[1-(4-Fluoro-benzoyl)-pyrrolidin-3-yl]-3-trifluoromethyl- | 40 |
| benzoyl}-guanidine | |
| N-{4-[1-(2-Imidazol-1-yl-acetyl)-piperidin-4-yl]-3-trifluoromethyl- | 6 |
| benzoyl}-guanidine | |
| N-{4-[1-(4-Amino-tetrahydro-pyran-4-carbonyl)-piperidin-4-yl]-3- | 18 |
| trifluoromethyl-benzoyl}-guanidine | |
| 4-(4-Guanidinocarbonyl-2-trifluoromethyl-phenyl)-piperidine-1- | 6 |
| carboxylic acid amide | |
| N-{4-[1-(4-Fluoro-benzoyl)-piperidin-4-yl]-3-trifluoromethyl- | 110 |
| benzoyl}-guanidine | |
| N-{4-[1-(4-Methyl-3,4-dihydro-2H-benzo[1,4]oxazine-7-carbonyl)- | 47 |
| piperidin-4-yl]-3-trifluoromethyl-benzoyl}-guanidine | |
| Acetic acid (R)-2-[4-(4-guanidinocarbonyl-5-methoxy-2- | 110 |
| trifluoromethyl-phenyl)-piperidin-1-yl]-2-oxo-1-phenyl-ethyl ester | |
| N-{4-[1-(6-Pyrazol-1-yl-pyridine-3-carbonyl)-piperidin-4-yl]-3- | 92 |
| trifluoromethyl-benzoyl}-guanidine | |
| N-{4-[1-(3-Oxazol-5-yl-benzoyl)-piperidin-4-yl]-3-trifluoromethyl- | 14 |
| benzoyl}-guanidine | |
| N-{4-[1-(Pyrrolidine-3-carbonyl)-piperidin-4-yl]-3-trifluoromethyl- | 5 |
| benzoyl}-guanidine | |
| N-[4-(1-Propionyl-piperidin-4-yl)-3-trifluoromethyl-benzoyl]- | 12 |
| guanidine | |
| N-{4-[1-(4-Pyrrolidin-1-yl-benzoyl)-piperidin-4-yl]-3- | 250 |
| trifluoromethyl-benzoyl}-guanidine | |
| N-{2-Methoxy-4-[1-(pyridine-2-carbonyl)-piperidin-4-yl]-5- | 43 |
| trifluoromethyl-benzoyl}-guanidine | |
| N-{4-[1-(Thiazole-4-carbonyl)-piperidin-4-yl]-3-trifluoromethyl- | 18 |
| benzoyl}-guanidine | |
| (S)—N-{4-[-1-(4-fluoro-benzoyl)-pyrrolidin-3-yl]-3-trifluoromethyl- | 18 |
| benzoyl}-guanidine | |
| N-{4-[1-(Quinoline-5-carbonyl)-piperidin-4-yl]-3-trifluoromethyl- | 46 |
| benzoyl}-guanidine | |
| N-{4-[1-(1H-Imidazole-2-carbonyl)-piperidin-4-yl]-2-methyl-5- | 4 |
| trifluoromethyl-benzoyl}-guanidine | |
| N-{4-[1-(2-Cyano-benzoyl)-piperidin-4-yl]-3-trifluoromethyl- | 62 |
| benzoyl}-guanidine | |
| N-{4-[1-(1-Amino-cyclobutanecarbonyl)-piperidin-4-yl]-3- | 31 |
| trifluoromethyl-benzoyl}-guanidine | |
| N-(4-{1-[6-(4-Methanesulfonyl-phenoxy)-pyridine-3-carbonyl]- | 54 |
| piperidin-4-yl}-3-trifluoromethyl-benzoyl)-guanidine | |
| N-{4-[1-(Piperidine-4-carbonyl)-piperidin-4-yl]-3-trifluoromethyl- | 6 |
| benzoyl}-guanidine | |
| N-{4-[1-(Quinoline-3-carbonyl)-piperidin-4-yl]-3-trifluoromethyl- | 70 |
| benzoyl}-guanidine | |
| N-[4-(1-Cyclopentanecarbonyl-piperidin-4-yl)-3-trifluoromethyl- | 77 |
| benzoyl]-guanidine | |
| 4-(4-Guanidinocarbonyl-2-trifluoromethyl-phenyl)-piperidine-1- | 10 |
| carboxylic acid dimethylamide | |
| N-{4-[1-(2-dimethylamino-acetyl)-piperidin-4-yl]-2-methyl-5- | 6 |
| trifluoromethyl-benzoyl}-guanidine | |
| N-{4-[1-(Pyrrolidine-2-carbonyl)-piperidin-4-yl]-3-trifluoromethyl- | 11 |
| benzoyl}-guanidine | |
| N-{4-[1-(4-Methanesulfinyl-benzoyl)-piperidin-4-yl]-3- | 28 |
| trifluoromethyl-benzoyl}-guanidine | |
| N-{4-[1-(1H-Pyrrole-2-carbonyl)-pyrrolidin-3-yl]-3-trifluoromethyl- | 19 |
| benzoyl}-guanidine | |
| N-{4-[1-(2-Methoxy-pyridine-3-carbonyl)-piperidin-4-yl]-3- | 42 |
| trifluoromethyl-benzoyl}-guanidine | |
| N-{4-[1-(4-Fluoro-benzoyl)-piperidin-4-yl]-5-methanesulfonyl-2- | 23 |
| methyl-benzoyl}-guanidine | |
| N-{5-methanesulfonyl-4-[1-(2-methoxy-acetyl)-piperidin-4-yl]-2- | 15 |
| methyl-benzoyl}-guanidine | |
| N-{4-[1-(4-Methanesulfonyl-benzoyl)-1,2,3,6-tetrahydro-pyridin-4- | 49 |
| yl]-3-trifluoromethyl-benzoyl}-guanidine | |
| N-{4-[1-(3-Methyl-butyryl)-piperidin-4-yl]-3-trifluoromethyl- | 49 |
| benzoyl}-guanidine | |
| N-{4-[1-(2-Methyl-pyrrolidine-2-carbonyl)-piperidin-4-yl]-3- | 12 |
| trifluoromethyl-benzoyl}-guanidine | |
| N-{4-[1-(3-Pyridin-3-yl-benzoyl)-piperidin-4-yl]-3-trifluoromethyl- | 140 |
| benzoyl}-guanidine | |
| N-{4-[1-(Piperidine-3-carbonyl)-piperidin-4-yl]-3-trifluoromethyl- | 17 |
| benzoyl}-guanidine | |
| N-{4-[1-(2-Amino-acetyl)-piperidin-4-yl]-3-trifluoromethyl- | 4 |
| benzoyl}-guanidine | |
| (S)-3-Amino-4-[4-(4-guanidinocarbonyl-2-trifluoromethyl-phenyl)- | 9 |
| piperidin-1-yl]-4-oxo-butyric acid | |
| N-{4-[1-(3-Trifluoromethanesulfonyl-benzoyl)-piperidin-4-yl]-3- | 280 |
| trifluoromethyl-benzoyl}-guanidine | |
| N-{4-[1-(4-Amino-1,1-dioxo-hexahydro-g-thiopyran-4-carbonyl)- | 11 |
| piperidin-4-yl]-3-trifluoromethyl-benzoyl}-guanidine | |
| N-{4-[1-(4-Pyrazol-1-yl-benzoyl)-piperidin-4-yl]-3-trifluoromethyl- | 88 |
| benzoyl}-guanidine | |
| 4-[4-(4-Guanidinocarbonyl-2-trifluoromethyl-phenyl)-piperidine-1- | 28 |
| carbonyl]-benzamide | |
| N-{4-[1-(2-Amino-propionyl)-piperidin-4-yl]-3-trifluoromethyl- | 5 |
| benzoyl}-guanidine | |
| N-{4-[1-(2-Amino-3-cyclopropyl-propionyl)-piperidin-4-yl]-3- | 19 |
| trifluoromethyl-benzoyl}-guanidine | |
| N-(4-{1-[2-(2-Methanesulfonyl-phenoxy)-pyridine-4-carbonyl]- | 76 |
| piperidin-4-yl}-3-trifluoromethyl-benzoyl)-guanidine | |
| N-{4-[1-(2,3-Dihydro-benzo[1,4]dioxin-6-carbonyl)-piperidin-4-yl]- | 130 |
| 3-trifluoromethyl-benzoyl}-guanidine | |
| N-{4-[1-(1H-Pyrrole-2-carbonyl)-piperidin-4-yl]-benzoyl}-guanidine | 385 |
| 4-[4-(4-Guanidinocarbonyl-2-trifluoromethyl-phenyl)-piperidine-1- | 51 |
| carbonyl]-N-methyl-benzenesulfonamide | |
| N-{4-[1-(4-Dimethylamino-benzoyl)-piperidin-4-yl]-3- | 180 |
| trifluoromethyl-benzoyl}-guanidine | |
| N-{4-[1-(2-Amino-3-hydroxy-propionyl)-piperidin-4-yl]-3- | 5 |
| trifluoromethyl-benzoyl}-guanidine | |
| N-{2-Methyl-4-[1-(6-methyl-pyridine-3-carbonyl)-piperidin-4-yl]-5- | 82 |
| trifluoromethyl-benzoyl}-guanidine | |
| N-{4-[1-(3-Fluoro-benzoyl)-piperidin-4-yl]-3-trifluoromethyl- | 105 |
| benzoyl}-guanidine | |
| (R)—N-{4-[1-(2-Amino-2-methyl-3-phenyl-propionyl)-piperidin-4- | 24 |
| yl]-3-trifluoromethyl-benzoyl}-guanidine | |
| N-{2-Methoxy-4-[1-(2-oxo-1,2,3,4-tetrahydro-quinoline-6-carbonyl)- | 4 |
| piperidin-4-yl]-5-trifluoromethyl-benzoyl}-guanidine | |
| N-(4-{1-[2-Amino-3-(4-methoxy-phenyl)-propionyl]-piperidin-4-yl}- | 37 |
| 3-trifluoromethyl-benzoyl)-guanidine | |
| N-{2-Methyl-4-[1-(oxazole-2-carbonyl)-piperidin-4-yl]-5- | 12 |
| trifluoromethyl-benzoyl}-guanidine | |
| N-{4-[1-(3-Amino-propionyl)-piperidin-4-yl]-3-trifluoromethyl- | 8 |
| benzoyl}-guanidine | |
| N-{4-[1-(Quinoline-6-carbonyl)-piperidin-4-yl]-3-trifluoromethyl- | 64 |
| benzoyl}-guanidine | |
| N-{4-[1-(1H-Pyrrole-2-carbonyl)-piperidin-4-yl]-3-trifluoromethyl- | 46 |
| benzoyl}-guanidine | |
| N-{3-methanesulfonyl-4-[1-(2-methoxy-acetyl)-piperidin-4-yl]- | 24 |
| benzoyl}-guanidine | |
| N-{4-[1-(2-Amino-3,3-dimethyl-butyryl)-piperidin-4-yl]-3- | 16 |
| trifluoromethyl-benzoyl}-guanidine | |
| N-{4-[1-(Pyridine-4-carbonyl)-piperidin-4-yl]-3-trifluoromethyl- | 9 |
| benzoyl}-guanidine | |
| N-{4-[1-(4-Methoxymethyl-benzoyl)-piperidin-4-yl]-3- | 122 |
| trifluoromethyl-benzoyl}-guanidine | |
| N-{4-[1-(2-Amino-4-methylsulfanyl-butyryl)-piperidin-4-yl]-3- | 13 |
| trifluoromethyl-benzoyl}-guanidine | |
| 2-Chloro-5-[4-(4-guanidinocarbonyl-2-trifluoromethyl-phenyl)- | 13 |
| piperidine-1-carbonyl]-benzenesulfonamide | |
| N-{4-[1-(2-Amino-3-thiophen-2-yl-propionyl)-piperidin-4-yl]-3- | 35 |
| trifluoromethyl-benzoyl}-guanidine | |
| N-{4-[1-(2-Amino-3-methyl-butyryl)-piperidin-4-yl]-3- | 12 |
| trifluoromethyl-benzoyl}-guanidine | |
| N-{3-methanesulfonyl-4-[1-propionyl-piperidin-4-yl]-benzoyl}- | 21 |
| guanidine | |
| N-[4-(1-Cyclopropanecarbonyl-piperidin-4-yl)-3-trifluoromethyl- | 16 |
| benzoyl]-guanidine | |
| N-{4-[1-(1-Methyl-piperidine-3-carbonyl)-piperidin-4-yl]-3- | 10 |
| trifluoromethyl-benzoyl}-guanidine | |
| N-{4-[1-(4-Trifluoromethanesulfonyl-benzoyl)-piperidin-4-yl]-3- | 230 |
| trifluoromethyl-benzoyl}-guanidine | |
| N-{4-[1-(6-Hydroxy-pyridine-3-carbonyl)-piperidin-4-yl]-3- | 21 |
| trifluoromethyl-benzoyl}-guanidine | |
| N-{4-[1-(2-Amino-2-methyl-propionyl)-piperidin-4-yl]-3- | 8 |
| trifluoromethyl-benzoyl}-guanidine | |

In all the compounds disclosed hereinabove in this application, in the event the nomenclature is in conflict with the structure, it shall be understood that the compound is defined by the structure.

The invention also relates to pharmaceutical preparations, containing as active substance one or more compounds of the invention, or the pharmaceutically acceptable derivatives thereof, optionally combined with conventional excipients and/or carriers.

Compounds of the invention also include their isotopically-labelled forms. An isotopically-labelled form of an active agent of a combination of the present invention is identical to said active agent but for the fact that one or more atoms of said active agent have been replaced by an atom or atoms having an atomic mass or mass number different from the atomic mass or mass number of said atom which is usually found in nature. Examples of isotopes which are readily available commercially and which can be incorporated into an active agent of a combination of the present invention in accordance with well established procedures, include isotopes of hydrogen, carbon, nitrogen, oxygen, phosphorous, fluorine and chlorine, e.g., ²H, ³H, ¹³C, ¹⁴C, ¹⁵N, ¹⁸O, ¹⁷O, ³¹P, ³², ³⁵S, ¹⁸F, and ³⁶Cl, respectively. An active agent of a combination of the present invention, a prodrug thereof, or a pharmaceutically acceptable salt of either which contains one or more of the above-mentioned isotopes and/or other isotopes of other atoms is contemplated to be within the scope of the present invention.

The invention includes the use of any compounds of described above containing one or more asymmetric carbon atoms may occur as racemates and racemic mixtures, single enantiomers, diastereomeric mixtures and individual diastereomers. Isomers shall be defined as being enantiomers and diastereomers. All such isomeric forms of these compounds are expressly included in the present invention. Each stereogenic carbon may be in the R or S configuration, or a combination of configurations.

Some of the compounds of the invention can exist in more than one tautomeric form.

The invention includes methods using all such tautomers.

All terms as used herein in this specification, unless otherwise stated, shall be understood in their ordinary meaning as known in the art. For example, “C₁₋₄alkoxy” is a C₁₋₄alkyl with a terminal oxygen, such as methoxy, ethoxy, propoxy, butoxy. All alkyl, alkenyl and alkynyl groups shall be understood as being branched or unbranched where structurally possible and unless otherwise specified. Other more specific definitions are as follows:

Carbocycles include hydrocarbon rings containing from three to twelve carbon atoms. These carbocycles may be either aromatic or non-aromatic ring systems. The non-aromatic ring systems may be mono- or polyunsaturated. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic or spirocyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure.

The term “heteroaryl” shall be understood to mean an aromatic 5-8 membered monocyclic or 8-11 membered bicyclic ring containing 1-4 heteroatoms such as N, O and S.

Unless otherwise stated, heterocycles and heteroaryl include but are not limited to, for example azetidinyl, furanyl, pyranyl, benzoxazolyl, benzothiazolyl, benzimidazolyl, tetrahydropyranyl, dioxanyl, tetrahydrofuranyl, oxazolyl, isoxazolyl, thiazolyl, pyrazolyl, pyrrolyl, pyrrolidinyl, pyrrolidinone, imidazolyl, thienyl, thiadiazolyl, thiomorpholinyl, 1,1-dioxo-1λ⁶-thiomorpholinyl, morpholinyl, pyridinyl, pyridinone, pyrimidinyl, pyridazinyl, pyrazinyl, triazinyl, pyrrolidinyl, piperidinyl, piperazinyl, purinyl, quinolinyl, Dihydro-2H-quinolinyl, tetrahydroquinolinyl, isoquinolinyl, quinazolinyl, indazolyl, indolyl, isoindolyl, benzofuranyl, benzopyranyl and benzodioxolyl.

The term “heteroatom” as used herein shall be understood to mean atoms other than carbon such as O, N, S and P.

In all alkyl groups or carbon chains one or more carbon atoms can be optionally replaced by heteroatoms: O, S or N, it shall be understood that if N is not substituted then it is NH, it shall also be understood that the heteroatoms may replace either to terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative. For example, quinolinyl may include decahydroquinolinyl and tetrahydroquinolinyl, naphthyl may include its hydrogenated derivatives such as tetrahydranaphthyl. Other partially or fully hydrogenated derivatives of the aryl and heteroaryl compounds described herein will be apparent to one of ordinary skill in the art.

As used herein, “nitrogen” and “sulfur” include any oxidized form of nitrogen and sulfur and the quaternized form of any basic nitrogen. For example, for an —S—C₁₋₆ alkyl radical, unless otherwise specified, this shall be understood to include —S(O)—C₁₋₆ alkyl and —S(O)₂—C₁₋₆ alkyl.

The term “alkyl” refers to a saturated aliphatic radical containing from one to ten carbon atoms or a mono- or polyunsaturated aliphatic hydrocarbon radical containing from two to twelve carbon atoms. The mono- or polyunsaturated aliphatic hydrocarbon radical containing at least one double or triple bond, respectively. “Alkyl” refers to both branched and unbranched alkyl groups. It should be understood that any combination term using an “alk” or “alkyl” prefix refers to analogs according to the above definition of “alkyl”. For example, terms such as “alkoxy”, “alkylhio” refer to alkyl groups linked to a second group via an oxygen or sulfur atom. “Alkanoyl” (or acyl) refers to an alkyl group linked to a carbonyl group (C═O).

The term “halogen” as used in the present specification shall be understood to mean bromine, chlorine, fluorine or iodine, preferably fluorine. The definitions “halogenated”, “partially or fully halogenated”; “partially or fully fluorinated”; “substituted by one or more halogen atoms”, includes for example, mono, di or tri halo derivatives on one or more carbon atoms. For alkyl, a nonlimiting example would be —CH₂CHF₂, —CF₃ etc.

Each alkyl (or any term using an “alk” or “alkyl” prefix), carbocycle, heterocycle or heteroaryl, or the analogs thereof, described herein shall be understood to be optionally partially or fully halogenated.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the invention.

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)-4+ salts.

In addition, within the scope of the invention is use of prodrugs of compounds of the invention. Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound to disclosed hereinabove, thereby imparting the desired pharmacological effect.

The compounds of formula I may be made using the general synthetic methods described below, which also constitute part of the invention.

### General Synthetic Methods

The invention additionally provides methods for making the compounds of formula I. The compounds of the invention may be prepared by the general methods and examples presented below, and methods known to those of ordinary skill in the art and reported in the chemical literature. Unless otherwise specified, solvents, temperatures, pressures, and other reaction conditions may be readily selected by one of ordinary skill in the art. Specific procedures are provided in the Synthetic Examples section. Starting materials used are either commercially available or easily prepared from commercially available materials by those skilled in the art. Reaction progress may be monitored by conventional methods such as thin layer chromatography (TLC) or high performance liquid chromatography (HPLC). Intermediates and products may be purified by methods known in the art, including flash chromatography, HPLC or recrystallization. Amide bond formations may be carried out by standard coupling conditions well-known in the art (see, for example, M. Bodanszky, The Practice of Peptide Synthesis (Springer-Verlag: 1984), which is hereby incorporated by reference in its entirety), for example, by reacting a carbocylic acid and an amine in the presence of 1-(3-dimethylaminopropyl)-3-ethylcarbodiimide (EDC) and 1-hydroxybenzotriazole. Racemic compounds of this invention may be prepared in enantiomerically pure or enriched form by methods known in the art, including separation using chiral HPLC, resolution using a chiral reagent or auxiliary, and other asymmetric synthesis methods known in the art. If certain functional groups are incompatible under the reaction conditions, protection/deprotection of these groups may be carried out using reagents and conditions readily selected by one of ordinary skill in the art (see, for example, P. G. M. Wuts and T. W. Greene, Greene's Protective Groups in Organic Synthesis (John Wiley & Sons: 2006), which is hereby incorporated by reference in its entirety).

The methods described below and in the Synthetic Examples section may be used to to prepare the compounds of formula I.

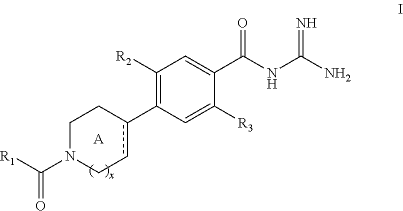

In the schemes below, R¹-R³ and x shall have the meanings defined in the detailed description of formula I.

Compounds of formula I may be prepared as shown in Scheme 1. As illustrated in Scheme I, a Suzuki reaction with a benzoic acid ester (R′ is alkyl) (II) substituted with R₂ and R₃ and bearing a suitable leaving group in the 4-position such as a bromine or a triflate group and a N-protected tetrahydropiperidine (III) bearing a boronic ester group in the 4-position such as the 4,4,5,5-tetramethyl[1,3,2]dioxaborolan-2-yl group shown on III, in the presence of a suitable catalyst such as (Ph₃P)₄Pd and a suitable base such as K₂CO₃ provides intermediate IV. Reduction of the olefin, for example by treatment with hydrogen in the presence of a suitable catalyst such as Pd/C provides V. Hydrolysis of the ester provides carboxylic acid VI. Coupling of V with a suitably protected guanidine such as N-carbobenzyloxyguanidine provides VII. Deprotection of the piperidine ring, for example by treatment with acid such as HCl for the t-Boc protecting group shown, provides the piperidine intermediate VIII. Coupling of the piperidine amine with the desired RiCO₂H provides IX. Removal of the protecting group on the guanidine, for example by treatment with hydrogen in the presence of a suitable catalyst such as Pd/C for the carbobenzyloxy protecting group shown, provides the desired compound of formula I. Compounds of formula I having a tetrahydropyridin-4-yl ring may be obtained by omitting the reduction step from intermendiate IV to intermediate V

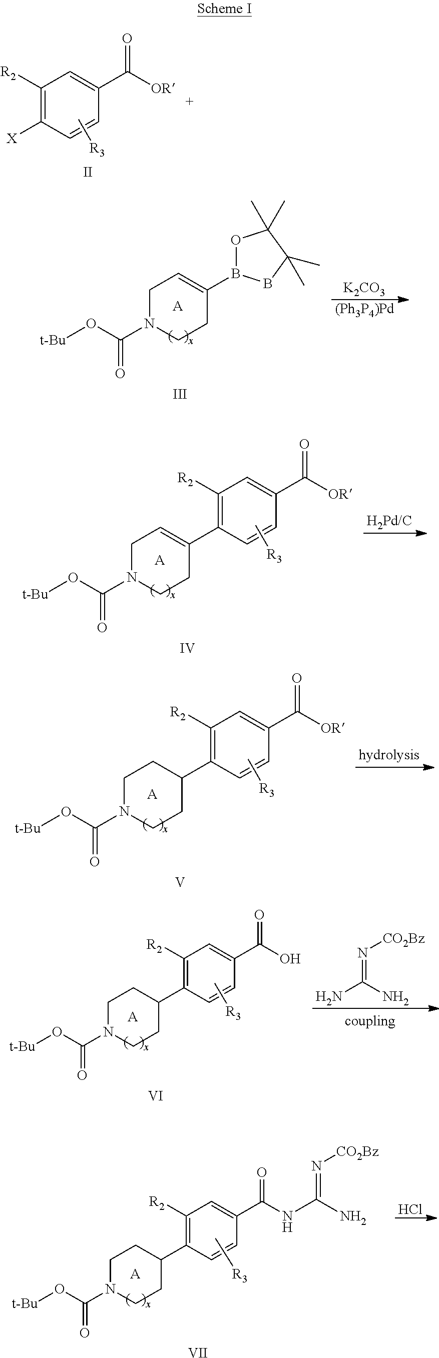

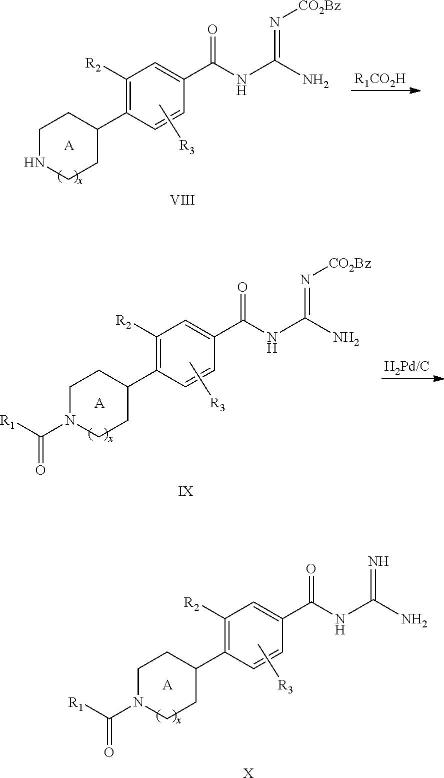

Intermediate III shown in Scheme I may be prepared as illustrated in Scheme II. An N-protected 4-oxoheterocycle such as the t-Boc intermediate XI shown is converted to the triflate ester XII for example by treatment with N-phenyltrifluoromethylsulfonimide in the presence of a suitable base such as lithium bis(trimethylsilyl)amide. Intermediate XII is treated with bis(pinocolato)diboron in the presence of a suitable catalyst such as 1,1′ bis(diphenylphosphino)ferrocenedichloropalladium and a suitable ligand such as di-phenyl phosphinoferrocene (dppf) to provide the desired intermediate III.

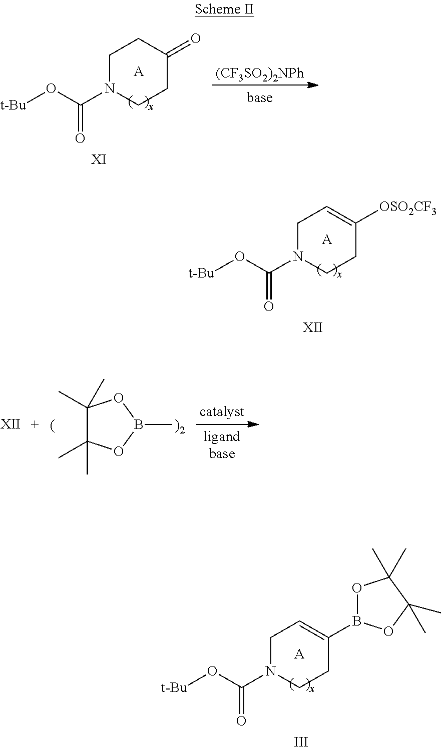

The order of the synthetic sequence shown in Scheme I could be modified as would be apparent to one skilled in the art and as illustrated in the synthetic examples provided below.

### SYNTHETIC EXAMPLES

### Example 1

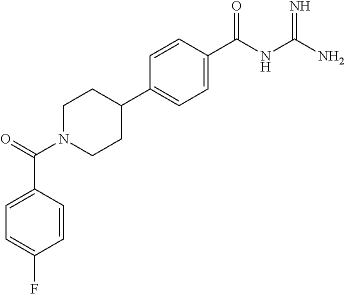

### N-{4-[1-(4-Fluoro-benzoyl)-piperidin-4-yl]-benzoyl}-guanidine

### Step A: 4-(4-(N-Carbobenzyloxy)-guanidinocarbonyl-phenyl)-piperidine-1-carboxylic acid tert-butyl ester

To a solution of 4-(4-carboxy-phenyl)-piperidine-1-carboxylic acid tert-butyl ester (1.00 g, 3.30 mmol) in N-methylpyrrolidinone (30 mL) is added 2-chloro-1-methylpyridinium iodide (1.08 g, 4.22 mmol) and the mixture is stirred for 90 minutes. N-carbobenzyloxy-guanidine (823 mg, 4.26 mmol) is then added followed by N,N-diisopropylethylamine (1.63 mL, 9.82 mmol) and the reaction is stirred overnight. The mixture is partitioned between water and ethyl acetate and the layers are separated. The combined organic phases are washed twice with water, then with brine, dried over Na₂SO₄, filtered, and the solvent is removed in vacuo to give the desired product as a glassy yellow solid (1.12 g, 2.33 mmol). LCMS: 481.49 (M+H⁺).

### Step B: N-(4-Piperidin-4-yl-benzoyl)-N′-carbonezyloxy-guanidine

To a solution of the product of Step A (1.12 g, 2.33 mmol) in dichloromethane (20 mL) is added 4 M hydrogen chloride in 1,4-dioxane (10 mL, 40 mmol) and the resulting mixture is stirred for 2 hours. The solvent is removed in vacuo to give the desired product hydrochloride salt as a colorless solid (970 mg, 2.33 mmol). LCMS: 381.42 (M+H⁺).

### Step C: N-{4-[1-(4-Fluoro-benzoyl)-piperidin-4-yl]-benzoyl}-N′-carbobenzyloxy-guanidine

To a solution of 4-fluorobenzoic acid (37 mg, 0.26 mmol) in N,N-dimethylformamide (3 mL) is added di-imidazol-1-yl-methanone (43 mg, 0.26 mmol) and the resulting mixture is stirred for 90 minutes. The product from Step B (100 mg, 0.24 mmol) and N,N-diisopropylethylamine (0.13 mL, 0.72 mmol) are then added and the mixture is stirred for 16 hours. The mixture is diluted with ethyl acetate, washed twice with water, once with brine, dried over Na₂SO₄, filtered, and the solvent is removed in vacuo. The crude material is purified via silica gel chromatography using a gradient elution of 0-70% ethyl acetate/hexane to afford the desired product as a colorless solid (92 mg, 76%). LCMS: 503.44 (M+H⁺).

### Step D: N-{4-[1-(4-Fluoro-benzoyl)-piperidin-4-yl]-benzoyl}-guanidine

To a suspension of the product of Step C (87 mg, 0.17 mmol) in ethanol (4 mL), methanol (2 mL) and dichloromethane (2 mL) under an argon atmosphere is added 10 wt % palladium on carbon (37 mg, 0.017 mmol) and the reaction is stirred under a hydrogen atmosphere for 6 hours. The mixture is filtered through Celite and the residue is purified via preparative HPLC using a gradient elution from 10-90% acetonitrile/water with 0.1% trifluoroacetic acid to obtain the desired product as a trifluoroacetic acid salt (52 mg, 62%). LCMS: 369.70 (M+H⁺).

The following compounds are prepared using the procedures of Example 1 and substituting for the appropriate starting materials.

| Example | | | m/z |
|---|---|---|---|
| No. | Structure | Name | [M + H⁺] |
| 2 | 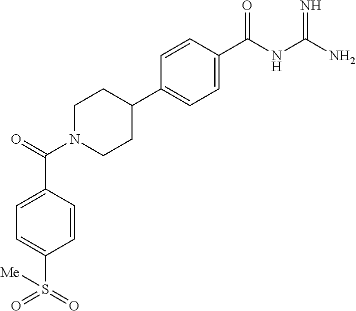 | N-{4-[1-(4- methanesulfonyl- benzoyl)-piperidin-4-yl]- benzoyl}-guanidine | 429.65 |
| 3 | 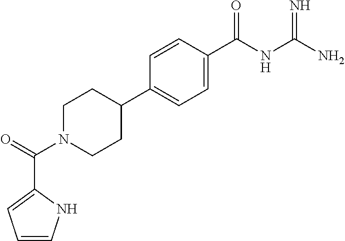 | N-{4-[1-(1H-pyrrole-2- carbonyl)-piperidin-4-yl]- benzoyl}-guanidine | 340.40 |
| 4 | 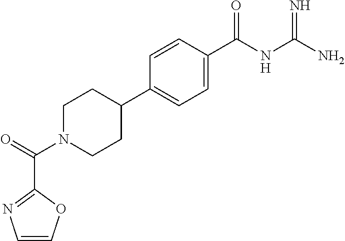 | N-{4-[1-(oxazole-2- carbonyl)-piperidin-4-yl]- benzoyl}-guanidine | 342.38 |

### Example 5

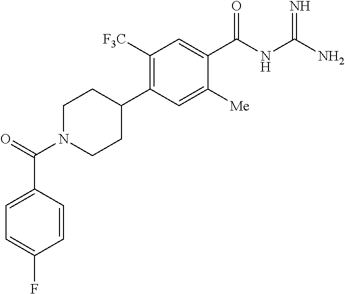

### N-{4-[1-(4-Fluoro-benzoyl)-piperidin-4-yl]-2-methyl-5-trifluoromethyl-benzoyl}-guanidine

### Step A: 4-Hydroxy-5-iodo-2-methyl-benzoic acid

To a solution of 4-hydroxy-2-methyl-benzoic acid (7.00 g, 46.0 mmol) in methanol (125 mL) is added sodium hydroxide (3.68 g, 92.0 mmol) and sodium iodide (15.86 g, 105.8 mmol) and the mixture is heated to reflux. While at reflux, a sodium hypochlorite solution containing approximately 4% of active chlorine (115 mL, 1.69 mol) is added dropwise over 30 min Reflux is continued for 30 min and the mixture is allowed to cool to rt. A solution of 10% sodium thiosulfate (92 mL) is added, followed by the addition of concentrated hydrochloric acid (21 mL). The mixture is extracted with ethyl acetate and the combined organic phases are washed with water until neutral pH is obtained. The organic phase is dried over Na₂SO₄ and the solvent is removed in vacuo. The crude material is purified via silica gel chromatography using a gradient elution of 0-30% ethyl acetate/hexanes to afford the desired product as an off-white solid (9.23 g, 72%).

### Step B: 4-Hydroxy-5-iodo-2-methyl-benzoic acid methyl ester

To a solution of the product from Step A (9.23 g, 33.2 mmol) in methanol (100 mL) is to added acetyl chloride (25.8 mL, 332 mmol). The mixture is stirred at 50° C. overnight. The mixture is evaporated to dryness and the residue is partitioned between ethyl acetate and saturated aqueous sodium bicarbonate. The layers are separated and the aqueous layer is further extracted with ethyl acetate. The combined organic phases are washed with saturated aqueous sodium bicarbonate, brine, dried over Na₂SO₄ and the solvent is removed in vacuo to afford the desired product as an off-white solid (9.70 g, quantitative).

### Step C: 5-Benzyloxy-5-iodo-2-methyl-benzoic acid methyl ester

To a solution of the product from Step B (9.70 g, 33.2 mmol) in N,N-dimethylformamide (130 mL) is added benzyl bromide (4.46 mL, 36.5 mmol) and potassium carbonate (13.8 g, 99.6 mmol). The mixture is stirred at 80° C. for 2 hours and is then cooled to room temperature. The mixture is partitioned between ethyl acetate and water. The layers are separated and the aqueous layer is further extracted with ethyl acetate. The combined organic phases are washed with brine, dried over Na₂SO₄ and the solvent is removed in vacuo. The crude material is purified via silica gel chromatography using a gradient elution of 0-8% ethyl acetate/hexanes to afford the desired product as a colorless solid (8.32 g, 66%). LCMS: 383.00 (M+H⁺).

### Step D: 4-Benzyloxy-2-methyl-5-trifluoromethyl-benzoic acid methyl ester

To a solution of the product from Step C (8.32 g, 21.8 mmol) in 1-methyl-2-pyrrolidone (100 mL) is added potassium trifluoroacetate (16.6 g, 108.8 mmol) and copper(I) iodide (20.7 g, 108.8 mmol). The reaction is stirred at 150° C. for 5 hours and then is allowed to cool to room temperature. The mixture is partitioned between ethyl acetate and saturated aqueous ammonium chloride and is filtered through Celite, washing the filter cake with ethyl acetate. The layers of the filtrate are separated and the aqueous phase is further extracted with ethyl acetate. The combined organic phases are washed with brine, dried over Na₂SO₄ and the solvent is removed in vacuo. The crude material is purified via silica gel chromatography using a gradient elution of 0-8% ethyl acetate/hexanes to afford the desired product as an off-white solid (6.30 g, 89%). LCMS: 325.20 (M+H⁺).

### Step E: 4-Hydroxy-2-methyl-5-trifluoromethyl-benzoic acid methyl ester

To a solution of the product from Step D (6.30 g, 19.4 mmol) in ethanol (150 mL) under an argon atmosphere is added 10% palladium on carbon (400 mg, 0.19 mmol, wet, Degussa type). The mixture is stirred under a hydrogen atmosphere overnight. The mixture is filtered through a pad of Celite rinsing with ethanol and the solvent is evaporated in vacuo to afford the desired product (4.34 g, 95%). LCMS: 235.20 (M+H⁺).

### Step F: 2-Methyl-4-trifluoromethanesulfonyloxy-5-trifluoromethyl-benzoic acid methyl ester

To a solution of the product from Step E (4.34 g, 18.5 mmol) in N,N-dimethylformamide is added N,N-diisopropylethylamine (3.55 mL, 20.4 mmol) followed by N-phenyltrifluoromethanesulfonimide. The reaction is stirred at room temperature overnight. The mixture is partitioned between ethyl acetate and saturated aqueous sodium bicarbonate and the aqueous layer is back-extracted with ethyl acetate. The combined organic phases are washed with brine, dried over Na₂SO₄ and the solvent is removed in vacuo to afford the desired crude product (9.49 g, approx. 70% purity) which is used in the next step without purification.

### Step G: 4-(4-Methoxycarbonyl-5-methyl-2-trifluoromethyl-phenyl)3,6-dihydro-2H-pyridine-1-carboxylic acid tert-butyl ester

Each reactant is divided into four equal portions and is placed into four 20 mL microwave reaction vials: To a solution of the product from Step F (9.49 g, approx. 70% purity, 18.1 mmol) in tetrahydrofuran (48 mL) is added 4-(4,4,5,5-tetramethyl-[1,3,2] dioxaborolan-2-yl)-3,6-dihydro-2H-pyridine-1-carboxylic acid tert-butyl ester (5.61 g, 18.1 mmol) and 2 M aqueous potassium carbonate (18.1 mL, 36.3 mmol). After the mixture is degassed, tetrakis(triphenylphosphine)palladium(0) (3.78 g, 3.27 mmol) is added. Each vial is sealed with a Teflon lined septum cap and is irradiated in a microwave reactor at 110° C. for 30 min. After cooling to room temperature, the mixtures from all of the vials are pooled together and are partitioned between ethyl acetate and water. The mixture is filtered to remove the remaining undissolved solid, rinsing with ethyl acetate and water. The layers are separated and the aqueous layer is extracted with ethyl acetate. The combined organic phases are dried over Na₂SO₄ and the solvent is removed in vacuo. The crude material is purified via silica gel chromatography using a gradient elution of 0-8% ethyl acetate/hexanes to afford the desired product as a light yellow oil (5.68 g, 78%). LCMS: 422.20 (M+Na⁺).

### Step H: 4-(4-Methoxycarbonyl-5-methyl-2-trifluoromethyl-phenyl)-piperidine-1-carboxylic acid tert-butyl ester

To a solution of the product from Step G (5.68 g, 14.2 mmol) in ethanol (70 mL) under an argon atmosphere is added 10% palladium on carbon (200 mg, 0.09 mmol, wet, Degussa type). The mixture is stirred under a hydrogen atmosphere for 16 hours. The mixture is filtered through a pad of Celite rinsing with dichloromethane. The solvent is removed in vacuo to afford the desired product as a colorless solid (5.15 g, 90%). LCMS: 346.20 (M+H⁺).

### Step I: 4-(4-Carboxy-5-methyl-2-trifluoromethyl-phenyl)-piperidine-1-carboxylic acid tert-butyl ester)

To a solution of the product from Step H (5.15 g, 12.8 mmol) in methanol (40 mL) and water (20 mL) is added lithium hydroxide monohydrate (807 mg, 19.2 mmol). The reaction is stirred at 40° C. overnight and then is cooled to room temperature. The methanol is removed in vacuo and the aqueous mixture is acidified to pH 5 with 1 N hydrochloric acid. The resulting solid is collected by filtration and dried in vacuo at 50° C. to afford the desired product as a colorless solid (5.00 g, quantitative). LCMS: 386.20 (M−H⁺).

Step J: 4-[5-Methyl-4-(N-(carbobenzyloxy)-guanidinocarbonyl)-2-trifluoromethyl-phenyl]-piperidine-1-carboxylic acid tert-butyl ester

To a solution of the product from Step I (5.00 g, 12.9 mmol) in 1-methyl-2-pyrrolidone (50 mL) is added 2-chloro-1-methylpyridinium iodide (3.63 g, 14.2 mmol). The mixture is stirred at room temperature for 30 minutes. N-carbobenzyloxy-guanidine to (2.99 g, 15.5 mmol) and N,N-diisopropylethylamine are added to the reaction mixture.

The mixture is allowed to stir at room temperature overnight. The mixture is partitioned between ethyl acetate and water and the layers are separated. The aqueous layer is further extracted with ethyl acetate and the combined organic phases are washed with water, brine, dried over Na₂SO₄ and the solvent is removed in vacuo. The crude material is purified via silica gel chromatography using a gradient elution of 10-20% ethyl acetate/hexanes to afford the desired product (5.68 g, 78%). LCMS: 563.20 (M+H⁺).

### Step K: N-(2-methyl-4-piperidin-4-yl-5-trifluoromethyl-benzoyl)-N′-(carbobenzyloxy) guanidine

To a solution of the product from Step J (5.68 g, 10.1 mmol) in methanol (20 mL) is added 4 M hydrogen chloride in 1,4-dioxane (10 mL). The mixture is stirred at room temperature for 6 hours. The solvent is removed in vacuo to afford the desired product as the hydrochloride salt (5.0 g, quantitative). LCMS: 463.20 (M+H⁺).

### Step L: N-{4-[1-(4-Fluoro-benzoyl)-piperidin-4-yl]-2-methyl-5-trifluoromethyl-benzoyl}-guanidine

To a solution of 4-fluorobenzoic acid (70 mg, 0.49 mmol) in N,N-dimethylformamide (3 mL) is added di-imidazol-1-yl-methanone (80 mg, 0.49 mmol). The mixture is stirred at room temperature for 90 minutes, after which the product from Step K (225 mg, 0.45 mmol) and N,N-diisopropylethylamine (0.24 mL, 1.35 mmol) are added. The mixture is allowed to stir at room temperature overnight. The mixture is partitioned between ethyl acetate and water. The layers are separated and the aqueous layer is further extracted with ethyl acetate. The combined organic phases are washed with brine, dried over Na₂SO₄, and the solvent is removed in vacuo. To a solution of the crude residue in ethanol (5 mL) purged with argon is added 10% palladium on carbon (25 mg, 0.01 mmol, wet, Degussa type). The mixture is stirred under a hydrogen atmosphere. After 2 hours, the mixture is filtered through glass filter paper rinsing with ethyl acetate. The solvent is evaporated and the residue is purified via preparative HPLC using a gradient elution from 10-75% acetonitrile/water with 0.1% trifluoroacetic acid to obtain the desired product as a trifluoroacetic acid salt (109 mg, 43%). LCMS: 451.20 (M+H⁺).

The following compounds are prepared using the procedures of Example 5 and substituting for the appropriate starting materials.

| Example | | | m/z |
|---|---|---|---|
| No. | Structure | Name | [M + H⁺] |
| 6 | 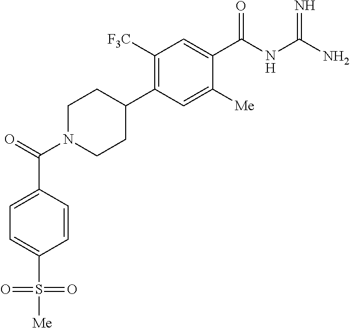 | N-{4-[1-(4- methanesulfonyl- benzoyl)-piperidin-4- yl]-2-methyl-5- trifluoromethyl- benzoyl}-guanidine | 511.31 |
| 7 | 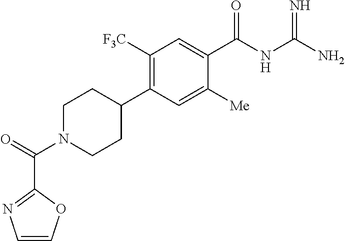 | N-{4-methyl-4-[1- (oxazole-2-carbonyl)- piperidin-4-yl]-5- trifluoromethyl- benzoyl}-guanidine | 424.20 |
| 8 | 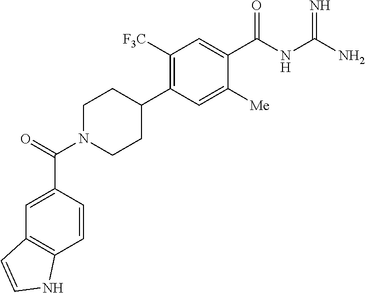 | N-{4-[1-(1H-indole-5- carbonyl)-piperidin-4- yl]-2-methyl-5- trifluoromethyl- benzoyl}-guanidine | 472.20 |
| 9 | 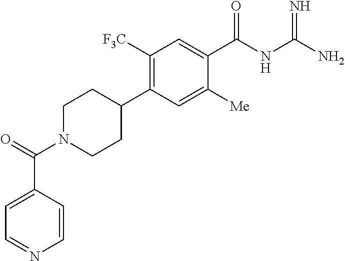 | N-{2-methyl-4-[1- (pyridine-4-carbonyl)- piperidin-4-yl]-5- trifluoromethyl- benzoyl}-guanidine | 434.34 |
| 10 | 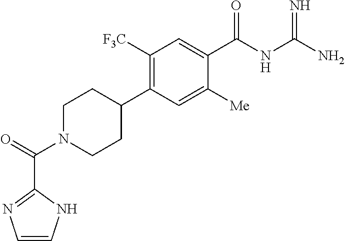 | N-{4-[1-(1H-imidazole- 2-carbonyl)-piperidin-4- yl]-2-methyl-5- trifluoromethyl- benzoyl}-guanidine | 423.33 |
| 11 | 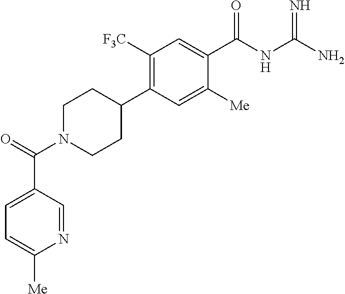 | N-{2-methyl-4-[1-(6- methyl-pyridine-3- carbonyl)-piperidin-4- yl]-5-trifluoromethyl- benzoyl}-guanidine | 448.20 |
| 12 | 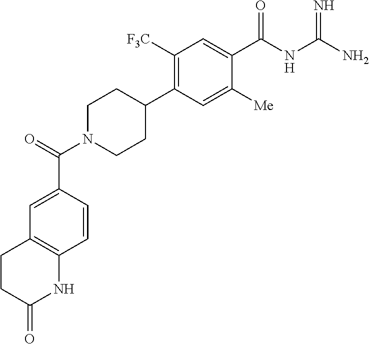 | N-{2-methyl-4-[1-(2- oxo-1,2,3,4-tetrahydro- quinoline-6-carbonyl)- piperidin-4-yl]-5- trifluoromethyl- benzoyl}-guanidine | 502.20 |
| 13 | 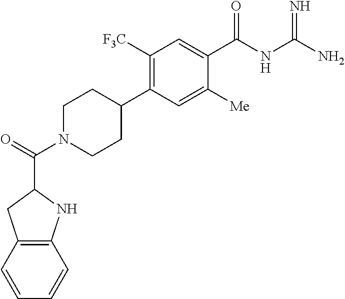 | N-{4-[1-(1H-indole-2- carbonyl)-piperidin-4- yl]-2-methyl- 5-trifluoromethyl- benzoyl}-guanidine | 472.20 |
| 14 | 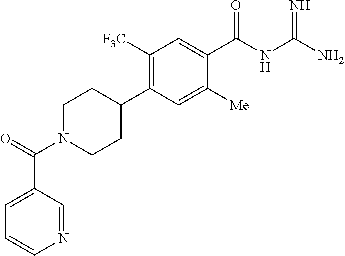 | N-{2-methyl-4-[1- (pyridine-3-carbonyl)- piperidin-4-yl]-5- trifluoromethyl- benzoyl}-guanidine | 434.20 |
| 15 | 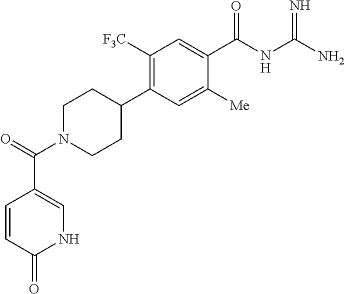 | N-{2-methyl-4-[1-(6- oxo-1,6-dihydro- pyridine-3-carbonyl)- piperidin-4-yl]-5- trifluoromethyl- benzoyl}-guanidine | 450.20 |
| 16 | 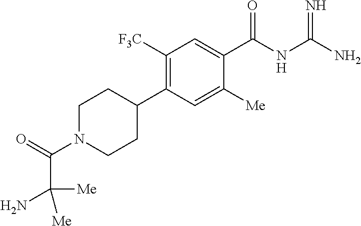 | N-[4-(1-(2-amino-2- methyl-propionyl)- piperidin-4-yl]-2- methyl-5- trifluoromethyl- benzoyl}-guanidine | 414.20 |
| 17 | 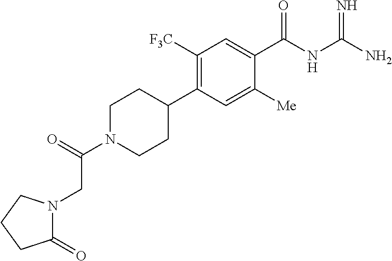 | N-(2-methyl-4-{l-[2-(2- oxo-pyrrolidin-1-yl)- acetyl]-piperidin-4-yl}- 2-methyl-5- trifluoromethyl- benzoyl)-guanidine | 454.20 |
| 18 | 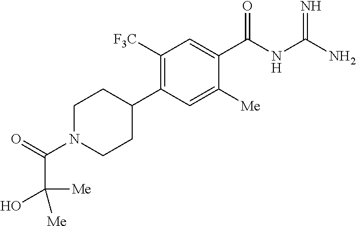 | N-[4-(1-(2-hydroxy-2- methyl-propionyl)- piperidin-4-yl]-2- methyl-5- trifluoromethyl- benzoyl}-guanidine | 415.20 |
| 19 | 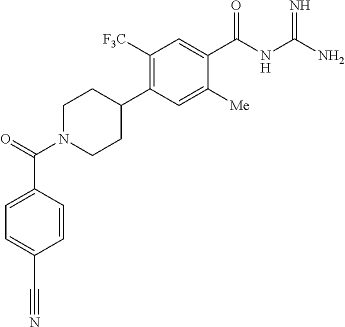 | N-{4-[1-(4-cyano- benzoyl)-piperidin-4- yl]-2-methyl-5- trifluoromethyl- benzoyl}-guanidine | 458.37 |
| 20 | 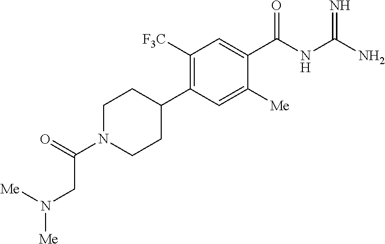 | N-{4-[1-(2- dimethylamino-acetyl)- piperidin-4-yl]-2- methyl-5- trifluoromethyl- benzoyl}-guanidine | 414.63 |

### Example 21

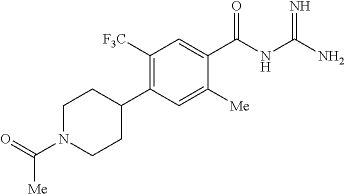

N-[4-(1-Acetyl-piperidin-4-yl]-2-methyl-5-trifluoromethyl-benzoyl}-guanidine

To a solution of the product of Example 5 Step K (250 mg, 0.50 mmol) in dichloromethane (2.5 mL) is added N,N-diisopropylethylamine (0.26 mL, 1.50 mmol) to followed by acetic anhydride (0.048 mL, 0.50 mmol). The mixture is stirred at room temperature for 45 minutes. The mixture is diluted with dichloromethane and is washed with saturated aqueous sodium bicarbonate. The aqueous layer is back extracted with dichloromethane and the combined organic phases are washed with brine, dried over Na₂SO₄ and the solvent is removed in vacuo. To a solution of the residue in a mixture of N,N-dimethylformamide (1 mL) and ethanol (10 mL) under an argon atmosphere is added 10% palladium on carbon (50 mg, 0.02 mmol, wet, Degussa type). The mixture is stirred under a hydrogen atmosphere overnight. The mixture is filtered through glass filter paper rinsing with ethyl acetate. The solvent is evaporated and the residue is purified via preparative HPLC using a gradient elution from 10-75% acetonitrile/water with 0.1% trifluoroacetic acid to obtain the desired product as a trifluoroacetic acid salt to (85 mg, 35%). LCMS: 371.20 (M+H⁺).

### Example 22

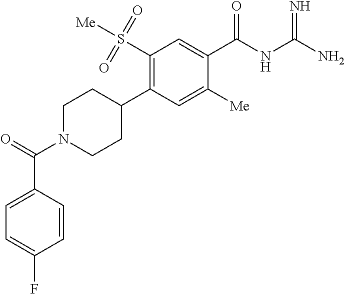

### N-{4-[1-(4-Fluorobenzoyl)-piperidin-4-yl]-5-methanesulfonyl-2-methyl-benzoyl}-guanidine

### Step A: 4-Bromo-5-chlorosulfonyl-2-methyl-benzoic acid

To chlorosulfonic acid (15.0 mL, 226 mmol) cooled to 0° C. is added 4-bromo-2-methyl-benzoic acid (5.00 g, 23.3 mmol), portionwise over 2 minutes. The reaction is allowed to warm to room temperature and is then heated at 100° C. for 2 hours. The reaction is cooled to room temperature and is added dropwise (very slowly) to ice (750 g). The resulting solid is collected by filtration and is dried in vacuo to afford the desired product as a colorless solid (6.36 g, 87%). LCMS: 313.00 (M−H⁺).

### Step B: 4-Bromo-5-methanesulfonyl-2-methyl-benzoic acid

To a stirred solution of sodium sulfite (3.58 g, 28.4 mmol) and sodium bicarbonate (8.52 g, 101.4 mmol) in water (75 mL) at 70° C. is added a solution of the product from Step A (6.36 g, 20.3 mmol) in tetrahydrofuran (25 mL), dropwise over 20 minutes. After the addition is complete, the mixture is stirred at 70° C. for 1 hour and then cooled to room temperature. Iodomethane (6.31 mL, 101.4 mmol) is added and the reaction is allowed to stir at 50° C. overnight and is then cooled to room temperature. The tetrahydrofuran is removed in vacuo and the aqueous mixture is acidified to pH 5 with 4 N hydrochloric acid. The resulting precipitate is collected by filtration and is dried in vacuo at 50° C. to afford the desired product as a colorless solid (4.88 g, 82%). LCMS: 293.00 (M+H⁺).

### Step C: 4-Bromo-5-methanesulfonyl-2-methyl-benzoic acid methyl ester

To the solution of the product from Step B (4.88 g, 16.6 mmol) in methanol (85 mL) is added acetyl chloride (12.9 mL, 166 mmol). The mixture is stirred at 50° C. overnight. The mixture is evaporated to dryness and the residue is partitioned between ethyl acetate and saturated aqueous sodium bicarbonate. The layers are separated and the aqueous layer is back extracted with ethyl acetate. The combined organic phases are washed with saturated aqueous sodium bicarbonate, brine, dried over Na₂SO₄ and the solvent is removed in vacuo to afford the desired product as an off-white solid (4.47 g, 87%). LCMS: 307.00 (M+H⁺).

Step D: 4-(2-Methansulfonyl-4-methoxycarbonyl-5-methyl-phenyl)-3,6-dihydro-2H-pyridine-1-carboxylic acid tert-butyl ester)

Each reactant is divided into three equal portions and is placed into three 20 mL

microwave reaction vials: To a solution of the product from Step C (3.50 g, 11.4 mmol) in 1,4-dioxane (8.5 mL) is added 4-(4,4,5,5-tetramethyl-[1,3,2] dioxaborolan-2-yl)-3,6-dihydro-2H-pyridine-1-carboxylic acid tert-butyl ester (3.52 g, 11.4 mmol) and 2 M aqueous potassium carbonate (11.4 mL, 22.8 mmol). After the mixture is degassed, tetrakis(triphenylphosphine)palladium(0) (1.32 g, 1.14 mmol) is added. Each vial is sealed with a Teflon lined septum cap and irradiated in a microwave reactor at 170° C. for 30 minutes. After cooling, the mixtures from all of the vials are pooled together and are partitioned between ethyl acetate and water. The mixture is filtered to remove undissolved solid, rinsing with ethyl acetate and water. The layers of the filtrate are separated and the aqueous layer is extracted with ethyl acetate. The combined organic phases are dried over Na₂SO₄ and the solvent is removed in vacuo. The crude material is purified via silica gel chromatography using a gradient elution of 10-40% ethyl acetate/hexanes to afford the desired product as a light yellow oil (4.8 g, quantitative). LCMS: 432.20 (M+Na⁺).

### Step E: 4-(2-Methanesulfonyl-4-methoxycarbonyl-5-methyl-phenyl)-piperidine-1-carboxylic acid tert-butyl ester

To a solution of the product from Step D (2.70 g, 6.59 mmol) in acetic acid (40 mL) under an argon atmosphere is added platinum (IV) oxide (750 mg). The mixture is stirred under a hydrogen atmosphere overnight. The mixture is filtered through a pad of Celite rinsing with ethyl acetate. The solvent is evaporated under reduced pressure and the residue is partitioned between ethyl acetate and saturated aqueous sodium bicarbonate. The layers are separated and the aqueous layer is further extracted with ethyl acetate. The combined organic phases are washed with brine, dried over Na₂SO₄ and the solvent is removed in vacuo to afford the desired product as an off-white solid (2.24 g, 83%). LCMS: 312.20 (M+H⁺).

### Step F: 4-(4-Carboxy-2-methanesulfonyl-5-methyl-phenyl)-piperidine-1-carboxylic acid tert-butyl ester)

The compound is prepared according to the procedure from Example 5 Step I, starting from the product of Example 22 Step E (2.24 g, 5.44 mmol) using lithium hydroxide monohydrate (343 mg, 8.17 mmol), to afford the desired product (1.85 g, 86%).

### Step G: 4-[2-Methanesulfonyl-5-methyl-4-(N-(carbobenzyloxy)-guanidinocarbonyl)-phenyl]-piperidine-1-carboxylic acid tert-butyl ester

The compound is prepared according to the procedure from Example 5 Step J, starting from the product of Example 22 Step F (1.85 g, 4.65 mmol), using 2-chloro-1-methylpyridinium iodide (1.31 g, 5.12 mmol), N-carbobenzyloxy-guanidine (1.08 g, 5.59 mmol), and N,N-diisopropylethylamine (2.43 mL, 13.9 mmol), and purified via silica gel chromatography using a gradient elution of 25-50% ethyl acetate in hexanes to afford the desired product (2.15 g, 81%). LCMS: 573.20 (M+H⁺).

### Step H: 4-[5-Methanesulfonyl-2-methyl-4-piperidine-4-yl-benzoyl)-N-(carbobenzyloxy)-guanidine

The compound is prepared according to the procedure from Example 5 Step K, starting from the product of Example 22 Step G (2.15 g, 3.75 mmol) and 4 M hydrochloric acid in 1,4-dioxane (7 mL), to afford the desired product as the hydrochloride salt (1.91 g, to quantitative). LCMS: 473.20 (M+H⁺).

### Step I: N-{4-[1-(4-Fluorobenzoyl)-piperidin-4-yl]-5-methanesulfonyl-2-methyl-benzoyl}-guanidine

The compound is prepared according to the procedure from Example 5 Step L, starting from the product of Example 22 Step H (175 mg, 0.29 mmol), using 4-fluorobenzoic acid (45 mg, 0.32 mmol), di-imidazol-1-yl-methanone (53 mg, 0.32 mmol), and N,N-diisopropylethylamine (0.15 mL, 0.88 mmol), and purified by preparative HPLC using a gradient elution from 10-75% acetonitrile/water with 0.1% trifluoroacetic acid as the eluent to give the desired intermediate which is deprotected using 10% palladium on carbon (50 mg, wet, Degussa type) to afford the desired product as the trifluoroacetic acid salt (93 mg, 55%). LCMS: 461.20 (M+H⁺).

### Example 23

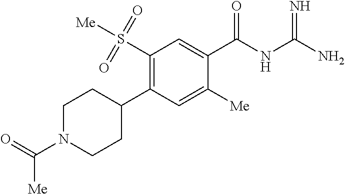

N-{4-[1-Acetyl-piperidin-4-yl]-5-methanesulfonyl-2-methyl-benzoyl}-guanidine

The compound is prepared according to the procedure from Example 19, starting from the product of Example 22 Step H (200 mg, 0.39 mmol) using acetic anhydride (0.038 mL, 0.39 mmol), and N,N-diisopropylethylamine (0.25 mL, 1.18 mmol). To a solution of the resulting crude residue in ethanol (5 mL) purged with argon is added 10% palladium on carbon (25 mg, 0.01 mmol, wet, Degussa type). The mixture is stirred under a hydrogen atmosphere. After 2 hours, the mixture is filtered through glass filter paper rinsing with ethyl acetate. The solvent is removed in vacuo and the residue is purified via preparative HPLC using a gradient elution from 1-50% acetonitrile/water with 0.1% trifluoroacetic acid to obtain the desired product as a trifluoroacetic acid salt (50 mg, 26%). LCMS: 381.20 (M+H⁺).

The following compounds are prepared using the procedures of Example 22 and substituting for the appropriate starting materials.

| Example | | | m/z |
|---|---|---|---|
| No. | Structure | Name | [M + H⁺] |
| 24 | 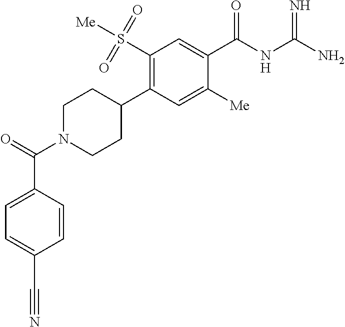 | N-{4-[1-(4-cyano- benzoyl)-piperidin-4-yl]- 5-methanesulfonyl-2- methyl-benzoyl}- guanidine | 509.42 |
| 25 | 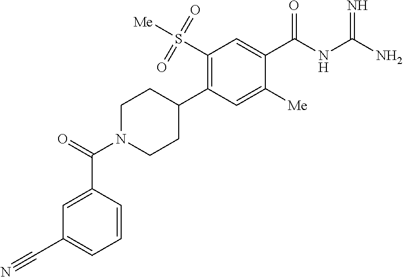 | N-{4-[1-(3-cyano- benzoyl)-piperidin-4-yl]- 5-methanesulfonyl-2- methyl-benzoyl}- guanidine | 468.20 |
| 26 | 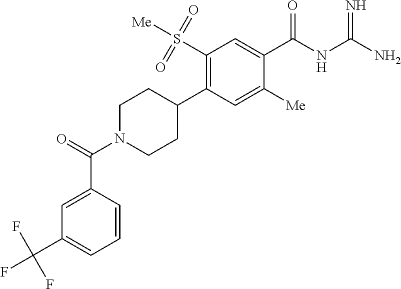 | N-{5-methanesulfonyl-2- methyl-4-[1-(3- trifluoromethyl- benzoyl)-piperidin-4-yl]- benzoyl}-guanidine | 511.20 |
| 27 | 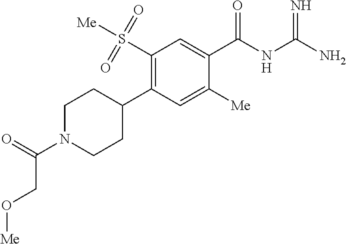 | N-{5-methanesulfonyl-4- [1-(2-methoxy-acetyl)- piperidin-4-yl]-2-methyl- benzoyl}-guanidine | 411.20 |
| 28 | 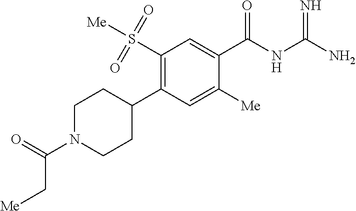 | N-{5-methanesulfonyl-4- [1-propionyl-piperidin-4- yl]-2-methyl-benzoyl}- guanidine | 395.20 |

### Example 29

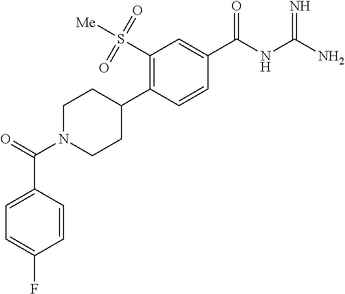

### N-{4-[1-(4-Fluorobenzoyl)-piperidin-4-yl]-3-methanesulfonyl-benzoyl}-guanidine

### Step A: 4-(2-Methansulfonyl-4-methoxycarbonyl-phenyl)-3,6-dihydro-2H-pyridine-1-carboxylic acid tert-butyl ester

The compound is prepared according to the procedure from Example 22 Step D, starting from 4-bromo-3-methanesulfonyl-benzoic acid methyl ester (which was prepared according to literature procedures) (3.00 g, 10.2 mmol) using 4-(4,4,5,5-tetramethyl-[1,3,2]dioxaborolan-2-yl)-3,6-dihydro-2H-pyridine-1-carboxylic acid tert-butyl ester (3.16 g, 10.2 mmol), 2 M aqueous potassium carbonate (10.2 mL, 20.5 mmol), and tetrakis(triphenylphosphine)palladium(0) (828 mg, 0.72 mmol), and purified via silica gel chromatography using a gradient elution of 10-40% ethyl acetate/hexanes to afford the desired product (2.73 g, 68%). LCMS: 296.20 (M+H⁺).

### Step B: 4-(2-Methanesulfonyl-4-methoxycarbonyl-phenyl)-piperidine-1-carboxylic acid tert-butyl ester

The compound is prepared according to the procedure from Example 22 Step E, starting from the product of Example 29 Step A (2.73 g, 6.90 mmol) and using platinum (IV) oxide (1.0 g) to afford the desired product (2.00 g, 73%). LCMS: 298.20 (M+H⁺).

### Step C: 4-(4-Carboxy-2-methanesulfonyl-phenyl)-piperidine-1-carboxylic acid tert-butyl ester

The compound is prepared according to the procedure from Example 5 Step I, starting from the product of Example 29 Step B (2.00 g, 5.03 mmol) and using lithium hydroxide monohydrate (422 mg, 10.1 mmol), to afford the desired product (1.90 g, 99%).

### Step D: 4-[2-Methanesulfonyl-4-(N′-carbobenzyloxy-guanidinocarbonyl)-phenyl]-piperidine-1-carboxylic acid tert-butyl ester

The compound is prepared according to the procedure from Example 5 Step J, starting from the product of Example 29 Step C (1.90 g, 4.96 mmol), 2-chloro-1-methylpyridinium iodide (1.39 g, 5.45 mmol), N-carbobenzyloxy-guanidine (1.15 g, 5.95 mmol), and N,N-diisopropylethylamine (2.59 mL, 14.9 mmol), and purified via silica gel chromatography using a gradient elution of 25-50% ethyl acetate in hexanes as the eluent to afford the desired product (2.80 g, quantitative). LCMS: 559.20 (M+H⁺).

### Step E: N-(3-Methanesulfonyl-4-piperidine-4-yl-benzoyl)-N′-(carbobenzyloxy)-guanidine

The compound is prepared according to the procedure from Example 5 Step K, starting from the product of Example 29 Step D (2.80 g, 5.01 mmol) and 4 M hydrochloric acid in 1,4-dioxane (14 mL) and heating at 50° C. to afford the desired product as the hydrochloride salt (2.00 g, 81%). LCMS: 459.20 (M+H⁺).

### Step F: N-{4-[1-(4-Fluorobenzoyl)-piperidin-4-yl]-3-methanesulfonyl-benzoyl}-guanidine

The compound is prepared according to the procedure from Example 5 Step L, starting from the product of Example 29 Step E (125 mg, 0.25 mmol), 4-fluorobenzoic acid (39 mg, 0.28 mmol), di-imidazol-1-yl-methanone (45 mg, 0.28 mmol), and N,N-diisopropylethylamine (0.13 mL, 0.76 mmol), and purified by preparative HPLC using a gradient elution from 5-65% acetonitrile/water with 0.1% trifluoroacetic acid as the eluent to give the desired intermediate which was deprotected using 10% palladium on carbon (30 mg, wet, Degussa type) to afford the desired product as the trifluoroacetic acid salt (49 mg, 35%). LCMS: 447.20 (M+H⁺)

### Example 30

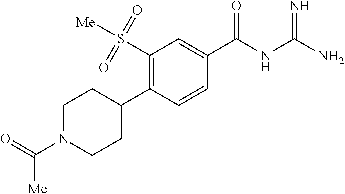

### N-[4-(1-Acetyl-piperidin-4-yl)-3-methanesulfonyl-benzoyl]-guanidine

The compound is prepared according to the procedure from Example 19, starting from the product of Example 2 Step E (200 mg, 0.40 mmol) using acetic anhydride (0.038 mL, 0.40 mmol), and N,N-diisopropylethylamine (0.21 mL, 1.21 mmol), and purified by preparative HPLC using a gradient elution from 1-50% acetonitrile/water with 0.1% trifluoroacetic acid as the eluent to give the desired intermediate which is deprotected using 10% palladium on carbon (40 mg, 0.02 mmol, wet, Degussa type) to afford the desired product as the trifluoroacetic acid salt (91 mg, 47%). LCMS: 367.20 (M+H⁺).

The following compounds are prepared using the procedures of Example 29 and substituting for the appropriate starting materials.

| Example | | | m/z |
|---|---|---|---|
| No. | Structure | Name | [M + H⁺] |
| 31 | 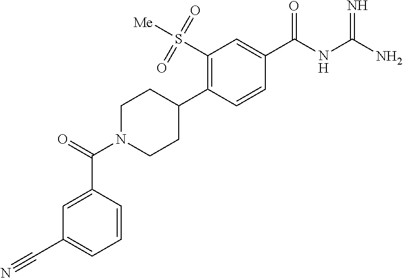 | N-{4-[1-(3-cyano- benzoyl)-piperidin-4-yl]- 3-methanesulfonyl- benzoyl)-guanidine | 454.20 |
| 32 | 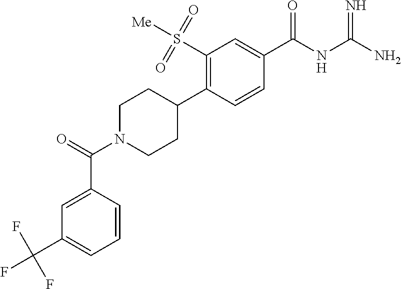 | N-{3-methanesulfonyl-4- [1-(3-trifluoromethyl- benzoyl)-piperidin-4-yl]- benzoyl)-guanidine | 497.20 |
| 33 | 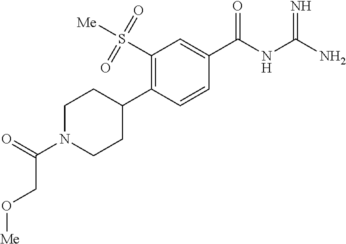 | N-{3-methanesulfonyl-4- [1-(2-methoxy-acetyl)- piperidin-4-yl]-benzoyl}- guanidine | 397.20 |
| 34 | 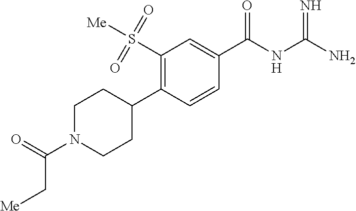 | N-{3-methanesulfonyl-4- [1-propionyl-piperidin-4- yl]-benzoyl}-guanidine | 381.20 |

### Example 35

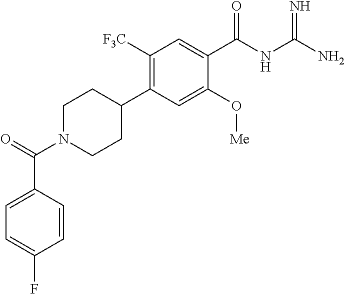

### N-{4-[1-(4-Fluoro-benzoyl)-piperidin-4-yl]-2-methoxy-5-trifluoromethyl-benzoyl}-guanidine

### Step A: 4-Benzyloxy-2-hydroxyoxy-benzoic acid methyl ester

To a solution of 2,4-dihydroxybenzoic acid methyl ester (25 g, 149 mmol) in acetone to (600 mL) is added potassium carbonate (22.6 g, 164 mmol). The mixture is stirred for 1 hour, then benzyl bromide (19.4 mL, 164 mmol) is added, the reaction is warmed to reflux for 3 hours and then cooled to room temperature. The mixture is filtered to remove solids and the filtrate is removed in vacuo. The residue is taken up in water and the resulting solids are isolated by filtration (29.1 g, 76%). LCMS: 259.00 (M+H⁺).

### Step B: 4-Benzyloxy-5-bromo-2-hydroxy-benzoic acid methyl ester

To a chilled (0° C.) solution of product from Step A (29.1 g, 112 mmol) in chloroform (300 mL) is added a solution of bromine (6.34 mL, 124 mmol) in chloroform (80 mL). The reaction is warmed to room temperature and stirred for 30 minutes. The reaction is washed with water, dried over Na₂SO₄, filtered and the solvent is removed in vacuo to afford a solid which is triturated with MeOH and isolated by filtration (30.75 g, 81%). LCMS: 337.00 (M+H⁺).

### Step C: 4-Benzyloxy-5-bromo-2-methoxy-benzoic acid methyl ester

To a chilled (0° C.) solution of product from Step B (10 g, 29.7 mmol) in tetrahydrofuran (250 mL) is added a solution of potassium tert-butoxide in tetrahydrofuran (36.5 mL, 36.5 mmol). After 30 minutes, iodomethane (2.4 mL, 38.6 mmol) is added and the reaction is stirred for 72 hours. The reaction is evaporated in vacuo and the residue is dissolved in water and neutralized with 1 N aqueous hydrochloric acid. The resulting solids are isolated by filtration, washed with copious amounts of water and dried. The solids are dissolved in ethyl acetate, washed with water, 1M aqueous sodium hydroxide, brine, dried over sodium sulfate, filtered and the solvent is removed to dryness. The residue is triturated with hexanes and the solids are isolated by filtration (8.1 g, 78%). NMR was consistent with desired product. LCMS: 351.00 (M+H⁺).

### Step D: 4-Hydroxy-5-trifluoromethyl-2-methoxy-benzoic acid methyl ester

The compound is prepared according to the procedure for Example 5 Step E, starting from the product from Step C (6.14 g, 18.0 mmol) using 10 wt % palladium on carbon (300 mg, 0.34 mmol, wet, Degussa type) and the desired product is isolated by trituration with ether as a brown solid (3.36 g, 74%). LCMS: 251.00 (M+H⁺).

### Step E: 2-Methoxy-4-trifluoromethanesulfonyloxy-5-trifluoromethyl-benzoic acid methyl ester

The compound is prepared according to the procedure for Example 5 Step F, starting from the product from Step D (3.36 g, 13.4 mmol) using N-phenyltrifluoromethanesulfonimide (5.04 g, 14.1 mmol) and N,N-diisopropylethylamine (2.57 mL, 14.8 mmol) which is used in the next step without further purification following aqueous workup.

### Step F: 4-(4-Carboxy-5-methoxy-2-trifluoromethyl-phenyl)3,6-dihydro-2H-pyridine-1-carboxylic acid tert-butyl ester)

A pressure flask is charged with tetrakis(triphenylphosphine)palladium(0) (1.58 g, 1.4 mmol), 4-(4,4,5,5-tetramethyl-[1,3,2] dioxaborolan-2-yl)-3,6-dihydro-2H-pyridine-1-carboxylic acid tert-butyl ester (4.22 g, 13.7 mmol) and potassium carbonate (3.53 g, 27.3 mmol) followed by a solution of product from Step E (5.22 g, 13.7 mmol) in dioxane (56 mL) and water (14 mL). The flask is sealed, warmed to 140° C. and stirred overnight. The mixture is cooled to room temperature, poured into ice water, and extracted with ethyl acetate. The combined organic phases are dried over Na₂SO₄, filtered, and the solvent is removed in vacuo to afford a brown oil. The crude material is purified via silica gel chromatography using a gradient elution of 0-100% ethyl acetate/hexanes to afford the desired product as a colorless solid after trituration with hexanes (3.6 g, 66%). LCMS: 402.00 (M+H⁺).

### Step G: 4-(4-Carboxy-5-methyl-2-trifluoromethyl-phenyl)-piperidine-1-carboxylic acid tert-butyl ester)

To a solution of product from Step F (3.6 g, 8.97 mmol) in acetic acid (40 mL) under a nitrogen atmosphere is added platinum(IV) oxide (150 mg, 0.66 mmol) and the reaction is placed under an atmosphere of hydrogen. After stirring overnight, the catalyst is removed by careful filtration through Celite, the filtrate is evaporated in vacuo. The residue is taken up in ether/hexanes and the resulting solids are isolated by filtration (3.1 g, 86%). LCMS: 404.00 (M+H⁺).

### Step H: 4-[5-Methyl-4-(N-(carbobenzyloxy)-guanidinocarbonyl)-2-trifluoromethyl-phenyl]-piperidine-1-carboxylic acid tert-butyl ester

The compound is prepared according to the procedure for Example 5 Step J, starting from the product from Step G (3.10 g, 7.69 mmol) using 2-chloro-1-methylpyridinium iodide (2.36 g, 9.22 mmol), N-carbobenzyloxy-guanidine (1.93 g, 10.0 mmol), and N,N-diisopropylethylamine (3.82 mL, 23.1 mmol). The crude reaction mixture is poured into a solution of formic acid (7 mL) in water (250 mL) and the desired product is isolated by filtration as a brown solid (4.08 g, 92%). LCMS: 579.00 (M+H⁺).

### Step I: N-(2-methoxy-4-piperidin-4-yl-5-trifluoromethyl-benzoyl)-N′-(carbobenzyloxy)-guanidine

To a solution of the product from Step H (4.1 g, 7.05 mmol) in dioxane (100 mL) is added 4 M hydrogen chloride in 1,4-dioxane (12 mL). The mixture is stirred at room temperature for 48 hours. The mixture is evaporated in vacuo and the residue is triturated with ether to afford the desired product as the hydrochloride salt (3.7 g, quantitative). LCMS: 479.70 (M+H⁺).

### Step J: N-{4-[1-(4-Fluoro-benzoyl)-piperidin-4-yl]-2-methoxy-5-trifluoromethyl-benzoyl}-guanidine

The compound is prepared according to Example 5 Step L, starting from the product from Step I (100 mg, 0.17 mmol) using di-imidazol-1-yl-methanone (35 mg, 0.21 to mmol), 4-fluorobenzoic acid (29.9 mg, 0.21 mmol), and N,N-diisopropylethylamine (105 μL, 0.58 mmol) to give the crude residue which is subjected to hydrogenation using 20 wt % palladium(II) hydroxide (10 mg, 0.007 mmol, 50% wet), and the product is purified via preparative HPLC using a gradient elution from 10-100% acetonitrile/water with 0.1% trifluoroacetic acid to obtain the desired product as a trifluoroacetic acid salt (44 mg, 47%). LCMS: 581.00 (M+H⁺).

The following compounds are prepared using the procedures of Example 33 and substituting for the appropriate starting materials.

| Example | | | m/z |
|---|---|---|---|
| No. | Structure | Name | [M + H⁺] |
| 36 | 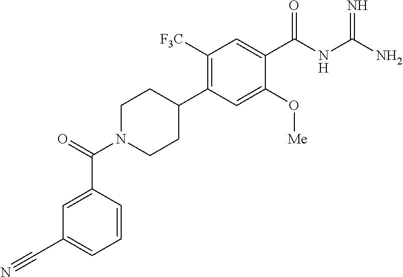 | N-{4-[1-(3-Cyano- benzoyl)-piperidin- 4-yl]-2-methoxy-5- trifluoromethyl- benzoyl}-guanidine | 474.20 |
| 37 | 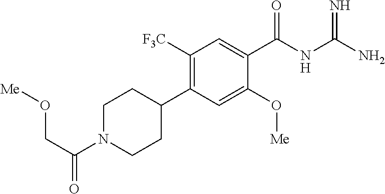 | N-{2-Methoxy-4- [1-(2-methoxy- acetyl)-piperidin-4- yl]-5- trifluoromethyl- benzoyl}-guanidine | 417.20 |
| 38 | 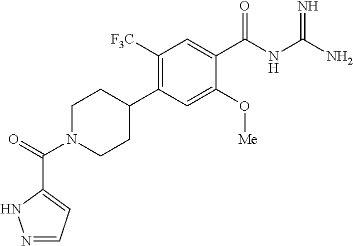 | N-{2-Methoxy-4- [1-(2H-pyrazole-3- carbonyl)- piperidin-4-yl]-5- trifluoromethyl- benzoyl}-guanidine | 439.20 |
| 39 | 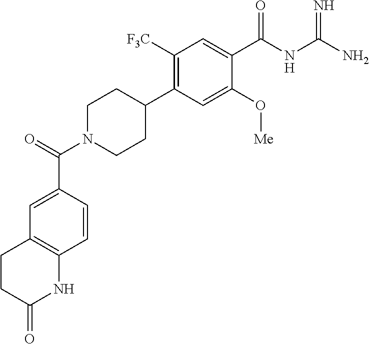 | N-{2-Methoxy-4- [1-(2-oxo-l,2,3,4- tetrahydro- quinoline-6- carbonyl)- piperidin-4-yl]-5- trifluoromethyl- benzoyl}-guanidine | 518.20 |
| 40 | 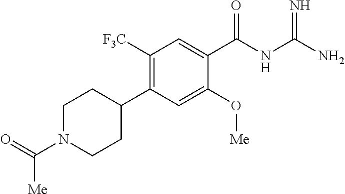 | N-[4-(1-Acetyl- piperidin-4-yl)-2- methoxy-5- trifluoromethyl- benzoyl]-guanidine | 387.20 |
| 41 | 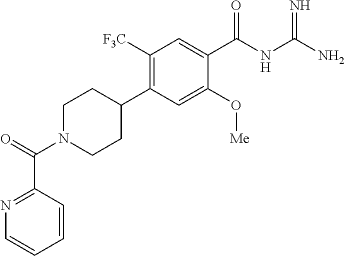 | N-{2-Methoxy-4- [1-(pyridine-2- carbonyl)- piperidin-4-yl]-5- trifluoromethyl- benzoyl}-guanidine | 450.20 |
| 42 | 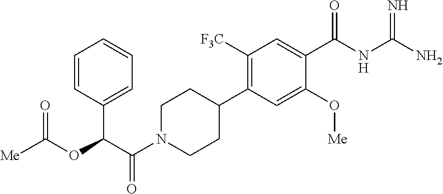 | Acetic acid (S)-2- [4-(4- guanidinocarbonyl- 5-methoxy-2- trifluoromethyl- phenyl)-piperidin- 1-yl]-2-oxo-l- phenyl-ethyl ester | 521.20 |
| 43 | 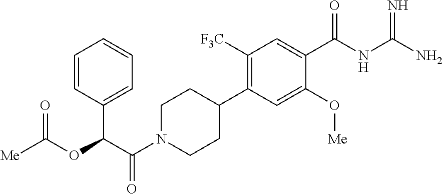 | Acetic acid (R)-2- [4-(4- guanidinocarbonyl- 5-methoxy-2- trifluoromethyl- phenyl)-piperidin- 1-yl]-2-oxo-1- phenyl-ethyl ester | 521.20 |
| 44 | 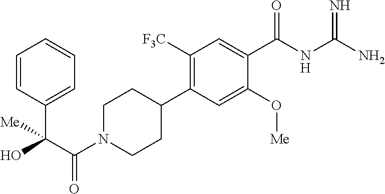 | N-{4-[1-((S)-2- Hydroxy-2-phenyl- propionyl)- piperidin-4-yl]-2- methoxy-5- trifluoromethyl- benzoyl}-guanidine | 493.20 |
| 45 | 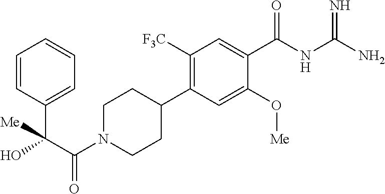 | N-{4-[1-((R)-2- Hydroxy-2-phenyl- propionyl)- piperidin-4-yl]-2- methoxy-5- trifluoromethyl- benzoyl}-guanidine | 493.20 |
| 46 | 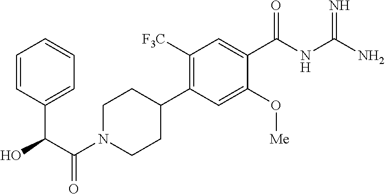 | N-{4-[1-((S)-2- Hydroxy-2-phenyl- acetyl)-piperidin-4- yl]-2-methoxy-5- trifluoromethyl- benzoyl}-guanidine | 479.20 |

### Example 47

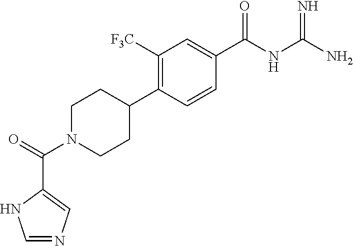

### N-{4-[1-(3H-Imidazole-4-carbonyl)-1,2,3,6-tetrahydro-pyridin-4-yl]-3-trifluoromethyl-benzoyl}-guanidine

### Step A: 4-Bromo-3-trifluoromethyl-benzoic acid methyl ester

To a solution of 4-bromo-3-trifluoromethyl-benzoic acid (115 g, 428 mmol) in methanol to (400 mL) is added concentrated sulfuric acid (2 mL). The mixture is sealed and heated to 80° C. overnight. The mixture is cooled to room temperature and the solvent is removed in vacuo. The residue is treated with water and the resulting solid is isolated by filtration and dried to afford the desired product as a colorless solid (121 g, 98%). LCMS: 283.00/285.00 (2 Br isotopes M+H⁺)

### Step B: 4-(4-Methoxycarbonyl-2-trifluoromethyl-phenyl)-3,6-dihydro-2H-pyridine-1-carboxylic acid tert-butyl ester

To a solution of the product from Step A (17.3 g, 61.2 mmol) in 1,4-dioxane is added 4-(4,4,5,5-tetramethyl-[1,3,2]dioxaborolan-2-yl)-3,6-dihydro-2H-pyridine-1-carboxylic acid tert-butyl ester (18.9 g, 61.2 mmol), 2 M aqueous potassium carbonate (61.2 mL, 122 mmol), and tetrakistriphenylphosphinepalladium(0) (7.07 g, 6.10 mmol). The reaction vessel is sealed and heated to 140° C. for 4 hours. The mixture is cooled to room temperature and the dioxane is removed in vacuo. The resulting residue is diluted with water and ethyl acetate, filtered to remove insoluble material, and the layers are separated. The aqueous layer is extracted twice with ethyl acetate and the combined organics are dried over Na₂SO₄, filtered, and the solvent is removed in vacuo. The brown residue is then passed through a short bed of silica rinsing first with 10% ethyl acetate in hexane, then 20% ethyl acetate in hexane. The material collected from the 20% ethyl acetate fraction is evaporated to afford the desired product as a light yellow solid (19.6 g, 83%). LCMS: 371.51 (M+CH₃CN-56).

### Step C: 4-(4-Carboxy-2-trifluoromethyl-phenyl)-3,6-dihydro-2H-pyridine-1-carboxylic acid tert-butyl ester

To a solution of the product from Step B (2.74 g, 7.10 mmol) in methanol (40 mL) is added water (4 mL) and potassium carbonate (2.46 g, 17.8 mmol) and the reaction mixture is heated at 60° C. for 2 hours. The mixture is then diluted with ethyl acetate (200 mL) and water (100 mL). The aqueous layer is brought to a pH of about 4 using 1 N aqueous hydrochloric acid. The layers are separated and the aqueous layer is extracted twice with ethyl acetate. The combined organic phase is dried over Na₂SO₄, filtered, and the solvent is removed in vacuo to yield the desired product as a colorless solid (2.48 g, 94%). LCMS: 357.46 (M+CH₃CN-56).

### Step D: 4-(4-(N-Carbobenzyloxy)-guanidinocarbonyl-2-trifluoromethyl-phenyl)-3,6-dihydro-2H-pyridine-1-carboxylic acid tert-butyl ester

To a solution of the product from Step C (2.48 g, 6.68 mmol) in N-methylpyrrolidinone (35 mL) is added 2-chloro-1-methylpyridinium iodide (2.05 g, 8.01 mmol) and the resulting solution is stirred for 75 minutes. N-carbobenzyloxy-guanidine (1.68 g, 8.68 mmol) is then added followed by N,N-diisopropylethylamine (3.32 mL, 20.0 mmol) and the reaction is stirred overnight. The mixture is partitioned between water and ethyl acetate and the layers are separated. The organic phase is washed twice with water, once with brine, dried over Na₂SO₄, filtered, and the solvent is removed in vacuo. The crude material is purified via silica gel chromatography using a gradient elution of 0-40% ethyl acetate/hexanes to afford the desired product (3.37 g, 92%) as a glassy solid. LCMS: 547.83 (M+H⁺).

### Step E: N-[(1,2,3,6-Tetrahydro-pyridin-4-yl)-3-trifluoromethyl-benzoyl]-N′-(carbobenzyloxy)-guanidine

To a solution of the product from Step D (3.37 g, 6.20 mmol) in dichloromethane (24 mL) is added 4 N hydrogen chloride in 1,4-dioxane (25 mL, 100 mmol) and the reaction is stirred for 2 hours. The mixture is diluted with ether (200 mL) and filtered to give the desired product as a colorless solid (2.80 g, 94%). LCMS: 447.77 (M+H⁺).

### Step F: N-{4-[1-(3H-Imidazole-4-carbonyl)-1,2,3,6-tetrahydro-pyridin-4-yl]-3-trifluoromethyl-benzoyl}-N′-(carbobenzyloxy)-guanidine

To a solution of the product from Step E (100 mg, 0.21 mmol) in dichloromethane is added 1-ethyl-3-(3-dimethylaminopropyl)carbodiimide hydrochloride (48 mg, 0.25 mmol), 1-hydroxybenzotriazole (34 mg, 0.25 mmol), and N,N-diisopropylethylamine (0.11 mL, 0.62 mmol) and the reaction is stirred overnight. The mixture is diluted with ethyl acetate and washed twice with water, saturated aqueous sodium bicarbonate, and brine. The organic phase is dried over Na₂SO₄, filtered, and the solvent is removed in vacuo. The crude material is purified via silica gel chromatography using a gradient elution of 0-9% methanol/dichloromethane to afford the desired product (34 mg, 31%). LCMS: 541.44 (M+H⁺).

### Step G: N-{4-[1-(3H-Imidazole-4-carbonyl)-1,2,3,6-tetrahydro-pyridin-4-yl]-3-trifluoromethyl-benzoyl}-guanidine

To a solution of the product from Step F (34 mg, 0.063 mmol) in ethanol (2 mL) under an argon atmosphere is added 10% palladium on carbon (13 mg, 0.006 mmol, wet, Degussa type) and the reaction is stirred under a hydrogen atmosphere for 3 hours. The mixture is filtered through Celite and the solvent is removed in vacuo. The residue is purified via preparative HPLC using a gradient elution from 10-90% acetonitrile/water with 0.1% trifluoroacetic acid to obtain the desired product as a bis-trifluoroacetic acid salt (10 mg, 25%). LCMS: 407.42 (M+H⁺).

The following compounds are prepared using the procedures of Example 45 and substituting for the appropriate starting materials.

| Example | | | m/z |
|---|---|---|---|
| No. | Structure | Name | [M + H⁺] |
| 48 | 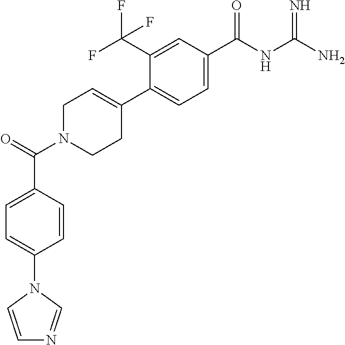 | N-{4-[1-(4-imidazol-1-yl- benzoyl)-1,2,3,6- tetrahydro-pyridin-4-yl]-3- trifluoromethyl-benzoyl}- guanidine | 438.45 |
| 49 | 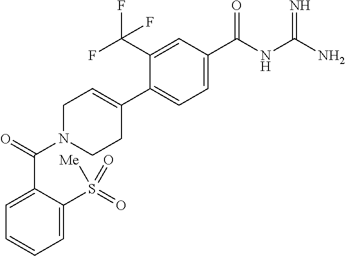 | N-{4-[1-(2- methanesulfonyl-benzoyl)- 1,2,3,6-tetrahydro-pyridin- 4-yl]-3-trifluoromethyl- benzoyl}-guanidine | 495.37 |

### Example 50

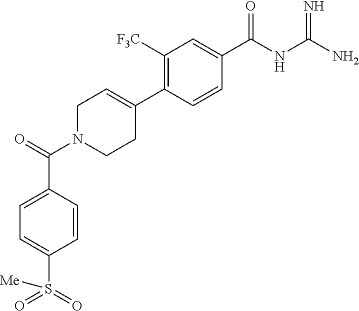

### N-{4-[1-(4-Methanesulfonyl-benzoyl)-1,2,3,6-tetrahydro-pyridin-4-yl]-3-trifluoromethyl-benzoyl}-guanidine

### Step A: N-{4-[1-(4-Methanesulfonyl-benzoyl)-1,2,3,6-tetrahydro-pyridin-4-yl]-3-trifluoromethyl-benzoyl}-N′-(carbobenzyloxy)-guanidine

To a solution of 4-methanesulfonyl-benzoic acid (46 mg, 0.23 mmol) in N,N-dimethylformamide (3.0 mL) is added di-imidazol-1-yl-methanone (37 mg, 0.23 mmol) and the reaction is stirred for 45 minutes. The product of Example 47 Step E (100 mg, 0.21 mmol) is added followed by N,N-diisopropylethylamine (0.13 mL, 0.73 mmol) and the reaction is stirred overnight. The mixture is diluted with ethyl acetate and washed twice with water, once with brine, dried over Na₂SO₄, filtered, and the solvent is removed in vacuo. The crude material is purified via silica gel chromatography using a gradient elution of 0-70% ethyl acetate/hexane to afford the desired product (90 mg, 69%). LCMS: 495.71 (M+H⁺).

### Step B: N-{4-[1-(4-Methanesulfonyl-benzoyl)-1,2,3,6-tetrahydro-pyridin-4-yl]-3-trifluoromethyl-benzoyl}-guanidine

To a solution of the product from Step A (90 mg, 0.14 mmol) in ethanol (2 mL) and ethyl acetate (1 mL) under an argon atmosphere is added 10 wt % palladium on carbon (31 mg, 0.014 mmol, wet, Degussa type) and the reaction is stirred under a hydrogen atmosphere for 3 hours. The mixture is filtered through Celite and the solvent is removed in vacuo. The residue is purified via preparative HPLC using a gradient elution from 10-90% acetonitrile/water with 0.1% trifluoroacetic acid to obtain the desired product as a trifluoroacetic acid salt (12 mg, 16%). LCMS: 407.42 (M+H⁺).

### Example 51

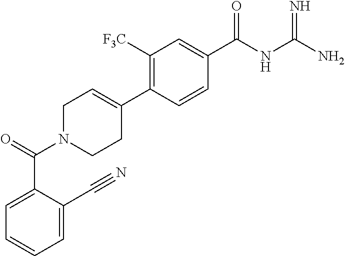

### N-{4-[1-(2-Cyano-benzoyl)-1,2,3,6-tetrahydro-pyridin-4-yl]-3-trifluoromethyl-benzoyl}-guanidine

### Step A: N-{4-[1-(2-Cyano-benzoyl)-1,2,3,6-tetrahydro-pyridin-4-yl]-3-trifluoromethyl-benzoyl}-N′-(carbobenzyloxy)-guanidine

To a solution of 4-cyano-benzoic acid (33 mg, 0.23 mmol) in N,N-dimethylformamide (3.0 mL) is added di-imidazol-1-yl-methanone (37 mg, 0.23 mmol) and the reaction is stirred for 47 minutes. The product of example 45 Step E (100 mg, 0.21 mmol) is then added followed by N,N-diisopropylethylamine (0.13 mL, 0.73 mmol) and the reaction is stirred overnight. The mixture is diluted with ethyl acetate, washed twice with water, once with brine, dried over Na₂SO₄, filtered, and the solvent is removed in vacuo. The crude material is purified via silica gel chromatography using a gradient elution of 0-80% ethyl acetate/hexane to afford the desired product (85 mg, 72%). LCMS: 495.71 (M+H⁺).

### Step B: N-{4-[1-(2-Cyano-benzoyl)-1,2,3,6-tetrahydro-pyridin-4-yl]-3-trifluoromethyl-benzoyl}-guanidine

To a solution of the product from Step A (10 mg, 0.017 mmol) in ethanol (1 mL) under an argon atmosphere is added 10 wt % palladium(II) hydroxide on carbon (1.9 mg, 0.002 mmol, 50% wet) and the reaction is stirred under a hydrogen atmosphere overnight. The mixture is filtered through Celite and the solvent is removed in vacuo. The crude residue is dissolved in dichloromethane and precipitated as the hydrochloride salt using 4 N hydrogen chloride in 1,4-dioxane and diluting with ether. The desired product is isolated by filtration as a colorless solid (5.0 mg, 59%). LCMS: 442.42 (M+H⁺).

### Example 52

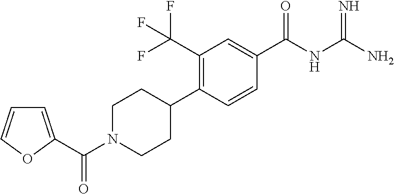

### N-{4-[1-(1H-Furan-2-carbonyl)-piperidin-4-yl]-3-trifluoromethyl-benzoyl}-guanidine; to hydrochloride

### Step A: 4-Trifluoromethanesulfonyloxy-3,6-dihydro-2H-pyridine-1-carboxylic acid tert-butyl ester

To a solution of 4-oxo-piperidine-1-carboxylic acid tert-butyl ester, (5.00 g, 25.13 mmol) in tetrahydrofuran (50 mL) is added 20% lithium bis(trimethylsilyl)amide solution in tetrahydrofuran (50 mL) at −75° C. and the mixture is stirred for 30 minutes.

A solution of N-phenyl trifluoromethanesulfonimide (9.85 g, 27.64 mmol) in tetrahydrofuran (100 mL) is added slowly at −75° C. to the reaction mixture and the temperature is raised to 0° C. The mixture is stirred at this temperature for 3 hours. To the reaction mixture ice cold water (100 mL) and ethyl acetate (100 mL) are added and the mixture is stirred for 10 minutes. The organic phase is separated and the aqueous layer is extracted with ethyl acetate (100 mL). The combined organic phase is dried and distilled. The crude material is purified via silica gel chromatography using a gradient elution of 10% ethyl acetate/petroleum ether to afford the desired product (5.30 g, 90%).

### Step B: 4-(4,4,5,5-Tetramethyl-[1,3,2]dioxaborolan-2-yl)-3,6-dihydro-2H-pyridine-1-carboxylic acid tert-butyl ester

To a solution of the product of Step A (5.00 g, 21.65 mmol) in degassed 1,4-dioxane (30 mL) is added 4,4,5,5,4′,4′,5′,5′-Octamethyl-[2,2]bi[[1,3,2]dioxaborolanyl], (6.00 g, 23.8 mmol), Potassium acetate (6.30 g, 64.95 mmol), 1,1′-bis (diphenylphosphino)ferrocenedichloropalladium(II) (0.500 g, 0.65 mmol) and di-phenyl phosphinoferrocene (dppf) (0.360 g, 0.65 mmol). The reaction mixture is heated at 80° C. over night. The mixture is cooled to room temperature and ethyl acetate (100 mL) is added and the mixture is stirred for 10 minutes. The organic phase is separated and the aqueous phase is extracted with ethyl acetate (2×100 mL). The combined organic fractions are washed with water (100 mL), brine (100 mL), dried over Na₂SO₄, and the solvent is removed. The crude material is purified via silica gel chromatography using a gradient elution of 5-8% ethyl acetate/hexane to afford the desired product (5.50 mg, 83%).

### Step C: 4-(4-Cyano-2-trifluoromethyl-phenyl)-3,6-dihydro-2H-pyridine-1-carboxylic acid tert-butyl ester

To a slurry of anhydrous potassium carbonate (3.50 g, 25.3 mmol) in dry degassed N,N-dimethylformamide (35 mL) is added 4-bromo-3-trifluoromethyl benzonitrile (2.00 g, 8.00 mmol) followed by bis(diphenylphosphino)ferrocenedichloropalladium(II) (0.92 g, 1.25 mmol) and the reaction mixture is heated at 80° C. overnight. The reaction mixture is cooled to room temperature, diluted with ethyl acetate, filtered through Celite, and the solvent is removed in vacuo. The crude material is purified via silica gel chromatography using 4% ethyl acetate/petroleum ether as eluent to afford the desired product (1.20 g, 56%).

### Step D: 4-(4-Carboxy-2-trifluoromethyl-phenyl)-3,6-dihydro-2H-pyridine-1-carboxylic acid tert butyl ester

To a solution of the product from Step C (1.15 g, 3.2 mmol) in ethanol (6 mL) is added water (12 mL) and sodium hydroxide (0.650 g, 16.3 mmol) (white precipitate is obtained). The reaction mixture is refluxed for 2 hours. The ethanol is removed in vacuo. The reaction mixture is acidified with 1 N hydrochloric acid and the aqueous layer is extracted with dichloromethane (2×60 mL). The organic layer is dried and the solvent is removed in vacuo to give the above product as colorless solid (1.10 g, 98%).

### Step E: 4-(4-Carboxy-2-trifluoromethyl-phenyl)-piperidine-1-carboxylic acid tert-butyl ester

To a solution of the product from Step D (1.10 g, 2.90 mmol) in methanol (50 mL) in a Parr hydrogenation apparatus is added palladium on carbon (10%, 0.10 g, mmol, wet, Degussa type) and the reaction is shaken at 60 psi over night. The reaction mixture is filtered through Celite and methanol is distilled in vacuo to give a colorless solid (1.00 g, 98%). The crude product is used as such to next step.

### Step F: 4-Piperidin-4-yl-3-trifluoromethyl-benzoic acid methyl ester

To the product from Step E (1.00 g, 2.68 mmol) is added methanolic hydrogen chloride (25 mL) and the resultant reaction mixture is stirred at room temperature overnight. The reaction mixture is distilled under vacuum. The crude product is triturated with anhydrous ethyl ether, filtered and dried in vacuo to afford the desired product hydrochloride salt as a light yellow solid (0.760 g, 88%)

### Step G: 4-[1-(Furan-2-carbonyl)-piperidin-4-yl]-3-trifluoromethyl-benzoic acid methyl ester

To a solution of the product from Step F (0.530 mg, 1.60 mmol) in dry tetrahydrofuran (20 mL) is added furan-2-carboxylic acid (0.275, 2.4 mmol) and N-[(dimethylamino)-1H-1,2,3-triazolo[4,5-B]pyridin-1-ylmethylene]-N-methylmethanaminium hexafluorophosphate N-oxide (0.625 mg, 1.6 mmol) at 0-5° C. Diisopropylethylamine (1.41 mL, 8 mmol) is then added and the resultant reaction mixture is stirred at room temperature overnight. Water (20 mL) is added to the mixture and it is extracted with ethyl acetate (2×25 mL). The organic phase is dried and the solvent is removed in vacuo. The crude material is purified via silica gel chromatography using 15% ethyl acetate/hexane as the eluent to afford the desired product (630 mg, 92%).

### Step H: 4-[1-(Furan-2-carbonyl)-piperidin-4-yl]-3-trifluoromethyl-benzoic acid

To a solution of the product from Step G (0.720 g, 1.80 mmol) in tetrahydrofuran:water (8:2, 10 mL) is added lithium hydroxide monohydrate (0.280 g, 6.6 mmol) and the reaction mixture is stirred at room temperature overnight. The reaction mixture is evaporated, water (10 mL) and ether (10 mL) are added and the layers are separated. The aqueous phase is acidified with 10 M citric acid solution (25 mL) and the resulting solid is filtered off. The compound is purified by triturating with 10% ether-hexanes to afford the desired product as a light tan solid (0.400 g, 0%).

### Step I: N-{4-[1-(Furan-2-carbonyl)-piperidin-4-yl]-3-trifluoromethyl-benzoyl}-N′-(carbobenzyloxy)-guanidine

To a solution of the product of Step H (0.200 mg, 0.544 mmol) in 1-methyl-2-pyrrolidone (3 mL) is added 2-chloro-1-methylpyridinium iodide (0.152 mg, 0.59 mmol). The mixture is stirred at room temperature for 90 minutes. To the reddish-brown mixture is added N-carbobenzyloxyguanidine (0.126 mg, 0.653 mmol) and N,N-diisopropylethylamine (0.28 mL, 1.63 mmol). The mixture is allowed to stir at room temperature overnight. The mixture is added to ice cold water which contains 0.1% formic acid to give a white solid. The solid is filtered and washed several times with water. Then resulting solid is dried under vacuum to provide the desired product (0.20 mg, 67.7%). LCMS: 543.31 (M+H⁺).

### Step J: N-{4-[1-(Furan-2-carbonyl)-piperidin-4-yl]-3-trifluoromethyl-benzoyl}-guanidine

To a solution of the product from Step I (0.180 mg, 0.306 mmol) in ethanol (10 mL) under an argon atmosphere is added 10 wt % palladium on carbon (0.150 mg, 0.07 mmol, wet, Degussa type). The mixture is stirred under a hydrogen atmosphere overnight. The mixture is filtered through Celite and washed several times with ethanol and the solvent is removed in vacuo. The residue is dissolved in ether and a couple of drops of methanol. 2 Equivalents of 1 N hydrogen chloride in ether is added and the resulting solid is filtered off, washed and dried in vacuo to give the desired product as a bis-hydrochloride salt (0.085 mg, 62.5%). LCMS: 408.81, 409.63, 410.32 (M+H⁺).

### Example 53

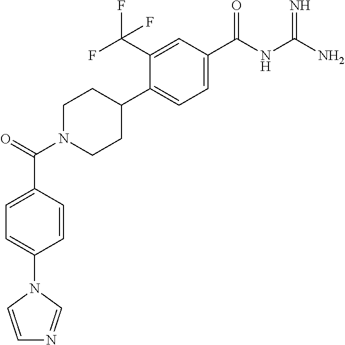

### N-{4-[1-(4-Imidazol-1-yl-benzoyl)-piperidin-4-yl]-3-trifluoromethyl-benzoyl}-guanidine

To a solution of the product from Example 48 (20 mg, 0.036 mmol) in ethanol (1.5 mL) under an argon atmosphere is added 10 wt % palladium on carbon (8.0 mg, 0.004 mmol, wet, Degussa type) and the mixture is stirred for 60 hours under a hydrogen atmosphere. The mixture is filtered through Celite, rinsing with 1% ammonia in methanol and the solvent is removed in vacuo. The residue is purified via preparative HPLC using a to gradient elution from 10-70% acetonitrile/water with 0.1% trifluoroacetic acid to obtain the desired product as a trifluoroacetic acid salt (14 mg, 55%). LCMS: 485.44 (M+H⁺).

### Example 54

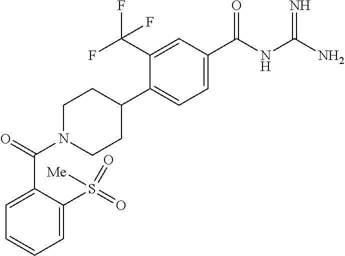

### N-{4-[1-(2-Methanesulfonyl-benzoyl)-piperidin-4-yl]-3-trifluoromethyl-benzoyl}-guanidine

The desired product is isolated from the reaction mixture of Example 49 Step G by preparative HPLC using a gradient elution from 10-80% acetonitrile/water with 0.1% trifluoroacetic acid to obtain the desired product as a trifluoroacetic acid salt (37 mg, 32%). LCMS: 497.38 (M+H⁺).

### Example 55

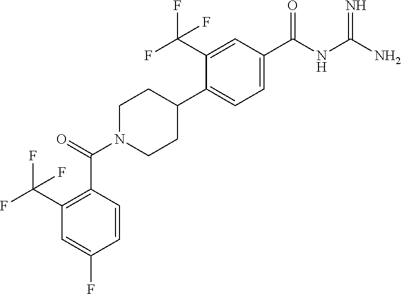

### N-{4-[1-(4-Fluoro-2-trifluoromethyl-benzoyl)-piperidin-4-yl]-3-trifluoromethyl-benzoyl}-guanidine

### Step A: N-{4-[1-(4-Fluoro-2-trifluoromenthyl-benzoyl)-piperidin-4-yl]-3-trifluoromethyl-benzoyl}-N′-(carbobenzyloxy)-guanidine

To a solution of 4-fluoro-2-trifluoromethylbenzoic acid (47 mg, 0.23 mmol) in N,N-dimethylformamide (3.0 mL) is added di-imidazol-1-yl-methanone (37 mg, 0.23 mmol) and the mixture is stirred for 90 minutes. The product of example 47 Step E (100 mg, 0.21 mmol) is then added followed by N,N-diisopropylethylamine (0.13 mL, 0.73 mmol) and the reaction is stirred overnight at 50° C. The mixture is diluted with ethyl acetate and washed twice with water, once with brine, dried over Na₂SO₄, filtered, and the solvent is removed in vacuo. The crude material is purified via silica gel chromatography using a gradient elution of 0-70% ethyl acetate/hexane to afford the desired product (65 mg, 49%). LCMS: 647.36 (M+H⁺).

### Step B: N-{4-[1-(4-Fluoro-2-trifluoromethyl-benzoyl)-piperidin-4-yl]-3-trifluoromethyl-benzoyl}-guanidine

To a solution of the product of Step A (65 mg, 0.10 mmol) in ethanol (2 mL) under an argon atmosphere is added 10 wt % palladium on carbon (22 mg, 0.010 mmol, wet, Degussa type) and the reaction is stirred under a hydrogen atmosphere for 16 hours. The mixture is filtered through Celite and the solvent is removed in vacuo. The residue is purified via preparative HPLC using a gradient elution from 10-90% acetonitrile/water with 0.1% trifluoroacetic acid to obtain the desired product as a trifluoroacetic acid salt (16 mg, 25%). LCMS: 505.39 (M+H⁺).

The following compounds are prepared using the procedures of Example 53 and substituting for the appropriate starting materials.

| Example | | | m/z |
|---|---|---|---|
| No. | Structure | Name | [M + H⁺] |
| 56 | 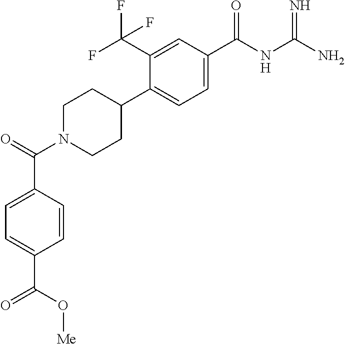 | 4-[4-(4- guanidinocarbonyl-2- trifluoromethyl-phenyl)- piperidine-1-carbonyl]- benzoic acid methyl ester | 477.70 |
| 57 | 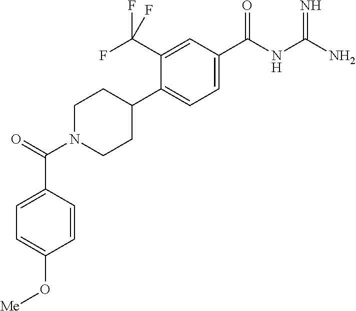 | N-{4-[1-(4-methoxy- benzoyl)-piperidin-4-yl]- 3-trifluoromethyl- benzoyl}-guanidine | 449.73 |

### Example 58

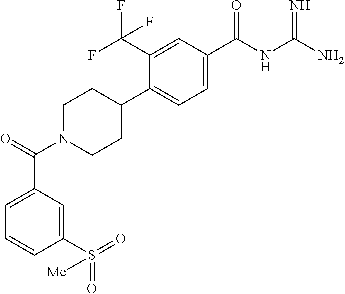

### N-{4-[1-(3-Methanesulfonyl-benzoyl)-piperidin-4-yl]-3-trifluoromethyl-benzoyl}-guanidine

### Step A: N-{4-[1-(3-Methanesulfonyl-benzoyl)-piperidin-4-yl]-3-trifluoromethyl-benzoyl}-N′-(carbobenzyloxy)-guanidine

To a solution of 3-methanesulfonylbenzoic acid (46 mg, 0.23 mmol) in N,N-dimethylformamide (3.0 mL) is added di-imidazol-1-yl-methanone (37 mg, 0.23 mmol) and the mixture is stirred for 90 minutes. The product of Example 47 Step E (100 mg, to 0.21 mmol) is then added followed by N,N-diisopropylethylamine (0.13 mL, 0.73 mmol) and the reaction is stirred overnight at room temperature. The mixture is diluted with ethyl acetate and washed twice with water and then brine. The organic phase is dried over Na₂SO₄, filtered, and the solvent is removed in vacuo. The crude material is purified via silica gel chromatography using a gradient elution of 0-100% ethyl acetate/hexane to afford the desired product (110 mg, 85%). LCMS: 629.41 (M+H⁺).

### Step B: N-{4-[1-(3-Methanesulfonyl-benzoyl)-piperidin-4-yl]-3-trifluoromethyl-benzoyl}-guanidine

To a solution of the product of Step A (110 mg, 0.18 mmol) in ethanol (3 mL) under an argon atmosphere is added 10 wt % palladium on carbon (37 mg, 0.017 mmol, wet, Degussa type) and the reaction is stirred under a hydrogen atmosphere for 36 hours. The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (60 mg, 56%). LCMS: 497.71 (M+H⁺).

### Example 59

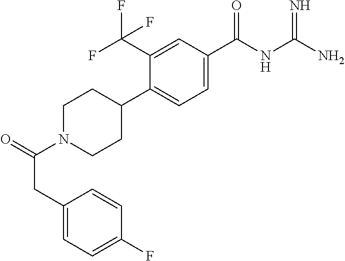

### N-(4-{1-[2-(4-Fluoro-phenyl)-acetyl]-piperidin-4-yl}-3-trifluoromethyl-benzoyl)-guanidine

The title compound is prepared according to the procedure for Example 58 using the appropriate starting materials. The crude mixture is purified via preparative HPLC using a gradient elution from 10-80% acetonitrile/water with 0.1% trifluoroacetic acid to obtain the desired product as a trifluoroacetic acid salt (62 mg, 64%). LCMS: 451.42 (M+H⁺).

### Example 60

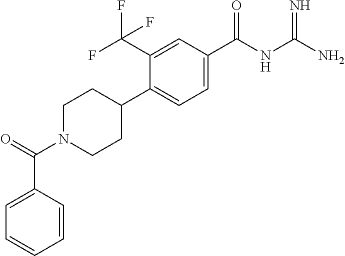

### N-[4-(1-Benzoyl-piperidin-4-yl)-3-trifluoromethyl-benzoyl]-guanidine

### Step A: N-[4-(1-Benzoyl-piperidin-4-yl)-3-trifluoromethyl-benzoyl]-N′-(carbobenzyloxy)-guanidine

To a solution of 4-benzoic acid (36 mg, 0.23 mmol) in N,N-dimethylformamide (3.0 mL) is added di-imidazol-1-yl-methanone (37 mg, 0.23 mmol) and the mixture is stirred for 90 minutes. The product from Example 47 Step E (100 mg, 0.21 mmol) is then added followed by N,N-diisopropylethylamine (0.13 mL, 0.73 mmol) and the reaction is stirred overnight at room temperature. The mixture is diluted with ethyl acetate and washed twice with wateronce with brine, dried over Na₂SO₄, filtered, and the solvent is removed in vacuo. The crude material is purified via silica gel chromatography using a gradient elution of 0-60% ethyl acetate/hexane to afford the desired product (106 mg, 88%). LCMS: 585.41 (M+H⁺).

### Step B: N-[4-(1-Benzoyl-piperidin-4-yl)-3-trifluoromethyl-benzoyl]-guanidine

To a solution of the product of Step A (106 mg, 0.18 mmol) in ethanol (3 mL) under an argon atmosphere is added 10 wt % palladium on carbon (39 mg, 0.018 mmol, wet, to Degussa type) and the reaction is stirred under a hydrogen atmosphere for 36 hours.

The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (70 mg, 73%). LCMS: 419.72 (M+H⁺).

### Example 61

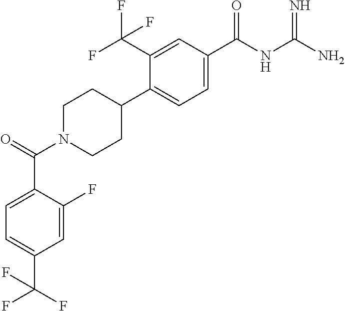

### N-{4-[1-(2-Fluoro-4-trifluoromethyl-benzoyl)-piperidin-4-yl]-3-trifluoromethyl-benzoyl}-guanidine

### Step A: N-{4-[1-2-Fluoro-4-trifluoromethyl-benzoyl)-piperidin-4-yl]-3-trifluoromethyl-benzoyl}-N′-(carbobenzyloxy)-guanidine

To a solution of 2-fluoro-4-trifluoromethylbenzoic acid (47 mg, 0.23 mmol) in N,N-dimethylformamide (3.0 mL) is added di-imidazol-1-yl-methanone (37 mg, 0.23 mmol) and the mixture is stirred for 90 minutes. The product of Example 47 Step E (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo. The crude material is purified via silica gel chromatography using a gradient elution of 0-50% ethyl acetate/hexane to afford the desired product (95 mg, 72%). LCMS: 637.42 (M+H⁺).

### Step B: N-{4-[1-(2-Fluoro-4-trifluoromethyl-benzoyl)-piperidin-4-yl]-3-trifluoromethyl-benzoyl}-guanidine

To a solution of the product of Step A (95 mg, 0.15 mmol) in ethanol (3 mL) under an argon atmosphere is added 10 wt % palladium on carbon (32 mg, 0.015 mmol, wet, Degussa type) and the reaction is stirred under a hydrogen atmosphere for 16 hours. The mixture is filtered through Celite and the solvent is removed in vacuo. The residue is dissolved in dichloromethane and precipitated using 4 M hydrogen chloride in 1,4-dioxane and ether. The desired product hydrochloride salt is isolated by filtration as a colorless solid (54 mg, 67%). LCMS: 505.39 (M+H⁺).

### Example 62

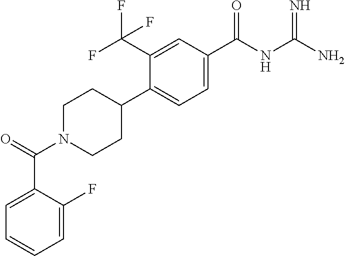

### N-{4-[1-(2-Fluoro-benzoyl)-piperidin-4-yl]-3-trifluoromethyl-benzoyl}-guanidine

### Step A: N-{4-[1-(2-fluoro-benzoyl)-piperidin-4-yl]-3-trifluoromethyl-benzoyl}-N′-(carbobenzyloxy)-guanidine

To a solution of 2-fluorobenzoic acid (32 mg, 0.23 mmol) in N,N-dimethylformamide (3.0 mL) is added di-imidazol-1-yl-methanone (37 mg, 0.23 mmol) and the mixture is stirred for 90 minutes. The product of example 47 Step E (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo. The crude material is purified via silica gel chromatography using a gradient elution of 0-60% ethyl acetate/hexane to afford the desired product (89 mg, 75%). LCMS: 569.49 (M+H⁺).

### Step B: N-{4-[1-(2-Fluoro-4-trifluoromethyl-benzoyl)-piperidin-4-yl]-3-trifluoromethyl-benzoyl}-guanidine

To a solution of the product of Step A (89 mg, 0.16 mmol) in ethanol (3 mL) under an argon atmosphere is added 10 wt % palladium on carbon (37 mg, 0.016 mmol, wet, Degussa type) and the reaction is stirred under a hydrogen atmosphere for 16 hours. The mixture is filtered through Celite and the solvent is removed in vacuo. The residue is purified via preparative HPLC using a gradient elution from 10-80% acetonitrile/water with 0.1% trifluoroacetic acid obtain the desired product as a trifluoroacetic acid salt (51 mg, 59%). LCMS: 437.73 (M+H⁺).

The following compounds are prepared using the procedures of Example 62 and substituting for the appropriate starting materials.

| Example | | | m/z |
|---|---|---|---|
| No. | Structure | Name | [M + H⁺] |
| 63 | 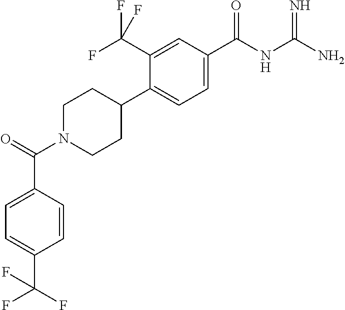 | N-{3-trifluoromethyl-4-[1- (4-trifluoromethyl- benzoyl)-piperidin-4-yl]- benzoyl}-guanidine | 487.74 |
| 64 | 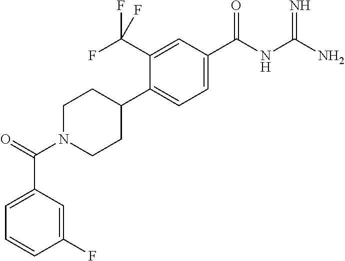 | N-{4-[1-(3-fluoro- benzoyl)-piperidin-4-yl]- 3-trifluoromethyl- benzoyl}-guanidine | 437.40 |

### Example 65

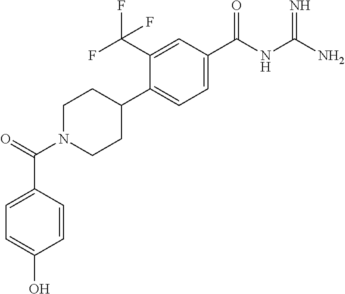

### N-{4-[1-(4-Hydroxy-benzoyl)-piperidin-4-yl]-3-trifluoromethyl-benzoyl}-guanidine

### Step A: N-{4-[1-(4-benzyloxy-benzoyl)-piperidin-4-yl]-3-trifluoromethyl-benzoyl}-N′-(carbobenzyloxy)-guanidine

To a solution of 4-(benzyloxy)-benzoic acid (52 mg, 0.23 mmol) in N,N-dimethylformamide (3.0 mL) is added di-imidazol-1-yl-methanone (37 mg, 0.23 mmol) and the mixture is stirred for 90 minutes. The product from Example 47 Step E (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo. The crude material is purified via silica gel chromatography using a gradient elution of 0-60% ethyl acetate/hexane to afford the desired product (78 mg, 57%). LCMS: 657.60 (M+H⁺).

### Step B: N-{4-[1-(4-Benzyloxy-benzoyl)-piperidin-4-yl]-3-trifluoromethyl-benzoyl}-guanidine

To a solution of the product from Step A (78 mg, 0.12 mmol) in ethanol (3 mL) under an argon atmosphere is added 10 wt % palladium on carbon (26 mg, 0.012 mmol, wet, Degussa type) and the reaction is stirred under a hydrogen atmosphere for 16 hours. The mixture is filtered through Celite and the solvent is removed in vacuo. The residue is purified via preparative HPLC using a gradient elution from 10-90% acetonitrile/water with 0.1% trifluoroacetic acid to obtain the desired product as a trifluoroacetic acid salt (39 mg, 59%). LCMS: 435.72 (M+H⁺).

### Example 66

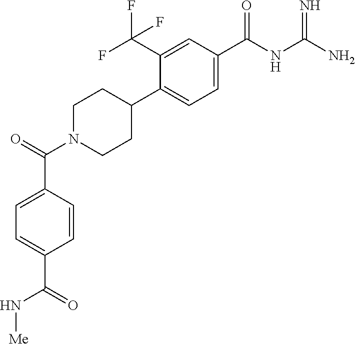

### 4-[4-(4-Guanidinocarbonyl-2-trifluoromethyl-phenyl)-piperidine-1-carbonyl]-N-methyl-benzamide

### Step A: N-Methyl-terephthalamic acid methyl ester

To a solution of 4-chlorocarbonyl-benzoic acid methyl ester (1.0 g, 5.0 mmol) in dichloromethane (10 mL) is added 2 M methylamine in methanol (5.29 mL, 10.6 mmol) and mixture stirred for 1 hour. The mixture is diluted with ethyl acetate, washed with water, 1 M hydrochloric acid, and brine. The organic phase is dried over Na₂SO₄, filtered, and the solvent is removed in vacuo to give the desired product as a colorless solid (805 mg, 83%). LCMS: 194.31 (M+H⁺).

### Step B: N-Methyl-terephthalamic acid

To a solution of the product from Step A (805 mg, 4.17 mmol) in methanol (30 mL) and water (10 mL) is added potassium carbonate (1.73 g, 12.5 mmol) and the mixture is stirred at 50° C. for 4 hours. The methanol is removed in vacuo and the residue is diluted with water and acidified with 1 M hydrochloric acid. The crude product is isolated by filtration and the residue is redissolved in 0.5 M aqueous sodium hydroxide and washed twice with ethyl acetate. The aqueous layer is acidified with 1 M hydrochloric acid and the product is isolated by filtration as a colorless solid (567 mg, 76%). LCMS: 180.29 (M+H⁺).

### Step C: 4-[4-(4-N′-Carbobenzyloxy-guanidinocarbonyl-2-trifluoromethyl-phenyl)-piperidine-1-carbonyl]-N-methyl-benzamide

To a solution of the product of Step B (61 mg, 0.34 mmol) in N,N-dimethylformamide (4.0 mL) is added di-imidazol-1-yl-methanone (55 mg, 0.34 mmol) and the mixture is stirred for 90 minutes. The product from example 45 Step E (150 mg, 0.31 mmol) is then added followed by N,N-diisopropylethylamine (0.16 mL, 0.93 mmol) and the reaction is stirred overnight at room temperature. The mixture is diluted with ethyl acetate, washed twice with water, once with brine, dried over Na₂SO₄, filtered, and the solvent is removed in vacuo to afford the desired product (150 mg, 72%). LCMS: 608.68 (M+H⁺).

### Step D: 4-[4-(4-Guanidinocarbonyl-2-trifluoromethyl-phenyl)-piperidine-1-carbonyl]-N-methyl-benzamide

To a solution of the product of Step C (135 mg, 0.22 mmol) in ethanol (4 mL) under an argon atmosphere is added 10 wt % palladium on carbon (96 mg, 0.044 mmol, wet, Degussa type) and the reaction is stirred under a hydrogen atmosphere for 36 hours.

The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (50 mg, 38%). LCMS: 435.72 (M+H⁺).

### Example 67

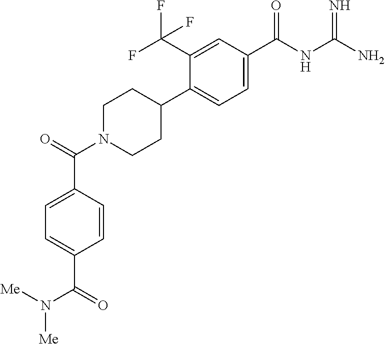

### 4-[4-(4-Guanidinocarbonyl-2-trifluoromethyl-phenyl)-piperidine-1-carbonyl]-N,N-dimethyl-benzamide

The title compound is prepared according to the procedure in Example 66 using the appropriate starting materials to afford the desired product as a glassy solid (100 mg, 70%). LCMS: 490.70 (M+H⁺).

### Example 68

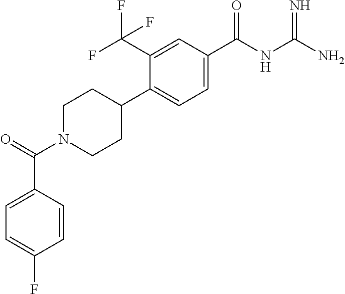

### N-{4-[1-(4-Fluoro-benzoyl)-piperidin-4-yl]-3-trifluoromethyl-benzoyl}-guanidine

### Step A: 4-(4-Methoxycarbonyl-2-trifluoromethyl-phenyl)-piperidine-1-carboxylic acid tert-butyl ester

To a solution of the product of Example 47 Step B (13.6 g, 35.3 mmol) in acetic acid (180 mL) under an argon atmosphere is added platinum(IV) oxide (750 mg, 3.3 mmol) and the mixture is stirred under a hydrogen atmosphere for 60 hours. The mixture is filtered through Celite and the solvent is removed in vacuo to give the desired product as a foam (13.5 g, 99%). LCMS: 332.20 (M+H⁺-56)

### Step B: 4-(4-Carboxy-2-trifluoromethyl-phenyl)-piperidine-1-carboxylic acid tert-butyl ester

To a solution of the product from Step A (5.10 g, 13.2 mmol) in methanol (39 mL) and water (13 mL) is added potassium carbonate (3.64 g, 26.3 mmol) and the reaction is stirred 16 hours at room temperature. The methanol is removed in vacuo and the residue is poured into dilute aqueous hydrochloric acid and the desired product is isolated by filtration (5.0 g, 100%). LCMS: 372.20 (M−H⁺).

### Step C: 4-(4-(N-Carbobenzyloxy)-guanidinocarbonyl-2-trifluoromethyl-phenyl)-piperidine-1-carboxylic acid tert-butyl ester

To a solution of the product from Step B (5.00 g, 13.2 mmol) in 1-methyl-2-pyrrolidinone (50 mL) is added 2-chloro-1-methylpyridinium iodide (4.11 g, 16.1 mmol) and the resulting solution is stirred for 90 minutes. N-carbobenzyloxy-guanidine (3.36 g, 17.4 mmol) is then added followed by N,N-diisopropylethylamine (6.66 mL, 40.2 mmol) and the reaction is stirred overnight. The mixture is poured into a mixture of 88% formic acid (6 mL) and water (250 mL) and the product is isolated as a colorless solid by filtration (6.63 g, 90%). LCMS: 549.20 (M+H⁺).

### Step D: N-(4-Piperidin-4-yl-3-trifluoromethyl-benzoyl)-guanidine

To a solution of the product from Step C (6.63 g, 12.1 mmol) in 1,4-dioxane (100 mL) is added 4 M hydrogen chloride in 1,4-dioxane (24.2 mL, 96 mmol) and the mixture is stirred for 16 hours at 50° C. The mixture is then diluted with ether (250 mL) and the desired product is isolated by filtration as a colorless solid (5.82 g, 99%). LCMS: 449.20 (M+H⁺).

### Step E: N-{4-[1-(4-fluoro-benzoyl)-piperidin-4-yl]-3-trifluoromethyl-benzoyl}-N′-(carbobenzyloxy)-guanidine

To a solution of 4-fluorobenzoic acid (1.85 g, 13.2 mmol) in N,N-dimethylformamide (120 mL) is added di-imidazol-1-yl-methanone (2.14 g, 13.2 mmol) and the mixture is stirred for 30 minutes. The product from Step D (5.82 g mg, 12.0 mmol) is added followed by N,N-diisopropylethylamine (6.46 mL, 36.0 mmol) and the reaction is stirred overnight at room temperature. The mixture is poured into an ice-cold dilute hydrochloric acid solution and the product is isolated by filtration (6.85 g, 100%). LCMS: 571.20 (M+H⁺).

### Step F: N-{4-[1-(4-Fluoro-benzoyl)-piperidin-4-yl]-3-trifluoromethyl-benzoyl}-guanidine

To a solution of the product from Step E (6.85 g, 12.0 mmol) in ethanol (75 mL) under an argon atmosphere is added 20 wt % Pd(OH)₂ on carbon (500 mg, 0.36 mmol, wet) and the mixture is stirred under a hydrogen atmosphere for 16 hours. The mixture is filtered through Celite and the solvent is removed in vacuo. The residue is dissolved in dioxane (100 mL) and treated with excess 4 M hydrogen chloride in 1,4-dioxane. The solvent is removed in vacuo and the resulting residue is triturated with ether. The resulting solid is recrystallized from acetonitrile to give the desired product as a colorless solid (3.77 g, 66%). LCMS: 437.20 (M+H⁺).

### Example 69

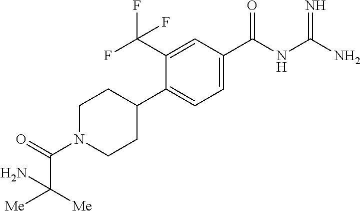

### N-{4-[1-(2-Amino-2-methyl-propionyl)-piperidin-4-yl]-3-trifluoromethyl-benzoyl}-guanidine

### Step A: {2-[4-(4-(N-Carbobenzyloxy)-guanidinocarbonyl-2-trifluoromethyl-phenyl)-piperidin-1-yl]-1,1-dimethyl-2-oxo-ethyl}-carbamic acid benzyl ester

To the product of Example 68 Step D (250 mg, 0.516 mmol) in N,N-dimethylformamide (5 mL) is added 2-benzyloxycarbonylamino-2-methyl-propionic acid (135 mg, 0.567 mmol), (2-7-aza-1H-benzotriazole-1-yl)-1,1,3,3-tetramethylumnium hexafluorophosphate (222 mg, 0.583 mmol), and N-methylmorpholine (0.115 mL, 1.05 mmol) and the mixture is stirred 16 hours at 40° C. The mixture is diluted with ethyl acetate, washed three times with water, once with brine, dried over Na₂SO₄, filtered, and the solvent is removed in vacuo. The crude material is purified via silica gel chromatography using a gradient elution of 0-70% ethyl acetate/hexanes to afford the desired product (80 mg, 23%). LCMS: 668.47 to (M+H⁺).

### Step B: N-{4-[1-(2-Amino-2-meth 1-propionyl)-piperidin-4-yl]-3-trifluoromethyl-benzoyl}-guanidine

To a solution of the product of Step A (80 mg, 0.12 mmol) in ethanol (4 mL) under an argon atmosphere is added 20 wt % palladium(II) hydroxide (17 mg, 0.012 mmol, 50% wet) and the mixture is stirred under an atmosphere of hydrogen for 16 hours. The mixture is filtered through Celite and the solvent is removed in vacuo. The residue is purified via preparative HPLC using a gradient elution from 10-70% acetonitrile/water with 0.1% trifluoroacetic acid to obtain the desired product as a bis-trifluoroacetic acid salt (35 mg, 47%). LCMS: 400.45 (M+H⁺).

### Example 70

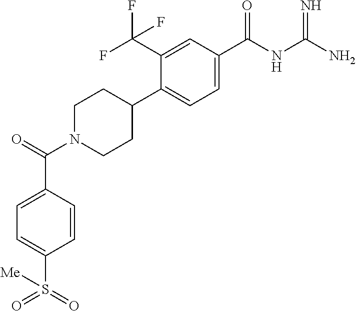

### N-{4-[1-(4-Methanesulfonyl-benzoyl)-piperidin-4-yl]-3-trifluoromethyl-benzoyl}-guanidine

### Step A: N-{4-[1-(4-methanesulfonyl-benzoyl)-piperidin-4-yl]-3-trifluoromethyl-benzoyl}-N′-(carbobenzyloxy)-guanidine

To a solution of 4-methanesulfonylbenzoic acid (136 mg, 0.681 mmol) in N,N-dimethylformamide (8.0 mL) is added di-imidazol-1-yl-methanone (110 mg, 0.681 mmol) and the mixture is stirred for 90 minutes. The product of Example 68 Step D (300 mg, 0.619 mmol) is then added followed by N,N-diisopropylethylamine (0.33 mL, 1.87 mmol) and the reaction is stirred overnight at room temperature. The mixture is diluted with ethyl acetate, washed twice with water, once with brine, dried over Na₂SO₄, filtered, and the solvent is removed in vacuo to afford the desired product (376 mg, 82%). LCMS: 631.34 (M+H⁺).

### Step B: N-{4-[1-(4-Methanesulfonyl-benzoyl)-piperidin-4-yl]-3-trifluoromethyl-benzoyl}-guanidine

To a solution of the product of Step A (320 mg, 0.51 mmol) in ethanol (3 mL) under an argon atmosphere is added 10 wt % palladium on carbon (108 mg, 0.051 mmol, wet, Degussa type) and the reaction is stirred under a hydrogen atmosphere for 16 hours. The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (253 mg, 82%). LCMS: 497.31 (M+H⁺).

### Example 71

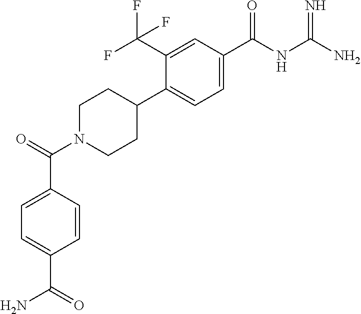

### 4-[4-(4-Guanidinocarbonyl-2-trifluoromethyl-phenyl)-piperidine-1-carbonyl]-benzamide

The title compound is prepared according to the procedure in Example 70 using the appropriate starting materials to afford the desired product as a glassy solid (35 mg, 37%). LCMS: 462.40 (M+H⁺).

### Example 72

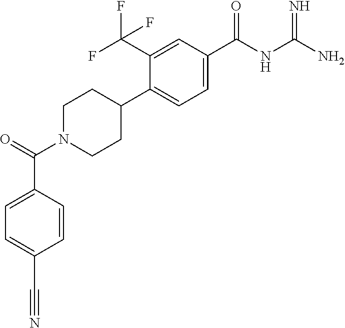

### N-{4-[1-(4-Cyano-benzoyl)-piperidin-4-yl]-3-trifluoromethyl-benzoyl}-guanidine

### Step A: N-{4-[1-(4-cyano-benzoyl)-piperidin-4-yl]-3-trifluoromethyl-benzoyl}-N′-(carbobenzyloxy)-guanidine

To a solution of 4-cyanobenzoic acid (33 mg, 0.23 mmol) in N,N-dimethylformamide (3.0 mL) is added di-imidazol-1-yl-methanone (37 mg, 0.23 mmol) and the mixture is stirred for 90 minutes. The product of Example 68 Step D (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo to afford the desired product (116 mg, 86%). LCMS: 578.41 (M+H⁺).

### Step B: N-{4-[1-(4-Cyano-benzoyl)-piperidin-4-yl]-3-trifluoromethyl-benzoyl}-guanidine

To a solution of the product from Step A (102 mg, 0.17 mmol) in ethanol (4 mL) under an argon atmosphere is added 20 wt % palladium(II) hydroxide on carbon (25 mg, 0.017 mmol, 50% wet) and the reaction is stirred under a hydrogen atmosphere for 16 hours. The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (66 mg, 67%). LCMS: 444.40 (M+H⁺).

The following compounds are prepared using the procedures of Example 72 and substituting for the appropriate starting materials.

| Example | | | m/z |
|---|---|---|---|
| No. | Structure | Name | [M + H⁺] |
| 73 | 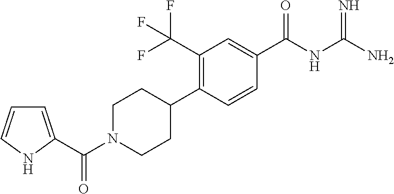 | N-{4-[1-(1H-Pyrrole- 2-carbonyl)-piperidin- 4-yl]-3- trifluoromethyl- benzoyl}-guanidine; dihydrochloride | 408.56 |
| 74 | 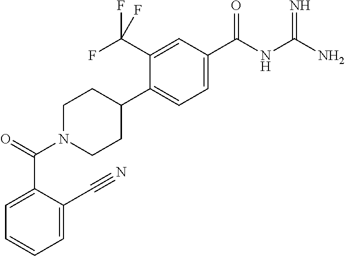 | N-{4-[1-(2-cyano- benzoyl)-piperidin-4- yl]-3-trifluoromethyl- benzoyl}-guanidine | 444.39 |
| 75 | 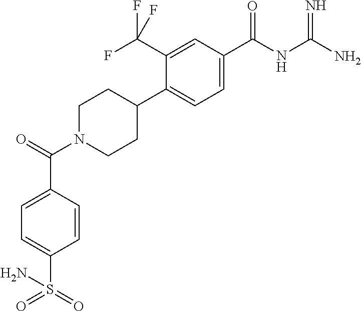 | 4-[4-(4- guanidinocarbonyl-2- trifluoromethyl- phenyl)-piperidine-1- carbonyl]- benzenesulfonamide | 498.61 |
| 76 | 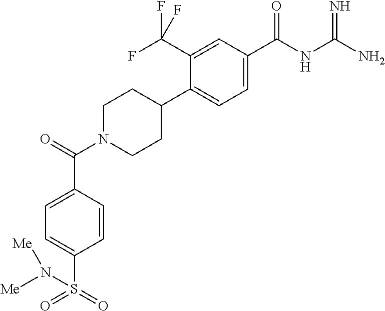 | 4-[4-(4- guanidinocarbonyl-2- trifluoromethyl- phenyl)-piperidine-1- carbonyl]-N,N- dimethyl- benzenesulfonamide | 526.38 |
| 77 | 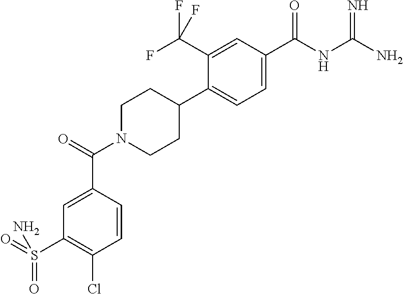 | 2-chloro-5-[4-(4- guanidinocarbonyl-2- trifluoromethyl- phenyl)-piperidine-1- carbonyl]- benzenesulfonamide | 532.32 |
| 79 | 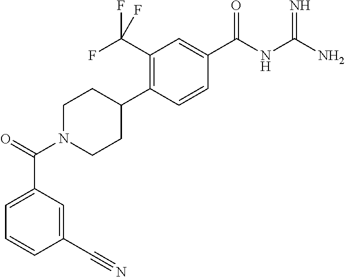 | N-{4-[1-(3-Cyano- benzoyl)-piperidin-4- yl]-3-trifluoromethyl- benzoyl}-guanidine | 444.39 |
| 79 | 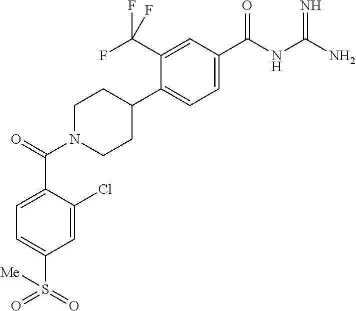 | N-{4-[1-(2-chloro-4- methanesulfonyl- benzoyl)-piperidin-4- yl]-3-trifluoromethyl- benzoyl}-guanidine | 529.00 |

### Example 80

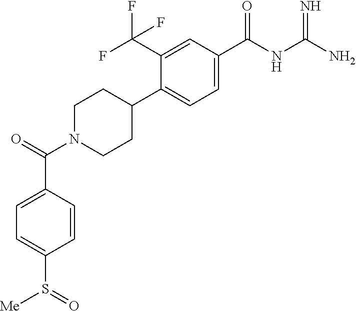

### N-{4-[1-(4-Methanesulfinyl-benzoyl)-piperidin-4-yl]-3-trifluoromethyl-benzoyl}-guanidine

### Step A: N-{4-[1-(4-methylsulfanyl-benzoyl)-piperidin-4-yl]-3-trifluoromethyl-benzoyl}-N′-(carbobenzyloxy)-guanidine

To a solution of 4-methylthiobenzoic acid (38 mg, 0.23 mmol) in N,N-dimethylformamide (3.0 mL) is added di-imidazol-1-yl-methanone (37 mg, 0.23 mmol) and the mixture is stirred for 90 minutes. The product of Example 68 Step D (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo to afford the desired product (114 mg, 85%). LCMS: 599.46 (M+H⁺).

### Step B: N-{4-[1-(4-Methylsulfanyl-benzoyl)-piperidin-4-yl]-3-trifluoromethyl-benzoyl}-guanidine

To a solution of the product from Step A (98 mg, 0.16 mmol) in trifluoroacetic acid (3 mL) is added thioanisole (0.90 mL) and the reaction mixture is stirred for 6 hours. The solvemt is removed in vacuo and the residue is purified by preparative TLC using 5% methanol in dichloromethane with 1% triethylamine as eluent to give the desired product as a white glassy solid (25 mg, 33%). LCMS: 465.43 (M+H⁺).

### Step C: N-{4-[1-(4-Methanesulfinyl-benzoyl)-piperidin-4-yl]-3-trifluoromethyl-benzoyl}-guanidine

To a solution of the product from Step B (25 mg, 0.054 mmol) in acetonitrile (1 mL) and water (1 mL) is added sodium periodate (15 mg, 0.07 mmol) and the mixture is stirred for 16 hours. The resulting solution is filtered through a 0.45 micron filter and the residue is purified via preparative HPLC using a gradient elution from 10-80% acetonitrile/water with 0.1% trifluoroacetic acid to obtain the desired product as a trifluoroacetic acid salt (22 mg, 69%). LCMS: 481.36 (M+H⁺).

### Example 81

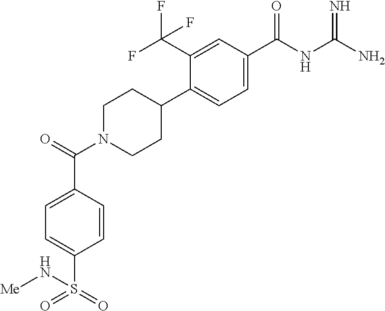

### 4-[4-(4-Guanidinocarbonyl-2-trifluoromethyl-phenyl)-piperidine-1-carbonyl]-N-methyl-benzenesulfonamide

### Step A: 4-[4-(4-(N′-carbobenzyloxy)-Guanidinocarbonyl-2-trifluoromethyl-phenyl)-piperidine-1-carbonyl]-N-methyl-benzenesulfonamide

To a solution of 4-methylsulfamoyl-benzoic acid (49 mg, 0.23 mmol) in N,N-dimethylformamide (3 mL) is added di-imidazol-1-yl-methanone (37 mg, 0.23 mmol) and the mixture is stirred for 90 minutes. The product from Example 68 Step D (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo to afford the desired product (123 mg, 80%). LCMS: 646.39 (M+H⁺).

### Step B: 4-[4-(4-Guanidinocarbonyl-2-trifluoromethyl-phenyl)-piperidine-1-carbonyl]-N-methyl-benzenesulfonamide

To a solution of the product of Step A (77 mg, 0.10 mmol) in acetic acid (1 mL) is added 30% hydrogen bromide in acetic acid (1 mL) and the mixture is heated at 50° C. for 4 hours. The mixture is diluted with dichloromethane and the crude product is precipitated with ether and isolated by filtration. The residue is purified via preparative HPLC using a gradient elution from 10-80% acetonitrile/water with 0.1% trifluoroacetic acid to obtain the desired product as a trifluoroacetic acid salt (10 mg, 16%). LCMS: 512.66 (M+H⁺).

### Example 82

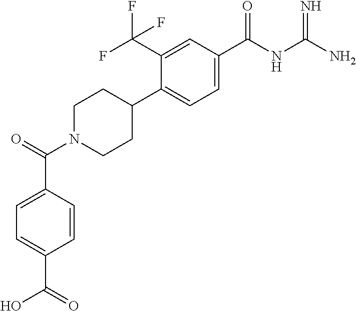

### 4-[4-(4-Guanidinocarbonyl-2-trifluoromethyl-phenyl)-piperidine-1-carbonyl]-benzoic acid

To a solution of Example 56 (32 mg, 0.054 mmol) in methanol (1.0 mL) and water (0.25 mL) is added potassium carbonate (26 mg, 0.19 mmol) and the mixture is stirred for 2 hours at 50° C. The solvent is removed in vacuo and the residue is purified via preparative HPLC using a gradient elution from 10-70% acetonitrile/water with 0.1% trifluoroacetic acid to obtain the desired product as a trifluoroacetic acid salt (23 mg, 74%). LCMS: 463.39 (M+H⁺).

### Example 83

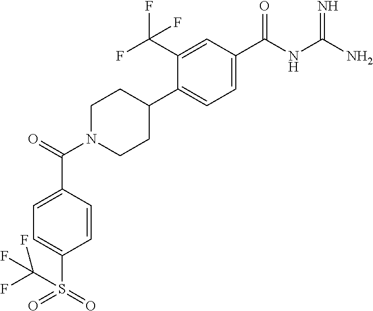

### N-{4-[1-(4-Trifluoromethanesulfonyl-benzoyl)-piperidin-4-yl]-3-trifluoromethyl-benzoyl}-guanidine

### Step A: 4-Trifluoromethanesulfonyl-benzoic acid

To a solution of 4-trifluoromethylsulfanyl-benzoic acid (250 mg, 1.13 mmol) in methanol (10 mL) and water (8 mL) is added oxone (5.59 g, 6.75 mmol) and the reaction is stirred first at 50° C. for 6 hours, then at room temperature for 4 days. The mixture is diluted with ethyl acetate and water and the layers are separated. The aqueous layer is extracted with ethyl acetate and the combined organic phase washed with brine, dried over Na₂SO₄, filtered, and the solvent is removed in vacuo to afford the desired product (245 mg, 86%). LCMS: 253.00 (M−H⁺).

### Step B: N-{4-[1-(4-Trifluoromethanesulfonyl-benzoyl)-piperidin-4-yl]-3-trifluoromethyl-benzoyl}-N′-(carbobenzyloxy)-guanidine

To a solution of the product from Step A (58 mg, 0.23 mmol) in N,N-dimethylformamide (3 mL) is added di-imidazol-1-yl-methanone (37 mg, 0.23 mmol) and the mixture is stirred for 90 minutes. The product of Example 68 Step D (100 mg, 0.21 mmol) is then added followed by N,N-diisopropylethylamine (0.13 mL, 0.73 mmol) and the reaction is stirred overnight at room temperature. The mixture is diluted with ethyl acetate, washed twice with water, once with brine, dried over Na₂SO₄, filtered, and the solvent is removed in vacuo to afford the desired product (131 mg, 84%). LCMS: 685.20 (M+H⁺).

### Step C: N-{4-[1-(4-Trifluoromethanesulfonyl-benzoyl)-piperidin-4-yl]-3-trifluoromethyl-benzoyl}-guanidine

To a solution of the product from Step B (131 mg, 0.19 mmol) in ethanol (4 mL) under an argon atmosphere is added 20 wt % palladium(II) hydroxide on carbon (36 mg, 0.026 mmol, 50% wet) and the reaction is stirred under a hydrogen atmosphere for 16 hours. The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (96 mg, 76%). LCMS: 551.20 (M+H⁺).

### Example 84

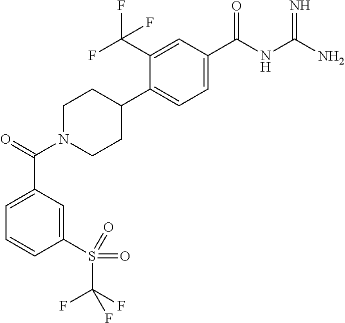

### N-{4-[1-(3-Trifluoromethanesulfonyl-benzoyl)-piperidin-4-yl]-3-trifluoromethyl-benzoyl}-guanidine

The title compound is prepared according to the procedure for Example 83 using the appropriate starting materials to afford the desired product as a colorless solid (83 mg, 0.125 mmol). LCMS: 551.20 (M+H⁺).

### Example 85

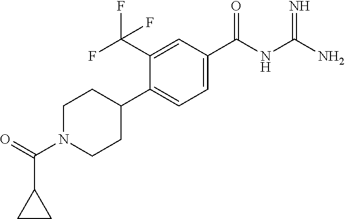

### N-[4-(1-Cyclopropanecarbonyl-piperidin-4-yl)-3-trifluoromethyl-benzoyl]-guanidine

### Step A: 1-(N-carbobenzyloxy)-3-[4-(1-cyclopropanecarbonyl-piperidin-4-yl)-3-trifluoromethyl-benzoyl]guanidine

To a suspension of the product from Example 68, Step D (100 mg, 0.21 mmol) in dichloromethane (2.0 mL) is added triethylamine (0.072 mL, 0.52 mmol). Cyclopropanecarboxylic acid chloride (0.019 mL, 0.21 mmol) is then added dropwise to and the mixture is stirred for 1 hour at room temperature. The reaction is quenched by the addition of a saturated aqueous solution of sodium bicarbonate (3.0 mL) and water (3.0 mL) and the mixture is extracted three times with dichloromethane (30 mL). The combined organic phase is dried over Na₂SO₄ and the solvent is removed to give the crude product (100 mg) which is used in the next step without purification.

### Step B: N-[4-(1-Cyclopropanecarbonyl-piperidin-4-yl)-3-trifluoromethyl-benzoyl]-guanidine

To a solution of the product from Step A (100 mg) in ethyl acetate (2.0 mL) is added 10% palladium on carbon (31 mg, 0.03 mmol, wet, Degussa type). The reaction mixture is stirred under a hydrogen atmosphere for 2 hours. The mixture is filtered through Celite and the solvent is removed to give crude product. The crude material is purified via silica gel chromatography using a gradient elution of 0-10% methanol/dichloromethane to afford the desired product as a colorless amorphous solid (65 mg, 88%). LCMS: 383.44 (M+H⁺).

The following compounds are prepared using the procedures of Example 85 and substituting for the appropriate starting materials.

| Example | | | m/z |
|---|---|---|---|
| No. | Structure | Name | [M + H⁺] |
| 86 | 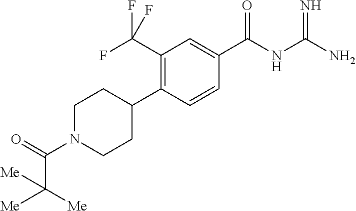 | N-{4-[1-(2,2-dimethyl- propionyl)-piperidin-4-yl]-3- trifluoromethyl-benzoyl}- guanidine | 399.69 |
| 87 | 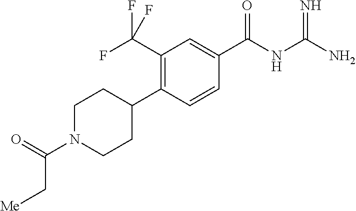 | N-[4-(1-propionyl-piperidin-4- yl)-3-tritluoromethyl-benzoyl]- guanidine | 371.62 |
| 88 | 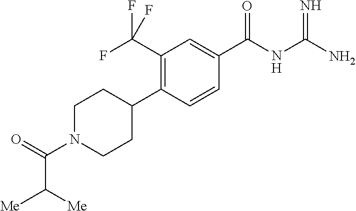 | N-[4-(1-isobutyryl-piperidin-4- yl)-3-trifluoromethyl-benzoyl]- guanidine | 385.58 |
| 89 | 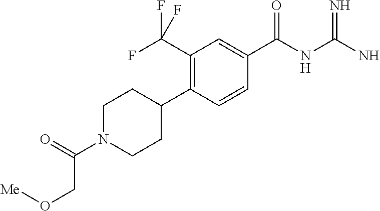 | N-{4-[1-(2-methoxy-acetyl)- piperidin-4-yl]-3- trifluoromethyl-benzoyl}- guanidine | 387.59 |
| 90 | 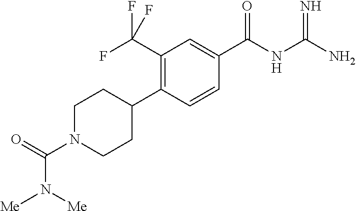 | 4-(4-Guanidinocarbonyl-2- trifluoromethyl-phenyl)- piperidine-1-carboxylic acid dimethylamide | 386.41 |

### Example 91

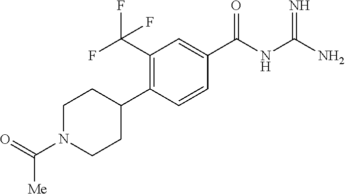

### N-[4-(1-acetyl-piperidin-4-yl)-3-trifluoromethyl-benzoyl]-guanidine

### Step A: 4-Piperidin-4-yl-3-trifluoromethyl-benzoic acid methyl ester

To a solution of the product of Example 68 Step A (74.85 g, 193 mmol) in 1,4-dioxane (1.1 L) is added concentrated aqueous hydrochloric acid (48.3 mL, 0.580 mmol) and the resulting solution is stirred at 50 ^{a} for 3 hours. The resulting mixture is cooled to room temperature and the solvent is removed in vacuo. The resulting white solid is suspended in diethyl ether and stirred for 10 minutes after which time the desired product is collected as a white solid by filtration (55.7 g, 89%).

### Step B: 4-(1-Acetyl-piperidin-4-yl)-3-trifluoromethyl-benzoic acid methyl ester

To a solution of the product of Step A (20.2 g, 62.0 mmol) in 1-methyl-2-pyrrolidinone (250 mL) at 0° C. is added triethylamine (17.4 mL, 125 mmol) followed by acetic anhydride (7.08 mL, 75 mmol) and the resulting mixture is warmed to room temperature and stirred for 1 hour. The reaction mixture is poured into water and extracted with ethyl acetate. The organic layer is washed four times with water, once with saturated aqueous sodium chloride, dried over MgSO₄, filtered, and the solvent is removed in vacuo to provide the desired product as a colorless oil (17.4 g, 85%).

### Step C: N-[4-(1-Acetyl-piperidin-4-yl)-3-trifluoromethyl-benzoyl]-guanidine

To a solution of guanidine hydrochloride (42.76 g, 448 mmol) in N,N-dimethylformamide (300 mL) at 4° C. is added sodium tert-pentoxide and the solution is warmed to room temperature and stirred 15 minutes. To this mixture is added the product of Step B (73.7 g, 224 mmol) in N,N-dimethylformamide (300 mL) and the mixture is warmed to room temperature and stirred for 21 hours. Water (200 mL) is then added and the mixture is stirred one hour. The mixture is then poured into water (500 mL) and extracted four times with dichloromethane and the combined organics are washed twice with a saturated solution of sodium bicarbonate then with saturated aqueous sodium chloride. The organiocs are separated and the solvent is removed in vacuo. The resulting oil is redissolved three times in dichloromethane removing the solvent in vacuo each time. The oil is then dissolved again in dichloromethane (150 mL) and upon standing a precipitate forms. The solid was collected by filtration to give the desired product as a white solid (67.8 g, 85%). LCMS: 357.20 (M+H⁺).

### Example 92

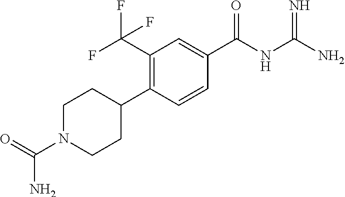

### 4-(4-Guanidinocarbonyl-2-trifluoromethyl-phenyl)-piperidine-1-carboxylic acid amide

### Step A: 1-(N-carbobenzyloxy)-3-[4-(1-carbamoyl-piperidin-4-yl)-3-trifluoromethyl-benzoyl]guanidine

To a suspension of the product from Example 68 Step D (100 mg, 0.21 mmol) in dichloromethane (5.0 mL) is added triethylamine (0.10 mL, 0.72 mmol). Trimethylsilyl isocyanate (0.66 mL, 4.1 mmol) is added dropwise and the mixture is stirred for 16 hours at room temperature. The reaction is quenched by the addition of a saturated aqueous solution of sodium bicarbonate (3.0 mL) and of water (50 mL) and the mixture is extracted three times with dichloromethane (50 mL). The combined organic phase is dried over Na₂SO₄ and the solvent is removed in vacuo to give crude product. The crude material is purified via silica gel chromatography using a gradient elution of 0-6% methanol/dichloromethane to afford the desired product (78 mg, 77%).

### Step B: 4-(4-guanidinocarbonyl-2-trifluoromethyl-phenyl)-piperidine-1-carboxylic acid amide

To a solution of the product from Step A (78 mg, 0.16 mmol) in methanol (2.0 mL) is added 10% palladium on carbon (25 mg, 0.02 mmol, wet, Degussa type). The reaction mixture is stirred under a hydrogen atmosphere for 16 hours. The reaction mixture is filtered through Celite and the solvent is removed in vacuo to give the crude product. The crude material is purified by crystallization using methanol and ethyl acetate to afford the pure product as a white solid (47 mg, 83%). LCMS: 358.38 (M+H⁺).

### Example 93

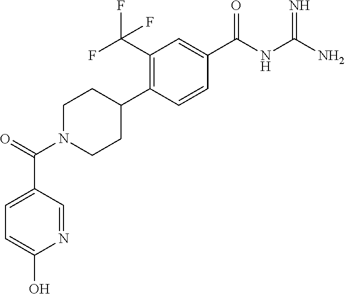

### N-{4-[1-(6-Hydroxy-pyridine-3-carbonyl)-piperidin-4-yl]-3-trifluoromethyl-benzoyl}-guanidine

### Step A: 6-Benzyloxy-nicotinonitrile

To a solution of benzyl alcohol (1.0 mL, 10 mmol) in dry N,N-dimethylformamide (15 mL) is added sodium hydride (400 mg, 10 mmol, 60%) at room temperature. The mixture is stirred for 30 minutes and 6-chloro-nicotinonitrile (714 mg, 5.0 mmol) is added. The mixture is stirred at room temperature for 2 hours. Then a saturated solution of aqueous ammonium chloride is added along with water (100 mL). The resulting solid is filtered off and washed with water, and dried in vacuo to give the desired product (420 mg, 40%).

### Step B: 6-Benzyloxy-nicotinic acid

To a solution of the product from Step A (420 mg, 2.0 mmol) in ethanol (4.0 mL) is added 10 M sodium hydroxide (2.0 mL, 20 mmol). The mixture is heated at 100° C. for 1 hour. The ethanol is removed in vacuo and water (50 mL) is added. The pH of the aqueous layer is adjusted to about 5 using 6.0 M hydrochloric acid. The resulting pale yellow solid is filtered off, washed with water and dried in vacuo to give the desired product (394 mg, 86%).

### Step C: 1-(N-carbobenzyloxy)-3-{4-[1-(6-benzyloxy-pyridine-3-carbonyl)-piperidin-4-yl]-3-trifluoromethyl-benzoyl}guanidine

To a solution of the product from Step B (52 mg, 0.23 mmol) in N,N-dimethylformamide (2.0 mL) is added di-imidazol-1-yl-methanone (37 mg, 0.23 mmol) and the mixture is stirred for 45 minutes. The product of Example 68, Step D (100 mg, 0.21 mmol) and triethylamine (0.06 mL, 0.41 mmol) is added and the mixture is stirred for 3 hours. Water (50 mL) is added and the resulting colorless solid is filtered off, washed with water, and dried in vacuo to give the product (132 mg, 97%).

### Step D: N-{4-[1-(6-Hydroxy-pyridine-3-carbonyl)-piperidin-4-yl]-3-trifluoromethyl-benzoyl}-guanidine

To a solution of the product from Step C (132 mg, 0.20 mmol) in methanol (2.0 mL) is added 10% palladium on carbon (32 mg, 0.03 mmol, wet, Degussa type), and the reaction mixture is stirred under a hydrogen atmosphere for 24 hours. The reaction mixture is filtered through Celite and the solvent is removed in vacuo. The crude product is dissolved in methanol and ether is added. The resulting colorless solid is filtered off and washed with ether. The crude material is purified by re-crystallization from methanol to give the desired product (23 mg, 29%). LCMS: 436.41 (M+H⁺).

### Example 94

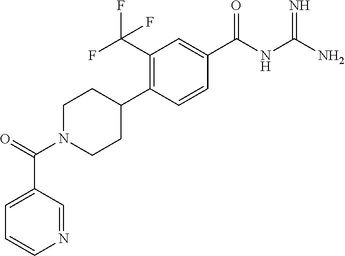

### N-{4-[1-(Pyridine-3-carbonyl)-piperidin-4-yl]-3-trifluoromethyl-benzoyl}-guanidine

### Step A: 1-(N-carbobenzyloxy)-3-{4-[1-(pyridine-3-carbonyl)-piperidin-4-yl]-3-trifluoromethyl-benzoyl}guanidine

To a solution of nicotinic acid (28 mg, 0.23 mmol) in N,N-dimethylformamide (2.0 mL) is added di-imidazol-1-yl-methanone (37 mg, 0.23 mmol) and the mixture is stirred for 45 minutes at room temperature. The product of Example 68, Step D (100 mg, 0.21 mmol) is then added followed by triethylamine (0.06 mL, 0.41 mmol) and the mixture is stirred for 16 hours. Water (50 mL) is added and the resulting colorless solid is filtered off, rinsed with water and dried in vacuo to give the crude product which is used in the next step without purification.

### Step B: N-{4-[1-(Pyridine-3-carbonyl)-piperidin-4-yl]-3-trifluoromethyl-benzoyl}-guanidine

To a solution of the product of step A in ethyl acetate (1.0 mL) and methanol (1.0 mL) is added 10% Pd on carbon (33 mg, 0.03 mmol, wet, Degussa type). The reaction mixture is stirred under a hydrogen atmosphere for 16 hours. The mixture is filtered through Celite and the filtrate is evaporated to give crude product. The crude material is purified via silica gel chromatography using a gradient elution of 0-10% methanol/dichloromethane to afford the desired product (71 mg, 82%). LCMS: 420.60 (M+H⁺).

The following compounds are prepared using the procedures of Example 94 and substituting for the appropriate starting materials.

| Example | | | m/z |
|---|---|---|---|
| No. | Structure | Name | [M + H⁺] |
| 95 | 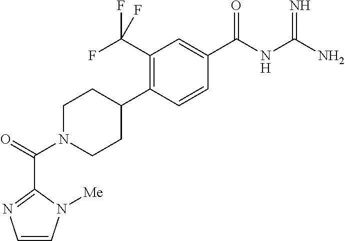 | N-{4-[1-(l-Methyl-1H- imidazole-2-carbonyl)- piperidin-4-yl]-3- trifluoromethyl-benzoyl}- guanidine | 423.55 |
| 96 | 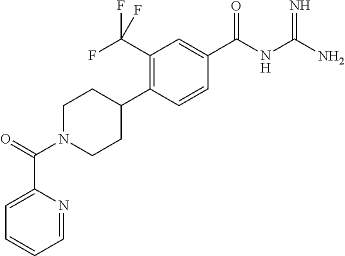 | N-{4-[1-(Pyridine-2-carbonyl)- piperidin-4-yl]-3- trifluoromethyl-benzoyl}- guanidine | 420.59 |
| 97 | 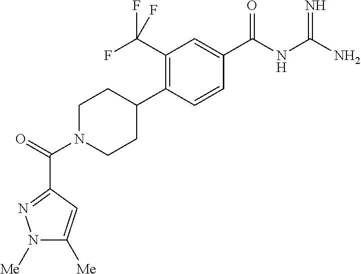 | N-{4-[1-(1,5-Dimethyl-1H- pyrazole-3-carbonyl)- piperidin-4-yl]-3- trifluoromethyl-benzoyl}- guanidine | 437.20 |
| 98 | 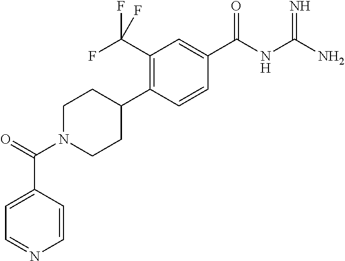 | N-{4-[1-(Pyridine-4-carbonyl)- piperidin-4-yl]-3- trifluoromethyl-benzoyl}- guanidine | 420.20 |
| 99 | 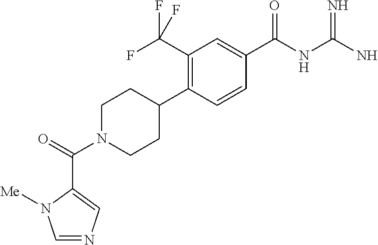 | N-{4-[1-(3-Methyl-3H- imidazole-4-carbonyl)- piperidin-4-yl]-3- trifluoromethyl-benzoyl}- guanidine | 423.76 |

### Example 100

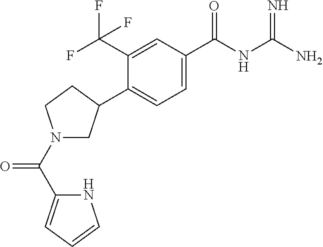

### N-{4-[1-(1H-Pyrrole-2-carbonyl)-pyrrolidin-3-yl]-3-trifluoromethyl-benzoyl}-guanidine

### Step A: 3-Oxo-pyrrolidine-1-carboxylic acid tert-butyl ester

To a solution of 3-hydroxy-pyrrolidine-1-carboxylic acid tert-butyl ester (10 g, 54 mmol) in dichloromethane (300 mL) is added Dess-Martin Reagent (45.9 g, 108 mmol) in three portions. The resulting mixture is stirred for 16 hours. The mixture is filtered through Celite and the solvent removed in vacuo. The crude material is purified via silica gel chromatography using a gradient elution of 0-8% ethyl acetate/hexanes to afford the desired product as a glassy solid (9.8 g, 97%).

### Step B: 3-Trifluoromethanesulfonyloxy-2,5-dihydro-pyrrole-1-carboxylic acid tert-butyl ester

To a solution of the product from Step A (5.0 g, 27 mmol) in tetrahydrofuran (50 mL) at −75° C. is added a 1M solution of sodium hexamethyldisilazane in tetrahydrofuran (50 mL) and the resulting mixture is stirred for 30 minutes. A solution of N-phenyl-bis-trifluoromethansulfonimide (10.7 g, 30.0 mmol) in tetrahydrofuran (100 mL) is then added slowly to the reaction. The mixture is warmed to 0° C. and stirred for 2 hours. Ice-water (100 mL) is added to the reaction mixture and the mixture is diluted with ethyl acetate. The layers are separated and the aqueous layer is extracted with ethyl acetate. The combined organic phase is dried over Na₂SO₄, filtered, and the solvent is removed in vacuo. The crude material is purified via neutral alumina chromatography using 10% ethyl acetate/petroleum ether as the eluent to afford the desired product as an oil (4.0 g, 62%). MS: 318.38 (M+H⁺).

### Step C: 3-(4,4,5,5-Tetramethyl-[1,3,2]dioxaborolan-2-yl)-2,5-dihydro-pyrrole-1-carboxylic acid tert-butyl ester

To a solution of the product from Step B (6.0 g, 18 mmol) in dioxane (40 mL) is added bis-pinacolatodiborane (5.2 g, 20 mmol), potassium acetate (5.5 g, 56 mmol), [1,1′-bis(diphenylphosphinoferrocene]dichloropalladium(II) (0.46 g, 0.56 mmol), and di-phenylphosphinoferrocene (0.29 g, 0.56 mmol). The resulting mixture is heated at 80° C. for 16 hours. The mixture is cooled to room temperature and diluted with water and the aqueous layer is extracted twice with ethyl acetate. The combined organic phase is washed with water, brine, dried over Na₂SO₄, filtered, and the solvent is removed in vacuo. The crude material is purified via silica gel chromatography using a gradient elution of 5-8% ethyl acetate/hexanes to afford the desired product as a pale yellow solid (6.2 g, 71%). MS: 296.44 (M+H⁺).

### Step D: 3-(4-cyano-2-trifluoromethyl-phenyl)-2,5-dihydro-pyrrole-1-carboxylic acid tert-butyl ester

To a solution of the product from Step C (4.8 g, 16 mmol) in anyhydrous N,N-dimethylformamide (30 mL) is added 4-bromo-3-trifluoromethyl benzonitrile (4.0 g, 16 mmol), [1,1′-bis(diphenylphosphinoferrocene]dichloropalladium(II) (1.9 g, 0.24 mmol), and potassium carbonate (6.7 g, 49 mmol). The resulting mixture is heated at 80° C. for 16 hours. The mixture is then cooled to room temperature, diluted with ether, filtered trhough Celite, and the solvent is removed in vacuo. The crude material is purified via silica gel chromatography using 5% ethyl acetate/petroleum ether as eluent to give the desired product as an oil (4.2 g, 56%). MS: 283.22 (M+H⁺-56).

### Step E 3-(4-Carboxy-2-trifluoromethyl-phenyl)-2,5-dihydro-pyrrole-1-carboxylic acid tert-butyl ester

To a solution of the product from Step D (4.2 g, 13 mmol) in ethanol (30 mL) is added water (60 mL) and sodium hydroxide (2.5 g, 64 mmol). The resulting mixture is refluxed for 2 hours. The solvent is removed in vacuo and the reaction is acidified with 1 N hydrochloric acid. The aqueous layer is extracted twice with dichloromethane and the combined organic phase is dried over Na₂SO₄, filtered, and the solvent is removed in vacuo to give the desired product as a white solid (3.2 g, 82%). MS: 358.27 (M+H⁺).

### Step F: 3-(4-Carboxy-2-trifluoromethyl-phenyl)-pyrrolidine-1-carboxylic acid tert-butyl ester

To a solution of the product of Step E (1.1 g, 3.1 mmol) in methanol (50 mL) is added 10% palladium on carbon (100 mg, 0.094 mmol, wet, Degussa type), and the mixture is reacted in a Parr hydrogenator under 60 psi hydrogen for 16 hours. The mixture is to filtered through Celite and the solvent is removed in vacuo to give the desired product as a white solid (1.1 g, 98%). MS: 358.36.9 (M−H⁺).

### Step G: 4-Pyrrolidin-3-yl-3-trifluoromethyl-benzoic acid methyl ester

To the product from Step F (1.1 g, 3.1 mmol) is added methanolic hydrogen chloride (20 mL) and the mixture is stirred for 16 hours. The mixture is evaporated in vacuo and the resulting residue is triturated with ether and filtered to give the desired product as light yellow solid (1.0 g, 92%).

### Step H: 4-[1-(1H-Pyrrole-2-carbonyl)-pyrrolidin-3-yl]-3-trifluoromethyl-benzoic acid methyl ester

To a solution of the product from Step G (1.0 g, 3.2 mmol) in tetrahydrofuran (15 mL) is added pyrrole 2-carboxylic acid (360 mg, 3.2 mmol), 2-(7-aza-1H-benzotriazole-1-yl)-1,1,3,3-tetramethyluronium hexafluorophosphate (1.84 g, 4.8 mmol), and N,N-diisopropylethylamine (1.67 mL, 10.0 mmol) at 0° C. The resulting mixture is stirred for 16 hours at room temperature. The mixture is diluted with water and extracted twice with ethyl acetate. The combined organic phase is dried over Na₂SO₄, filtered, and the solvent is removed in vacuo. The crude material is purified via silica gel chromatography using 2% methanol/dichloromethane as eluent to give the desired product as a white solid (1.1 g, 70%). MS: 367.1 (M+H⁺).

### Step I: 4-[1-(1H-Pyrrole-2-carbonyl)-pyrrolidin-3-yl]-3-trifluoromethyl-benzoic acid

To a solution of the product from Step H (1.1 g, 3.0 mmol) is added lithium hydroxide hydrate (378 mg, 9.0 mmol) and the mixture is stirred for 16 hours. The solvent is removed in vacuo and the residue is partitioned between water and ether. The layers are separated and the aqueous layer is acidified with 10 M citric acid solution and the desired product is isolated by filtration (380 mg, 55%). MS: 353.28 (M+H⁺).

### Step J: N-{4-[1-(1H-pyrrole-2-carbonyl)-pyrrolidin-3-yl]-3-trifluoromethyl-benzoyl}-N′-(carbobenzyloxy)-guanidine

To a solution of the product from Step I (100 mg, 0.28 mmol) in N-methylpyrrolidinone (3 mL) is added 2-chloro-1-methylpyridinium iodide (94 mg, 0.37 mmol) and the mixture is stirred for 90 minutes. N-carbobenzyloxy-guanidine (60 mg, 0.31 mmol) is then added followed by N,N-diisopropylethylamine (0.16 mL, 0.99 mmol) and the reaction is stirred overnight. The mixture is then partitioned between water and ethyl acetate and the organic phase is washed twice with water, once with brine, dried over Na₂SO₄, filtered, and the solvent is removed in vacuo to give the desired product as a glassy yellow solid. LCMS: 528.37 (M+H⁺).

### Step K: N-{4-[1-(1H-Pyrrole-2-carbonyl)-pyrrolidin-3-yl]-3-trifluoromethyl-benzoyl}-guanidine

To a solution of the product from Step J (149 mg, 0.282 mmol) in ethanol (4 mL) under an argon atmosphere is added 10 wt % palladium on carbon (60 mg, 0.028 mmol, wet, Degussa type) and the reaction is stirred under a hydrogen atmosphere for 16 hours. The mixture is filtered through Celite and the solvent is removed in vacuo. The residue is purified via preparative HPLC using a gradient elution from 10-80% acetonitrile/water with 0.1% trifluoroacetic acid to obtain the desired product as a trifluoroacetic acid salt (62 mg, 43%). LCMS: 435.40 (M+H⁺).

The following compounds are prepared using the procedure in Example 99 and substituting for the appropriate starting materials.

| Example | | | m/z |
|---|---|---|---|
| No. | Structure | Name | [M + H⁺] |
| 101 | 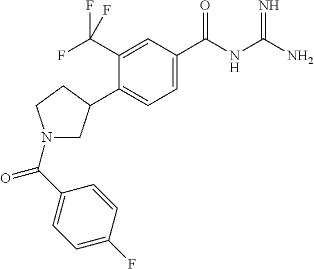 | N-{4-[1-(4-fluoro-benzoyl)- pyrrolidin-3-yl]-3- trifluoromethyl-benzoyl}- guanidine | 423.40 |
| 102 | 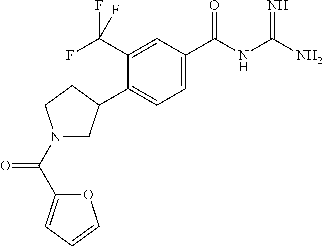 | N-{4-[1-(furan-2-carbonyl)- pyrrolidin-3-yl]-3- trifluoromethyl-benzoyl}- guanidine | 436.37 |

### Examples 103 and 104

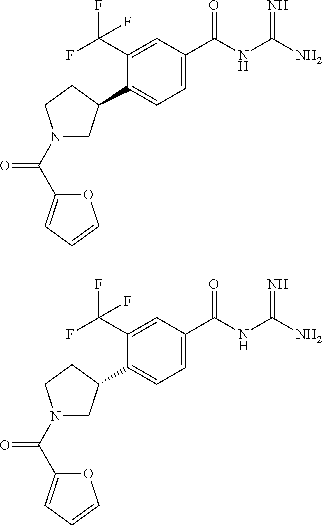

### N-{4-[(S)-1-(Furan-2-carbonyl)-pyrrolidin-3-yl]-3-trifluoromethyl-benzoyl}-guanidine and N-{4-[(R)-1-(Furan-2-carbonyl)-pyrrolidin-3-yl]-3-trifluoromethyl-benzoyl}-guanidine

A sample of the title product from Example 99 is separated using preparative chiral

HPLC with an OD-H column eluting 20% isopropyl/hexane to give the two pure enantiomers, the first eluting at 15.8 min and the second at 23.2 min LCMS: 436.37 (M+H⁺).

### Examples 105 and 106

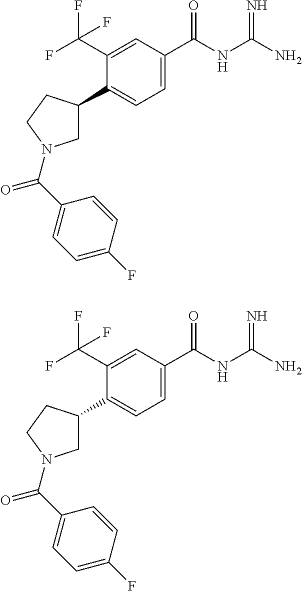

### N-{4-[(S)-1-(4-fluoro-benzoyl)-pyrrolidin-3-yl]-3-trifluoromethyl-benzoyl}-guanidine and

### N-{4-[(R)-1-(4-fluoro-benzoyl)-pyrrolidin-3-yl]-3-trifluoromethyl-benzoyl}-guanidine

A sample of the title product from Example 98 is separated using preparative chiral to HPLC with an OD-H column eluting 20% isopropyl/hexane to give the two pure enantiomers, the first eluting at 23.2 min and the second at 29.5 min LCMS: 423.40 (M+H⁺).

### Example 107

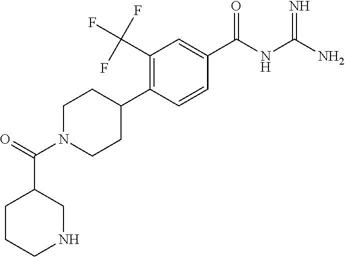

### N-{4-[1-(Piperidine-3-carbonyl)-piperidin-4-yl]-3-trifluoromethyl-benzoyl}-guanidine

### Step A: 4-Piperidin-4-yl-3-trifluoromethyl-benzoic acid methyl ester

To a solution of the product of Example 66 Step A (14.17 g, 36.6 mmol) in 1,4-dioxane (180 mL) is added 4 M hydrochloric acid in 1,4-dioxane (45 mL, 180 mmol) and the resulting mixture is stirred for 18 hours and then 6 hours at 50° C. The reaction is cooled to room temperature and diluted with ether. The desired product is isolated by filtration as a colorless solid (10.10 g, 85%). LCMS: 288.20 (M+H⁺).

### Step B: 4-(4-Methoxycarbonyl-2-trifluoromethyl-phenyl)-piperidine-1-carboxylic acid benzyl ester

To a solution of the product from Step A (7.10 g, 21.9 mmol) in dichloromethane (250 mL) at 0° C. is added benzylchloroformate (3.49 mL, 24.6 mmol) followed by N,N-diisopropylethylamine (9.81 mL, 56.1 mmol) and the mixture is warmed to room to temperature and stirred 16 hours. The reaction mixture is washed with 1 N hydrochloric acid, water, and dried over Na₂SO₄ filtered, and the solvent is removed in vacuo to give the desired product as an oil (9.15 g, 99%). LCMS: 422.20 (M+H⁺).

### Step C: 4-(4-Carboxy-2-trifluoromethyl-phenyl)-piperidine-1-carboxylic acid benzyl ester

To a solution of the product from step B (9.15 g, 21.7 mmol) in a mixture of methanol (150 mL) and water (50 mL) is added potassium carbonate and the mixture is stirred for 16 hours at room temperature. The solvent is removed in vacuo and the resulting aqueous residue is poured into a solution of dilute hydrochloric acid. The desired product is isolated by filtration as a colorless solid (8.75 g, 99%). LCMS: 408.20 (M+H⁺).

### Step D: 4-(4-(N-(tert-Butoxycarbonyl)-guanidinocarbonyl-2-trifluoromethyl-phenyl)-piperidine-1-carboxylic acid benzyl ester

To a solution of the product from Step C (8.75 g, 21.5 mmol) in 1-methyl-2-pyrrolidinone (90 mL) is added 2-chloro-1-methylpyridinium iodide (6.58 g, 25.8 mmol) and the resulting mixture is stirred for 90 minutes. N-butoxycarbonylguanidine (4.44 g, 27.9 mmol) is then added and the reaction is stirred for 2 hours. The mixture is then poured into a stirred solution of formic acid (6 mL) in water (500 mL) and the resulting solid is collected by filtration. The crude solids are then suspended in dilute hydrochloric acid (200 mL), stirred for 30 minutes, and the desired product is isolated by filtration as a colorless solid (11.0 g, 93%). LCMS: 549.20 (M+H⁺).

### Step E: N-(4-Piperidin-4-yl-3-trifluoromethyl-benzoyl)-(N′-tert-butoxycarbonyl)-guanidine

To a solution of the product from Step D (3.11 g, 5.67 mmol) in ethanol (75 mL) under and argon atmosphere is added 20 wt % palladium(II) hydroxide on carbon (250 mg, 0.18 mmol, 50% wet) and the reaction is stirred under a hydrogen atmosphere for 16 hours. The resulting mixture is filtered through Celite and the solvent is removed in vacuo to afford a crude foamy solid. The crude solids are redissolved in dichlormethane to and trated with 1 N hydrogen chloride in ether (7 mL) and the resulting mixture is diluted with hexane. The resulting crude solid is triturated twice with dichloromethane and twice with ether to afford the desired product as a colorless solid (2.15 g, 84%). LCMS: 415.72 (M+H⁺).

### Step F: N-{4-[1-(Piperidine-3-carbonyl)-piperidin-4-yl]-3-trifluoromethyl-benzoyl}-guanidine

To a vial containing Piperidine-1,3-dicarboxylic acid 1-tert-butyl ester (32 mg, 0.14 mmol) is added 2-(7-aza-1H-benzotriazole-1-yl)-1,1,3,3-tetramethyluronium hexafluorophosphate (38 mg, 0.1 mmol) followed by N,N-dimethylacetamide (0.5 mL). The vial is shaken at room temp for ˜15 minutes. To a separate suspension of the product from Step E (40 mg, 0.089 mmol) in N,N-dimethylacetamide (0.5 mL) is added N-methylmorpholine (20 μL, 0.18 mmol) and the resulting solution is added to the carboxylic acid containing mixture. The mixture is then shaken at room temp for 16 hours. The resulting solution is filtered through an SPE cartridge containing 500 mg of basic alumina to remove the coupling reagent byproducts and any excess acid. The reaction vial is then rinsed with 10:1 N,N-dimethylacetamide:methanol (3×500 μL) and each rinse is used to wash the alumina plug. The resulting yellow solution is evaporated in the Genevac. The residue obtained is dissolved in dichloromethane (1 mL) and 20% trifluoroacetic acid in dichloromethane (1 mL) is added. The resulting solution is shaken overnight at room temperature. The resulting mixture is evaporated and the residue is purified by HPLC to afford the desired product (23 mg, 60%). LCMS: 426.10 (M+H⁺).

The examples in the table below are synthesized according to the procedure for Example 107 using the appropriate carboxylic acid starting material.

| Example | | | m/z |
|---|---|---|---|
| No. | Structure | Name | [M + H⁺] |
| 108 | 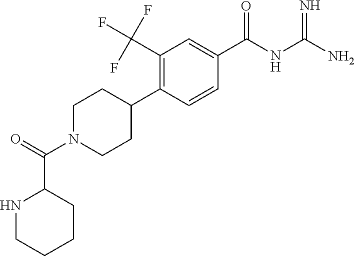 | N-{4-[1-(Piperidine-2- carbonyl)-piperidin-4- yl]-3-trifluoromethyl- benzoyl}-guanidine | 426.13 |
| 109 | 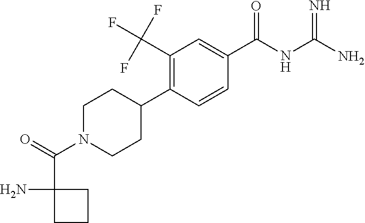 | N-{4-[1-(1-Amino- cyclobutanecarbonyl)- piperidin-4-yl]-3- trifluoromethyl- benzoyl}-guanidine | 412.1 |
| 110 | 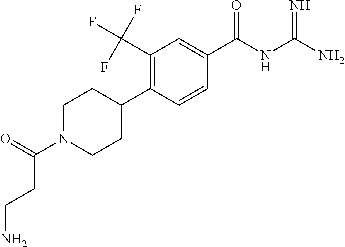 | N-{4-[1-(3-Amino- propionyl)-piperidin-4- yl]-3-trifluoromethyl- benzoyl}-guanidine | 386.12 |
| 111 | 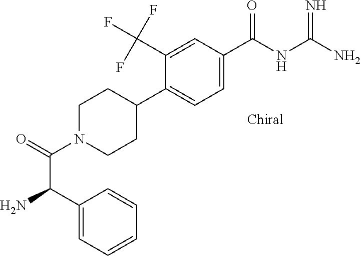 | N-{4-[1-((R)-2-Amino- 2-phenyl -acetyl)- piperidin-4-yl]-3- trifluoromethyl- benzoyl}-guanidine | 448.07 |
| 112 | 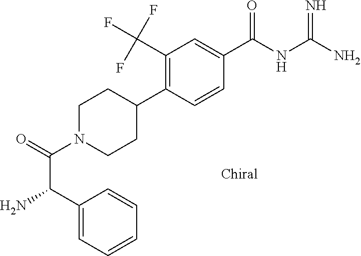 | N-{4-[1-((S)-2-Amino- 2-phenyl-acetyl)- piperidin-4-yl]-3- trifluoromethyl- benzoyl}-guanidine | 448.08 |
| 113 | 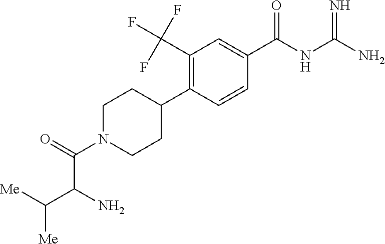 | N-{4-[1-(2-Amino-3- methyl-butyryl)- piperidin-4-yl]-3- trifluoromethyl- benzoyl}-guanidine | 414.13 |
| 114 | 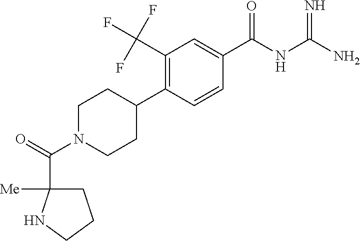 | N-{4-[1-(2-Methyl- pyrrolidine-2- carbonyl)-piperidin-4- yl]-3-trifluoromethyl- benzoyl}-guanidine | 426.1 |
| 115 | 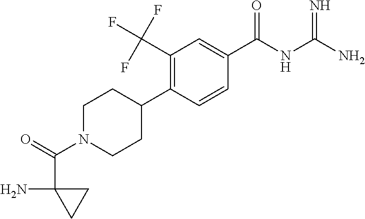 | N-{4-[1-(1-Amino- cyclopropanecarbonyl)- piperidin-4-yl]-3- trifluoromethyl- benzoyl}-guanidine | 398.09 |
| 116 | 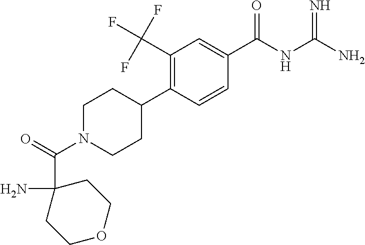 | N-{4-[1-(4-Amino- tetrahydro-pyran-4- carbonyl)-piperidin-4- yl]-3-trifluoromethyl- benzoyl}-guanidine | 442.07 |
| 117 | 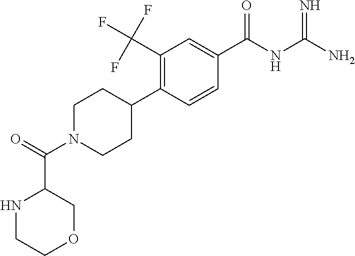 | N-{4-[1-(Morpholine- 3-carbonyl)-piperidin- 4-yl]-3-trifluoromethyl- benzoyl}-guanidine | 428.08 |
| 118 | 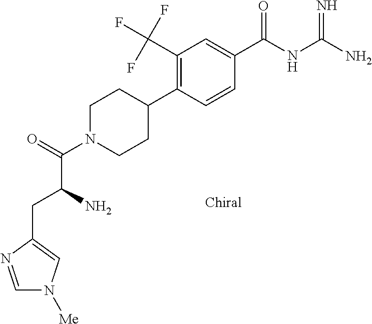 | N-(4-{1-[(S)-2-Amino- 3-(1-methyl-1H- imidazol-4-yl)- propionyl]-piperidin-4- yl}-3-trifluoromethyl- benzoyl)-guanidine | 466.11 |
| 119 | 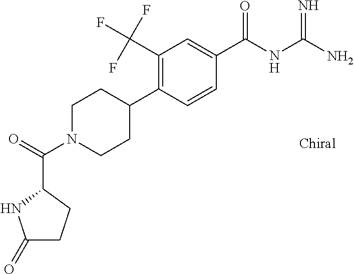 | N-{4-[1-((S)-5-Oxo- pyrrolidine-2- carbonyl)-piperidin-4- yl]-3-trifluoromethyl- benzoyl}-guanidine | 426.82 |
| 120 | 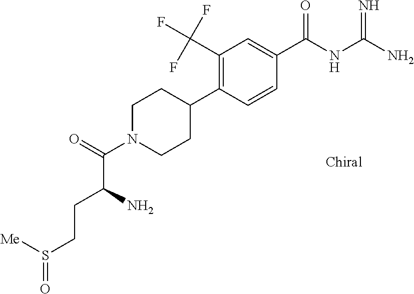 | N-{4-[1-((S)-2-Amino- 4-methanesulfinyl- butyryl)-piperidin-4- yl]-3-trifluoromethyl- benzoyl}-guanidine | 462.07 |
| 121 | 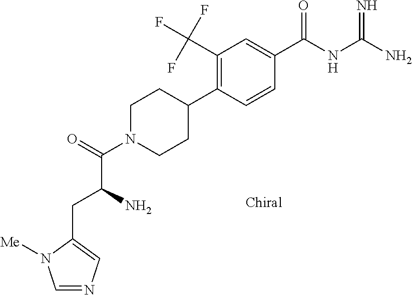 | N-(4-{1-[(S)-2-Amino- 3-(3-methyl-3H- imidazol-4-yl)- propionyl]-piperidin-4- yl}-3-trifluoromethyl- benzoyl)-guanidine | 466.12 |
| 122 | 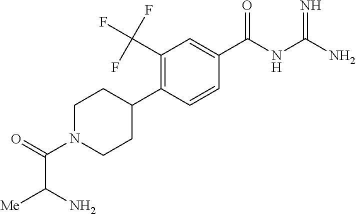 | N-{4-[1-(2-Amino- propionyl)-piperidin-4- yl]-3-trifluoromethyl- benzoyl}-guanidine | 386.11 |
| 123 | 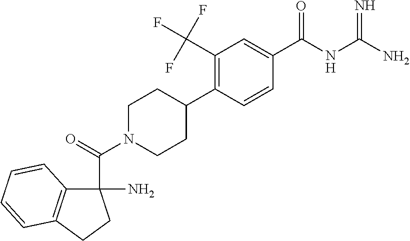 | N-{4-[1-(1-Amino- indane-1-carbonyl)- piperidin-4-yl]-3- trifluoromethyl- benzoyl)-guanidine | 474.11 |
| 124 | 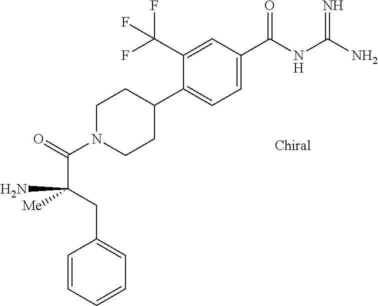 | N-{4-[1-((R)-2-Amino- 2-methyl-3-phenyl- propionyl)-piperidin-4- yl]-3-trifluoromethyl- benzoyl}-guanidine | 476.12 |
| 125 | 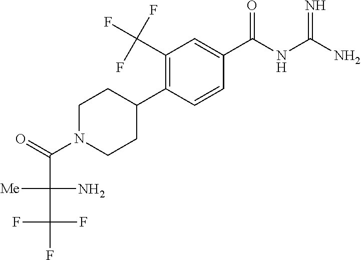 | N-{4-[1-(2-Amino- 3,3,3-trifluoro-2- methyl-propionyl)- piperidin-4-yl]-3- trifluoromethyl- benzoyl)-guanidine | 454.04 |
| 126 | 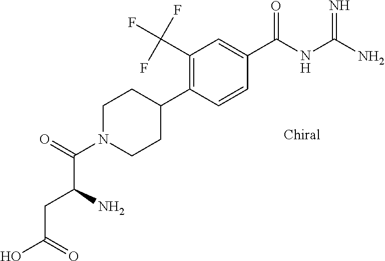 | (S)-3-Amino-4-[4-(4- guanidinocarbonyl-2- trifluoromethyl- phenyl)-piperidin-1- yl]-4-oxo-butyric acid | 430.06 |
| 127 | 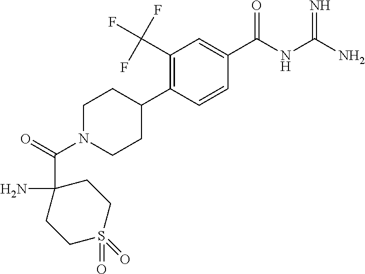 | N-{4-[1-(4-Amino-1,1- dioxo-hexahydro-γ- thiopyran-4-carbonyl)- piperidin-4-yl]-3- trifluoromethyl- benzoyl}-guanidine | 490.07 |
| 128 | 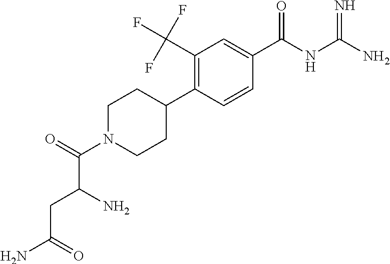 | 3-Amino-4-[4-(4- guanidinocarbonyl-2- trifluoromethyl- phenyl)-piperidin-1- yl]-4-oxo-butyramide | 429.09 |
| 129 | 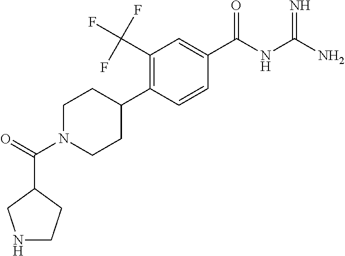 | N-{4-[1-(Pyrrolidine-3- carbonyl)-piperidin-4- yl]-3-trifluoromethyl- benzoyl}-guanidine | 412.11 |
| 130 | 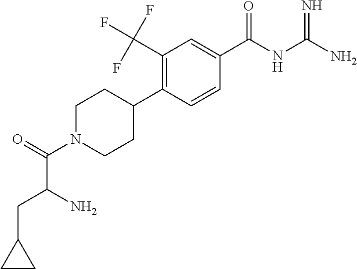 | N-{4-[1-(2-Amino-3- cyclopropyl- propionyl)-piperidin-4- yl]-3-trifluoromethyl- benzoyl}-guanidine | 426.08 |
| 131 | 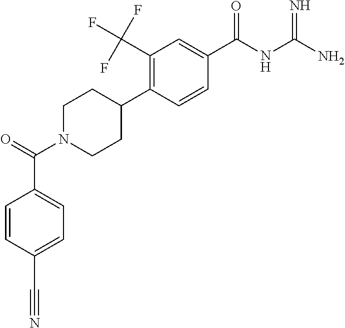 | N-(4-{1-[2-Amino-3- (4-cyano-phenyl)- propionyl]-piperidin-4- yl}-3-trifluoromethyl- benzoyl)-guanidine | 487.11 |
| 132 | 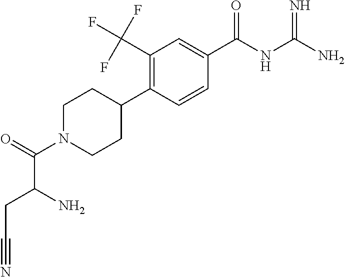 | N-{4-[1-(2-Amino-3- cyano-propionyl)- piperidin-4-yl]-3- trifluoromethyl- benzoyl}-guanidine | 411.08 |
| 133 | 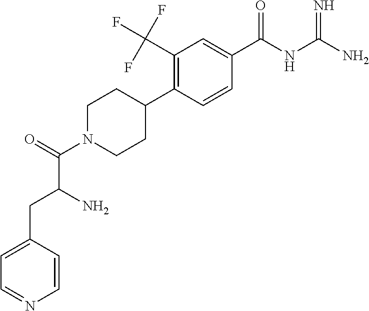 | N-{4-[1-(2-Amino-3- pyridin-4-yl- propionyl)-piperidin-4- yl]-3-trifluoromethyl- benzoyl}-guanidine | 463.13 |
| 134 | 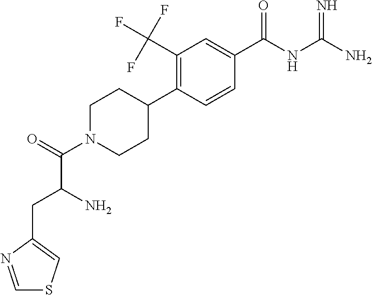 | N-{4-[1-(2-Amino-3- thiazol-4-yl-propionyl)- piperidin-4-yl]-3- trifluoromethyl- benzoyl}-guanidine | 469.01 |
| 135 | 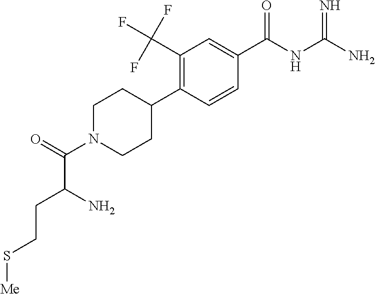 | N-{4-[1-(2-Amino-4- methylsulfanyl- butyryl)-piperidin-4- yl]-3-trifluoromethyl- benzoyl}-guanidine | 446 |
| 136 | 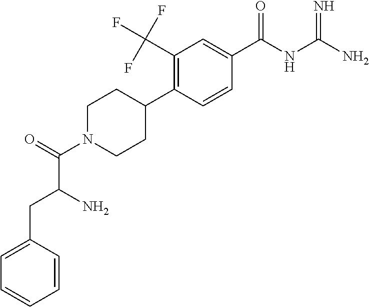 | N-{4-[1-(2-Amino-3- phenyl-propionyl)- piperidin-4-yl]-3- trifluoromethyl- benzoyl}-guanidine | 462.12 |
| 137 | 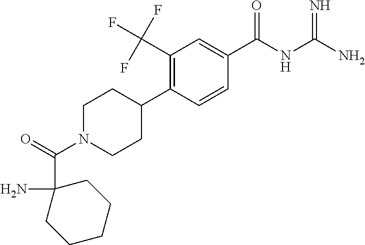 | N-{4-[1-(1-Amino- cyclohexanecarbonyl)- piperidin-4-yl]-3- trifluoromethyl- benzoyl}-guanidine | 440.13 |
| 138 | 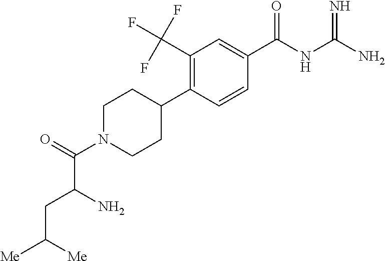 | N-{4-[1-(2-Amino-4- methyl-pentanoyl)- piperidin-4-yl]-3- trifluoromethyl- benzoyl}-guanidine | 428.13 |
| 139 | 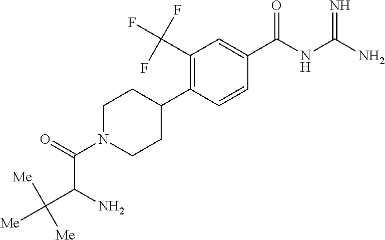 | N-{4-[1-(2-Amino-3,3- dimethyl-butyryl)- piperidin-4-yl]-3- trifluoromethyl- benzoyl)-guanidine | 428.14 |
| 140 | 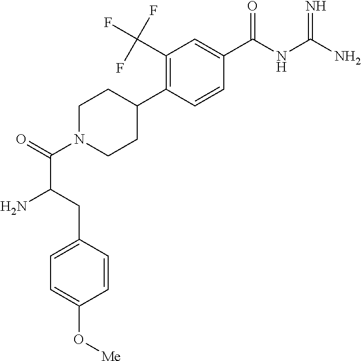 | N-(4-{1-[2-Amino-3- (4-methoxy-phenyl)- propionyl]-piperidin-4- yl}-3-trifluoromethyl- benzoyl)-guanidine | 492.09 |
| 141 | 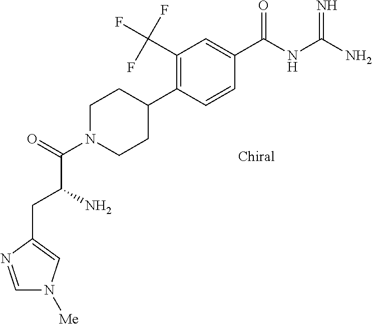 | N-(4-{1-[(R)-2-Amino- 3-(1-methyl-1H- imidazol-4-yl)- propionyl]-piperidin-4- yl}-3-trifluoromethyl- benzoyl)-guanidine | 466.09 |
| 142 | 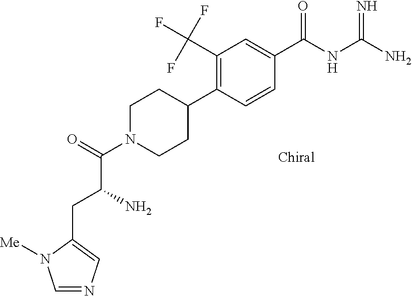 | N-(4-{1-[(R)-2-Amino- 3-(3-methyl-3H- imidazol-4-yl)- propionyl] -piperidin-4- yl}-3-trifluoromethyl- benzoyl)-guanidine | 466.12 |
| 143 | 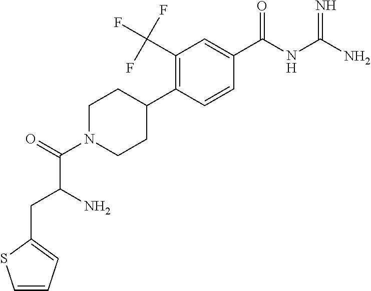 | N-{4-[1-(2-Amino-3- thiophen-2-yl- propionyl)-piperidin-4- yl]-3-trifluoromethyl- benzoyl}-guanidine | 468.03 |
| 144 | 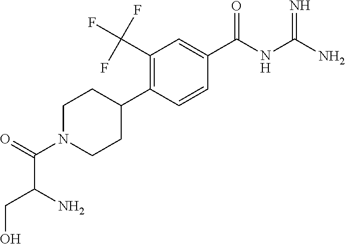 | N-{4-[1-(2-Amino-3- hydroxy-propionyl)- piperidin-4-yl]-3- trifluoromethyl- benzoyl}-guanidine | 402.1 |
| 145 | 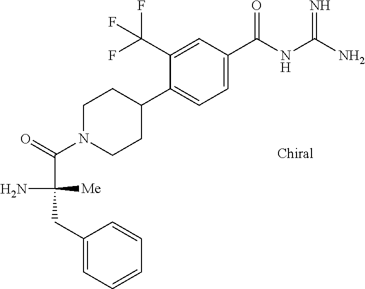 | N-{4-[1-((S)-2-Amino- 2-methyl-3-phenyl- propionyl)-piperidin-4- yl]-3-trifluoromethyl- benzoyl}-guanidine | 476.12 |
| 146 | 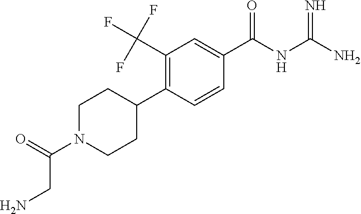 | N-{4-[1-(2-Amino- acetyl)-piperidin-4-yl]- 3-trifluoromethyl- benzoyl}-guanidine | 372.1 |
| 147 | 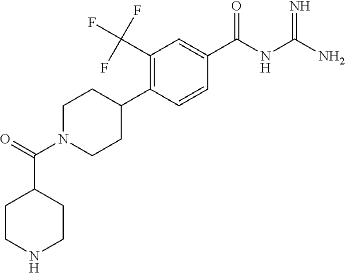 | N-{4-[1-(Piperidine-4- carbonyl)-piperidin-4- yl]-3-trifluoromethyl- benzoyl}-guanidine | 426.09 |
| 148 | 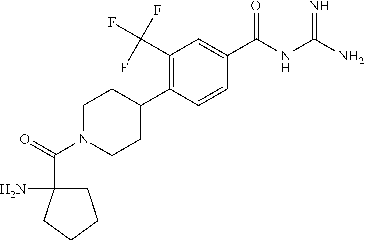 | N-{4-[1-(1-Amino- cyclopentanecarbonyl)- piperidin-4-yl]-3- trifluoromethyl- benzoyl}-guanidine | 426.08 |
| 149 | 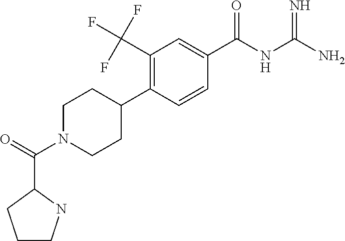 | N-{4-[1-(Pyrrolidine-2- carbonyl)-piperidin-4- yl]-3-trifluoromethyl- benzoyl}-guanidine | 412.09 |
| 150 | 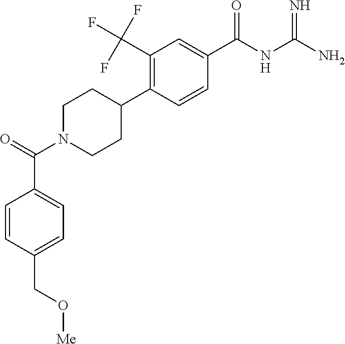 | N-{4-[1-(4- Methoxymethyl- benzoyl)-piperidin-4- yl]-3-trifluoromethyl- benzoyl}-guanidine | 462.92 |
| 151 | 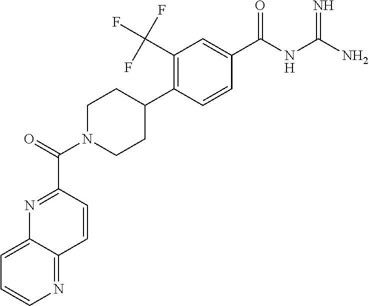 | N-{4-[1- ([1,5]Naphthyridine-2- carbonyl)-piperidin-4- yl]-3-trifluoromethyl- benzoyl}-guanidine | 471.15 |
| 152 | 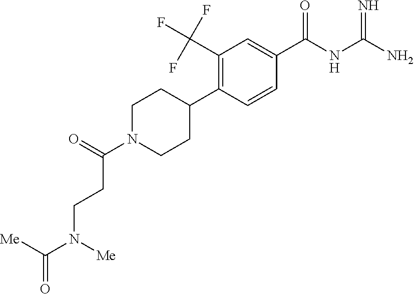 | N-{3-[4-(4- Guanidinocarbonyl-2- trifluoromethyl- phenyl)-piperidin-1- yl]-3-oxo-propyl}-N- methyl-acetamide | 442.18 |
| 153 | 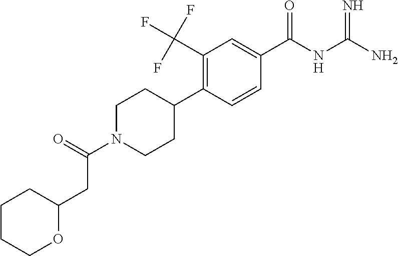 | N-{4-[1-(2-Tetrahydro- pyran-2-yl-acetyl)- piperidin-4-yl]-3- trifluoromethyl- benzoyl}-guanidine | 441.18 |
| 154 | 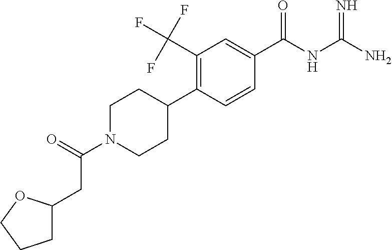 | N-{4-[1-(2-Tetrahydro- furan-2-yl-acetyl)- piperidin-4-yl]-3- trifluoromethyl- benzoyl}-guanidine | 426.96 |
| 155 | 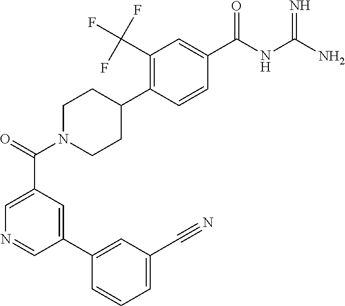 | N-(4-{1-[5-(3-Cyano- phenyl)-pyridine-3- carbonyl]-piperidin-4- yl}-3-trifluoromethyl- benzoyl)-guanidine | 521.2 |

The examples in the table below are synthesized according to the procedure of Example 107 using the appropriate carboxylic acid starting material and the hydrochloride salt of the product of Example 107 Step E.

| Example | | | m/z |
|---|---|---|---|
| No. | Structure | Name | [M + H⁺] |
| 156 | 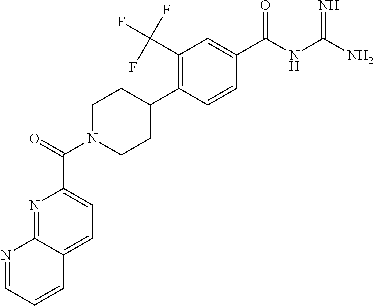 | N-{4-[1- ([1,8]Naphthyridine-2- carbonyl)-piperidin-4- yl]-3-trifluoromethyl- benzoyl}-guanidine | 471.14 |
| 157 | 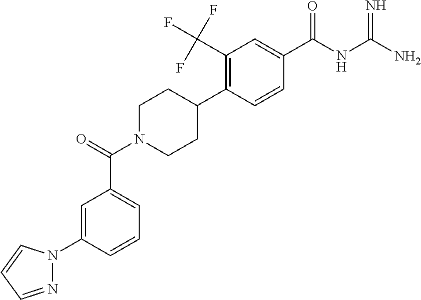 | N-{4-[1-(3-Pyrazol-1- yl-benzoyl)-piperidin- 4-yl]-3- trifluoromethyl- benzoyl}-guanidine | 484.91 |
| 158 | 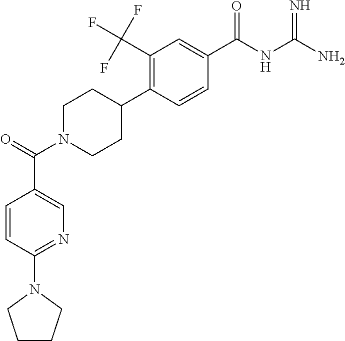 | N-{4-[1-(6-Pyrrolidin- 1-yl-pyridine-3- carbonyl)-piperidin-4- yl]-3-trifluoromethyl- benzoyl}-guanidine | 489.17 |
| 159 | 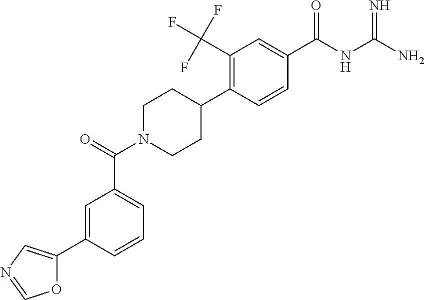 | N-{4-[1-(3-Oxazol-5- yl-benzoyl)-piperidin- 4-yl]-3- trifluoromethyl- benzoyl}-guanidine | 485.9 |
| 160 | 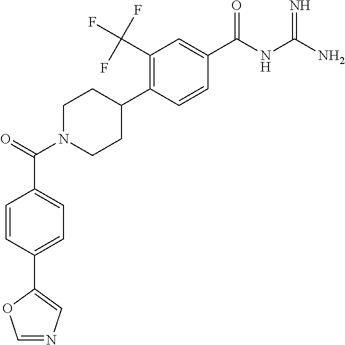 | N-{4-[1-(4-Oxazol-5- yl-benzoyl)-piperidin- 4-yl]-3- trifluoromethyl- benzoyl}-guanidine | 486.17 |
| 161 | 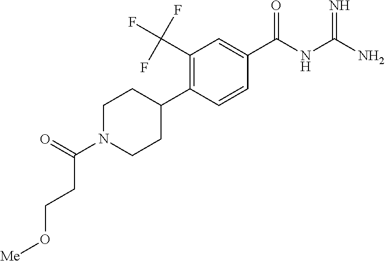 | N-{4-[1-(3-Methoxy- propionyl)-piperidin-4- yl]-3-trifluoromethyl- benzoyl}-guanidine | 401.47 |
| 162 | 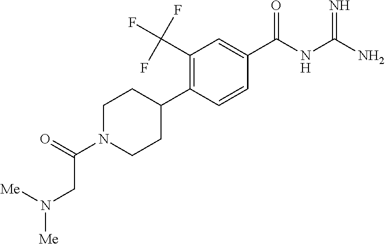 | N-{4-[1-(2- Dimethylamino- acetyl)-piperidin-4-yl]- 3-trifluoromethyl- benzoyl}-guanidine | 400.14 |
| 163 | 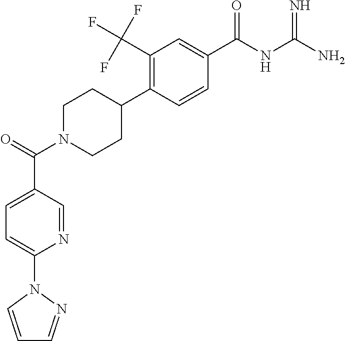 | N-{4-[1-(6-Pyrazol-1- yl-pyridine-3- carbonyl)-piperidin-4- yl]-3-trifluoromethyl- benzoyl}-guanidine | 485.85 |
| 164 | 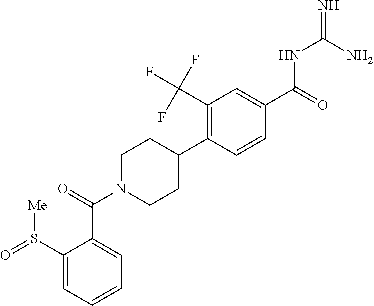 | N-{4-[1-(2- Methanesulfinyl- benzoyl)-piperidin-4- yl]-3-trifluoromethyl- benzoyl}-guanidine | 480.86 |
| 165 | 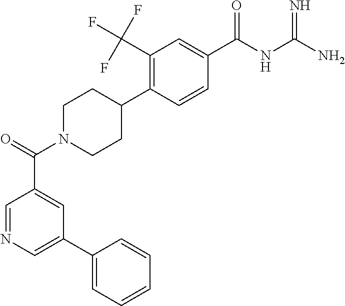 | N-{4-[1-(5-Phenyl- pyridine-3-carbonyl)- piperidin-4-yl]-3- trifluoromethyl- benzoyl}-guanidine | 496.16 |
| 166 | 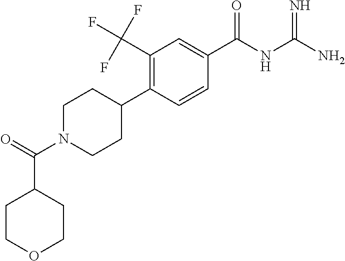 | N-{4-[1-(Tetrahydro- pyran-4-carbonyl)- piperidin-4-yl]-3- trifluoromethyl- benzoyl}-guanidine | 426.89 |
| 167 | 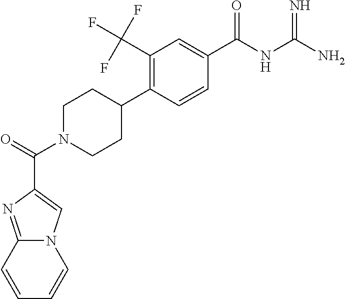 | N-{4-[1-(Imidazo[1,2- a]pyridine-2- carbonyl)-piperidin-4- yl]-3-trifluoromethyl- benzoyl}-guanidine | 459.11 |
| 168 | 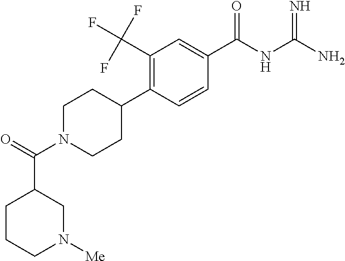 | N-{4-[1-(1-Methyl- piperidine-3-carbonyl)- piperidin-4-yl]-3- trifluoromethyl- benzoyl}-guanidine | 440.17 |
| 169 | 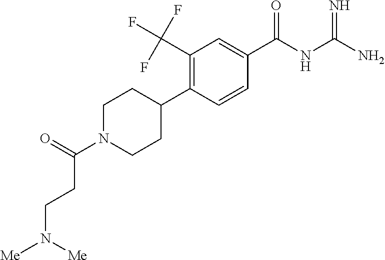 | N-{4-[1-(3- Dimethylamino- propionyl)-piperidin-4- yl]-3-trifluoromethyl- benzoyl}-guanidine | 414.16 |
| 170 | 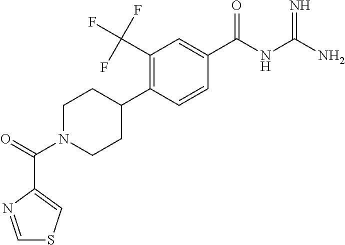 | N-{4-[1-(Thiazole-4- carbonyl)-piperidin-4- yl]-3-trifluoromethyl- benzoyl}-guanidine | 425.74 |
| 171 | 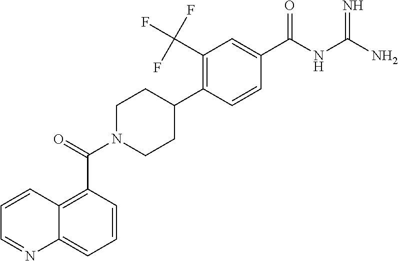 | N-{4-[1-(Quinoline-5- carbonyl)-piperidin-4- yl]-3-trifluoromethyl- benzoyl}-guanidine | 470.12 |
| 172 | 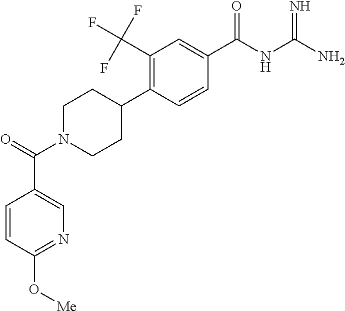 | N-{4-[1-(6-Methoxy- pyridine-3-carbonyl)- piperidin-4-yl]-3- trifluoromethyl- benzoyl}-guanidine | 449.92 |
| 173 | 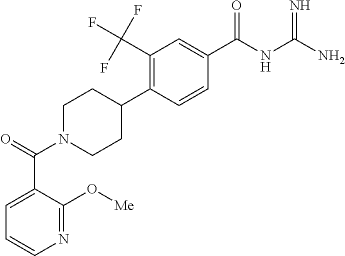 | N-{4-[1-(2-Methoxy- pyridine-3-carbonyl)- piperidin-4-yl]-3- trifluoromethyl- benzoyl}-guanidine | 449.79 |
| 174 | 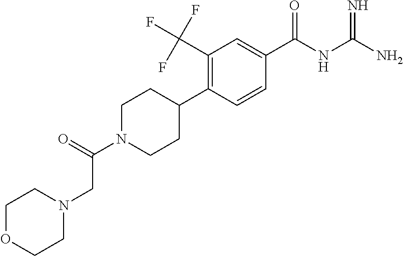 | N-{4-[1-(2-Morpholin- 4-yl-acetyl)-piperidin- 4-yl]-3- trifluoromethyl- benzoyl}-guanidine | 442.15 |
| 175 | 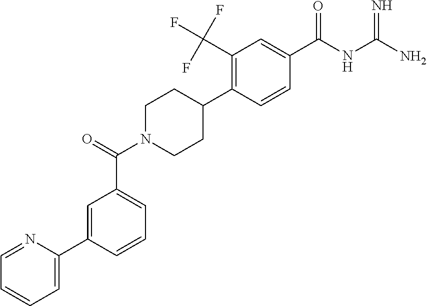 | N-{4-[1-(3-Pyridin-2- yl-benzoyl)-piperidin- 4-yl]-3- trifluoromethyl- benzoyl}-guanidine | 496.16 |
| 176 | 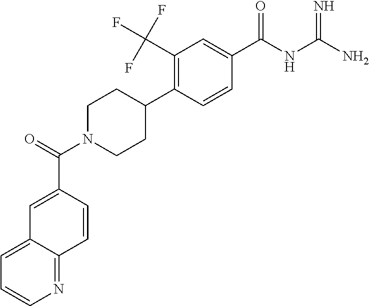 | N-{4-[1-(Quinoline-6- carbonyl)-piperidin-4- yl]-3-trifluoromethyl- benzoyl}-guanidine | 470.11 |
| 177 | 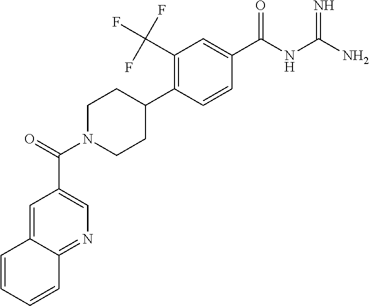 | N-{4-[1-(Quinoline-3- carbonyl)-piperidin-4- yl]-3-trifluoromethyl- benzoyl}-guanidine | 470.14 |
| 178 | 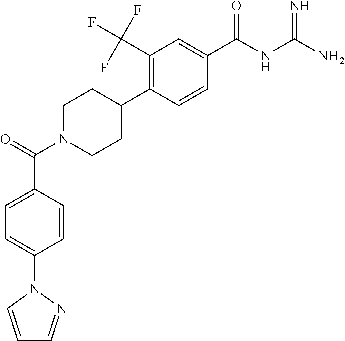 | N-{4-[1-(4-Pyrazol-1- yl-benzoyl)-piperidin- 4-yl]-3- trifluoromethyl- benzoyl}-guanidine | 484.9 |
| 179 | 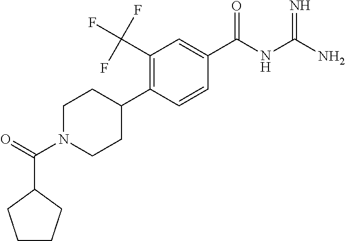 | N-[4-(1- Cyclopentanecarbonyl- piperidin-4-yl)-3- trifluoromethyl- benzoyl]-guanidine | 410.84 |
| 180 | 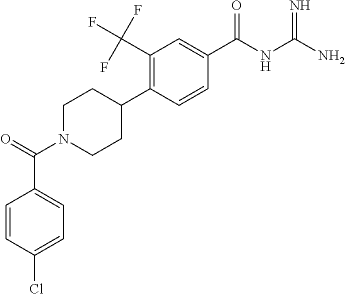 | N-{4-[1-(4-Chloro- benzoyl)-piperidin-4- yl]-3-trifluoromethyl- benzoyl}-guanidine | 455.04 |
| 181 | 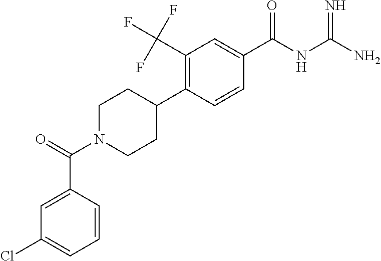 | N-{4-[1-(3-Chloro- benzoyl)-piperidin-4- yl]-3-trifluoromethyl- benzoyl}-guanidine | 455.05 |
| 182 | 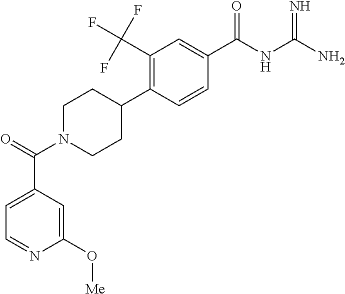 | N-{4-[1-(2-Methoxy- pyridine-4-carbonyl)- piperidin-4-yl]-3- trifluoromethyl- benzoyl}-guanidine | 450.18 |
| 183 | 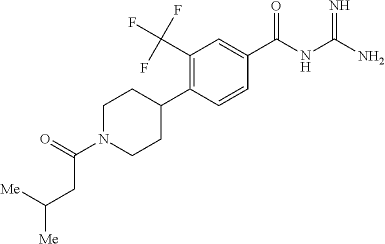 | N-{4-[1-(3-Methyl- butyryl)-piperidin-4- yl]-3-trifluoromethyl- benzoyl}-guanidine | 398.92 |
| 184 | 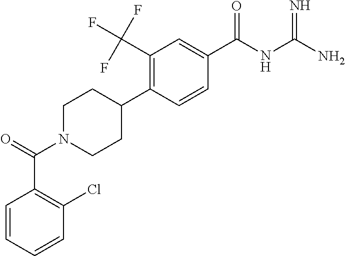 | N-{4-[1-(2-Chloro- benzoyl)-piperidin-4- yl]-3-trifluoromethyl- benzoyl}-guanidine | 455.04 |
| 185 | 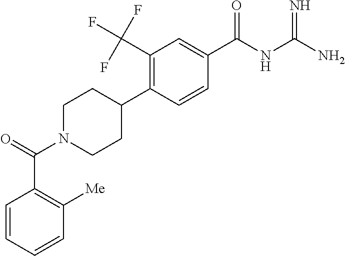 | N-{4-[1-(2-Methyl- benzoyl)-piperidin-4- yl]-3-trifluoromethyl- benzoyl}-guanidine | 432.93 |
| 186 | 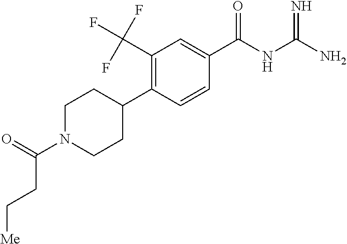 | N-[4-[1-Butyryl- piperidin-4-yl)-3 trifluoromethyl- benzoyl]-guanidine | 384.93 |
| 187 | 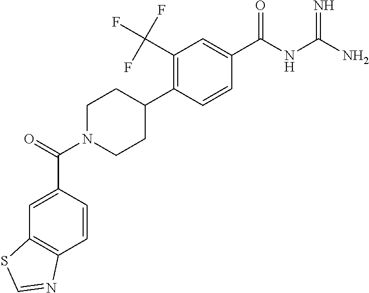 | N-{4-[1- (Benzothiazole-6- carbonyl)-piperidin-4- yl]-3-trifluoromethyl- benzoyl}-guanidine | 477.11 |
| 188 | 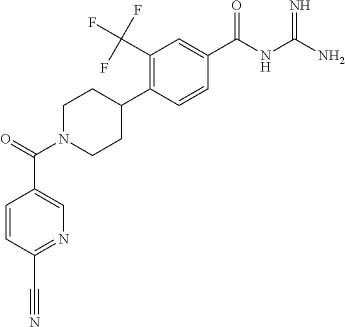 | N-{4-[1-(6-Cyano- pyridine-3-carbonyl)- piperidin-4-yl]-3- trifluoromethyl- benzoyl}-guanidine | 444.9 |
| 189 | 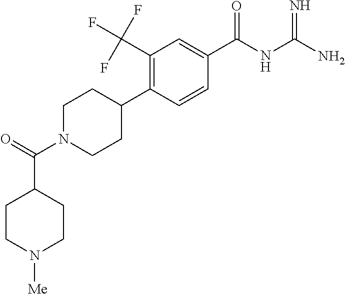 | N-{4-[1-(1-Methyl- piperidine-4-carbonyl)- piperidin-4-yl]-3- trifluoromethyl- benzoyl}-guanidine | 440.14 |
| 190 | 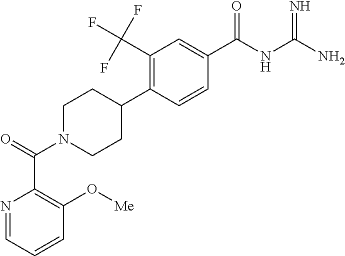 | N-{4-[1-(3-Methoxy- pyridine-2-carbonyl)- piperidin-4-yl]-3- trifluoromethyl- benzoyl}-guanidine | 450.09 |
| 191 | 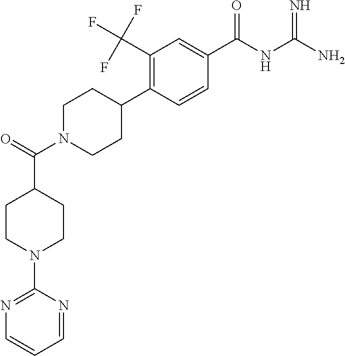 | N-{4-[1-(1-Pyrimidin- 2-yl-piperidine-4- carbonyl)-piperidin-4- yl]-3-trifluoromethyl- benzoyl}-guanidine | 504.2 |
| 192 | 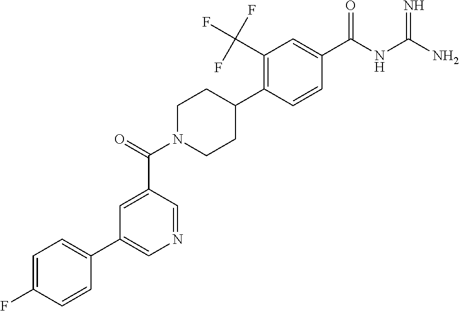 | N-(4-{1-[5-(4-Fluoro- phenyl)-pyridine-3- carbonyl]-piperidin-4- yl}-3-trifluoromethyl- benzoyl)-guanidine | 514.15 |
| 193 | 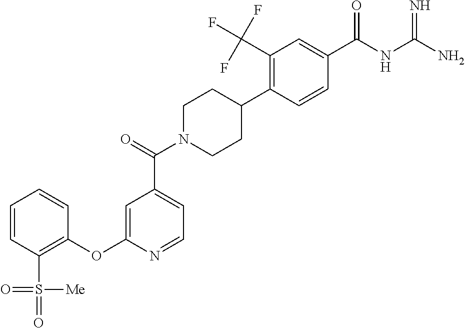 | N-(4-{1-[2-(2- Methanesulfonyl- phenoxy)-pyridine-4- carbonyl]-piperidin-4- yl}-3-trifluoromethyl- benzoyl)-guanidine | 590.21 |
| 194 | 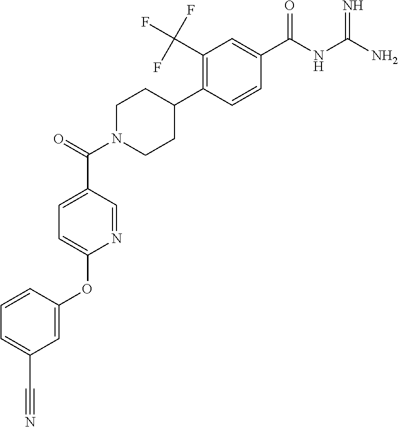 | N-(4-{1-[6-(3-Cyano- phenoxy)-pyridine-3- carbonyl]-piperidin-4- yl}-3-trifluoromethyl- benzoyl)-guanidine | 537.25 |
| 195 | 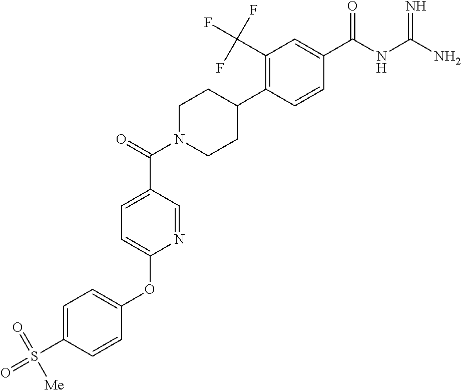 | N-(4-{1-[6-(4- Methanesulfonyl- phenoxy)-pyridine-3- carbonyl]-piperidin-4- yl}-3-trifluoromethyl- benzoyl)-guanidine | 590.2 |
| 196 | 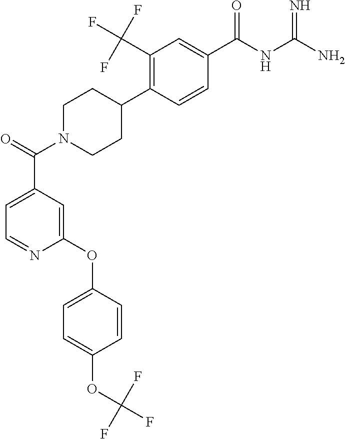 | N-(4-{1-[2-(4- Trifluoromethoxy- phenoxy)-pyridine-4- carbonyl]-piperidin-4- yl}-3-trifluoromethyl- benzoyl)-guanidine | 596.25 |
| 197 | 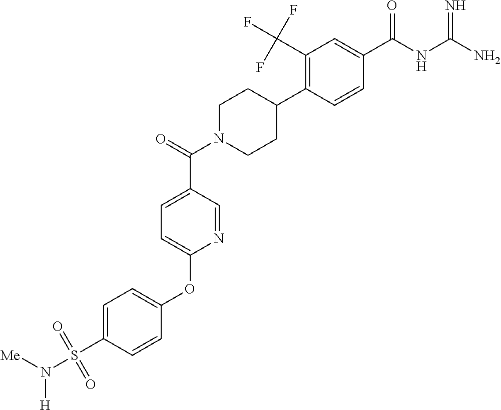 | 4-{5-[4-(4- Guanidinocarbonyl-2- trifluoromethyl- phenyl)-piperidine-1- carbonyl]-pyridin-2- yloxy}-N-methyl- benzenesulfonamide | 605.31 |
| 198 | 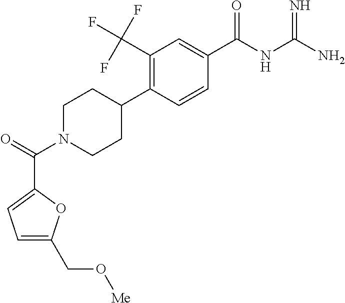 | N-{4-[1-(5- Methoxymethyl-furan- 2-carbonyl)-piperidin- 4-yl]-3- trifluoromethyl- benzoyl}-guanidine | 453.02 |
| 199 | 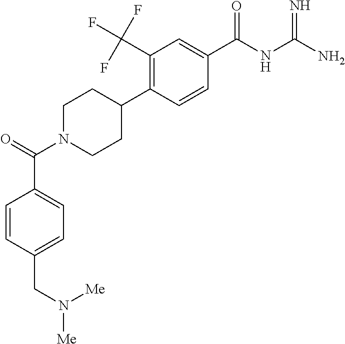 | N-{4-[1-(4- Dimethylaminomethyl- benzoyl)-piperidin-4- yl]-3-trifluoromethyl- benzoyl}-guanidine | 476.29 |
| 200 | 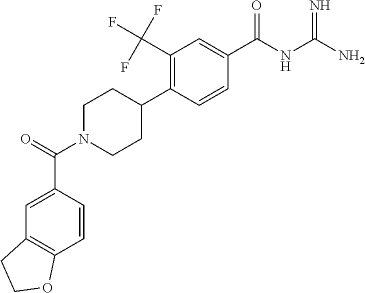 | N-{4-[1-(2,3-Dihydro- benzofuran-5- carbonyl)-piperidin-4- yl]-3-trifluoromethyl- benzoyl}-guanidine | 460.92 |
| 201 | 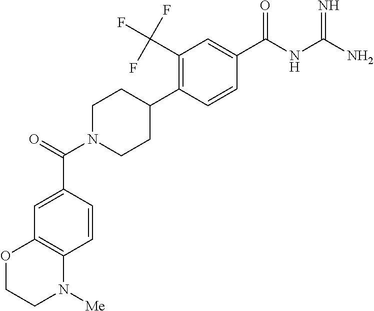 | N-{4-[1-(4-Methyl- 3,4-dihydro-2H- benzo[1,4]oxazine-7- carbonyl)-piperidin-4- yl]-3-trifluoromethyl- benzoyl}-guanidine | 491.06 |
| 202 | 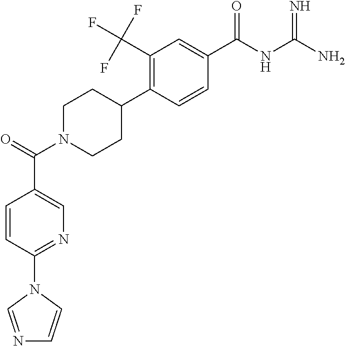 | N-{4-[1-(6-Imidazol-1- yl-pyridine-3- carbonyl)-piperidin-4- yl]-3-trifluoromethyl- benzoyl}-guanidine | 486.26 |
| 203 | 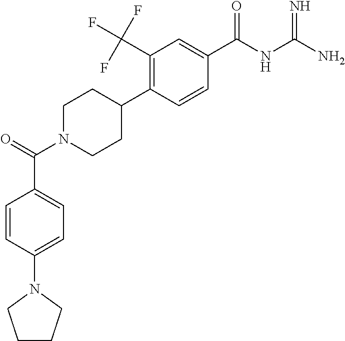 | N-{4-[1-(4-Pyrrolidin- 1-yl-benzoyl)- piperidin-4-yl]-3- trifluoromethyl- benzoyl}-guanidine | 488.39 |
| 204 | 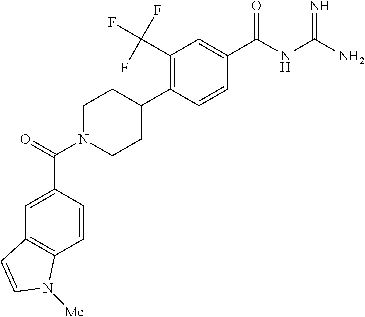 | N-{4-[1-(1-Methyl- 1H-indole-5-carbonyl)- piperidin-4-yl]-3- trifluoromethyl- benzoyl}-guanidine | 472.11 |
| 205 | 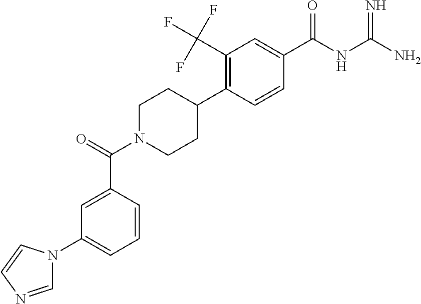 | N-{4-[1-(3-Imidazol-1- yl-benzoyl)-piperidin- 4-yl]-3- trifluoromethyl- benzoyl}-guanidine | 485.29 |
| 206 | 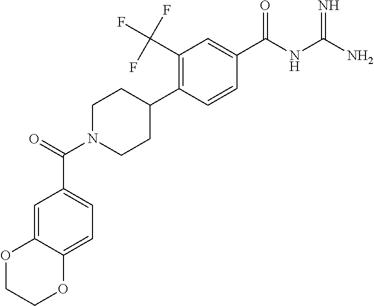 | N-{4-[1-(2,3-Dihydro- benzo[1,4]dioxine-6- carbonyl)-piperidin-4- yl]-3-trifluoromethyl- benzoyl}-guanidine | 477.07 |
| 207 | 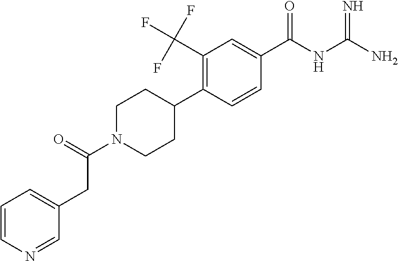 | N-{4-[1-(2-Pyridin-3- yl-acetyl)-piperidin-4- yl]-3-trifluoromethyl- benzoyl}-guanidine | 434.19 |
| 208 | 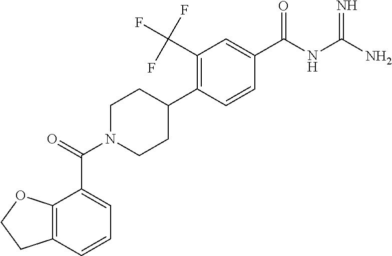 | N-{4-[1-(2,3-Dihydro- benzofuran-7- carbonyl)-piperidin-4- yl]-3-trifluoromethyl- benzoyl}-guanidine | 461.05 |
| 209 | 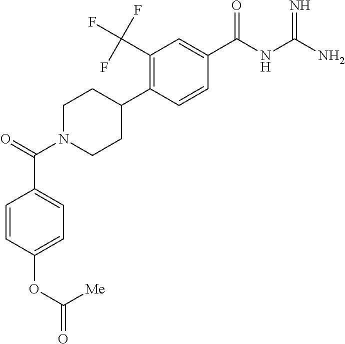 | Acetic acid 4-[4-(4- guanidinocarbonyl-2- trifluoromethyl- phenyl)-piperidine-1- carbonyl]-phenyl ester | 477.28 |
| 210 | 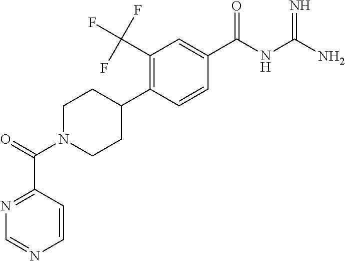 | N-{4-[1-(Pyrimidine- 4-carbonyl)-piperidin- 4-yl]-3- trifluoromethyl- benzoyl}-guanidine | 420.92 |
| 211 | 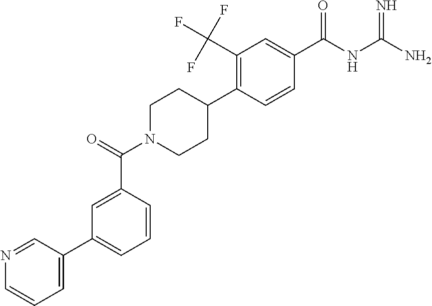 | N-{4-[1-(3-Pyridin-3- yl-benzoyl)-piperidin- 4-yl]-3- trifluoromethyl- benzoyl}-guanidine | 496.29 |
| 212 | 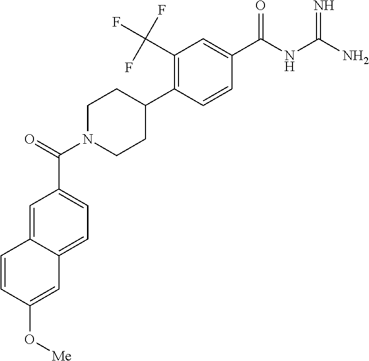 | N-{4-[1-(6-Methoxy- naphthalene-2- carbonyl)-piperidin-4- yl]-3-trifluoromethyl- benzoyl}-guanidine | 500.18 |
| 213 | 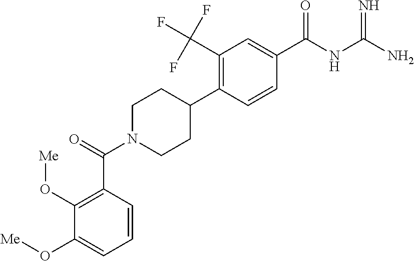 | N-{4-[1-(2,3- Dimethoxy-benzoyl)- piperidin-4-yl]-3- trifluoromethyl- benzoyl}-guanidine | 478.99 |
| 214 | 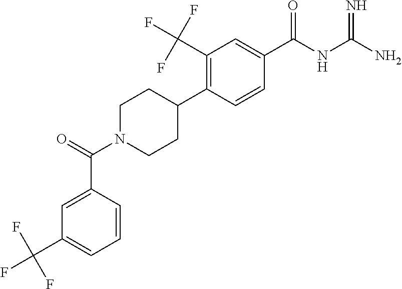 | N-{3-Trifluoromethyl- 4-[1-(3- trifluoromethyl- benzoyl)-piperidin-4- yl]-benzoyl}-guanidine | 486.92 |
| 215 | 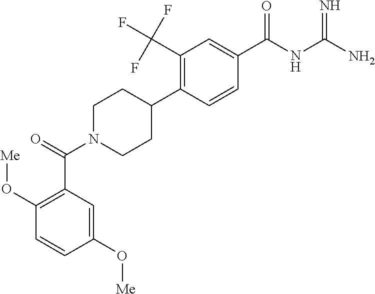 | N-{4-[1-(2,5- Dimethoxy-benzoyl)- piperidin-4-yl]-3- trifluoromethyl- benzoyl}-guanidine | 478.92 |
| 216 | 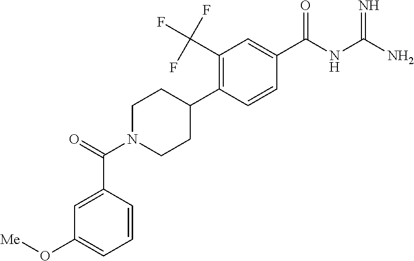 | N-{4-[1-(3-Methoxy- benzoyl)-piperidin-4- yl]-3-trifluoromethyl- benzoyl}-guanidine | 450.34 |
| 217 | 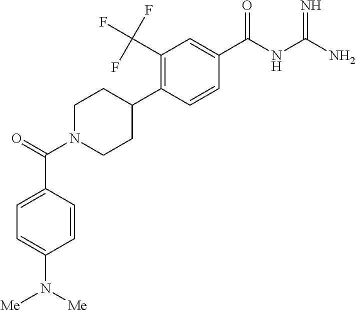 | N-{4-[1-(4- Dimethylamino- benzoyl)-piperidin-4- yl]-3-trifluoromethyl- benzoyl}-guanidine | 462.31 |
| 218 | 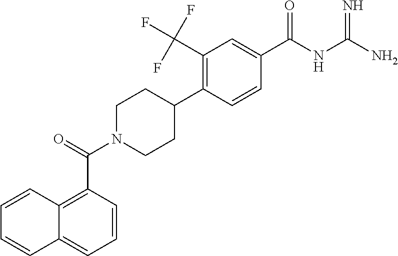 | N-{4-[1-(Naphthalene- 1-carbonyl)-piperidin- 4-yl]-3- trifluoromethyl- benzoyl}-guanidine | 468.94 |
| 219 | 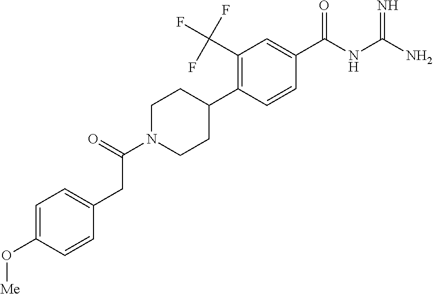 | N-(4-{1-[2-(4 Methoxy-phenyl)- acetyl]-piperidin-4- yl}-3-trifluoromethyl- benzoyl)-guanidine | 462.91 |
| 220 | 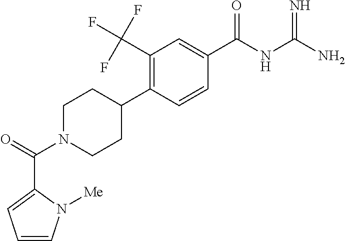 | N-{4-[1-(1-Methyl- 1H-pyrrole-2- carbonyl)-piperidin-4- yl]-3-trifluoromethyl- benzoyl}-guanidine | 421.86 |
| 221 | 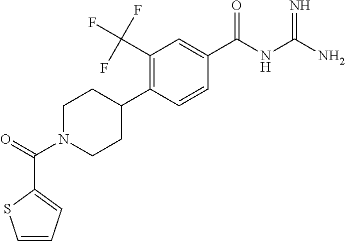 | N-{4-[1-(Thiophene-2- carbonyl)-piperidin-4- yl]-3-trifluoromethyl- benzoyl}-guanidine | 424.88 |
| 222 | 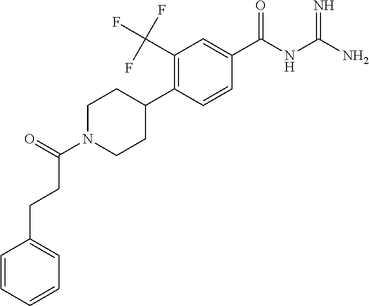 | N-{4-[1-(3-Phenyl- propionyl)-piperidin-4- yl]-3-trifluoromethyl- benzoyl}-guanidine | 446.89 |
| 223 | 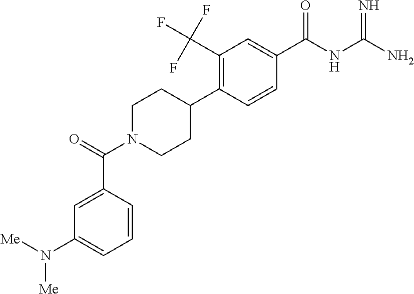 | N-{4-[1-(3- Dimethylamino- benzoyl)-piperidin-4- yl]-3-trifluoromethyl- benzoyl}-guanidine | 462.29 |
| 224 | 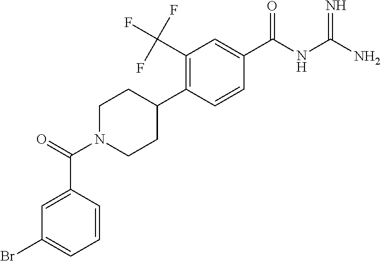 | N-{4-[1-(3-Bromo- benzoyl)-piperidin-4- yl]-3-trifluoromethyl- benzoyl}-guanidine | 497.21, 499.13 |
| 225 | 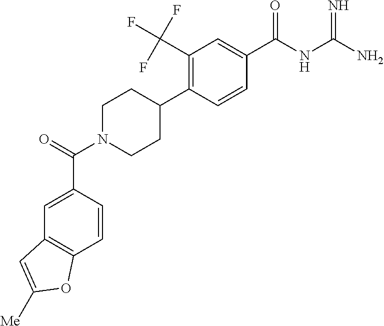 | N-{4-[1-(2-Methyl- benzofuran-5- carbonyl)-piperidin-4- yl]-3-trifluoromethyl- benzoyl}-guanidine | 474.37 |
| 226 | 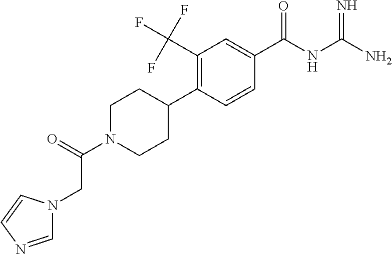 | N-{4-[1-(2-Imidazol-1- yl-acetyl)-piperidin-4- yl]-3-trifluoromethyl- benzoyl}-guanidine | 423.17 |
| 227 | 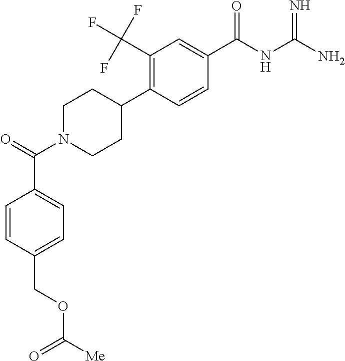 | Acetic acid 4-[4-(4- guanidinocarbonyl-2- trifluoromethyl- phenyl)-piperidine-1- carbonyl]-benzyl ester | 491.12 |
| 228 | 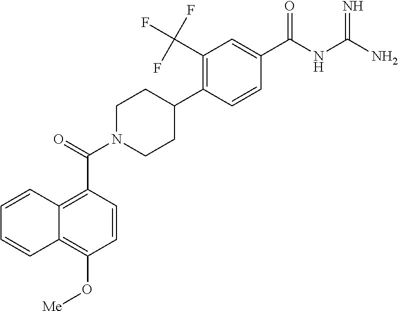 | N-{4-[1-(4-Methoxy- naphthalene-1- carbonyl)-piperidin-4- yl]-3-trifluoromethyl- benzoyl}-guanidine | 499.01 |
| 229 | 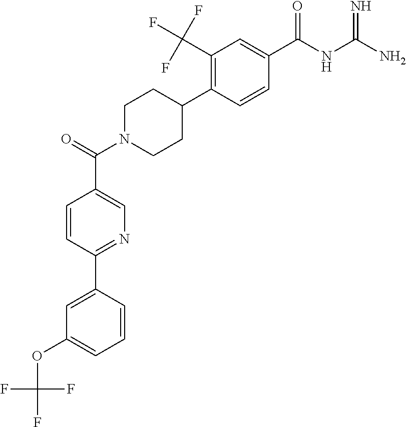 | N-(4-{1-[6-(3- Trifluoromethoxy- phenyl)-pyridine-3- carbonyl]-piperidin-4- yl}-3-trifluoromethyl- benzoyl)-guanidine | 579.91 |
| 230 | 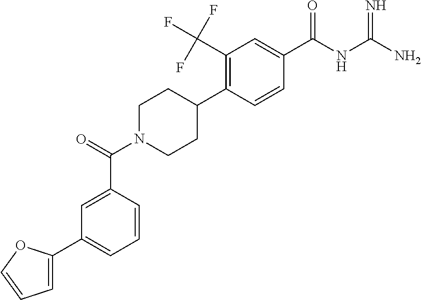 | N-{4-[1-(3-Furan-2-yl- benzoyl)-piperidin-4- yl]-3-trifluoromethyl- benzoyl}-guanidine | 484.93 |
| 231 | 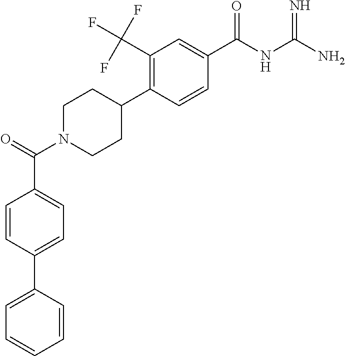 | N-{4-[1-(Biphenyl-4- carbonyl)-piperidin-4- yl]-3-trifluoromethyl- benzoyl}-guanidine | 495.01 |
| 232 | 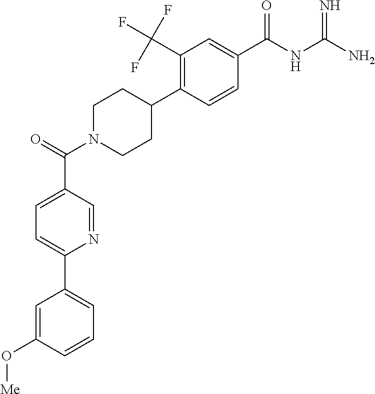 | N-(4-{1-[6-(3- Methoxy-phenyl)- pyridine-3-carbonyl]- piperidin-4-yl}-3- trifluoromethyl- benzoyl)-guanidine | 526.34 |
| 233 | 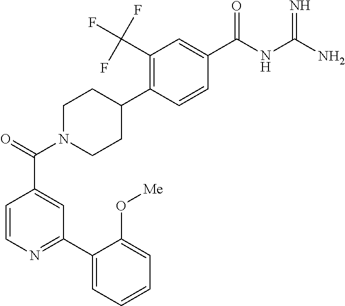 | N-(4-{1-[2-(2- Methoxy-phenyl)- pyridine-4-carbonyl]- piperidin-4-yl}-3- trifluoromethyl- benzoyl)-guanidine | 526.3 |
| 234 | 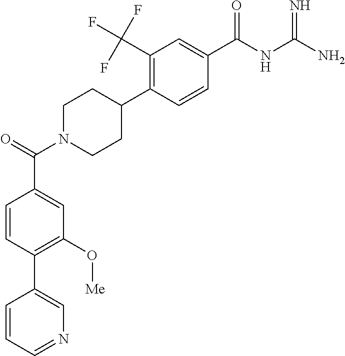 | N-{4-[1-(3-Methoxy- 4-pyridin-3-yl- benzoyl)-piperidin-4- yl]-3-trifluoromethyl- benzoyl}-guanidine | 526.3 |
| 235 | 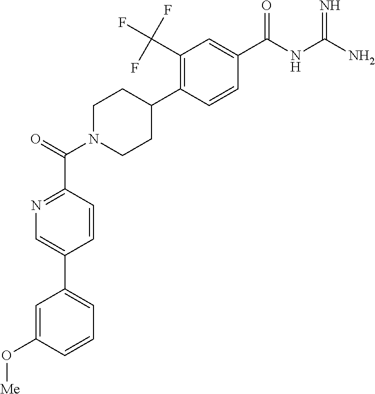 | N-(4-{1-[5-(3- Methoxy-phenyl)- pyridine-2-carbonyl]- piperidin-4-yl}-3- trifluoromethyl- benzoyl)-guanidine | 526.33 |
| 236 | 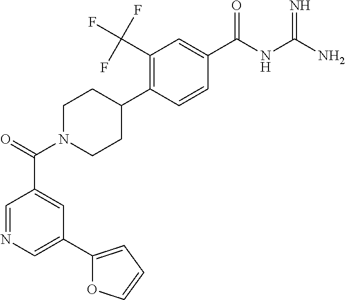 | N-{4-[1-(5-Furan-2-yl- pyridine-3-carbonyl)- piperidin-4-yl]-3- trifluoromethyl- benzoyl}-guanidine | 486.3 |

### Assessment of Biological Properties

The biological properties of the compounds of the formula I were assessed using the assays described below.

Evaluation of NHE-1 Inhibition (pHi)

HT-29 cells resuspended in DMEM supplemented with 10% FBS, 1% NEAA, and 1% Penn-Strep are seeded at 10,000 cells/well in collagen coated 384 well plates which are then incubated for 24 hours at 37° C. The next day, the medium is removed and the cells are dye (Invitrogen's BCECF) loaded for 30 minutes at 37° C., washed three times with an acid loading buffer (10 mM NH₄Cl, 1.8 mM CaCl₂, 90 mM Choline Cl, 5 mM Glucose, 15 mM Hepes, 5 mM KCl, 1 mM MgCl₂, adjusted to pH 7.5 with KOH) and further incubated at RT for 30 minutes.

At the end of the incubation, the buffer is removed and 5 uL of fresh acid loading buffer is added to each well to prevent the dessication of the cells. The plate is placed in a Hamamatsu FDSS6000 instrument and candidate compounds are added to the cells. The plates are read and the compound 1050's are calculated as measurements of the 50% inhibition of the intracellular pH recovery.

Preferred compounds will have an 1050 of <400 nM.

Evaluation of Functional Potency in Human Platelets (hPSA)

Blood (Human) was collected into 10 ml K₂ EDTA tubes (BD, #366643) at room temperature and centrifuged at 150 g for 10 minutes at room temperature and platelet rich plasma (PRP) that comprised the upper two-thirds of the plasma layer was used for the assessment of platelet swelling. The remaining plasma was further centrifuged at 1400×g to obtain platelet poor plasma (PPP). 24 μl test compounds(10×) and vehicle controls were added to 96 well plates to which 28 μl/well PRP was then added 172 μl/well of Propionate medium (PM), (sodium propionate 140 mM, HEPES 20 mM, glucose 10 mM, KCL 5 mM, MgCl₂ 1 mM and CaCl₂ 1 mM; pH6.7) was placed into 96 well plates to initiate platelet swelling. Platelet swelling was measured as a decrease in optical density at 680 mM measured ever 6 seconds over 5 mM using a microplate reader (Molecular Devices VMAX). Slope values were calculated and POC was calculated using the changes in slope from control values.

### Therapeutic Use

As can be demonstrated by the assays described above, the compounds of the invention are useful in inhibiting the NHE-1. Compounds of formula I are therefore useful in the treatment of acute responses to organ (e.g., myocardial, hepatic, cerebral) injury and are furthermore useful in the treatment of chronic post-infarct, hypertension-, and age-related responses resulting in the development of heart failure. They can be used in patients as drugs, particularly in the form of pharmaceutical compositions as set forth herein.

In another aspect of the invention, the present compounds my be useful in treating the following additional indications: acute and chronic inflammation in the lung caused by inhalation of smoke, endometriosis, Behcet's disease, uveitis and ankylosing spondylitis, pancreatitis, Lyme disease, rheumatoid arthritis, inflammatory bowel disease, septic shock, osteoarthritis, Crohn's disease, ulcerative colitis, multiple sclerosis, Guillain-Barre syndrome, psoriasis, graft versus host disease, systemic lupus erythematosus, restenosis following percutaneous transluminal coronary angioplasty, diabetes, toxic shock syndrome, Alzheimer's disease, acute and chronic pain, contact dermatitis, atherosclerosis, traumatic arthritis, glomerulonephritis, reperfusion injury, sepsis, bone resorption diseases, chronic obstructive pulmonary disease, asthma, stroke, thermal injury, adult respiratory distress syndrome (ARDS), multiple organ injury secondary to trauma, dermatoses with acute inflammatory components, acute purulent meningitis, necrotizing enterocolitis and syndromes associated with hemodialysis, leukopherisis and granulocyte transfusion.

Finally, there is an ever growing body of evidence that different fields of cancer research, from etiopathogenesis, cancer cell metabolism and neovascularization, to multiple drug resistance (MDR), selective apoptosis, modern cancer chemotherapy and the spontaneous regression of cancer (SRC) all appear to have in common a pivotal characteristic, the aberrant regulation of hydrogen ion dynamics. Thus, cancer cells have an acid-base disturbance that is completely different than observed in normal tissues and that increases in correspondence with increasing neoplastic state: an interstitial acid microenvironment linked to an intracellular alkalosis. In addition, its anti-cytokine/anti-inflammation actions stated above added to the benefit in cancer. Therefore, a list of cancer indications include: breast cancer, colon/colorectal cancer, leukemia, lung cancer, lymphoma, melanoma, and prostate cancer

Besides being useful for human treatment, these compounds are also useful for veterinary treatment of companion animals, exotic animals and farm animals, including mammals, rodents, and the like.

For treatment of the above-described diseases and conditions, a therapeutically effective dose will generally be in the range from about 0.01 mg to about 100 mg/kg of body to weight per dosage of a compound of the invention; preferably, from about 0.1 mg to about 20 mg/kg of body weight per dosage. For example, for administration to a 70 kg person, the dosage range would be from about 0.7 mg to about 7000 mg per dosage of a compound of the invention, preferably from about 7.0 mg to about 1400 mg per dosage. Some degree of routine dose optimization may be required to determine an optimal dosing level and pattern. The active ingredient may be administered from 1 to 6 times a day.

### General Administration and Pharmaceutical Compositions

When used as pharmaceuticals, the compounds of the invention are typically administered in the form of a pharmaceutical composition. Such compositions can be prepared using procedures well known in the pharmaceutical art and comprise at least one compound of the invention. The compounds of the invention may also be administered alone or in combination with adjuvants that enhance stability of the compounds of the invention, facilitate administration of pharmaceutical compositions containing them in certain embodiments, provide increased dissolution or dispersion, increased inhibitory activity, provide adjunct therapy, and the like. The compounds according to the invention may be used on their own or in conjunction with other active substances according to the invention, optionally also in conjunction with other pharmacologically active substances. In general, the compounds of this invention are administered in a therapeutically or pharmaceutically effective amount, but may be administered in lower amounts for diagnostic or other purposes.

Administration of the compounds of the invention, in pure form or in an appropriate pharmaceutical composition, can be carried out using any of the accepted modes of administration of pharmaceutical compositions. Thus, administration can be, for example, orally, buccally (e.g., sublingually), nasally, parenterally, topically, transdermally, vaginally, or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, preferably in unit dosage forms suitable for simple administration of precise dosages. The pharmaceutical compositions will generally include a conventional to pharmaceutical carrier or excipient and a compound of the invention as the/an active agent, and, in addition, may include other medicinal agents, pharmaceutical agents, carriers, adjuvants, diluents, vehicles, or combinations thereof. Such pharmaceutically acceptable excipients, carriers, or additives as well as methods of making pharmaceutical compositions for various modes or administration are well-known to those of skill in the art.

As one of skill in the art would expect, the forms of the compounds of the invention utilized in a particular pharmaceutical formulation will be selected (e.g., salts) that possess suitable physical characteristics (e.g., water solubility) that is required for the formulation to be efficacious.

1. A compound of the formula (I): 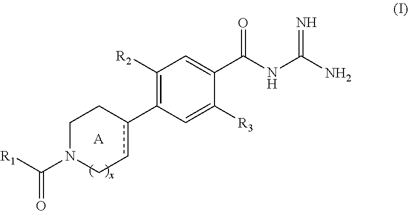
wherein:
X is 0 or 1 such that the A ring in the formula I is either a piperidinyl ring, a tetrahydropyridine ring or a pyrroldinyl ring;
R₁ is chosen from amino, C₁₋₅ alkyl, carbocycle-(CH₂)ₙ—, heterocyclyl-(CH₂)ₙ— and heteroaryl-(CH₂)ₙ— each R₁ is optionally substituted with up to three substituents independently chosen from halogen, oxo, hydroxyl, cyano, carboxy, carboxamido, C₁₋₄ alkyl, C₁₋₄ alkoxycarbonyl, C₁₋₄ alkylaminocarbonyl, C₁₋₄ dialkylaminocarbonyl, C₁₋₄ alkoxy-(CH₂)ₙ—, C₁₋₄ acyl, C₁₋₄acyloxy-(CH₂)ₙ—, C₁₋₄ alkyl-S(O)ₙ—, C₁₋₄ alkyl-S(O)ₘ—N(R₄)—, R₅—N(R₄)—S(O)ₘ—, C₃₋₇ cycloalkyl-(CH₂)ₙ—, heterocyclyl-(CH₂)ₙ—, aryl-(CH₂)ₙ-optionally substituted by C₁₋₄ alkyl, halogen, methoxy, trifluoromethoxy or cyano, heteroaryl-(CH₂)ₙ—, phenoxy optionally substituted by halogen, methoxy, C₁₋₄ alkyl-S(O)ₙ, C₁₋₄ alkyl-S(O)ₘ—N(R₄)—, cyano or trifluoromethoxy, —C(O)N(R₆)(R₇) and —(CH₂)ₙN(R₆)(R₇) each substituent on R₁ is optionally partially or fully halogenated where possible;
R₂ is chosen from halogen, hydrogen, C₁₋₅ alkyl, C₁₋₄ alkyl S(O)ₘ—N(R₄)—, C₁₋₄ alkyl-N(R₄)—S(O)ₘ— and C₁₋₄ alkyl-S(O)ₙ— each R₂ is optionally partially or fully halogenated where possible;
R₃ is chosen from hydrogen, C₁₋₅ alkyl, C₁₋₅ alkoxy, C₁₋₅ thioalkyl, C₁₋₅ acyl, C₁₋₅ alkoxycarbonyl, halogen, hydroxyl and amino optionally mono- or di-substituted by C₁₋₅ alkyl, C₁₋₅ acyl or C₃₋₇ cycloalkyl-(CH₂)ₙ—;
each R₄ and R₅ are independently chosen from hydrogen, C₁₋₅ alkyl, C₁₋₅ acyl, C₃₋₇ cycloalkyl-(CH₂)ₙ—, phenyl and benzyl, or
R₄ and R₅ taken together with the nitrogen to which they are attached form a heterocyclyl ring;
each R₆ and R₇ are independently chosen from hydrogen, hydroxyl, C₁₋₅ alkyl, C₁₋₅ acyl, C₃₋₇ cycloalkyl-(CH₂)ₙ—, phenyl and benzyl, or
R₆ and R₇ taken together with the nitrogen to which they are attached form a heterocyclyl ring;
m is 1 or 2;
n is 0-2;
or a pharmaceutically acceptable salt thereof.

2. The compound according to claim 1 and wherein
R₂ is chosen from hydrogen, C₁₋₅ alkyl and C₁₋₄ alkyl-S(O)ₙ— each R₂ is optionally partially or fully halogenated where possible;
R₃ is chosen from hydrogen, C₁₋₅ alkyl, C₁₋₅ alkoxy, halogen and hydroxyl;
each R₄ and R₅ are independently chosen from hydrogen, C₁₋₅ alkyl, C₁₋₅ acyl, C₃₋₇ cycloalkyl-(CH₂)ₙ—, phenyl and benzyl;
each R₆ and R₇ are independently chosen from hydrogen, hydroxyl, C₁₋₅ alkyl, C₁₋₅ acyl, C₃₋₇ cycloalkyl-(CH₂)ₙ—, phenyl and benzyl.

3. The compound according to claim 2 and wherein
R₁ is chosen from amino, C₁₋₅ alkyl, C₃₋₇ cycloalkyl-(CH₂)ₙ—, phenyl-(CH₂)ₙ—, indanyl-(CH₂)ₙ, naphthyl-(CH₂)ₙ, -heterocyclyl-(CH₂)ₙ— wherein the heterocyclyl is azetidinyl, tetrahydrofuranyl, pyrrolidinyl, pyrrolidinonyl, tetrahydropyranyl, piperidinyl, piperazinyl, thiomorpholinyl, 1,1-dioxo-1λ⁶-thiomorpholinyl, tetrahydrothiopyran 1,1-dioxide or morpholinyl and heteroaryl-(CH₂)ₙ— wherein the heteroaryl is pyridyl, pyrimidinyl, pyridazinyl, pyrazinyl, triazinyl, tetrazinyl, pyrrolyl, pyridinonyl, imidazolyl, oxazolyl, isoxazolyl, thiazolyl, thienyl, thiadiazolyl, pyrazolyl, furanyl, pyranyl, indolyl, indolizinyl, purinyl, quinolinyl, dihydro-2H-quinolinyl, 2-oxo-1,2,3,4-tetrahydroquinolinyl, tetrahydroquinolinyl, isoquinolinyl, quinazolinyl, indazolyl, isoindolyl, benzoxazolyl, benzothiazolyl, benzimidazolyl, benzofuranyl, benzopyranyl, 2,3-dihydro-1,4-benzodioxinyl, benzodioxolyl, 1,8-napthyridyl, 1,5-napthyridyl, 2,3-dihydrobenzofuryl, imidazo[1,2-a]pyridyl or 4-methyl-3,4-dihydro-2H-benzo[1,4]oxazinyl each R₁ is optionally substituted with up to three substituents independently chosen from halogen, hydroxyl, cyano, carboxy, carboxamido, acetoxy-(CH₂)ₙ—, C₁₋₄ alkyl, C₁₋₄ alkoxycarbonyl, C₁₋₄ alkoxy-(CH₂)ₙ—, C₁₋₄ acyl, C₁₋₄ alkyl-S(O)ₙ—, C₁₋₄ alkyl S(O)ₘ—N(R₄)—, R₅—N(R₄)—S(O)ₘ—, —C(O)N(R₆)(R₇), —(CH₂)ₙN(R₆)(R₇), pyridyl, pyrimidinyl, imidazolyl, pyrazolyl, pyrrolidinyl, oxazolyl, furyl, phenyl optionally substituted by halogen, methoxy, trifluoromethoxy or cyano and phenoxy optionally substituted by C₁₋₄ alkyl-S(O)ₙ—, C₁₋₄ alkyl S(O)ₘ—N(R₄)—, cyano or trifluoromethoxy, each substituent on R₁ is optionally partially or fully halogenated where possible;
each R₄ and R₅ are independently chosen from hydrogen, C₁₋₅ alkyl and C₃₋₇ cycloalkyl,
each R₆ and R₇ are independently chosen from hydrogen, C₁₋₅ alkyl and C₃₋₇ cycloalkyl.

4. The compound according to claim 3 and wherein
R₁ is chosen from amino, C₁₋₅ alkyl, C₃₋₇ cycloalkyl-(CH₂)ₙ—, phenyl-(CH₂)ₙ—, indanyl-(CH₂)ₙ, naphthyl-(CH₂)ₙ, -heterocyclyl-(CH₂)ₙ— wherein the heterocyclyl is tetrahydrofuranyl, pyrrolidinyl, pyrrolidinonyl, tetrahydropyranyl, piperidinyl, tetrahydrothiopyran 1,1-dioxide or morpholinyl and heteroaryl-(CH₂)ₙ— wherein the heteroaryl is pyridyl, pyrimidinyl, pyrrolyl, pyridinonyl, imidazolyl, oxazolyl, thiazolyl, thienyl, pyrazolyl, furanyl, indolyl, quinolinyl, 2-oxo-1,2,3,4-tetrahydroquinolinyl, benzothiazolyl, 2,3-dihydro-1,4-benzodioxinyl, benzodioxolyl, 1,8-napthyridyl, 1,5-napthyridyl, 2,3-dihydrobenzofuryl, imidazo[1,2-a]pyridyl or 4-methyl-3,4-dihydro-2H-benzo[1,4]oxazinyl each R₁ is optionally substituted with up to three substituents independently chosen from halogen, hydroxyl, cyano, carboxy, carboxamido, acetoxy-(CH₂)ₙ—, C₁₋₄ alkyl, C₁₋₄ alkoxycarbonyl, C₁₋₄ alkoxy-(CH₂)ₙ—, C₁₋₄ alkyl-S(O)ₙ—, R₃—N(R₄)—S(O)ₘ—, —C(O)N(R₆)(R₇), —(CH₂)₆N(R₆)(R₇), pyridyl, pyrimidinyl, imidazolyl, pyrazolyl, pyrrolidinyl, oxazolyl, furyl, phenyl optionally substituted by halogen, methoxy, trifluoromethoxy or cyano and phenoxy optionally substituted by C₁₋₄ alkyl-S(O)ₘ—, C₁₋₄ alkyl S(O)ₘ—N(R₄)—, cyano or trifluoromethoxy; each substituent on R₁ is optionally partially or fully halogenated where possible;
R₂ is chosen from halogenated C₁₋₃ alkyl and C₁₋₄ alkyl-S(O)ₘ—;
R₃ is chosen from hydrogen, C₁₋₅ alkyl and C₁₋₅ alkoxy;
each R₄, R₃, R₆, and R₇ are independently chosen from hydrogen and C₁₋₅ alkyl.

5. The compound according to claim 4 and wherein
X is 0 or 1 such that the A ring in the formula I is either a piperidinyl ring or a pyrrolidinyl ring;
R₁ is chosen from amino, C₁₋₅ alkyl, C₃₋₇ cycloalkyl-(CH₂)ₙ—, phenyl-(CH₂)ₙ—, indanyl-(CH₂)ₙ, naphthyl-(CH₂)ₙ, -heterocyclyl-(CH₂)ₙ— wherein the heterocyclyl is pyrrolidinyl, pyrrolidinonyl, tetrahydropyranyl, piperidinyl, tetrahydrothiopyran 1,1-dioxide or morpholinyl and heteroaryl-(CH₂)ₙ— wherein the heteroaryl is pyridyl, pyrimidinyl, pyrrolyl, pyridinonyl, imidazolyl, oxazolyl, thiazolyl, thienyl, pyrazolyl, furanyl, indolyl, quinolinyl, 2-oxo-1,2,3,4-tetrahydroquinolinyl, benzothiazolyl, 2,3-dihydro-1,4-benzodioxinyl, 1,8-napthyridyl, 2,3-dihydrobenzofuryl, imidazo[1,2-a]pyridyl or 4-methyl-3,4-dihydro-2H-benzol[1,4]oxazinyl each R₁ is optionally substituted with up to three substituents independently chosen from halogen, hydroxyl, cyano, carboxy, carboxamido, acetoxy, C₁₋₄ alkyl, C₁₋₄ alkoxycarbonyl, C₁₋₄ alkoxy-(CH₂)ₙ—, C₁₋₄ alkyl-S(O)ₙ—, R₅—N(R₄)—S(O)ₘ—, —C(O)N(R₆)(R₇), —N(R₆)(R₇), pyridyl, pyrimidinyl, imidazolyl, pyrazolyl, pyrrolidinyl, oxazolyl, phenyl optionally substituted by halogen to or methoxy and phenoxy optionally substituted by C₁₋₄ alkyl-S(O)ₙ— or C₁₋₄ alkyl-S(O)ₘ—N(R₄)—; each substituent on R₁ is optionally partially or fully halogenated where possible;
R₂ is trifluoromethyl or methylsulfonyl;
R₃ is chosen from hydrogen, methyl and methoxy;
each R₄, R₅, R₆, and R₇ are independently chosen from hydrogen and methyl.

6. The compound according to claim 5 and wherein
R₁ is chosen from

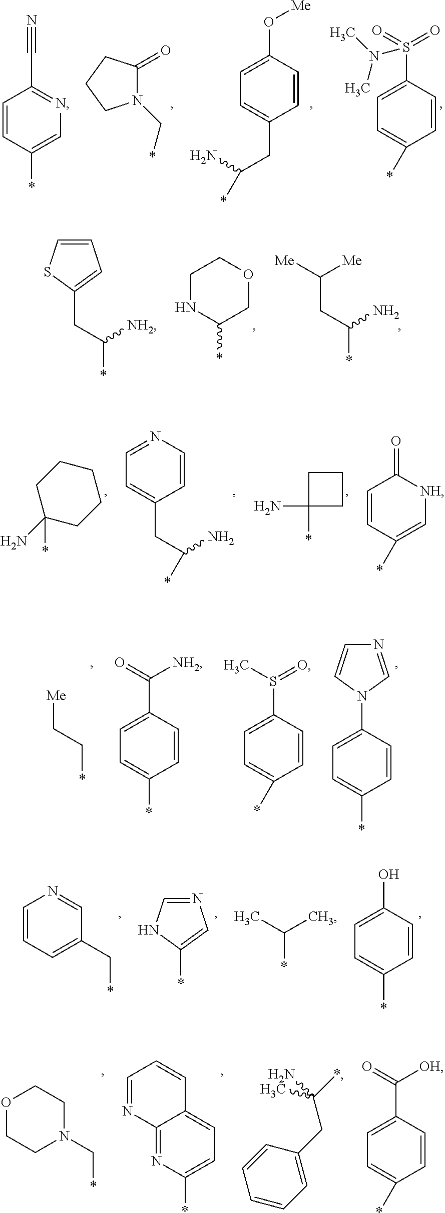

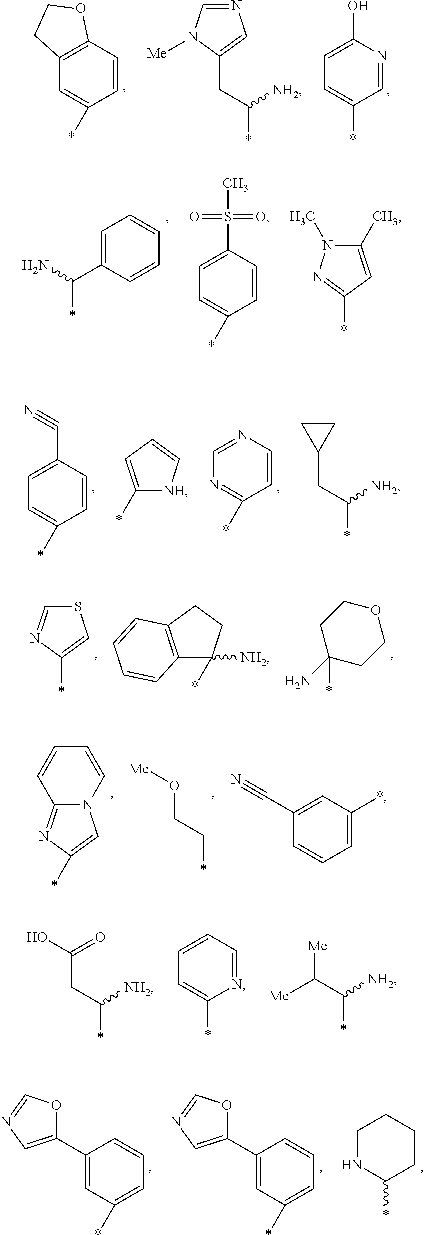

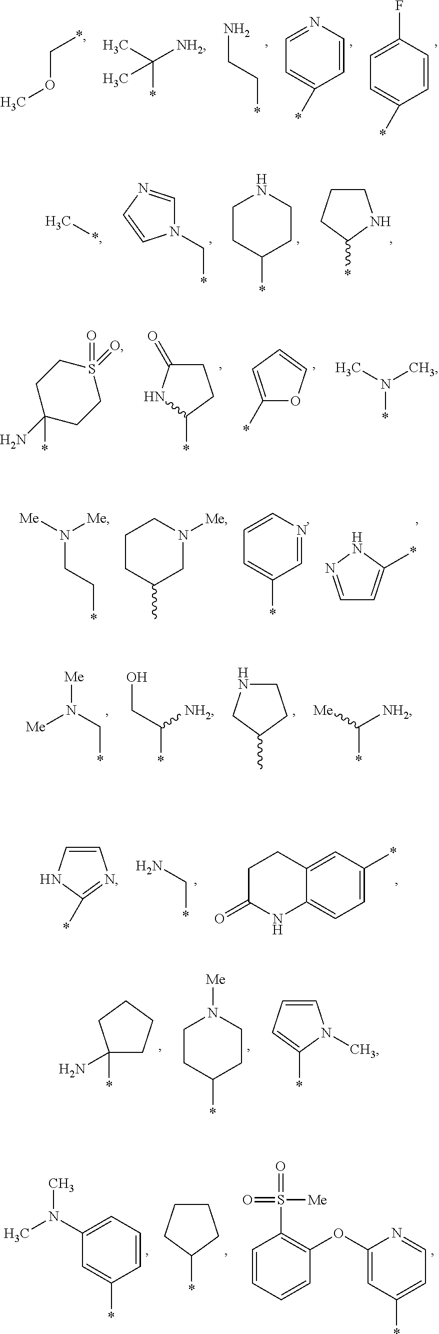

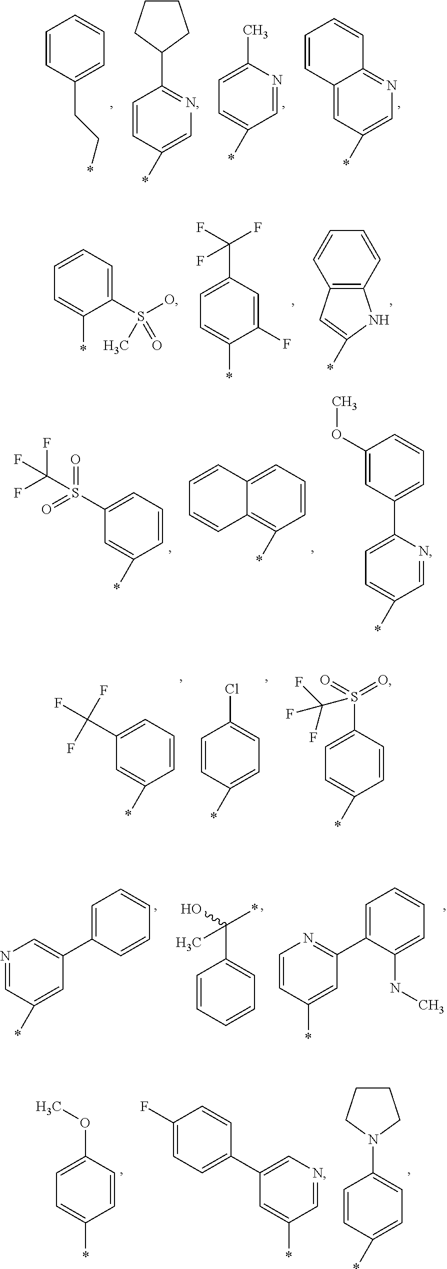

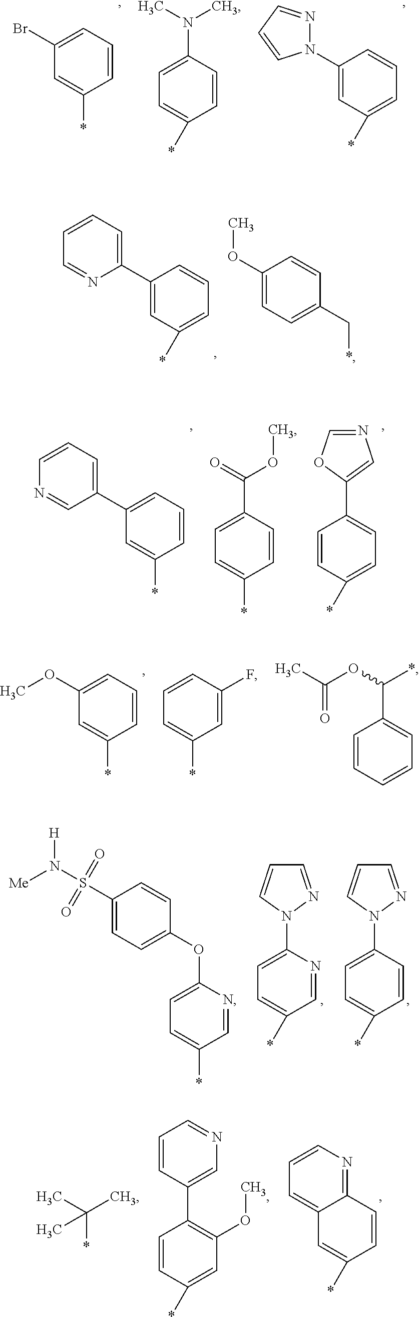

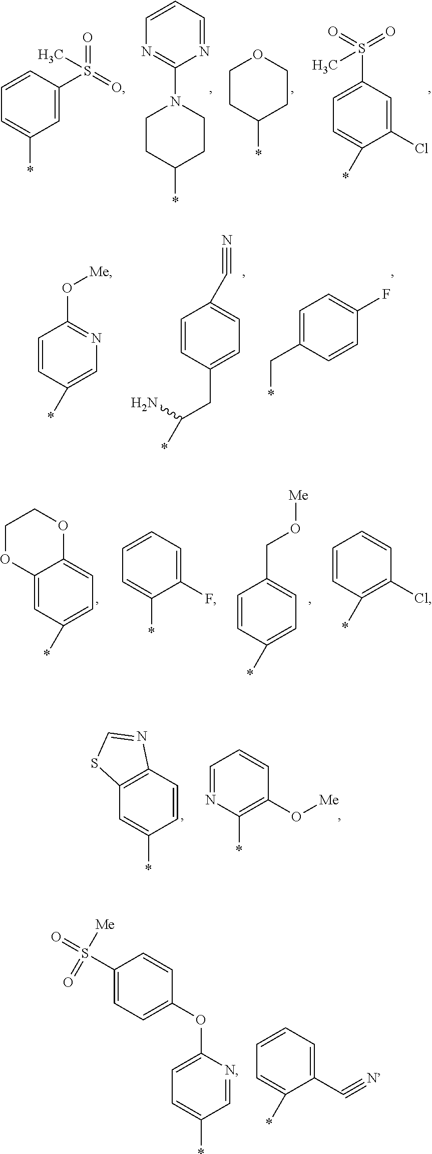

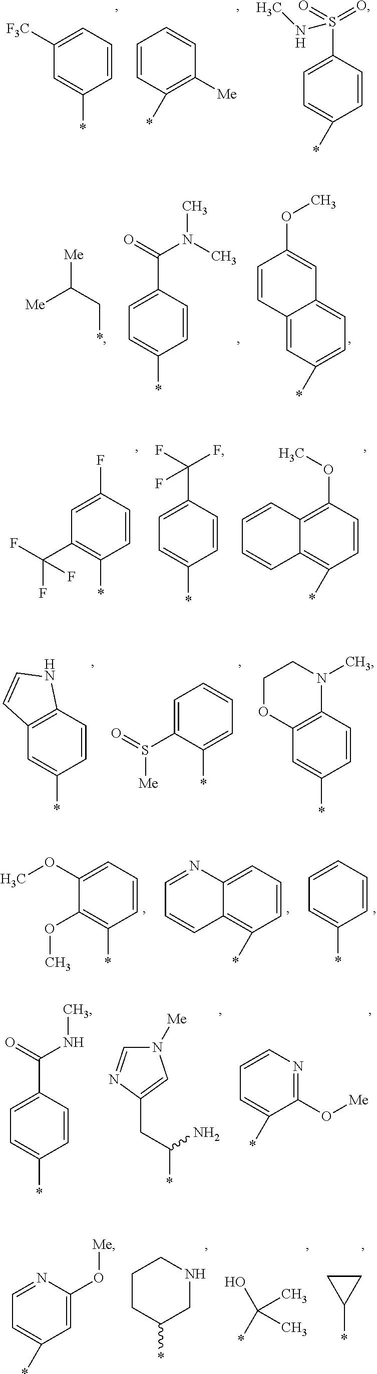

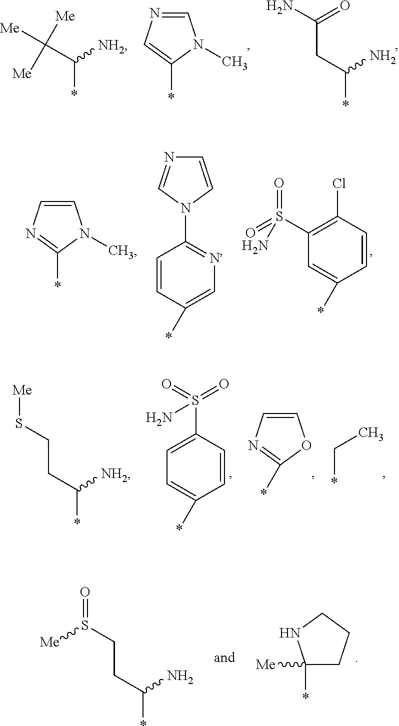

7. The compound according to claim 6 and wherein
X is 0 such that the A ring in the formula I is a pyrroldinyl ring.

8. The compound according to claim 6 and wherein
X is 1 such that the A ring in the formula I is a piperidinyl ring.

9. A pharmaceutical composition comprising a therapeutically effective amount of compound according to claim 1 and one or more pharmaceutically acceptable carriers and/or adjuvants.

10. A method of treating a disease or condition chosen from acute responses to myocardial, hepatic or cerebral injury, chronic post-infarct, hypertension and age-related responses resulting in the development of heart failure comprising administering to a mammal a therapeutically effective amount of compound according to claim 1.
